(19) United States
(12) Patent Application Publication
(10) Pub. No.: US 2025/0281631 A1
(43) Pub. Date: Sep. 11, 2025

(54) ANTIBODY-DRUG CONJUGATES AND PREPARATION METHODS AND USE THEREOF

(71) Applicant: SICHUAN KELUN-BIOTECH BIOPHARMACEUTICAL CO., LTD, Chengdu, Sichuan (CN)

(72) Inventors: Jiangjiang HU, Chengdu, Sichuan (CN); Qiang TIAN, Chengdu, Sichuan (CN); Xiaoxi YUAN, Chengdu, Sichuan (CN); Xiaobei WANG, Chengdu, Sichuan (CN); Yun BAI, Chengdu, Sichuan (CN); Lirong LI, Chengdu, Sichuan (CN); Hu LONG, Chengdu, Sichuan (CN); Yitao ZHANG, Chengdu, Sichuan (CN); Yong ZHENG, Chengdu, Sichuan (CN); Ruibin HU, Chengdu, Sichuan (CN); Yuzhi PU, Chengdu, Sichuan (CN); Xiuying HUANG, Chengdu, Sichuan (CN); Hongmei SONG, Chengdu, Sichuan (CN)

(21) Appl. No.: 18/859,078

(22) PCT Filed: Apr. 28, 2023
(86) PCT No.: PCT/CN2023/091744

§ 371 Date: Oct. 22, 2024

(30) Foreign Application Priority Data
Apr. 29, 2022 (CN) 202210467789.6
Apr. 23, 2023 (CN) 202310444522.X

(51) Int. Cl. A61K 47/68; A61P 35/00; C07K 16/28; C07K 16/32

(52) U.S. Cl. CPC A61K 47/68037; A61K 47/6849; A61K 47/6855; A61K 47/6889; A61P 35/00; C07K 16/2863; C07K 16/32

(57) ABSTRACT

The present application relates to an antibody-drug conjugate and its preparation methods and use, and specifically relates to an antibody-drug conjugate for treating HER3-positive cancers. The present application provides a fully human HER3 antibody, which has excellent binding activity to HER3-positive cells and can efficiently deliver drugs to HER3-positive cells. The present application also provides a drug-linker molecule coupled to the antibody, and the drug comprises a DNA topoisomerase inhibitor. The obtained antibody-drug conjugate has a better drug-to-antibody ratio, and has a good targeted killing effect on colon cancer, gastric cancer, breast cancer, and lung cancer (e.g., non-small cell lung cancer, specifically, lung adenocarcinoma). Therefore, the present application further provides a preparation method for the antibody-drug conjugate and application of the antibody-drug conjugate in the treatment of a HER3-positive cancer.

### CROSS REFERENCE TO RELATED APPLICATIONS

This application claims the benefit of Chinese Application No. 202310444522.X, filed Apr. 23, 2023 and Chinese Application No. 202210467789.6, filed Apr. 29, 2022 the disclosure of each of which is incorporated herein by reference in its entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “14463-050-228_SEQ_LISTING.xml”, was created on Apr. 20, 2023, and is 59,247 bytes in size.

### BACKGROUND OF THE INVENTION

### (1) Field of the Invention

The present application relates to the field of targeted therapy, in particular to antibody drug conjugates and their preparation, and methods and use of same.

### (2) Description of Related Art

Cancer is one of the leading causes of death in modern times. It is a class of diseases caused by the malignant transformation of healthy cells, which is caused by genetic changes, such as chromosomal translocations, mutations in tumor suppressor genes and growth factor receptors that lead to malignant proliferation of cells. Defective apoptosis or programmed cell death further promotes malignant transformation of cells that leads to cancer.

Human epidermal growth factor receptor 3 (also known as HER3 and ErbB3) is a receptor protein tyrosine kinase that belongs to the epidermal growth factor receptor (EGFR) subfamily of receptor protein tyrosine kinases. The subfamily also includes HER1 (also known as EGFR), HER2, and HER4. Similar to an epidermal growth factor receptor, the transmembrane receptor HER3 consists of a ligand-binding extracellular domain (ECD), a dimerization domain within the ECD, a transmembrane domain, and a carboxy-terminal phosphorylation domain. In addition to these domains, HER1, HER2, and HER4 carry an intracellular protein tyrosine kinase domain (TKD), whereas HER3 lacks this domain and is therefore unable to undergo autophosphorylation.

Ligand regulatory protein (HRG) binds to the extracellular domain of HER3 and activates receptor-mediated signaling pathways by promoting the dimerization with other members of human epidermal growth factor receptor (HER) family and the phosphorylation of its intracellular domain. Dimerization of HER3 with other HER family members expands the signaling potential of HER3, and serves not only as a means of signal diversification but also as a means of signal amplification. For example, the HER2/HER3 heterodimer induces one of the most important mitogenic signals among HER family members. HER3 is overexpressed in many types of cancers, such as breast cancer, gastrointestinal cancer, and pancreatic cancer. Interestingly, the relationship between the expression of HER2/HER3 and progression from the non-invasive stage to the invasive stage has been shown. Accordingly, agents capable of interfering with HER3-mediated signaling are desired.

The HER3 targeted indications currently under clinical research cover hematological tumors and solid tumors. The main therapy strategies focus on several directions such as monoclonal antibody, bispecific antibody, and antibody-drug conjugate (ADC). Among them, with respect to anti-HER3 antibody-drug conjugates, two are under international research, and one is at clinical research stage (Daiichi Sankyo U3-1402) for the treatment of non-small cell lung cancer (NSCLC), metastatic breast cancer (MBC), and colorectal cancer (CRC).

An ADC drug is composed of an antibody, a bioactive molecule and a linker. The bioactive molecule is covalently conjugated to the antibody through the linker; the antibody (e.g., monoclonal antibody) can specifically recognize a specific target on the surface of tumor cells, so as to guide the ADC to the surface of cancer cells, and enable the ADC to enter the cancer cells through endocytosis effect; the bioactive molecule is then released in the cancer cells to kill the cancer cells without damaging the normal tissue cells as much as possible.

U3-1402 was developed by Daiichi Sankyo, using GGFG tetrapeptide as an enzyme-cleavable linker, and the cytotoxin is Dxd. According to the reports, in the treatment of NSCLC with EGFR mutations, the phase I with dose expansion (5.6 mg/kg, Q3W) showed an objective response rate (ORR) of 39%, a disease control rate (DCR) of 72%, a median progression-free survival (mPFS) of 8.2 months, similar effect on patients with brain metastases was shown; in terms of safety, adverse events mainly involved hematological toxicity (thrombocytopenia, neutropenia, etc.) and interstitial pneumonia (5-7%).

### BRIEF SUMMARY OF THE INVENTION

In one aspect, the present application provides an antibody-drug conjugate, which has a structure as shown in formula Ab-[M-L-E-D]ₓ, wherein:
Ab is an antibody or antigen-binding fragment thereof that specifically binds to human epidermal growth factor receptor 3 (HER3, also known as Erbb3);
M is a joint site to bind the antibody or antigen-binding fragment thereof;
L is a connector that connects the joint site M and E;
E is a fragment connecting L and D;
D is a fragment of a cytotoxic drug;
x is selected from 1 to 10.

In some embodiments, the antibody or antigen-binding fragment thereof comprises:
(1) a heavy chain variable region (VH) and/or a light chain variable region (VL) as follows, wherein the CDRs are defined by the Chothia numbering system:
(1a) a heavy chain variable region (VH) comprising the following 3 CDRs: CDR-H1 having a sequence as set forth in SEQ ID NO: 1 or a variant thereof, CDR-H2 having a sequence as set forth in SEQ ID NO: 2 or a variant thereof, CDR-H3 having a sequence as set forth in SEQ ID NO: 3 or a variant thereof; and/or, a light chain variable region (VL) comprising the following 3 CDRs: CDR-L1 having a sequence as set forth in SEQ ID NO: 4 or a variant thereof, CDR-L2 having a sequence as set forth in SEQ ID NO: 5 or a variant thereof, CDR-L3 having a sequence as set forth in SEQ ID NO: 6 or a variant thereof; or,
(1b) a heavy chain variable region (VH) comprising the following 3 CDRs: CDR-H1 having a sequence as set forth in SEQ ID NO: 19 or a variant thereof, CDR-H2 having a sequence as set forth in SEQ ID NO: 20 or a variant thereof, CDR-H3 having a sequence as set forth in SEQ ID NO: 21 or a variant thereof; and/or, a light chain variable region (VL) comprising the following 3 CDRs: CDR-L1 having a sequence as set forth in SEQ ID NO: 22 or a variant thereof, CDR-L2 having a sequence as set forth in SEQ ID NO: 23 or a variant thereof, CDR-L3 having a sequence as set forth in SEQ ID NO: 24 or a variant thereof; or,
(1c) a heavy chain variable region (VH) comprising the following 3 CDRs: CDR-H1 having a sequence as set forth in SEQ ID NO: 36 or a variant thereof, CDR-H2 having a sequence as set forth in SEQ ID NO: 37 or a variant thereof, CDR-H3 having a sequence as set forth in SEQ ID NO: 38 or a variant thereof; and/or, a light chain variable region (VL) comprising the following 3 CDRs: CDR-L1 having a sequence as set forth in SEQ ID NO: 45 or a variant thereof, CDR-L2 having a sequence as set forth in SEQ ID NO: 23 or a variant thereof, and CDR-L3 having a sequence as set forth in SEQ ID NO: 52 or a variant thereof;
wherein, the variant described in any item of (1a), (1b), and (1c) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2 or 3 amino acids) as compared to the sequence from which it is derived; preferably, the substitution is a conservative substitution with the proviso that the amino acid sequences of the CDRs of the variant have 100% sequence identity to the amino acid sequences of the respective CDRs of the VH and VL of (1a), (1b), and (1c) and the variant binds HER3;
or,
(2) a heavy chain variable region (VH) and/or a light chain variable region (VL) as follows, wherein the CDRs are defined according to the Kabat numbering system:
(2a) a heavy chain variable region (VH) comprising the following 3 CDRs: CDR-H1 having a sequence as set forth in SEQ ID NO: 7 or a variant thereof, CDR-H2 having a sequence as set forth in SEQ ID NO: 8 or a variant thereof, CDR-H3 having a sequence as set forth in SEQ ID NO: 9 or a variant thereof; and/or, a light chain variable region (VL) comprising the following 3 CDRs: CDR-L1 having a sequence as set forth in SEQ ID NO: 4 or a variant thereof, CDR-L2 having a sequence as set forth in SEQ ID NO: 5 or a variant thereof, CDR-L3 having a sequence as set forth in SEQ ID NO: 6 or a variant thereof; or,
(2b) a heavy chain variable region (VH) comprising the following 3 CDRs: CDR-H1 having a sequence as set forth in SEQ ID NO: 25 or a variant thereof, CDR-H2 having a sequence as set forth in SEQ ID NO: 26 or a variant thereof, CDR-H3 having a sequence as set forth in SEQ ID NO: 21 or a variant thereof; and/or, a light chain variable region (VL) comprising the following 3 CDRs: CDR-L1 having a sequence as set forth in SEQ ID NO: 22 or a variant thereof, CDR-L2 having a sequence as set forth in SEQ ID NO: 23 or a variant thereof, CDR-L3 having a sequence as set forth in SEQ ID NO: 24 or a variant thereof; or,
(2c) a heavy chain variable region (VH) comprising the following 3 CDRs: CDR-H1 having a sequence as set forth in SEQ ID NO: 39 or a variant thereof, CDR-H2 having a sequence as set forth in SEQ ID NO: 40 or a variant thereof, CDR-H3 having a sequence as set forth in SEQ ID NO: 38 or a variant thereof; and/or, a light chain variable region (VL) comprising the following 3 CDRs: CDR-L1 having a sequence as set forth in SEQ ID NO: 45 or a variant thereof, CDR-L2 having a sequence as set forth in SEQ ID NO: 23 or a variant thereof, and CDR-L3 having a sequence as set forth in SEQ ID NO: 52 or a variant thereof;
wherein, the variant described in any item of (2a), (2b), and (2c) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2 or 3 amino acids) as compared to the sequence from which it is derived; preferably, the substitution is a conservative substitution with the proviso that the amino acid sequences of the CDRs of the variant have 100% sequence identity to the amino acid sequences of the respective CDRs of the VH and VL of (2a), (2b), and (2c) and the variant binds HER3;
or,
(3) a heavy chain variable region (VH) and/or a light chain variable region (VL) as follows, wherein the CDRs are defined by the IMGT numbering system:
(3a) a heavy chain variable region (VH) comprising the following 3 CDRs: CDR-H1 having a sequence as set forth in SEQ ID NO: 10 or a variant thereof, CDR-H2 having a sequence as set forth in SEQ ID NO: 11 or a variant thereof, CDR-H3 having a sequence as set forth in SEQ ID NO: 12 or a variant thereof; and/or, a light chain variable region (VL) comprising the following 3 CDRs: CDR-L1 having a sequence as set forth in SEQ ID NO: 13 or a variant thereof, CDR-L2 having a sequence as set forth in SEQ ID NO: 14 or a variant thereof, CDR-L3 having a sequence as set forth in SEQ ID NO: 6 or a variant thereof; or,
(3b) a heavy chain variable region (VH) comprising the following 3 CDRs: CDR-H1 having a sequence as set forth in SEQ ID NO: 27 or a variant thereof, CDR-H2 having a sequence as set forth in SEQ ID NO: 28 or a variant thereof, CDR-H3 having a sequence as set forth in SEQ ID NO: 29 or a variant thereof; and/or, a light chain variable region (VL) comprising the following 3 CDRs: CDR-L1 having a sequence as set forth in SEQ ID NO: 30 or a variant thereof, CDR-L2 having a sequence as set forth in SEQ ID NO: 31 or a variant thereof, CDR-L3 having a sequence as set forth in SEQ ID NO: 24 or a variant thereof; or,
(3c) a heavy chain variable region (VH) comprising the following 3 CDRs: CDR-H1 having a sequence as set forth in SEQ ID NO: 41 or a variant thereof, CDR-H2 having a sequence as set forth in SEQ ID NO: 42 or a variant thereof, CDR-H3 having a sequence as set forth in SEQ ID NO: 43 or a variant thereof; and/or, a light chain variable region (VL) comprising the following 3 CDRs: CDR-L1 having a sequence as set forth in SEQ ID NO: 44 or a variant thereof, CDR-L2 having a sequence as set forth in SEQ ID NO: 31 or a variant thereof, and CDR-L3 having a sequence as set forth in SEQ ID NO: 52 or a variant thereof;
wherein, the variant described in any item of (3a), (3b), and (3c) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2 or 3 amino acids) as compared to the sequence from which it is derived; preferably, the substitution is a conservative substitution with the proviso that the amino acid sequences of the CDRs of the variant have 100% sequence identity to the amino acid sequences of the respective CDRs of the VH and VL of (3a), (3b), and (3c) and the variant binds HER3.

In the antibody-drug conjugate, the cytotoxic drug can be conjugated to the antibody or antigen-binding fragment through a linker (e.g., the “M-L-E” fragment of the present application).

In some embodiments, M is

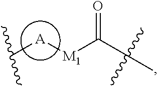

wherein Ring A is a 5- to 6-membered heteroaliphatic ring, or a 5- to 20-membered aromatic ring system, and the heteroaliphatic ring and aromatic ring system are optionally substituted with one or more groups selected from the group consisting of oxy (═O), halogen, cyano, amino, carboxyl, sulfhydryl group and C₁₋₆ alkyl; M₁ is selected from the group consisting of single bond, and C₁₋₂₀ alkylene, C₂₋₂₀ alkenylene or C₂₋₂₀ alkynylene;

L is selected from the following: C₁₋₆ alkylene, —N(R′)—, carbonyl, —O—, Val, Cit, Phe, Lys, Lys (COCH₂CH₂ (OCH₂CH₂)ₛOCH₃), D-Val, Leu, Gly, Ala, Asn, Val-Cit, Val-Ala, Val-Lys, Val-Lys (Ac), Phe-Lys, Phe-Lys (Ac), D-Val-Leu-Lys, Gly-Gly-Arg, Ala-Ala-Asn, Ala-Ala-Ala, Val-Lys-Ala, Val-Lys-Gly, Gly-Gly-Gly, Gly-Gly-Phe-Gly, Gly-Gly-Gly-Gly-Gly,

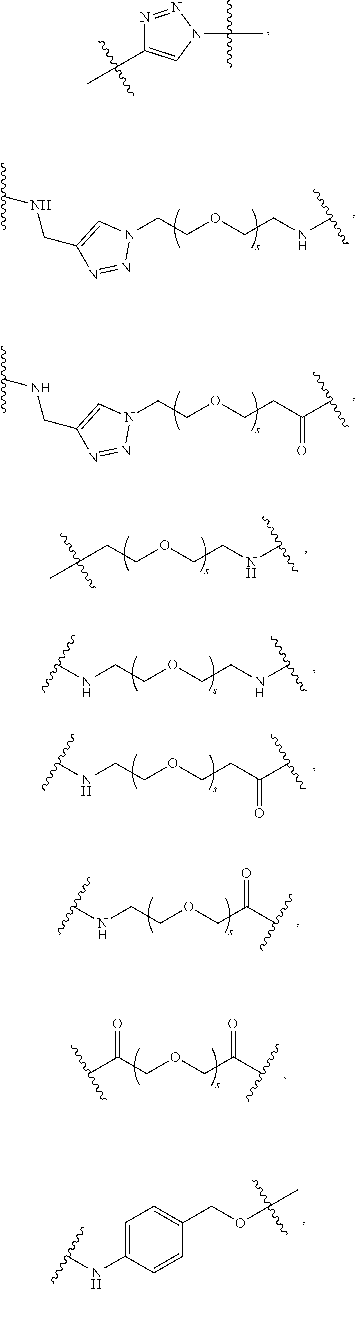

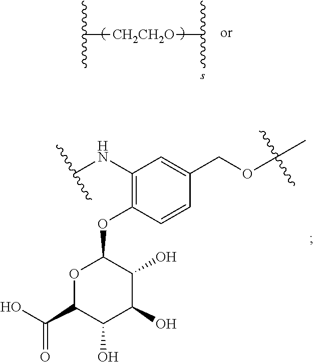

wherein R′ represents hydrogen, C₁₋₆ alkyl or alkyl containing —(CH₂CH₂O)ᵣ—; r is an integer selected from 1 to 10; s is an integer selected from 1 to 20; and
wherein E is a single bond, —NH—CH₂—, —NH—CH₂—O—CH₂—CO— or is selected from the following structures:

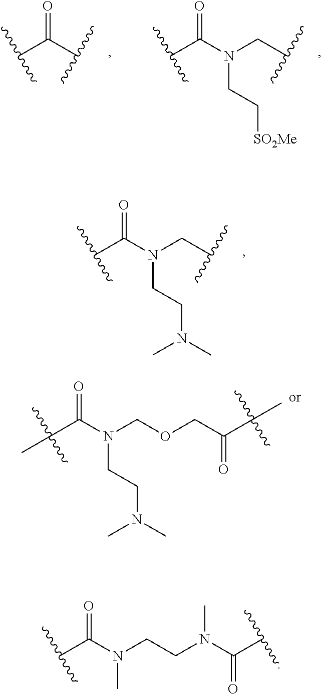

In some embodiments,

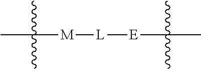

is selected from the following structures:

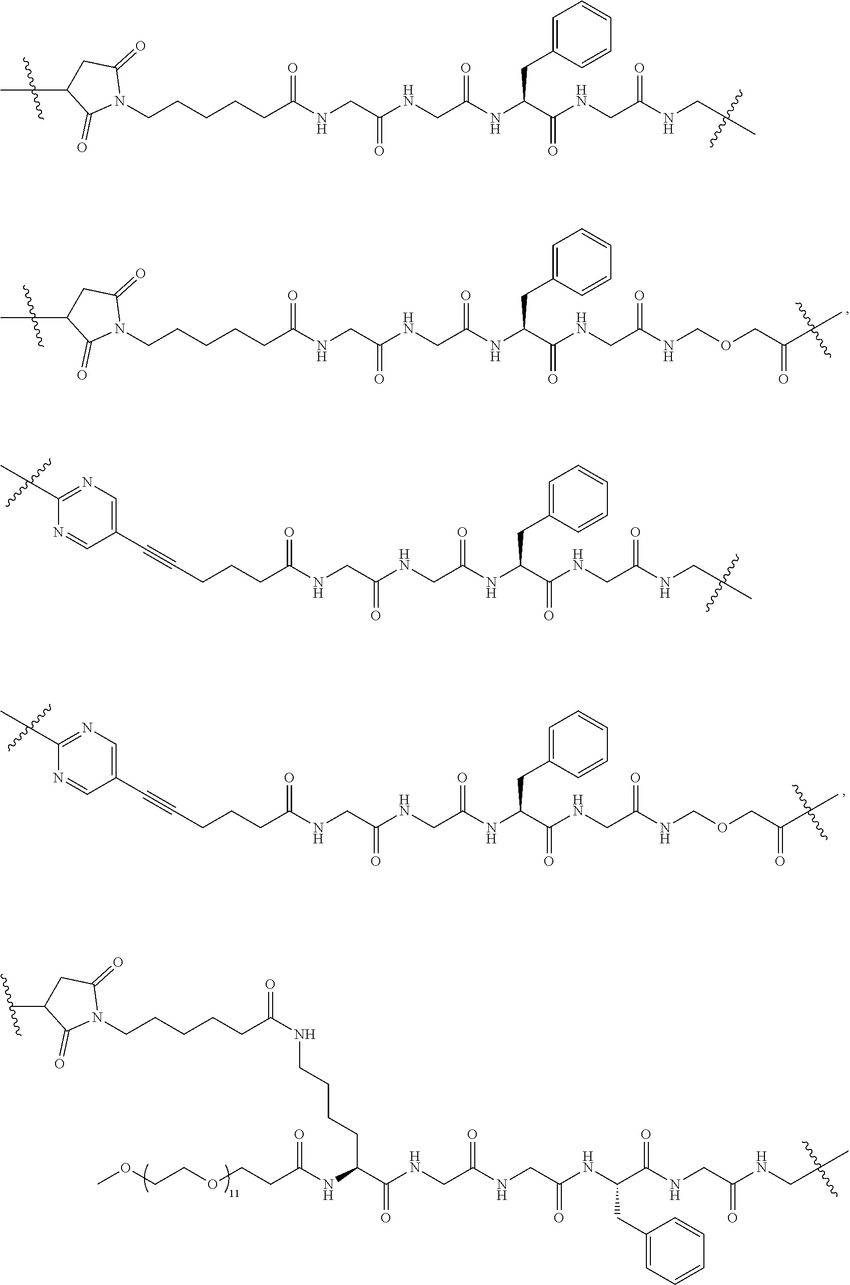

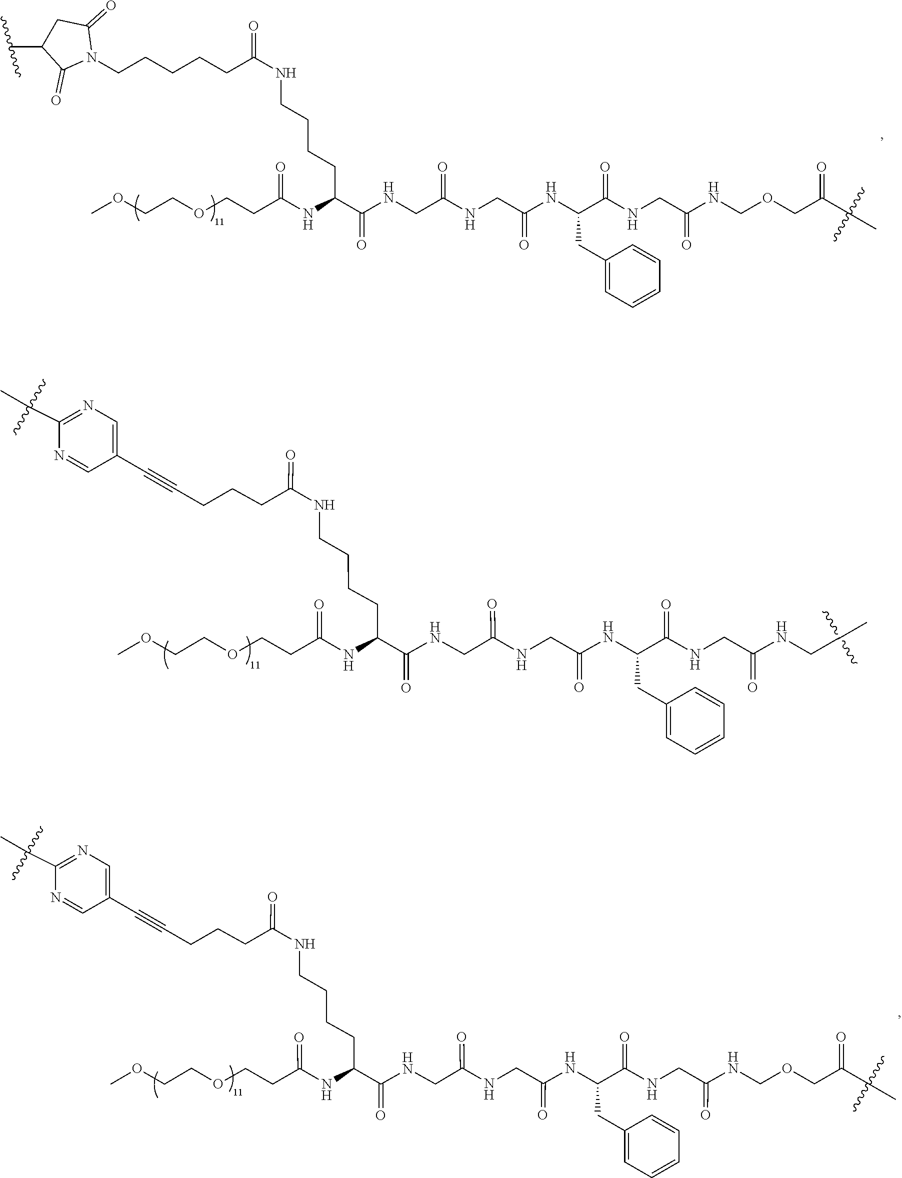

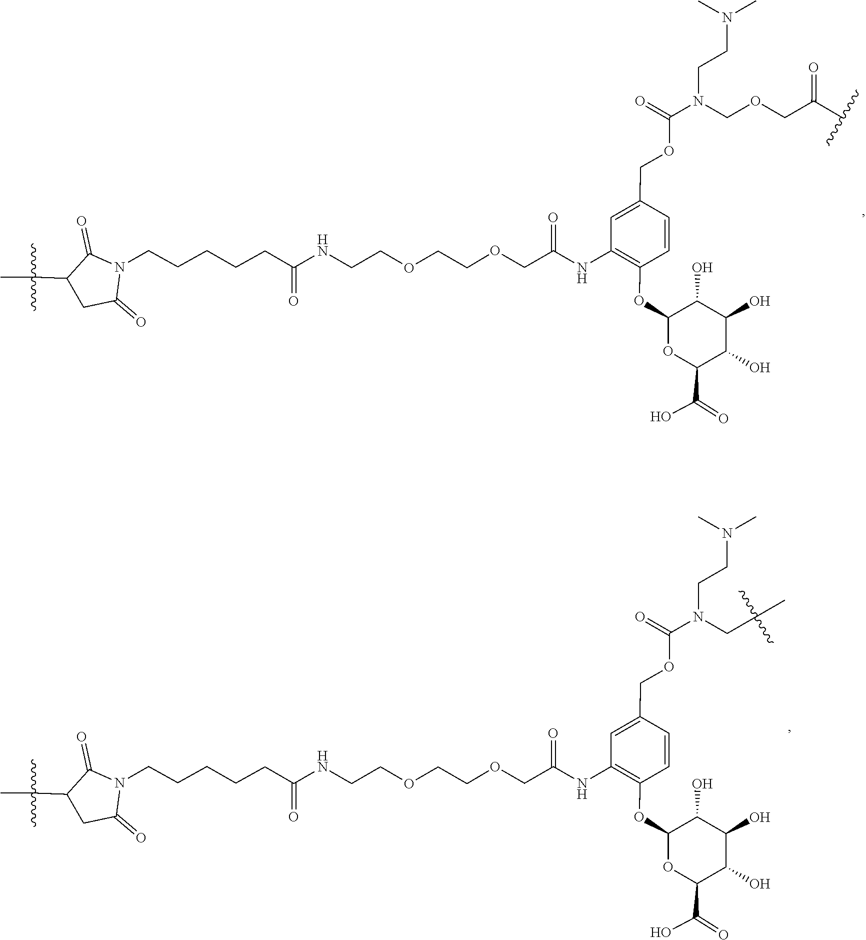 [SMILES: *C(=O)COCN(CCN(C)C)C(=O)OCc1ccc(O[C@@H]2O[C@H](C(=O)O)[C@@H](O)[C@H](O)[C@H]2O)c(NC(=O)COCCOCCNC(=O)CCCCCN2C(=O)CC(*)C2=O)c1 ; *CN(CCN(C)C)C(=O)OCc1ccc(O[C@@H]2O[C@H](C(=O)O)[C@@H](O)[C@H](O)[C@H]2O)c(NC(=O)COCCOCCNC(=O)CCCCCN2C(=O)CC(*)C2=O)c1]

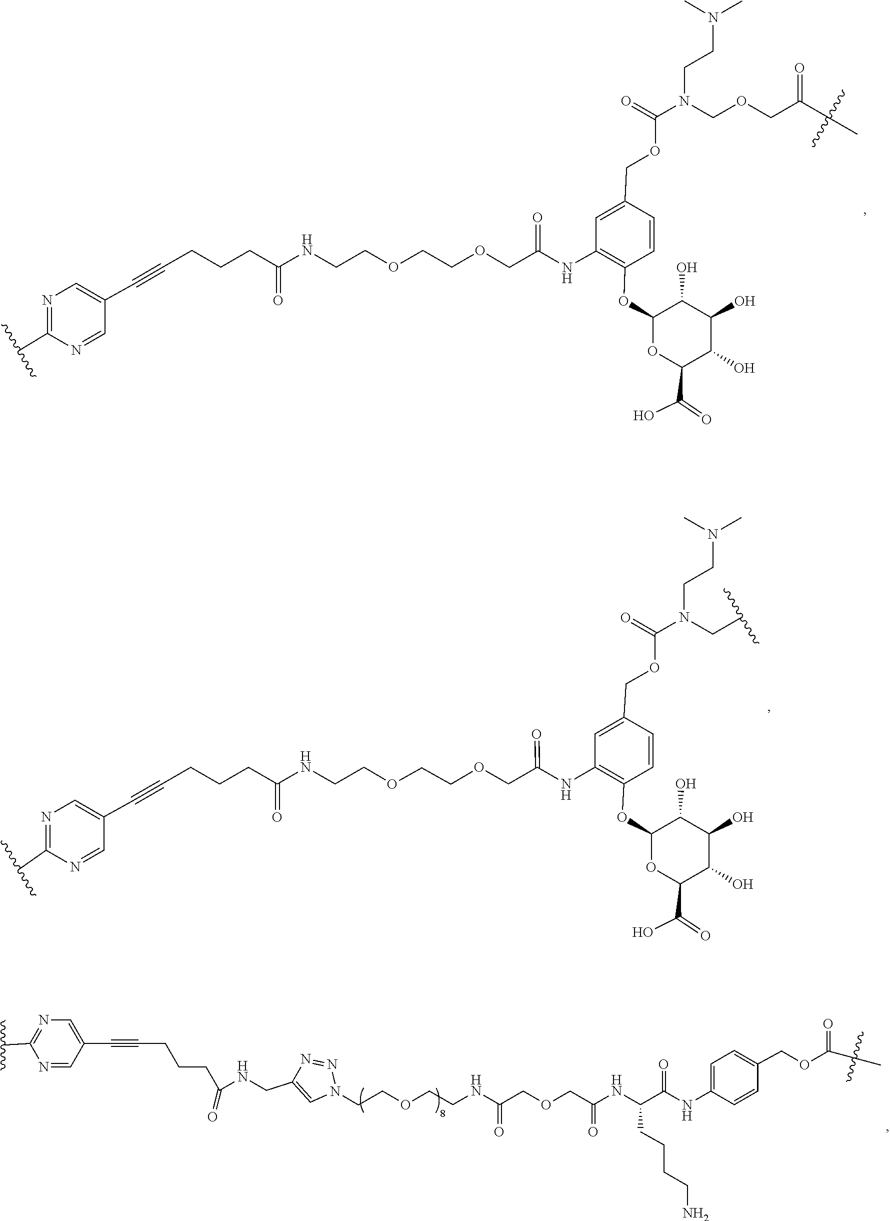

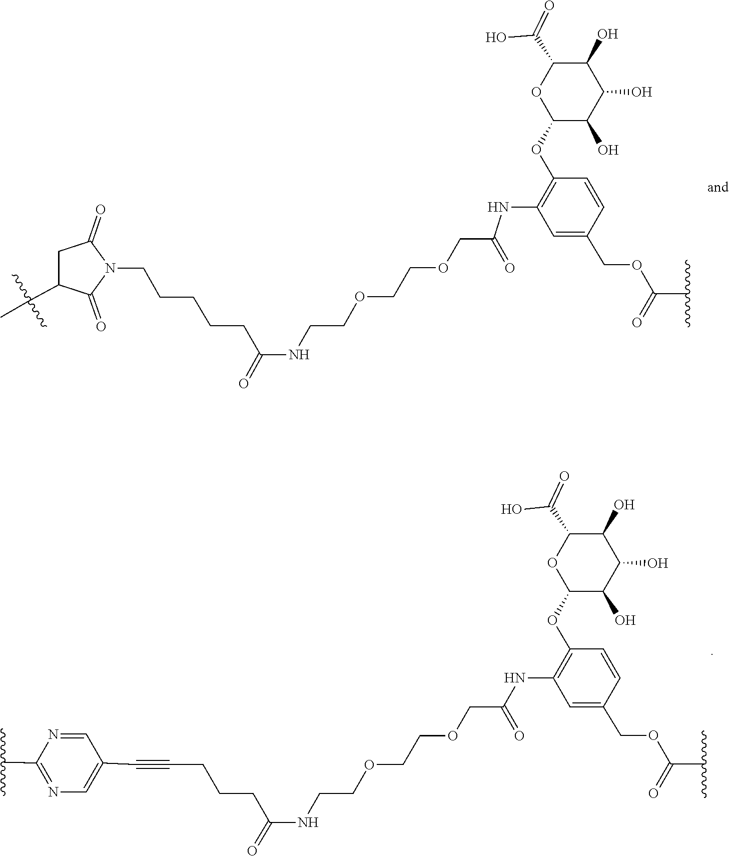 [SMILES: *C(=O)OCc1ccc(O[C@@H]2O[C@H](C(=O)O)[C@@H](O)[C@H](O)[C@H]2O)c(NC(=O)COCCOCCNC(=O)CCCCCN2C(=O)CC(*)C2=O)c1 ; *C(=O)OCc1ccc(O[C@@H]2O[C@H](C(=O)O)[C@@H](O)[C@H](O)[C@H]2O)c(NC(=O)COCCOCCNC(=O)CCCC#Cc2cnc(*)nc2)c1]

In some embodiments, the cytotoxic drug is selected from the group consisting of tubulin inhibitors, DNA intercalators, DNA topoisomerase inhibitors, and RNA polymerase inhibitors. In some embodiments, the tubulin inhibitor is an auristatin compound or a maytansine compound. In some embodiments, the DNA intercalator is pyrrolobenzodiazepine (PBD). In some embodiments, the DNA topoisomerase inhibitor is a topoisomerase I inhibitor (e.g., camptothecin, hydroxycamptothecin, 9-aminocamptothecin, SN-38, irinotecan, topotecan, belotecan, or rubitecan) or a topoisomerase II inhibitor (e.g., doxorubicin, PNU-159682, duocarmycin, daunorubicin, mitoxantrone, podophyllotoxin, or etoposide). In some embodiments, the RNA polymerase inhibitor is α-amanitin or a pharmaceutically acceptable salt, ester or analog thereof.

In some embodiments, the cytotoxic drug is selected from the compounds as shown in Formulas I and II:

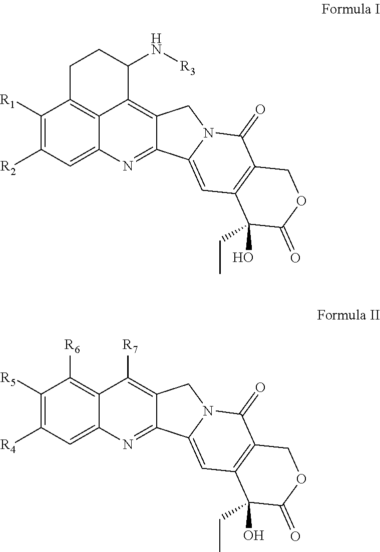

wherein, R₁ and R₂ are each independently selected from the group consisting of C₁₋₆ alkyl and halogen;
R₃ is selected from the group consisting of H and —CO—CH₂OH;
R₄ and R₅ are each independently selected from the group consisting of H, halogen and hydroxyl; or R₄ and R₅ together with the carbon atoms to which they are connected form a 5- to 6-membered oxygen-containing heterocycle;
R₆ is selected from the group consisting of hydrogen and —C₁₋₄ alkylene-NRₐR_{b};
R₇ is selected from the group consisting of C₁₋₆ alkyl and —C₁₋₄ alkylene-NRₐR_{b};
wherein, at each occurrence, Rₐ and R_{b} are each independently selected from the group consisting of H, C₁₋₆ alkyl, —SO₂—C₁₋₆ alkyl and —CO—C₁₋₆ alkyl.

In further embodiments, the cytotoxic drug is selected from the following structures:

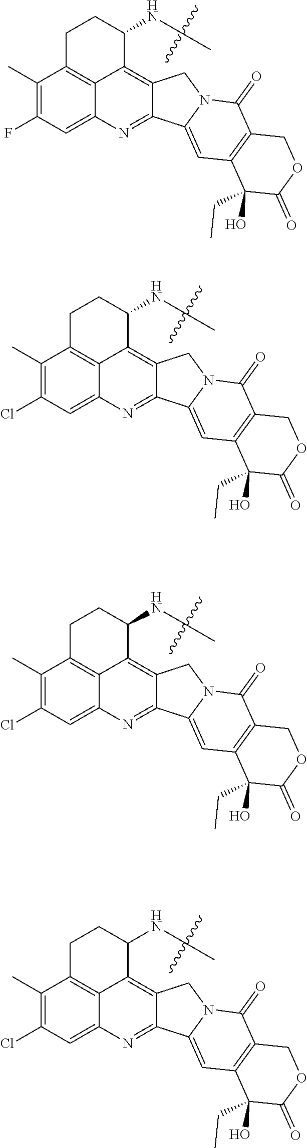 [SMILES: *N[C@H]1CCc2c(C)c(F)cc3nc4c(c1c23)Cn1c-4cc2c(c1=O)COC(=O)[C@]2(O)CC ; *N[C@H]1CCc2c(C)c(Cl)cc3nc4c(c1c23)Cn1c-4cc2c(c1=O)COC(=O)[C@]2(O)CC ; *N[C@@H]1CCc2c(C)c(Cl)cc3nc4c(c1c23)Cn1c-4cc2c(c1=O)COC(=O)[C@]2(O)CC ; *NC1CCc2c(C)c(Cl)cc3nc4c(c1c23)Cn1c-4cc2c(c1=O)COC(=O)[C@]2(O)CC]

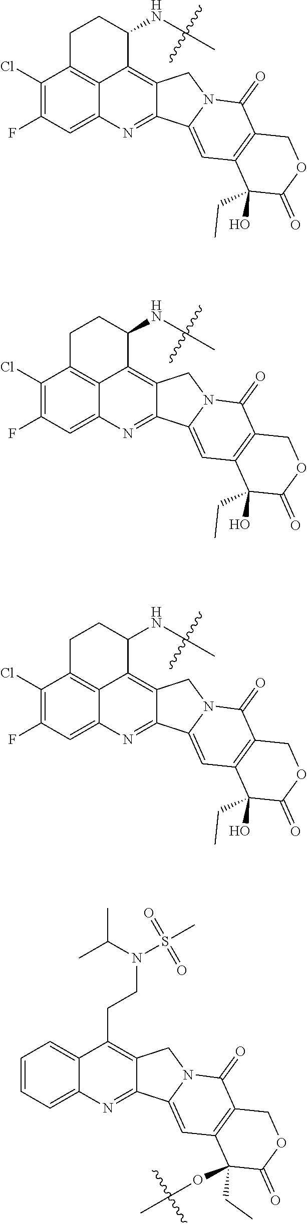

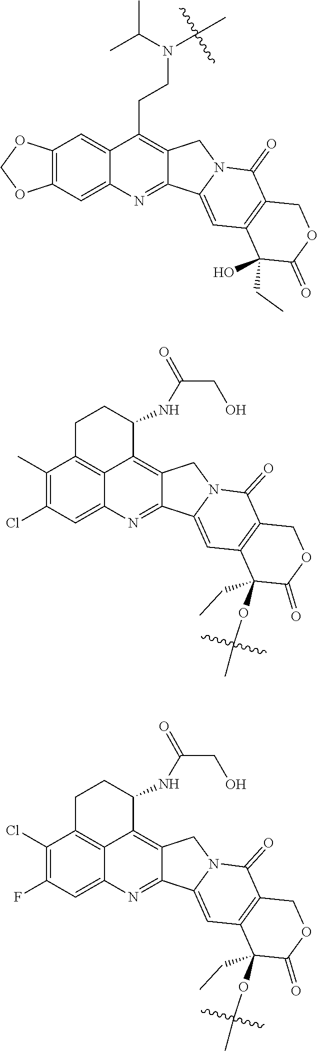 [SMILES: *N(CCc1c2c(nc3cc4c(cc13)OCO4)-c1cc3c(c(=O)n1C2)COC(=O)[C@]3(O)CC)C(C)C ; *O[C@]1(CC)C(=O)OCc2c1cc1n(c2=O)Cc2c-1nc1cc(Cl)c(C)c3c1c2[C@@H](NC(=O)CO)CC3 ; *O[C@]1(CC)C(=O)OCc2c1cc1n(c2=O)Cc2c-1nc1cc(F)c(Cl)c3c1c2[C@@H](NC(=O)CO)CC3]

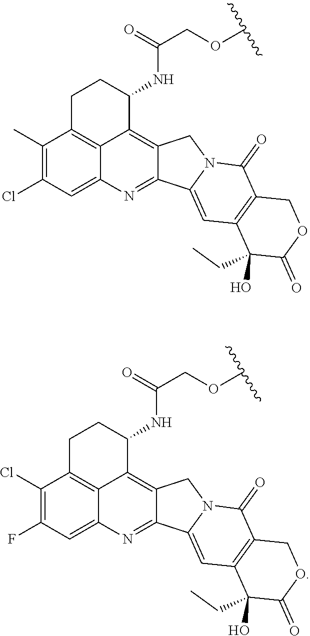 [SMILES: *OCC(=O)N[C@H]1CCc2c(C)c(Cl)cc3nc4c(c1c23)Cn1c-4cc2c(c1=O)COC(=O)[C@]2(O)CC ; *OCC(=O)N[C@H]1CCc2c(Cl)c(F)cc3nc4c(c1c23)Cn1c-4cc2c(c1=O)COC(=O)[C@]2(O)CC]

In some embodiments, the antibody-drug conjugate is selected from the group consisting of ADC A-01 to ADC A-25, ADC B-01 to ADC B-05 as shown below:

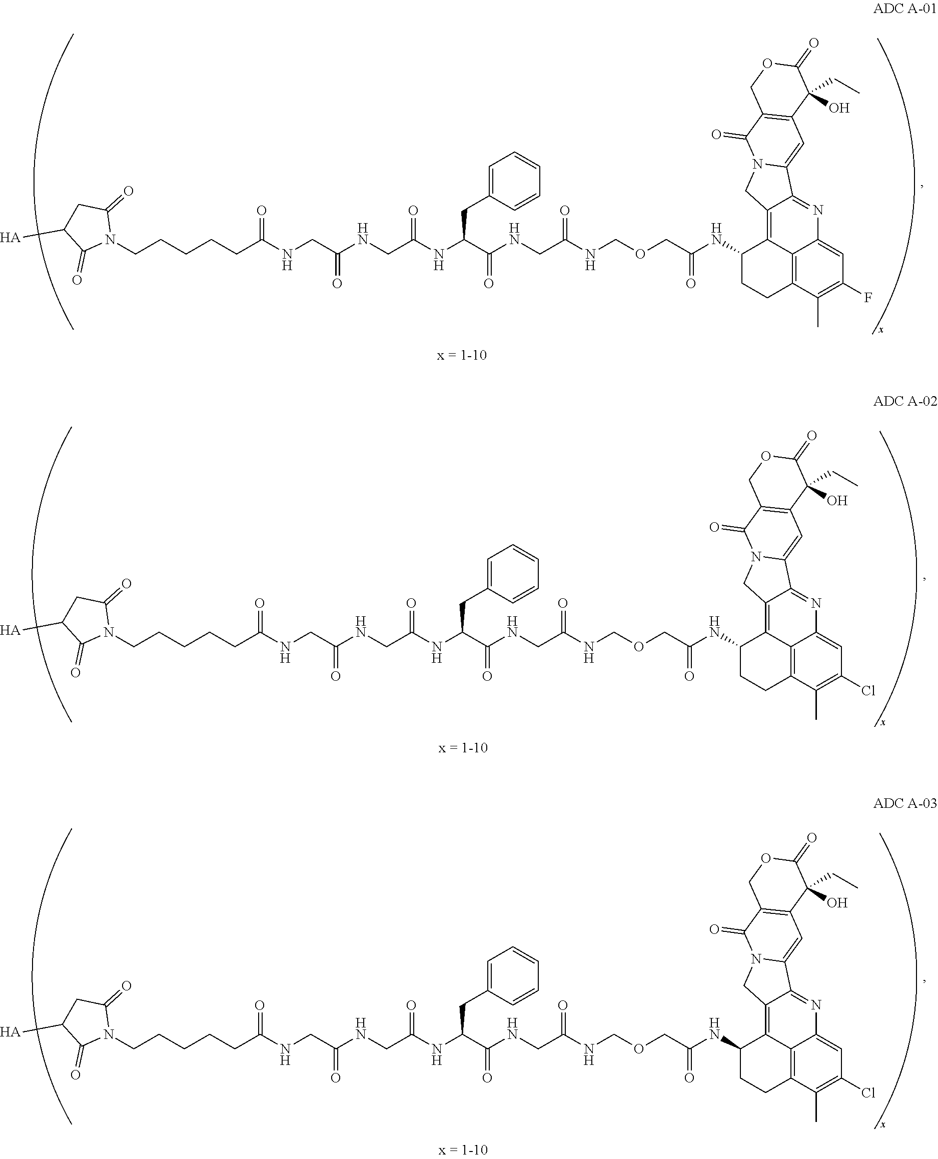

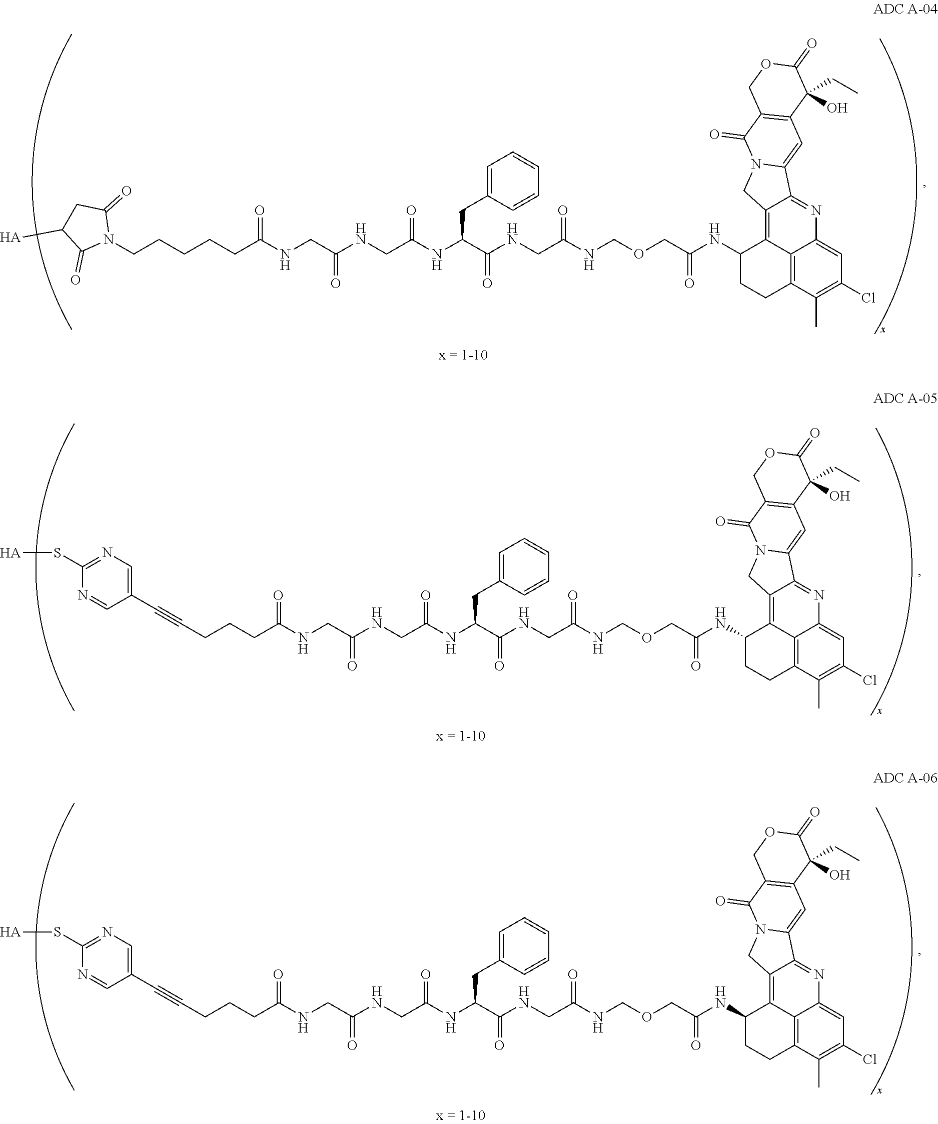

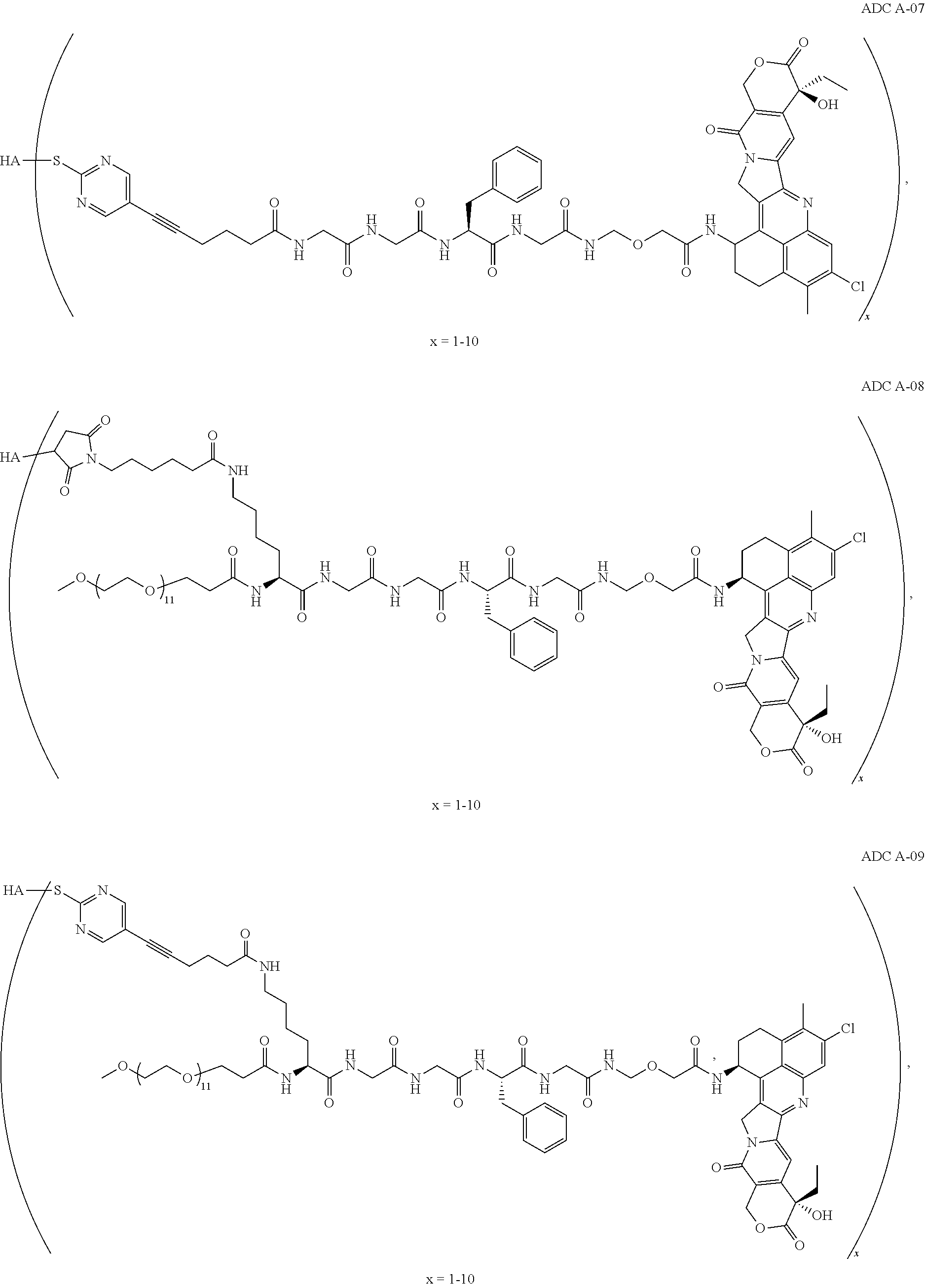

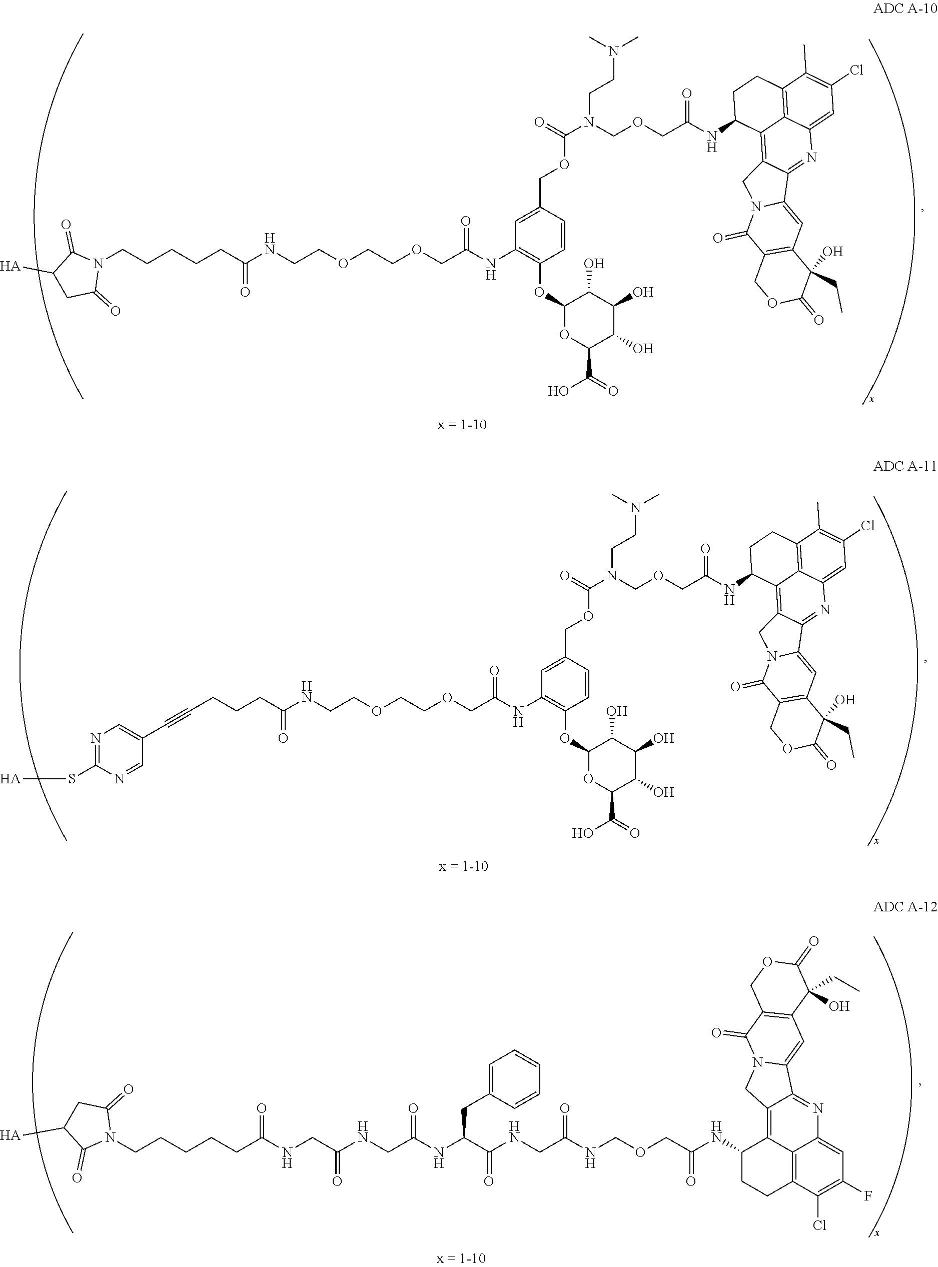

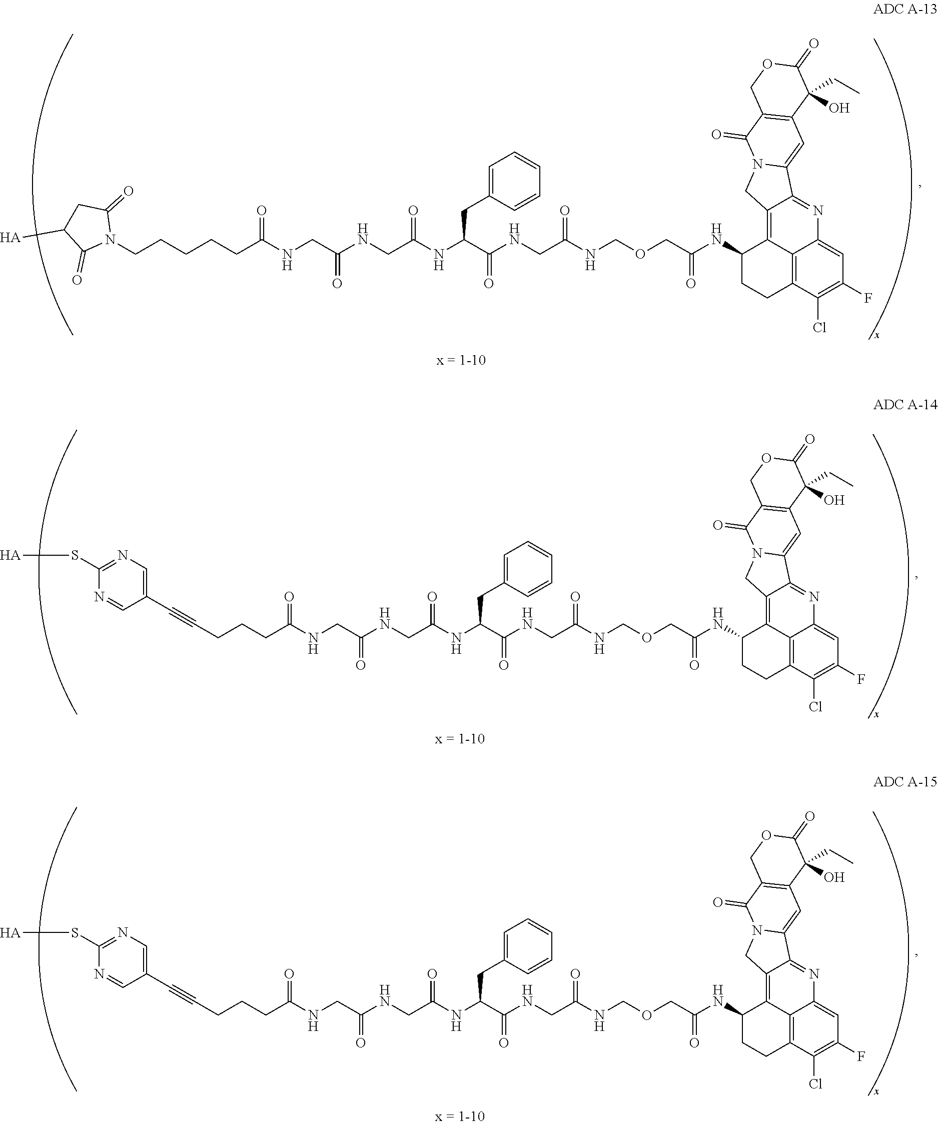

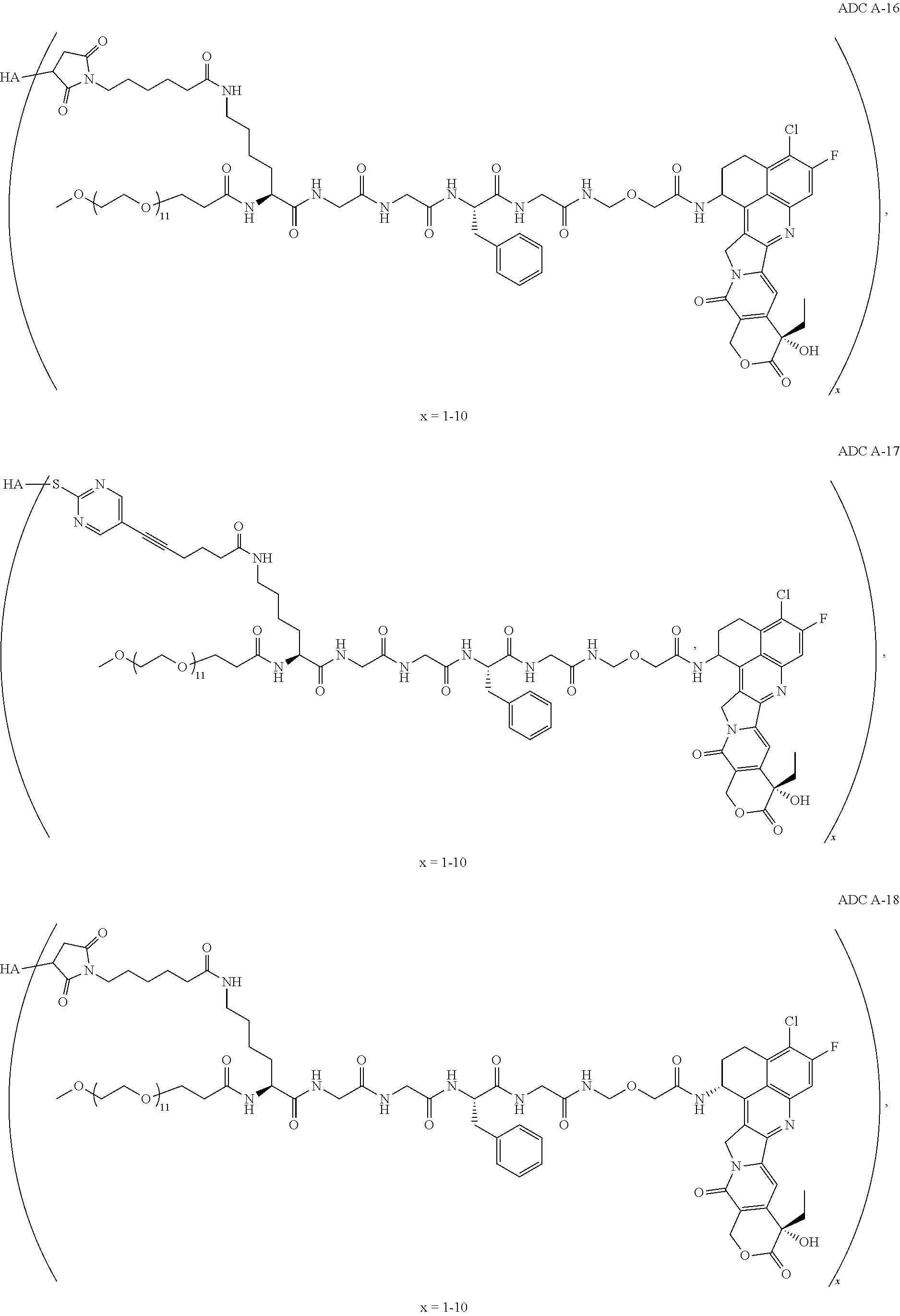

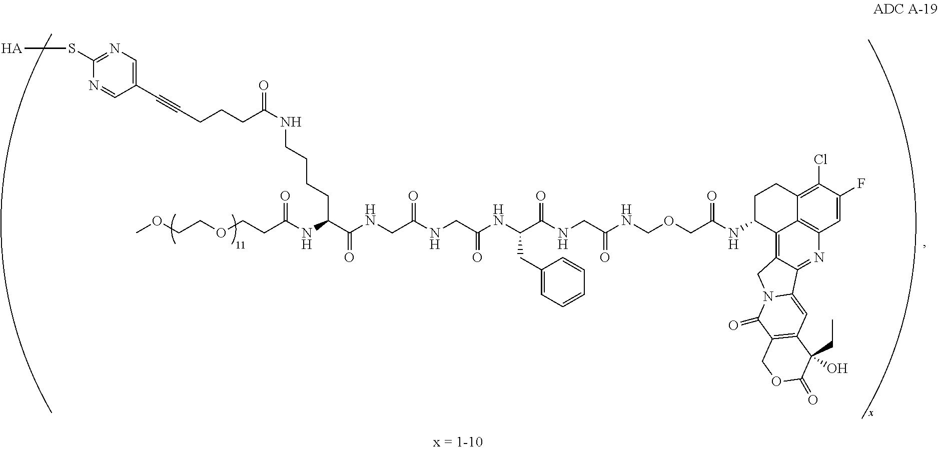

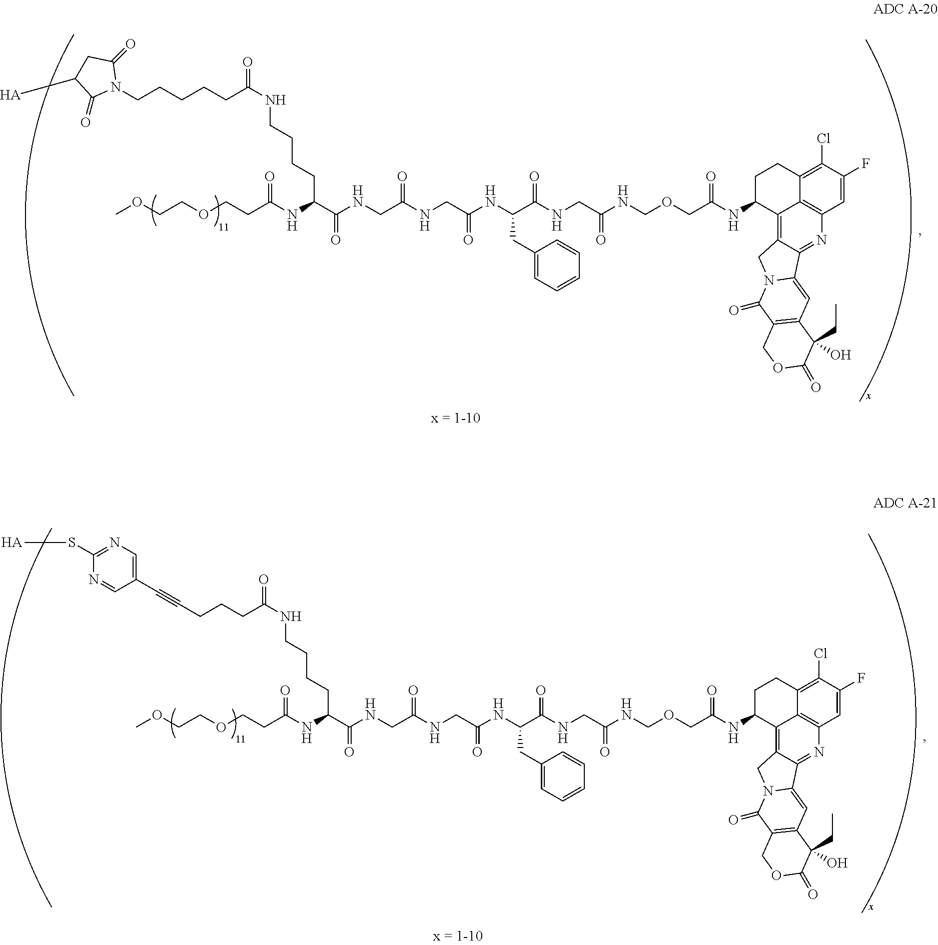

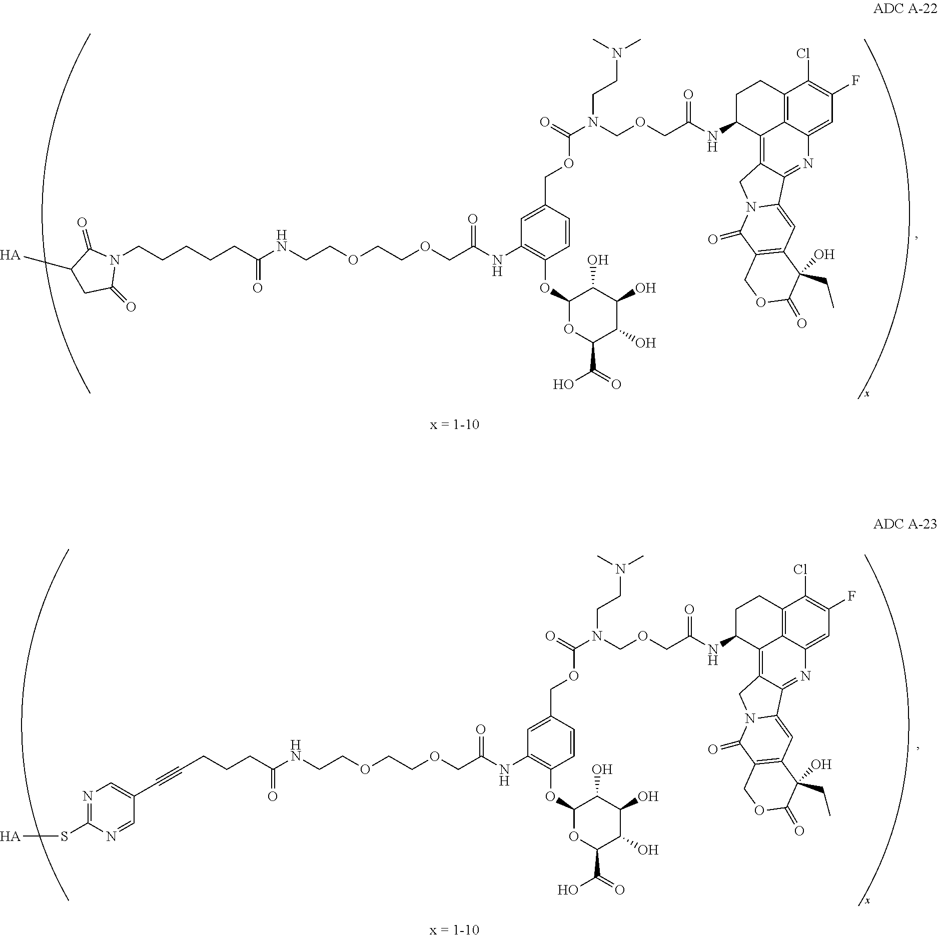

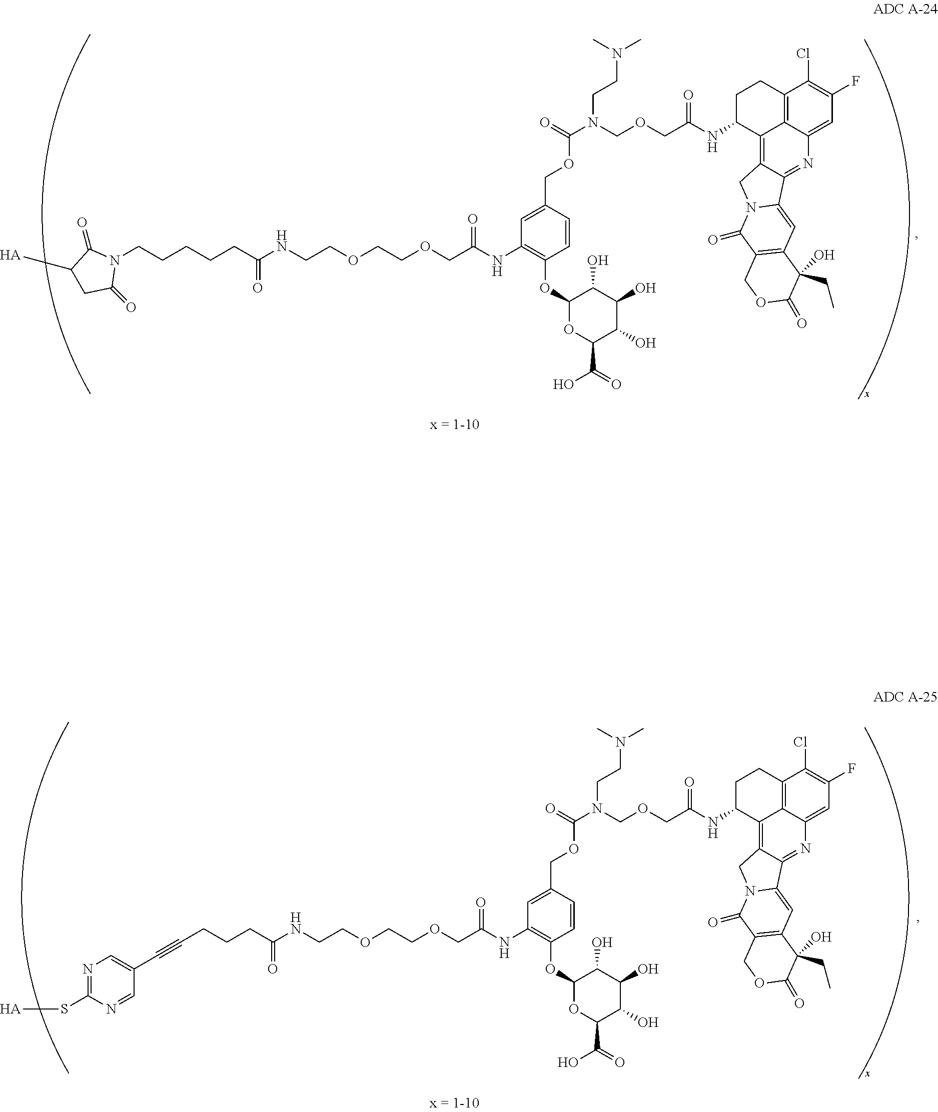

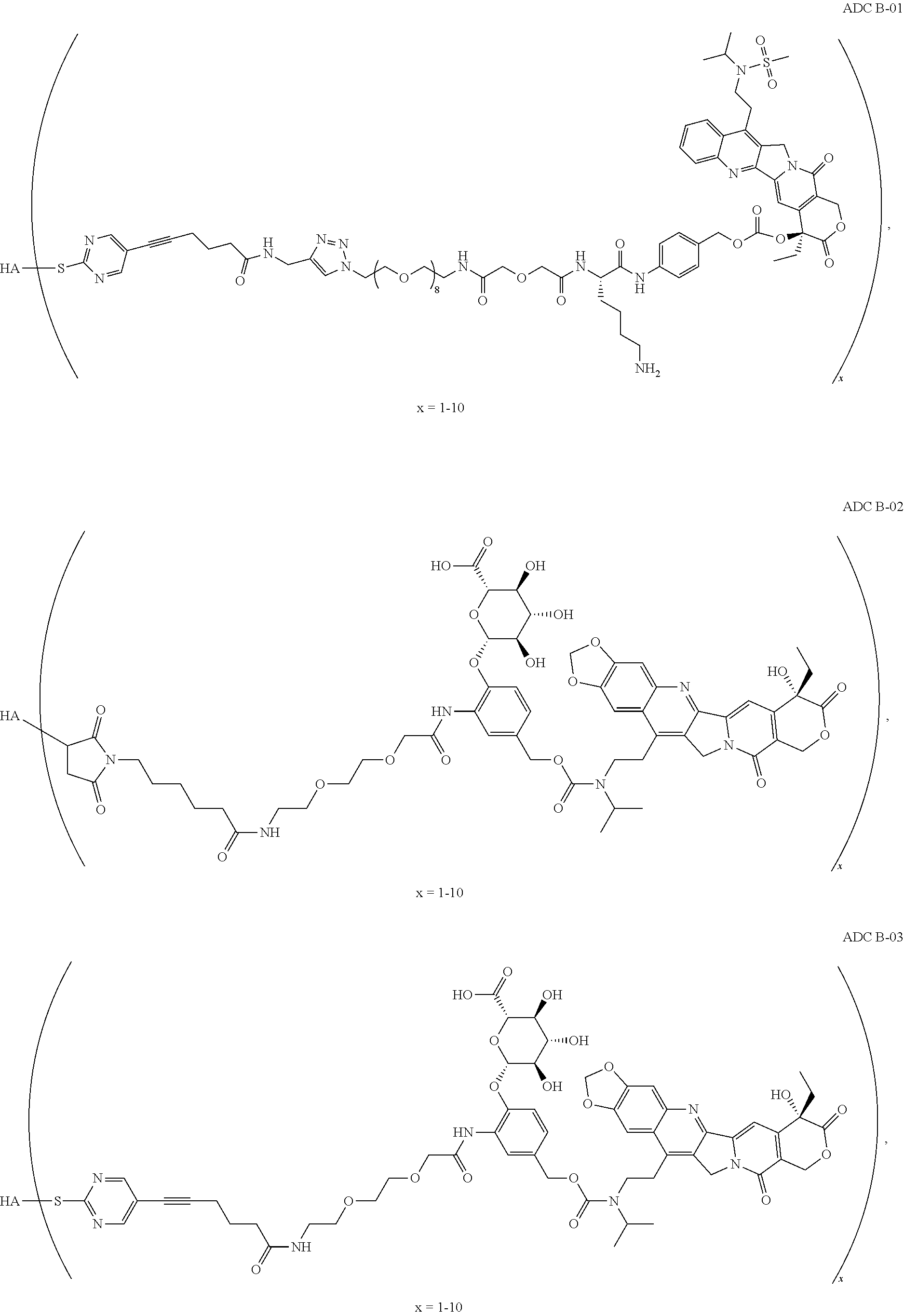

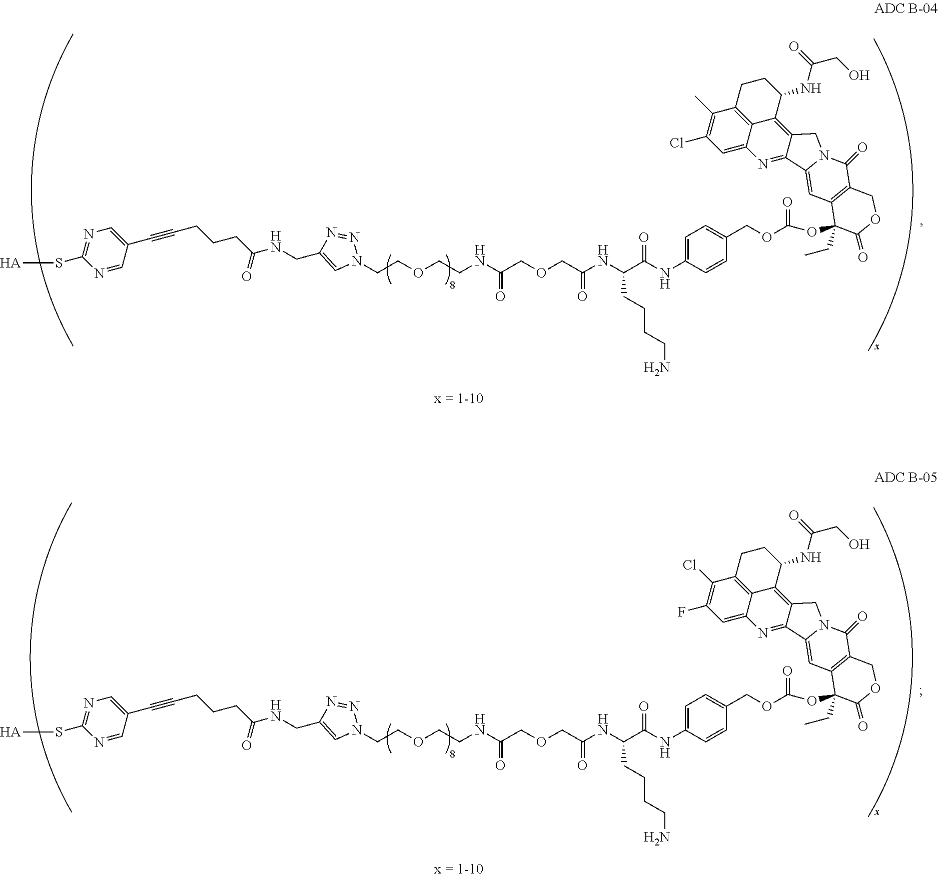

wherein HA is any one of the antibodies disclosed herein and S is a sulfur atom of the side chain of a cysteine residue of the antibody.

In a further embodiment, HA is an antibody comprising:
(1a) a heavy chain variable region (VH) comprising the following 3 CDRs: CDR-H1 having the amino acid sequence as set forth in SEQ ID NO: 1, CDR-H2 having the amino acid sequence as set forth in SEQ ID NO: 2, CDR-H3 having the amino acid sequence as set forth in SEQ ID NO: 3; and, a light chain variable region (VL) comprising the following 3 CDRs: CDR-L1 having the amino acid sequence as set forth in SEQ ID NO: 4, CDR-L2 having the amino acid sequence as set forth in SEQ ID NO: 5, CDR-L3 having a sequence as set forth in SEQ ID NO: 6; or,
(1b) a heavy chain variable region (VH) comprising the following 3 CDRs: CDR-H1 having the amino acid sequence as set forth in SEQ ID NO: 19, CDR-H2 having the amino acid sequence as set forth in SEQ ID NO: 20, CDR-H3 having the amino acid sequence as set forth in SEQ ID NO: 21; and, a light chain variable region (VL) comprising the following 3 CDRs: CDR-L1 having the amino acid sequence as set forth in SEQ ID NO: 22, CDR-L2 having the amino acid sequence as set forth in SEQ ID NO: 23, CDR-L3 having the amino acid sequence as set forth in SEQ ID NO: 24; or,
(1c) a heavy chain variable region (VH) comprising the following 3 CDRs: CDR-H1 having the amino acid sequence as set forth in SEQ ID NO: 36, CDR-H2 having the amino acid sequence as set forth in SEQ ID NO: 37, CDR-H3 having the amino acid sequence as set forth in SEQ ID NO: 38; and, a light chain variable region (VL) comprising the following 3 CDRs: CDR-L1 having the amino acid sequence as set forth in SEQ ID NO: 45, CDR-L2 having the amino acid sequence as set forth in SEQ ID NO: 23, and CDR-L3 having the amino acid sequence as set forth in SEQ ID NO: 52;
(2a) a heavy chain variable region (VH) comprising the following 3 CDRs: CDR-H1 having the amino acid sequence as set forth in SEQ ID NO: 7, CDR-H2 having the amino acid sequence as set forth in SEQ ID NO: 8, CDR-H3 having the amino acid sequence as set forth in SEQ ID NO: 9; and, a light chain variable region (VL) comprising the following 3 CDRs: CDR-L1 having the amino acid sequence as set forth in SEQ ID NO: 4, CDR-L2 having the amino acid sequence as set forth in SEQ ID NO: 5, CDR-L3 having the amino acid sequence as set forth in SEQ ID NO: 6; or,
(2b) a heavy chain variable region (VH) comprising the following 3 CDRs: CDR-H1 having the amino acid sequence as set forth in SEQ ID NO: 25, CDR-H2 having the amino acid sequence as set forth in SEQ ID NO: 26, CDR-H3 having the amino acid sequence as set forth in SEQ ID NO: 21; and, a light chain variable region (VL) comprising the following 3 CDRs: CDR-L1 having the amino acid sequence as set forth in SEQ ID NO: 22, CDR-L2 having the amino acid sequence as set forth in SEQ ID NO: 23, CDR-L3 having the amino acid sequence as set forth in SEQ ID NO: 24; or,
(2c) a heavy chain variable region (VH) comprising the following 3 CDRs: CDR-H1 having the amino acid sequence as set forth in SEQ ID NO: 39, CDR-H2 having the amino acid sequence as set forth in SEQ ID NO: 40, CDR-H3 having the amino acid sequence as set forth in SEQ ID NO: 38; and, a light chain variable region (VL) comprising the following 3 CDRs: CDR-L1 having the amino acid sequence as set forth in SEQ ID NO: 45, CDR-L2 having the amino acid sequence as set forth in SEQ ID NO: 23, and CDR-L3 having the amino acid sequence as set forth in SEQ ID NO: 52;
or,
(3a) a heavy chain variable region (VH) comprising the following 3 CDRs: CDR-H1 having the amino acid sequence as set forth in SEQ ID NO: 10, CDR-H2 having the amino acid sequence as set forth in SEQ ID NO: 11, CDR-H3 having the amino acid sequence as set forth in SEQ ID NO: 12; and, a light chain variable region (VL) comprising the following 3 CDRs: CDR-L1 having the amino acid sequence as set forth in SEQ ID NO: 13, CDR-L2 having the amino acid sequence as set forth in SEQ ID NO: 14, CDR-L3 having the amino acid sequence as set forth in SEQ ID NO: 6; or,
(3b) a heavy chain variable region (VH) comprising the following 3 CDRs: CDR-H1 having the amino acid sequence as set forth in SEQ ID NO: 27, CDR-H2 having the amino acid sequence as set forth in SEQ ID NO: 28, CDR-H3 having the amino acid sequence as set forth in SEQ ID NO: 29; and, a light chain variable region (VL) comprising the following 3 CDRs: CDR-L1 having the amino acid sequence as set forth in SEQ ID NO: 30, CDR-L2 having the amino acid sequence as set forth in SEQ ID NO: 31, CDR-L3 having the amino acid sequence as set forth in SEQ ID NO: 24; or,
(3c) a heavy chain variable region (VH) comprising the following 3 CDRs: CDR-H1 having the amino acid sequence as set forth in SEQ ID NO: 41, CDR-H2 having the amino acid sequence as set forth in SEQ ID NO: 42, CDR-H3 having the amino acid sequence as set forth in SEQ ID NO: 43; and, a light chain variable region (VL) comprising the following 3 CDRs: CDR-L1 having the amino acid sequence as set forth in SEQ ID NO: 44, CDR-L2 having the amino acid sequence as set forth in SEQ ID NO: 31, and CDR-L3 having the amino acid sequence as set forth in SEQ ID NO: 52.

In a further embodiment, HA comprises: a VH comprising the amino acid sequence as set forth in SEQ ID NO: 15 and a VL comprising the amino acid sequence as set forth in SEQ ID NO: 16.

In a further embodiment, HA comprises: a VH comprising the amino acid sequence as set forth in SEQ ID NO: 32 and a VL comprising the amino acid sequence as set forth in SEQ ID NO: 33.

In a further embodiment, HA comprises: a VH comprising the amino acid sequence as set forth in SEQ ID NO: 46 and a VL comprising the amino acid sequence as set forth in SEQ ID NO: 47.

In a further embodiment, HA comprises: (1) a heavy chain comprising a VH comprising the amino acid sequence as set forth in SEQ ID NO: 15 and a heavy chain constant region (CH) as set forth in SEQ ID NO: 50, and, a light chain comprising a VL comprising the amino acid sequence as set forth in SEQ ID NO: 16 and a light chain constant region (CL) as set forth in SEQ ID NO: 51.

In a further embodiment, HA comprises: (2) a heavy chain comprising a VH comprising the amino acid sequence as set forth in SEQ ID NO: 32 and a heavy chain constant region (CH) as set forth in SEQ ID NO: 50, and, a light chain comprising a VL comprising the amino acid sequence as set forth in SEQ ID NO: 33 and a light chain constant region (CL) as set forth in SEQ ID NO: 51; or

In a further embodiment, HA comprises: (3) a heavy chain comprising a VH comprising the amino acid sequence as set forth in SEQ ID NO: 46 and a heavy chain constant region (CH) as set forth in SEQ ID NO: 50, and, a light chain comprising a VL comprising the amino acid sequence as set forth in SEQ ID NO: 47 and a light chain constant region (CL) as set forth in SEQ ID NO: 51.

In a further embodiment, HA comprises a heavy chain (HC) comprising the amino acid sequence as set forth in SEQ ID NO: 17, and a light chain (LC) comprising the amino acid sequence as set forth in SEQ ID NO: 18.

In a further embodiment, HA comprises a heavy chain (HC) comprising the amino acid sequence as set forth in SEQ ID NO: 34, and a light chain (LC) comprising the amino acid sequence as set forth in SEQ ID NO: 35.

In a further embodiment, HA comprises a heavy chain (HC) comprising the amino acid sequence as set forth in SEQ ID NO: 48, and a light chain (LC) comprising the amino acid sequence as set forth in SEQ ID NO: 49.

In particular embodiments of HA or antibody-drug conjugate disclosed herein, the heavy chain constant domains may comprise a C-terminal lysine or lack either a C-terminal lysine or a C-terminal glycine-lysine dipeptide. In some embodiments of the antibody or antigen binding fragment thereof, the N-terminal amino acid of the antibody or antigen binding fragment thereof variable domains may undergo cyclization to pyroglutamate.

Thus, a composition may comprise a population of antibody-drug conjugate species wherein each species may independently comprise a C-terminal lysine, lack a C-terminal lysine, lack a C-terminal glycine-lysine and/or comprise an N-terminal glutamine or glutamic acid or cyclization of the N-terminal amino acid to pyroglutamate.

Therefore, in particular embodiments, the present invention further provides compositions comprising an ADC disclosed herein wherein the predominant ADC species in the composition comprises (i) antibodies in which the heavy chain C-terminus lacks a lysine residue; (ii) antibodies in which the heavy chain N-terminus is glutamine, glutamic acid, or pyroglutamate; or, (iii) antibodies in which the heavy chain C-terminus lacks a lysine residue and the heavy chain N-terminus is glutamine, glutamic acid, or pyroglutamate.

Also provided herein are pharmaceutical compositions comprising the antibody-drug conjugates described herein and one or more pharmaceutically acceptable excipients.

Also provided herein are methods of treating a cancer in a subject with high expression of HER3 comprising administering a therapeutic effective amount of the antibody-drug conjugate described herein, or the pharmaceutical composition thereof to the subject. In particular embodiments, the cancer comprises solid tumors or hematological malignancies. In a further embodiment, the cancer is colon cancer, gastric cancer, breast cancer, lung cancer, or lymphoma. In a further embodiment, the lung cancer is a non-small cell lung cancer.

Also provided herein is the use of the antibody-drug conjugate described herein, or the pharmaceutical composition thereof, in the manufacture of a medicament for the treatment of a cancer with high expression of HER3. In particular embodiments, the cancer comprises solid tumors or hematological malignancies. In a further embodiment, the cancer is colon cancer, gastric cancer, breast cancer, lung cancer, or lymphoma. In a further embodiment, the lung cancer is a non-small cell lung cancer.

Also provided herein is the use of the antibody-drug conjugate described herein, or the pharmaceutical composition thereof, in the treatment of a cancer with high expression of HER3.

In particular embodiments, the cancer comprises solid tumors or hematological malignancies. In a further embodiment, the cancer is colon cancer, gastric cancer, breast cancer, lung cancer, or lymphoma. In a further embodiment, the lung cancer is a non-small cell lung cancer.

Also provided herein is an antibody-drug conjugate described herein, or a pharmaceutical composition described herein, for treatment of a cancer with high expression of HER3. In particular embodiments, the cancer comprises solid tumors or hematological malignancies. In a further embodiment, the cancer is colon cancer, gastric cancer, breast cancer, lung cancer, or lymphoma. In a further embodiment, the lung cancer is a non-small cell lung cancer.

In one embodiment, the cancer with high expression of HER3 comprises solid tumors or hematological malignancies, such as colon cancer, gastric cancer, breast cancer, lung cancer (e.g., non-small cell lung cancer, specifically lung adenocarcinoma), or lymphoma.

The present invention further provides pharmaceutically acceptable salts or solvates of any one of the aforementioned antibody-drug conjugates.

The present invention further provides compositions comprising any one of the aforementioned antibody drug conjugates and a pharmaceutically acceptable carrier or excipient.

### BRIEF DESCRIPTION OF THE DRAWINGS

FIG. 1A shows the detection results of affinity of anti-human HER3 antibody-drug conjugates to MDA-MB-453 cells.

FIG. 1B shows the detection results of affinity of anti-human HER3 antibody-drug conjugates to NCI-H358 cells.

FIG. 1C shows the detection results of affinity of anti-human HER3 antibody-drug conjugates to KPL-4 cells.

FIG. 1D shows the detection results of affinity of anti-human HER3 antibody-drug conjugate to A431 cells.

FIG. 2A shows the detection results of in vitro cytotoxicity of anti-human HER3 antibody-drug conjugates on MDA-MB-453 cells.

FIG. 2B shows the detection results of in vitro cytotoxicity of anti-human HER3 antibody-drug conjugates on HCC1569 cells.

FIG. 2C shows the detection results of in vitro cytotoxicity of anti-human HER3 antibody-drug conjugates on HEK293T-h HER3 cells.

FIGS. 3A to 3C shows the detection results of bystander effect of anti-human HER3 antibody-drug conjugates on HEK293T and HEK293T-h HER3 cells.

FIG. 4 shows the detection results of plasma stability of anti-human HER3 antibody-drug conjugates.

FIG. 5A shows the changes in mouse tumor volume caused by anti-human HER3 antibody-drug conjugates in each group of the NCI-H358 CDX model.

FIG. 5B shows the changes in mouse body weight caused by anti-human HER3 antibody-drug conjugates in each group of the NCI-H358 CDX model.

FIG. 6A shows the changes in mouse tumor volume caused by anti-human HER3 antibody-drug conjugates in each group of the NCI-N87 CDX model.

FIG. 6B shows the changes in mouse body weight caused by anti-human HER3 antibody-drug conjugates in each group of the NCI-N87 CDX model.

FIG. 7A shows the changes in mouse tumor volume caused by anti-human HER3 antibody-drug conjugates in each group of the MDA-MB-453 CDX model.

FIG. 7B shows the changes in mouse body weight caused by anti-human HER3 antibody-drug conjugates in each group of the MDA-MB-453 CDX model.

FIG. 8A shows the HPLC profiles of the compounds A-07-A and A-07-B prepared in Example 3

FIG. 8B shows the HPLC profiles of the compound A-07-A prepared in Example 3, and the compound A-05 in Example 9.

FIG. 9 shows the results of the detection of cell affinity of anti-human HER3 antibody drug conjugates to MDA-MB-453 cells.

FIG. 10 shows the results of the detection of endocytosis effects of anti-human HER3 antibody drug conjugates in MDA-MB-453 cells.

FIG. 11 shows the results of the detection of endocytosis effects of anti-human HER3 antibody drug conjugates in HCC1569 cells.

FIG. 12 shows the results of the cell killing effects of anti-human HER3 antibody drug conjugate.

### DETAILED DESCRIPTION OF THE INVENTION

### Definitions

Unless defined otherwise hereinafter, all technical and scientific terms used herein have the same meaning as commonly understood by one of ordinary skill in the art. References to techniques used herein are intended to refer to techniques commonly understood in the art, including those changes in technology or substitutions of equivalent technologies obvious to the skilled person. Moreover, the laboratory operation steps of genomics, nucleic acid chemistry, and molecular biology used herein are all routine steps widely used in the corresponding fields. Although the following terms are believed to be well understood by those skilled in the art, the following definitions are set forth for better explanation of the present invention.

The term “antibody” refers to an immunoglobulin molecule usually composed of two pairs of polypeptide chains (each pair has a light chain (LC) and a heavy chain (HC)). Antibody light chains can be classified into κ (kappa) and λ (lambda) light chains. Heavy chains can be classified into μ, δ, γ, α or ε heavy chains, and the isotypes of antibody are therefore defined as IgM, IgD, IgG, IgA and IgE, respectively. Within the light and heavy chains, the variable and constant regions are connected by a “J” region of about 12 or more amino acids, and the heavy chain also includes a “D” region of about 3 or more amino acids. Each heavy chain is composed of a heavy chain variable region (VH) and a heavy chain constant region (CH). The heavy chain constant region is composed of 3 domains (CH1, CH2 and CH3). Each light chain is composed of a light chain variable region (VL) and a light chain constant region (CL). The light chain constant region consists of one domain CL. The constant domains are not directly involved in the binding of antibodies and antigens, but exhibit a variety of effector functions, such as mediating the binding of immunoglobulins to host tissues or factors, including various cells (for example, effector cells) of immune system and the first component (C1q) of classical complement system. The VH and VL regions can also be subdivided into hypervariable regions (called complementarity determining regions (CDR)), interspersed with more conservative regions (called framework regions (FR)). Each VH and VL consists of 3 CDRs and 4 FRs arranged in the following order: FR1, CDR1, FR2, CDR2, FR3, CDR3, FR4 from the amino terminus to the carboxy terminus. The variable regions (VH and VL) of each heavy chain/light chain pair form antigen binding sites respectively. The assignment of amino acids to regions or domains may follow various numbering systems known in the art.

The term “antibody” further includes embodiments in which heavy chain constant domains may comprise a C-terminal lysine or lack either a C-terminal lysine or a C-terminal glycine-lysine dipeptide. The term further includes embodiments in which the N-terminal amino acid of the antibody variable domains has undergone cyclization to pyroglutamate. Thus, in a composition comprising antibodies as disclosed herein, various species of the antibodies therein may independently comprise a C-terminal lysine, lack a C-terminal lysine, lack a C-terminal glycine-lysine and/or comprise an N-terminal glutamine or glutamic acid or cyclization of the N-terminal amino acid to pyroglutamate.

The term “complementarity determining region” or “CDR” refers to the amino acid residues in antibody variable region that are responsible for antigen binding. Each of the variable regions of heavy and light chains contain 3 CDRs, named CDR1, CDR2 and CDR3. The precise boundaries of these CDRs can be defined according to various numbering systems known in the art, for example, according to the Kabat numbering system (Kabat et al., Sequences of Proteins of Immunological Interest, 5th Ed. Public Health Service, National Institutes of Health, Bethesda, Md., 1991), Chothia numbering system (Chothia & Lesk (1987) J. Mol. Biol. 196:901-917; Chothia et al. (1989) Nature 342:878-883), IMGT numbering system (Lefranc et al., Dev. Comparat. Immunol. 27:55-77, 2003), or AbM numbering system (Martin A C R, Cheetham J C, Rees A R (1989) Modelling antibody hypervariable loops: A combined algorithm. Proc Natl Acad Sci USA 86:9268-9272). For a given antibody, those skilled in the art will easily identify the CDRs defined by each numbering system. Moreover, the correspondence between different numbering systems is well known to those skilled in the art (for example, see Lefranc et al., Dev. Comparat. Immunol. 27:55-77, 2003).

In the present invention, the CDRs contained in the antibody or antigen-binding fragment thereof of the present invention can be determined according to various numbering systems known in the art, such as Kabat, Chothia, IMGT or AbM numbering systems. In certain embodiments, the CDRs contained in the antibody or antigen-binding fragment thereof are determined by the Chothia numbering system.

The following general rules disclosed in www.bioinf.org.uk: Prof. Andrew C. R. Martin's Group and reproduced below may be used to define the CDRs in an antibody sequence that includes those amino acids that specifically interact with the amino acids comprising the epitope in the antigen to which the antibody binds. There are rare examples where these generally constant features do not occur; however, the Cys residues are the most conserved feature.

| Loop | Kabat | AbM | Chothia¹ | Contact² | IMGT |
|---|---|---|---|---|---|
| L1 | L24--L34 | L24--L34 | L24--L34 | L30--L36 | L27--L32 |
| L2 | L50--L56 | L50--L56 | L50--L56 | L46--L55 | L50--L52 |
| L3 | L89--L97 | L89--L97 | L89--L97 | L89--L96 | L89--L97 |
| H1 | H31-- | H26-- | H26-- | H30-- | H26-- |
| | H35B | H35B | H32 . . . 34 | H35B | H35B |
| | (Kabat | | | | |
| | Numbering)³ | | | | |
| H1 | H31--H35 | H26--H35 | H26--H32 | H30--H35 | H26--H33 |
| | (Chothia | | | | |
| | Numbering) | | | | |
| H2 | H50--H65 | H50--H58 | H52--H56 | H47--H58 | H51--H56 |
| H3 | H95--H102 | H95--H102 | H95--H102 | H93-- | H93-- |
| | | | | H101 | H102 |

¹Some of these numbering schemes (particularly for Chothia loops) vary depending on the individual publication examined.
²Any of the numbering schemes can be used for these CDR definitions, except the Contact numbering scheme uses the Chothia or Martin (Enhanced Chothia) definition.
³The end of the Chothia CDR-H1 loop when numbered using the Kabat numbering convention varies between H32 and H34 depending on the length of the loop. (This is because the Kabat numbering scheme places the insertions at H35A and H35B.)
If neither H35A nor H35B is present, the loop ends at H32
If only H35A is present, the loop ends at H33
If both H35A and H35B are present, the loop ends at H34

The entire amino acid sequence of the VH is commonly numbered according to Kabat while the three CDRs within the variable region may be defined according to any one of the aforementioned numbering schemes. In particular embodiments, the numbering of the amino acid positions in the VH may be sequential beginning with amino acid position 1 and continuing sequentially to the end of the sequence or according to Kabat. Unless specified otherwise, the amino acid positions in the VH and VL herein are defined according to sequential numbering.

The numbering of the amino acid positions in the heavy chain constant domain may be sequential beginning with amino acid position 1 and continuing sequentially to the end of the sequence or according to Eu numbering. The IgG1 heavy chain constant domain amino acid sequence has 330 amino acids sequentially numbered 1 to 330. The corresponding sequence numbered according to Eu begins with position number 118 and ends with position number 447. Unless specified otherwise, the amino acid positions in the heavy and light chains herein are defined according to sequential numbering.

The term “framework region” or “FR” residues refers to the amino acid residues in the variable region of the antibody other than the CDR residues as defined above.

The term “antigen-binding fragment” of an antibody refers to a polypeptide of antibody fragment, such as a polypeptide of fragment of full-length antibody, which retains the ability to specifically bind to the same antigen to which the full-length antibody binds, and/or compete with the full-length antibody for specific binding to the antigen, which is also called “antigen-binding portion”. See generally, Fundamental Immunology, Ch. 7 (Paul, W., ed., 2nd edition, Raven Press, NY (1989), which is incorporated herein by reference in its entirety for all purposes. Recombinant DNA technology or enzymatic or chemical cleavage of an intact antibody can be used to produce an antigen-binding fragment of the antibody. Non-limiting examples of antigen-binding fragment include Fab fragment, Fab′ fragment, F(ab′)₂ fragment, F(ab′)₃ fragment, Fd, Fv, scFv, di-scFv, (scFv) 2, disulfide bond-stabilized Fv protein (“dsFv”), single domain antibody (sdAb, nanobody) and such polypeptides, which contain at least a portion of antibody that is sufficient to confer the polypeptide a specific antigen binding ability. Engineered antibody variants are reviewed by Holliger et al., 2005; Nat Biotechnol, 23:1126-1136.

The term “Fd” refers to an antibody fragment composed of VH and CH1 domains; the term “dAb fragment” refers to an antibody fragment composed of VH domains (Ward et al., Nature 341:544 546 (1989)); the term “Fab fragment” refers to an antibody fragment composed of VL, VH, CL and CH1 domains; the term “F(ab′)₂ fragment” refers to an antibody fragment comprising two Fab fragments connected by a disulfide bridge of the hinge region; the term “Fab′ fragment” refers to a fragment obtained by reducing the disulfide bond connecting the two heavy chain fragments in F(ab′)₂ fragment, consisting of an intact light chain and a heavy chain Fd fragment (consisting of VH and CH1 domains).

The term “Fv” refers to an antibody fragment composed of the VL and VH domains of a single arm of antibody. Fv fragment is generally considered to be the smallest antibody fragment that can form a complete antigen binding site. It is generally believed that six CDRs can confer antigen binding specificity to antibody. However, even a variable region (e.g., a Fd fragment, which contains only three antigen-specific CDRs) can recognize and bind an antigen, although its affinity may be lower than that of the complete binding site.

The term “Fc” refers to an antibody fragment formed with the second and third constant regions of first heavy chain and the second and third constant regions of second heavy chain of an antibody that are bound through a disulfide bond. The Fc fragment of antibody has many different functions, but does not participate in antigen binding.

The term “scFv” refers to a single polypeptide chain comprising VL and VH domains, wherein the VL and VH are connected by a linker (see, for example, Bird et al., Science 242:423-426 (1988); Huston et al., Proc. Natl. Acad. Sci. USA 85:5879-5883 (1988); and Pluckthun, The Pharmacology of Monoclonal Antibodies, Volume 113, edited by Roseburg and Moore, Springer-Verlag, New York, pp. 269-315 (1994)). Such scFv molecule may have a general structure: NH₂-VL-linker-VH-COOH or NH₂-VH-linker-VL-COOH. A suitable linker of prior art consists of a repeated GGGGS (SEQ ID NO:53) amino acid sequence or variants thereof. For example, a linker having amino acid sequence (GGGGS)₄ (SEQ ID NO:54) can be used, but variants thereof can also be used (Holliger et al. (1993), Proc. Natl. Acad. Sci. USA 90:6444-6448). Other linkers that can be used in the present invention are described by Alfthan et al. (1995), Protein Eng. 8:725-731, Choi et al. (2001), Eur. J. Immunol. 31:94-106, Hu et al. (1996), Cancer Res. 56:3055-3061, Kipriyanov et al. (1999), J. Mol. Biol. 293:41-56 and Roovers et al. (2001), Cancer Immunol. In some cases, there may also be a disulfide bond between the VH and VL of scFv. In certain embodiments, the VH and VL domains may be relatively positioned to each other in any suitable arrangement, for example, a scFv comprising NH₂—VH—VH-COOH, NH₂-VL-VL-COOH.

The term “single-domain antibody (sdAb)” has the meaning generally understood by those skilled in the art, which refers to an antibody fragment composed of a single monomer variable antibody domain (e.g., a single heavy chain variable region), which retains the ability to specifically bind the same antigen to which the full-length antibody binds (Holt, L. et al., Trends in Biotechnology, 21 (11): 484-490, 2003). Single-domain antibody is also called as nanobody.

Each of the above antibody fragments maintains the ability to specifically bind to the same antigen to which the full-length antibody binds, and/or competes with the full-length antibody for specific binding to the antigen.

Herein, unless the context clearly dictates otherwise, when the term “antibody” is referred to, it includes not only an intact antibody but also an antigen-binding fragment of the antibody.

The antigen-binding fragment (e.g., the above-mentioned antibody fragment) of antibody can be obtained from a given antibody (e.g., the antibody provided by the present invention) using a conventional technique known to those skilled in the art (e.g., recombinant DNA technology or enzymatic or chemical fragmentation methods), and can be screened for specificity in the same manner by which intact antibodies are screened.

The terms “monoclonal antibody” and “mAb” have the same meaning and are used interchangeably to refer to one antibody molecule from a group of highly homogeneous antibody molecules or a fragment of an antibody, that is, a population of antibody molecules that are identical except for natural mutations that may arise spontaneously. mAbs are highly specific for a single epitope on an antigen. Polyclonal antibodies are relative to monoclonal antibodies, which usually contain at least 2 or more different antibodies, and these different antibodies usually recognize different epitopes on the antigen. In addition, the modifier “monoclonal” only indicates that the antibody is characterized as being obtained from a highly homogeneous antibody population, and should not be construed as requiring that the antibody be prepared by any particular method.

The term “chimeric antibody” refers to an antibody whose light chain and/or heavy chain are partly derived from an antibody (which may be derived from a particular species or belong to a certain a specific antibody class or subclass), and the other part of the light chain or/and heavy chain is derived from another antibody (which may be derived from the same or different species or belong to the same or different antibody class or subclass), but regardless However, it still retains binding activity for the target antigen. For example, the term “chimeric antibody” may include antibodies whose heavy and light chain variable regions are derived from a first antibody (e.g., human) and whose heavy and light chain constant regions are derived from a second antibody (e.g., murine). For example, antibodies produced by immunizing fully human transgenic mice may be referred to as chimeric antibodies, which consist of fully human variable regions and murine constant regions.

The term “murine antibody” refers to an antibody that is obtained by the following method: fusing B cells of immunized mice with myeloma cells, selecting murine hybrid fusion cells that can proliferate indefinitely and secrete the antibody, followed by screening, antibody preparation and antibody purification; or an antibody that is secreted by plasma cells which is formed by differentiation and proliferation of B cells after antigen invades the mouse body.

The term “humanized antibody” refers to a genetically engineered non-human antibody whose amino acid sequence has been modified to increase homology to the sequence of a human antibody. Generally, all or part of the CDR regions of a humanized antibody are derived from a non-human antibody (donor antibody), and all or part of the non-CDR regions (e.g., variable region FRs and/or constant region) are derived from a human immunoglobulin (receptor antibody). Humanized antibodies typically retain the desired properties of the donor antibody, including, but not limited to, antigen specificity, affinity, reactivity, ability to increase immune cell activity, ability to enhance an immune response, and the like. A donor antibody can be an antibody from mouse, rat, rabbit, or non-human primate (e.g., cynomolgus) having desirable properties (e.g., antigen specificity, affinity, reactivity, ability to increase immune cell activity and/or enhance immune response).

The term “identity” is used to refer to the match degree between two polypeptides or between two nucleic acids. When two sequences for comparison have the same monomer sub-unit of base or amino acid at a certain site (e.g., two DNA molecules each have an adenine at a certain site, or two polypeptides each have a lysine at a certain site), the two molecules are identical at the site. The percent identity between two sequences is a function of the number of identical sites shared by the two sequences over the total number of sites for comparison×100. For example, if 6 of 10 sites of two sequences are matched, these two sequences have an identity of 60%. For example, DNA sequences: CTGACT and CAGGTT share an identity of 50% (3 of 6 sites are matched). Generally, the comparison of two sequences is conducted in a manner to produce maximum identity. Such alignment can be conducted by using a computer program such as Align program (DNAstar, Inc.) which is based on the method of Needleman, et al. (J. Mol. Biol. 48:443-453, 1970). The percent identity between two amino acid sequences can also be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percentage of identity between two amino acid sequences can be determined by the algorithm of Needleman and Wunsch (J. Mol. Biol. 48:444-453 (1970)) which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The term “conservative substitution” means an amino acid substitution that does not adversely affect or alter the expected properties of a protein/polypeptide comprising an amino acid sequence. For example, conservative substitutions can be introduced by standard techniques known in the art, such as site-directed mutagenesis and PCR-mediated mutagenesis. Conservative amino acid substitutions include substitutions wherein an amino acid residue is substituted with another amino acid residue having a similar side chain, for example, a residue physically or functionally similar (e.g., having similar size, shape, charge, chemical properties, including ability of forming a covalent bond or a hydrogen bond, etc.) to the corresponding amino acid residue. A family of amino acid residues having similar side chains has been defined in the art. These families include amino acids having basic side chains (e.g., lysine, arginine, and histidine), acidic side chains (e.g., aspartic acid, glutamic acid), uncharged polar side chains (e.g., glycine, asparagine, glutamine, serine, threonine, tyrosine, cysteine, tryptophan), non-polar side chains (e.g. alanine, valine, leucine, isoleucine, valine, phenylalanine, methionine), beta branch side chains (e.g., threonine, valine, isoleucine) and aromatic side chains (e.g., tyrosine, phenylalanine, tryptophan, histidine). Therefore, it is preferred to replace the corresponding amino acid residue with another amino acid residue from the same side chain family. Methods for identifying conservative substitutions of amino acids are well known in the art (see, for example, Brummell et al, Biochem. 32:1180-1187 (1993); Kobayashi et al., Protein Eng. 12 (10): 879-884 (1999); and Burks et al. Proc. Natl Acad. Set USA 94:412-417 (1997), which is incorporated herein by reference).

The twenty conventional amino acids involved herein are expressed in routine manners. See, for example, Immunology-A Synthesis (2nd Edition, E. S. Golub and D. R. Gren, Eds., Sinauer Associates, Sunderland, Mass. (1991)), which is incorporated herein by reference. In the present invention, amino acids are generally represented by single-letter and three-letter abbreviations known in the art. For example, alanine can be represented by A or Ala.

The term “pharmaceutically acceptable carrier and/or excipient” refers to a carrier and/or excipient that is pharmacologically and/or physiologically compatible with the subject and the active ingredient, It is well known in the art (see e.g. Remington's Pharmaceutical Sciences. Edited by Gennaro A R, 19th ed. Pennsylvania: Mack Publishing Company, 1995), and includes but is not limited to: pH adjusters, surfactants, adjuvants, ionic strength enhancers, diluents, agents to maintain osmotic pressure, agents to delay absorption, preservatives. For example, pH adjusting agents include, but are not limited to, phosphate buffers. Surfactants include but are not limited to cationic, anionic or nonionic surfactants such as Tween-80. Ionic strength enhancers include, but are not limited to, sodium chloride. Preservatives include, but are not limited to, various antibacterial and antifungal agents, such as parabens, chlorobutanol, phenol, sorbic acid, and the like. Agents to maintain osmotic pressure include, but are not limited to, sugars, NaCl, and the like. Agents that delay absorption include, but are not limited to, monostearates and gelatin. Diluents include, but are not limited to, water, aqueous buffers (such as buffered saline), alcohols and polyols (such as glycerol), and the like. Preservatives include, but are not limited to, various antibacterial and antifungal agents, such as thimerosal, 2-phenoxyethanol, parabens, chlorobutanol, phenol, sorbic acid, and the like. Stabilizer has the meaning generally understood by those skilled in the art, and it can stabilize the desired activity of the active ingredient in the medicine, including but not limited to sodium glutamate, gelatin, SPGA, sugars (such as sorbitol, mannitol, starch, sucrose, lactose, dextran, or glucose), amino acids (such as glutamic acid, glycine), proteins (such as dry whey, albumin or casein) or their degradation products (such as lactalbumin hydrolyzate), etc.

As used herein, the term “solvate” means a physical association of an ADC disclosed herein with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. A “hydrate” is a solvate wherein the solvent molecule is water.

One or more ADCs disclosed herein may optionally be converted to a solvate. Preparation of solvates is generally known. Thus, for example, M. Caira et al., J. Pharmaceutical Sci., 93 (3), 601-611 (2004) describe the preparation of the solvates of the antifungal fluconazole in ethyl acetate as well as from water. Similar preparations of solvates, hemisolvate, hydrates and the like are described by E. C. van Tonder et al., AAPS PharmSciTechours., 5 (1), article 12 (2004); and A. L. Bingham et al., Chem. Commun., 603-604 (2001). A typical, non-limiting, process involves dissolving the inventive compound in desired amounts of the desired solvent (organic or water or mixtures thereof) at a higher than room temperature, and cooling the solution at a rate sufficient to form crystals which are then isolated by standard methods. Analytical techniques such as, for example IR spectroscopy, show the presence of the solvent (or water) in the crystals as a solvate (or hydrate).

As used herein, the term “pharmaceutically acceptable salt” includes acid addition salts and basic salts.

Exemplary acid addition salts include acetates, ammonium, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (also known as mesylates), naphthalenesulfonates, nitrates, oxalates, phosphates, propionates, salicylates, succinates, sulfates, tartarates, thiocyanates, toluenesulfonates (also known as tosylates), and the like. Additionally, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. 2^{nd} Revised Ed. (2011) Zurich: Wiley-VCH; S. Berge et al., Journal of Pharmaceutical Sciences (1977) 66 (1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference thereto. In one embodiment, an acid salt is an ammonium salt or a di-ammonium salt.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the present disclosure and all acid and base salts are considered equivalent to the free forms of the corresponding compounds for purposes of the present disclosure.

The terms “including”, “comprising”, “having”, “containing” or “involving” and other variations thereof herein are inclusive or open-ended and do not exclude other unlisted elements or steps of methods.

The term “effective amount” refers to an amount sufficient to achieve, or at least partially achieve, the desired effect. For example, an effective amount for preventing a disease (such as a tumor) refers to an amount sufficient to prevent, arrest, or delay the occurrence of a disease (such as a tumor); an effective amount for treating a disease refers to an amount sufficient to cure or at least partially prevent an existing disease. The patient's disease and the amount of its complications. Determining such an effective amount is well within the capability of those skilled in the art. For example, amounts effective for therapeutic use will depend on the severity of the disease to be treated, the general state of the patient's own immune system, the general condition of the patient such as age, weight and sex, the mode of administration of the drug, and other treatments administered concomitantly etc. A therapeutically effective amount of an ADC may vary according to the following factors: The severity of the disease to be treated, the general state of the patient's own immune system, the general condition of the patient such as age, weight and sex, the mode of administration of the drug, and other treatments administered at the same time, etc.

The term “treatment” refers to a method performed to obtain a beneficial or desired clinical result. For the purposes of this invention, a beneficial or desired clinical outcome includes, but is not limited to, relief of symptoms, reduction of the extent of the disease, stabilization (i.e., no longer worsening) of the disease state, delay or slowing of the progression of the disease, amelioration or palliation of the disease status, and relief of symptoms (whether partial or total), whether detectable or not. Additionally, “treating” can also refer to prolonging survival as compared to expected survival if not receiving treatment. Treating or treatment may be therapeutic or prophylactic.

As used herein, the term “subject” refers to a mammal, such as a primate mammal, such as a human. In certain embodiments, the subject (e.g., a human) has a tumor, or is at risk of having a disease as described above.

The terms “cancer” and “tumor” are used interchangeably and refer to a broad class of diseases characterized by the uncontrolled growth of abnormal cells in the body. Unregulated cell division may lead to the formation of malignant tumors or cells that invade adjacent tissues and may metastasize to distant sites in the body through the lymphatic system or bloodstream. Cancer includes benign and malignant cancers as well as dormant tumors or micrometastases. Cancer also includes hematological malignancies.

The term “hematologic malignancies” includes lymphomas, leukemias, myelomas or lymphoid malignancies, as well as splenic and lymph node neoplasms. Exemplary lymphomas include B-cell lymphoma and T-cell lymphoma. B-cell lymphomas, including, for example, Hodgkin's lymphoma. T-cell lymphomas, including, for example, cutaneous T-cell lymphomas. Hematological malignancies also include leukemias, such as secondary leukemia or acute lymphoblastic leukemia. Hematological malignancies also include myeloma (e.g., multiple myeloma) and other hematological and/or B- or T-cell-related cancers. The term “alkyl” refers to a group obtained by losing one hydrogen atom from a linear or branched hydrocarbon group, such as “C₁₋₂₀ alkyl”, “C₁₋₁₀ alkyl”, “C₁₋₆ alkyl”, “C₁₋₄ alkyl”, “C₁₋₃ alkyl”, etc., and specific examples thereof include but are not limited to: methyl, ethyl, n-propyl, isopropyl, n-butyl, isobutyl, sec-butyl, tert-butyl, n-pentyl, isopentyl, 2-methylbutyl, neopentyl, 1-ethylpropyl, n-hexyl, isohexyl, 3-methylpentyl, 2-methylpentyl, 1-methylpentyl, 3,3-dimethylbutyl, 2,2-dimethylbutyl, 1,1-dimethylbutyl, 1,2-dimethylbutyl, 1,3-dimethylbutyl, 2,3-dimethylbutyl, 2-ethylbutyl, 1,2-dimethylpropyl, etc.

The term “alkylene” refers to a group obtained by losing two hydrogen atoms from a linear or branched hydrocarbon group, such as “C₁₋₂₀ alkylene”, “C₁₋₁₀ alkylene”, “C₃₋₁₀ alkylene”, “C₅₋₈ alkylene”, “C₁₋₆ alkylene”, “C₁₋₄ alkylene”, “C₁₋₃ alkylene”, etc., and specific examples thereof include but are not limited to: methylene, ethylene, 1,3-propylene, 1,4-butylene, 1,5-pentylene or 1,6-hexylene, etc.

The term “alkenylene” refers to a divalent group obtained by losing two hydrogen atoms from a linear or branched hydrocarbon group containing at least one carbon-carbon double bond, including, for example, “C₂₋₂₀ alkenylene”, “C₃₋₁₀ alkenylene”, “C₅₋₈ alkenylene”, etc., and examples thereof include, but not limited to: ethenylene, 1-propenylene, 2-propenylene, 1-butenylene, 2-butenylene, 1,3-butadienylene, 1-pentenylene, 2-pentenylene, 3-pentenylene, 1,3-pentadienylene, 1,4-pentadienylene, 1-hexenylene, 2-hexenylene, 3-hexenylene, 1,4-hexadienylene, etc.

The term “alkynylene” refers to a divalent group obtained by losing two hydrogen atoms from a linear or branched hydrocarbon group containing at least one carbon-carbon triple bond, including, for example, “C₂₋₂₀ alkynylene”, “C₃₋₁₀ alkynylene”, “C₅₋₈ alkynylene”, etc., and examples thereof include, but are not limited to: ethynylene, 1-propynylene, 2-propynylene, 1-butynylene, 2-butynylene, 1,3-butynylene, 1-pentynylene, 2-pentynylene, 3-pentynylene, 1,3-pentynylene, 1,4-pentynylene, 1-hexynylene, 2-hexynylene, 3-hexynylene, 1,4-hexadiynylene, etc.

The term “heteroaliphatic ring” refers to a saturated or partially saturated cyclic structure containing at least one ring member selected from the group consisting of N, O and S. Specific examples thereof include, but not limited to, 5- to 6-membered heteroaliphatic ring, 5- to 6-membered nitrogen-containing heteroaliphatic ring, 5- to 6-membered oxygen-containing heteroaliphatic ring, etc., for example, tetrahydrofuran, pyrrolidine, piperidine, tetrahydropyran, etc.

The term “heteroaromatic ring” refers to an aromatic ring structure containing at least one ring member selected from the group consisting of N, O and S. Specific examples thereof include but are not limited to 5- to 6-membered heteroaromatic ring, 5- to 6-membered nitrogen-containing heteroaromatic ring, 5- to 6-membered oxygen-containing heteroaromatic rings, etc., for example, furan, thiophene, pyrrole, thiazole, isothiazole, thiadiazole, oxazole, isoxazole, oxadiazole, imidazole, pyrazole, 1,2,3-triazole, 1,2,4-triazole, 1,2,3-oxadiazole, 1,2,4-oxadiazole, 1,2,5-oxadiazole, 1,3,4-oxadiazole, pyridine, pyrimidine, pyridazine, pyrazine, 1,2,3-triazine, 1,3,5-triazine, 1,2,4,5-tetrazine, etc.

The term “aromatic ring system” refers to a monocyclic or polycyclic ring system comprising at least one aromatic ring (e.g., benzene ring, etc.) or heteroaromatic ring (e.g., pyrimidine ring, etc.), wherein two or more aromatic rings and/or heteroaromatic rings can form a fused ring or be connected by a single bond (e.g., bipyrimidinylphenyl, etc.), and the aromatic ring system can be divalent or multi-valent (e.g., trivalent or tetravalent), for example, 5- to 20-membered aromatic ring system.

As used herein, the term “about” or “approximately” when used in conjunction with a numerical variable generally means that the value of the variable is within experimental error (e.g., within a 95% confidence interval for the mean) or within ±10% or wider range.

It should be noted that if there is a discrepancy between a depicted structure and a name for that structure, the depicted structure is to be accorded more weight.

The present application relates to an antibody-drug conjugate (ADC) for the treatment of a HER3-positive cancer, and exemplarily discloses an antibody-drug conjugate that has a structure as shown in the general Formula Ab-[M-L-E-D]ₓ and uses the fully human antibody 22B6D2-hIgG1 as the targeting moiety. The ADCs (or conjugate) of the present invention have an average drug-to-antibody ratio (DAR) that can be as high as 7.99 and demonstrate targeted killing effect on a HER3-positive cancer, such as colon cancer, gastric cancer, breast cancer, lung cancer (e.g., non-small cell lung cancer, specifically lung adenocarcinoma). The results herein show that the conjugate has a better drug-to-antibody ratio, and the conjugate has excellent binding activity to HER3-positive cells, and has very good targeted killing effect on a HER3-positive cancer, such as colon cancer, gastric cancer, breast cancer, lung cancer (e.g., non-small cell lung cancer, specifically lung adenocarcinoma). Therefore, the present application provides an antibody-drug conjugate for treating a cancer with high expression of HER3, a pharmaceutical composition comprising the antibody-drug conjugate, and application thereof in the treatment of a cancer high expression of HER3.

### Antibody-Drug Conjugates

In one aspect, the present application provides an antibody-drug conjugate, which has a structure as shown in formula Ab-[M-L-E-D]ₓ, wherein:
Ab is an antibody or antigen-binding fragment thereof that specifically binds to human epidermal growth factor receptor 3 (HER3, also known as Erbb3);
M is a joint site to bind the antibody or antigen-binding fragment thereof;
L is a connector that connects the joint site M and E;
E is a fragment connecting L and D;
D is a fragment of a cytotoxic drug;
x is selected from 1 to 10.

In some embodiments, the antibody or antigen-binding fragment thereof comprises:
(1) a heavy chain variable region (VH) and/or a light chain variable region (VL) as follows, wherein the CDRs are defined by the Chothia numbering system:
(1a) a heavy chain variable region (VH) comprising the following 3 CDRs: CDR-H1 having a sequence as set forth in SEQ ID NO: 1 or a variant thereof, CDR-H2 having a sequence as set forth in SEQ ID NO: 2 or a variant thereof, CDR-H3 having a sequence as set forth in SEQ ID NO: 3 or a variant thereof; and/or, a light chain variable region (VL) comprising the following 3 CDRs: CDR-L1 having a sequence as set forth in SEQ ID NO: 4 or a variant thereof, CDR-L2 having a sequence as set forth in SEQ ID NO: 5 or a variant thereof, CDR-L3 having a sequence as set forth in SEQ ID NO: 6 or a variant thereof; or,
(1b) a heavy chain variable region (VH) comprising the following 3 CDRs: CDR-H1 having a sequence as set forth in SEQ ID NO: 19 or a variant thereof, CDR-H2 having a sequence as set forth in SEQ ID NO: 20 or a variant thereof, CDR-H3 having a sequence as set forth in SEQ ID NO: 21 or a variant thereof; and/or, a light chain variable region (VL) comprising the following 3 CDRs: CDR-L1 having a sequence as set forth in SEQ ID NO: 22 or a variant thereof, CDR-L2 having a sequence as set forth in SEQ ID NO: 23 or a variant thereof, CDR-L3 having a sequence as set forth in SEQ ID NO: 24 or a variant thereof; or,
(1c) a heavy chain variable region (VH) comprising the following 3 CDRs: CDR-H1 having a sequence as set forth in SEQ ID NO: 36 or a variant thereof, CDR-H2 having a sequence as set forth in SEQ ID NO: 37 or a variant thereof, CDR-H3 having a sequence as set forth in SEQ ID NO: 38 or a variant thereof; and/or, a light chain variable region (VL) comprising the following 3 CDRs: CDR-L1 having a sequence as set forth in SEQ ID NO: 45 or a variant thereof, CDR-L2 having a sequence as set forth in SEQ ID NO: 23 or a variant thereof, and CDR-L3 having a sequence as set forth in SEQ ID NO: 52 or a variant thereof;
wherein, the variant described in any item of (1a), (1b), and (1c) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2 or 3 amino acids) as compared to the sequence from which it is derived; preferably, the substitution is a conservative substitution with the proviso that the amino acid sequences of the CDRs of the variant have 100% sequence identity to the amino acid sequences of the respective CDRs of the VH and VL of (1a), (1b), and (1c) and the variant binds HER3;
or,
(2) a heavy chain variable region (VH) and/or a light chain variable region (VL) as follows, wherein the CDRs are defined according to the Kabat numbering system:
(2a) a heavy chain variable region (VH) comprising the following 3 CDRs: CDR-H1 having a sequence as set forth in SEQ ID NO: 7 or a variant thereof, CDR-H2 having a sequence as set forth in SEQ ID NO: 8 or a variant thereof, CDR-H3 having a sequence as set forth in SEQ ID NO: 9 or a variant thereof; and/or, a light chain variable region (VL) comprising the following 3 CDRs: CDR-L1 having a sequence as set forth in SEQ ID NO: 4 or a variant thereof, CDR-L2 having a sequence as set forth in SEQ ID NO: 5 or a variant thereof, CDR-L3 having a sequence as set forth in SEQ ID NO: 6 or a variant thereof; or,
(2b) a heavy chain variable region (VH) comprising the following 3 CDRs: CDR-H1 having a sequence as set forth in SEQ ID NO: 25 or a variant thereof, CDR-H2 having a sequence as set forth in SEQ ID NO: 26 or a variant thereof, CDR-H3 having a sequence as set forth in SEQ ID NO: 21 or a variant thereof; and/or, a light chain variable region (VL) comprising the following 3 CDRs: CDR-L1 having a sequence as set forth in SEQ ID NO: 22 or a variant thereof, CDR-L2 having a sequence as set forth in SEQ ID NO: 23 or a variant thereof, CDR-L3 having a sequence as set forth in SEQ ID NO: 24 or a variant thereof; or,
(2c) a heavy chain variable region (VH) comprising the following 3 CDRs: CDR-H1 having a sequence as set forth in SEQ ID NO: 39 or a variant thereof, CDR-H2 having a sequence as set forth in SEQ ID NO: 40 or a variant thereof, CDR-H3 having a sequence as set forth in SEQ ID NO: 38 or a variant thereof; and/or, a light chain variable region (VL) comprising the following 3 CDRs: CDR-L1 having a sequence as set forth in SEQ ID NO: 45 or a variant thereof, CDR-L2 having a sequence as set forth in SEQ ID NO: 23 or a variant thereof, and CDR-L3 having a sequence as set forth in SEQ ID NO: 52 or a variant thereof;
wherein, the variant described in any item of (2a), (2b), and (2c) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2 or 3 amino acids) as compared to the sequence from which it is derived; preferably, the substitution is a conservative substitution with the proviso that the amino acid sequences of the CDRs of the variant have 100% sequence identity to the amino acid sequences of the respective CDRs of the VH and VL of (2a), (2b), and (2c) and the variant binds HER3;
or,
(3) a heavy chain variable region (VH) and/or a light chain variable region (VL) as follows, wherein the CDRs are defined by the IMGT numbering system:
(3a) a heavy chain variable region (VH) comprising the following 3 CDRs: CDR-H1 having a sequence as set forth in SEQ ID NO: 10 or a variant thereof, CDR-H2 having a sequence as set forth in SEQ ID NO: 11 or a variant thereof, CDR-H3 having a sequence as set forth in SEQ ID NO: 12 or a variant thereof; and/or, a light chain variable region (VL) comprising the following 3 CDRs: CDR-L1 having a sequence as set forth in SEQ ID NO: 13 or a variant thereof, CDR-L2 having a sequence as set forth in SEQ ID NO: 14 or a variant thereof, CDR-L3 having a sequence as set forth in SEQ ID NO: 6 or a variant thereof; or,
(3b) a heavy chain variable region (VH) comprising the following 3 CDRs: CDR-H1 having a sequence as set forth in SEQ ID NO: 27 or a variant thereof, CDR-H2 having a sequence as set forth in SEQ ID NO: 28 or a variant thereof, CDR-H3 having a sequence as set forth in SEQ ID NO: 29 or a variant thereof; and/or, a light chain variable region (VL) comprising the following 3 CDRs: CDR-L1 having a sequence as set forth in SEQ ID NO: 30 or a variant thereof, CDR-L2 having a sequence as set forth in SEQ ID NO: 31 or a variant thereof, CDR-L3 having a sequence as set forth in SEQ ID NO: 24 or a variant thereof; or,
(3c) a heavy chain variable region (VH) comprising the following 3 CDRs: CDR-H1 having a sequence as set forth in SEQ ID NO: 41 or a variant thereof, CDR-H2 having a sequence as set forth in SEQ ID NO: 42 or a variant thereof, CDR-H3 having a sequence as set forth in SEQ ID NO: 43 or a variant thereof; and/or, a light chain variable region (VL) comprising the following 3 CDRs: CDR-L1 having a sequence as set forth in SEQ ID NO: 44 or a variant thereof, CDR-L2 having a sequence as set forth in SEQ ID NO: 31 or a variant thereof, and CDR-L3 having a sequence as set forth in SEQ ID NO: 52 or a variant thereof;
wherein, the variant described in any item of (3a), (3b), and (3c) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2 or 3 amino acids) as compared to the sequence from which it is derived; preferably, the substitution is a conservative substitution with the proviso that the amino acid sequences of the CDRs of the variant have 100% sequence identity to the amino acid sequences of the respective CDRs of the VH and VL of (3a), (3b), and (3c) and the variant binds HER3.

In some embodiments, the antibody or antigen-binding fragment thereof comprises:
(1) a heavy chain variable region (VH) and/or a light chain variable region (VL) as follows, wherein the CDRs are defined by the Chothia numbering system:
(1a) a heavy chain variable region (VH) comprising the following 3 CDRs: CDR-H1 having the amino acid sequence as set forth in SEQ ID NO: 1 or a variant thereof, CDR-H2 having the amino acid sequence as set forth in SEQ ID NO: 2 or a variant thereof, CDR-H3 having the amino acid sequence as set forth in SEQ ID NO: 3 or a variant thereof; and/or, a light chain variable region (VL) comprising the following 3 CDRs: CDR-L1 having the amino acid sequence as set forth in SEQ ID NO: 4 or a variant thereof, CDR-L2 having the amino acid sequence as set forth in SEQ ID NO: 5 or a variant thereof, CDR-L3 having the amino acid sequence as set forth in SEQ ID NO: 6 or a variant thereof; or,
(1b) a heavy chain variable region (VH) comprising the following 3 CDRs: CDR-H1 having the amino acid sequence as set forth in SEQ ID NO: 19 or a variant thereof, CDR-H2 having the amino acid sequence as set forth in SEQ ID NO: 20 or a variant thereof, CDR-H3 having the amino acid sequence as set forth in SEQ ID NO: 21 or a variant thereof; and/or, a light chain variable region (VL) comprising the following 3 CDRs: CDR-L1 having the amino acid sequence as set forth in SEQ ID NO: 22 or a variant thereof, CDR-L2 having the amino acid sequence as set forth in SEQ ID NO: 23 or a variant thereof, CDR-L3 having the amino acid sequence as set forth in SEQ ID NO: 24 or a variant thereof; or,
(1c) a heavy chain variable region (VH) comprising the following 3 CDRs: CDR-H1 having the amino acid sequence as set forth in SEQ ID NO: 36 or a variant thereof, CDR-H2 having the amino acid sequence as set forth in SEQ ID NO: 37 or a variant thereof, CDR-H3 having the amino acid sequence as set forth in SEQ ID NO: 38 or a variant thereof; and/or, a light chain variable region (VL) comprising the following 3 CDRs: CDR-L1 having the amino acid sequence as set forth in SEQ ID NO: 45 or a variant thereof, CDR-L2 having the amino acid sequence as set forth in SEQ ID NO: 23 or a variant thereof, and CDR-L3 having the amino acid sequence as set forth in SEQ ID NO: 52 or a variant thereof;
wherein, the variant described in any item of (1a), (1b), and (1c) has the amino acid sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2 or 3 amino acids) as compared to the sequence from which it is derived; preferably, the substitution is a conservative substitution;
or,
(2) a heavy chain variable region (VH) and/or a light chain variable region (VL) as follows, wherein the CDRs are defined according to the Kabat numbering system:
(2a) a heavy chain variable region (VH) comprising the following 3 CDRs: CDR-H1 having the amino acid sequence as set forth in SEQ ID NO: 7 or a variant thereof, CDR-H2 having the amino acid sequence as set forth in SEQ ID NO: 8 or a variant thereof, CDR-H3 having the amino acid sequence as set forth in SEQ ID NO: 9 or a variant thereof; and/or, a light chain variable region (VL) comprising the following 3 CDRs: CDR-L1 having the amino acid sequence as set forth in SEQ ID NO: 4 or a variant thereof, CDR-L2 having the amino acid sequence as set forth in SEQ ID NO: 5 or a variant thereof, CDR-L3 having the amino acid sequence as set forth in SEQ ID NO: 6 or a variant thereof; or,
(2b) a heavy chain variable region (VH) comprising the following 3 CDRs: CDR-H1 having the amino acid sequence as set forth in SEQ ID NO: 25 or a variant thereof, CDR-H2 having the amino acid sequence as set forth in SEQ ID NO: 26 or a variant thereof, CDR-H3 having the amino acid sequence as set forth in SEQ ID NO: 21 or a variant thereof; and/or, a light chain variable region (VL) comprising the following 3 CDRs: CDR-L1 having the amino acid sequence as set forth in SEQ ID NO: 22 or a variant thereof, CDR-L2 having the amino acid sequence as set forth in SEQ ID NO: 23 or a variant thereof, CDR-L3 having the amino acid sequence as set forth in SEQ ID NO: 24 or a variant thereof; or,
(2c) a heavy chain variable region (VH) comprising the following 3 CDRs: CDR-H1 having the amino acid sequence as set forth in SEQ ID NO: 39 or a variant thereof, CDR-H2 having the amino acid sequence as set forth in SEQ ID NO: 40 or a variant thereof, CDR-H3 having the amino acid sequence as set forth in SEQ ID NO: 38 or a variant thereof; and/or, a light chain variable region (VL) comprising the following 3 CDRs: CDR-L1 having the amino acid sequence as set forth in SEQ ID NO: 45 or a variant thereof, CDR-L2 having the amino acid sequence as set forth in SEQ ID NO: 23 or a variant thereof, and CDR-L3 having the amino acid sequence as set forth in SEQ ID NO: 52 or a variant thereof;
wherein, the variant described in any item of (2a), (2b), and (2c) has the amino acid sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2 or 3 amino acids) as compared to the sequence from which it is derived; preferably, the substitution is a conservative substitution;
or,
(3) a heavy chain variable region (VH) and/or a light chain variable region (VL) as follows, wherein the CDRs are defined by the IMGT numbering system:
(3a) a heavy chain variable region (VH) comprising the following 3 CDRs: CDR-H1 having the amino acid sequence as set forth in SEQ ID NO: 10 or a variant thereof, CDR-H2 having the amino acid sequence as set forth in SEQ ID NO: 11 or a variant thereof, CDR-H3 having the amino acid sequence as set forth in SEQ ID NO: 12 or a variant thereof; and/or, a light chain variable region (VL) comprising the following 3 CDRs: CDR-L1 having the amino acid sequence as set forth in SEQ ID NO: 13 or a variant thereof, CDR-L2 having the amino acid sequence as set forth in SEQ ID NO: 14 or a variant thereof, CDR-L3 having the amino acid sequence as set forth in SEQ ID NO: 6 or a variant thereof; or,
(3b) a heavy chain variable region (VH) comprising the following 3 CDRs: CDR-H1 having the amino acid sequence as set forth in SEQ ID NO: 27 or a variant thereof, CDR-H2 having the amino acid sequence as set forth in SEQ ID NO: 28 or a variant thereof, CDR-H3 having the amino acid sequence as set forth in SEQ ID NO: 29 or a variant thereof; and/or, a light chain variable region (VL) comprising the following 3 CDRs: CDR-L1 having the amino acid sequence as set forth in SEQ ID NO: 30 or a variant thereof, CDR-L2 having the amino acid sequence as set forth in SEQ ID NO: 31 or a variant thereof, CDR-L3 having the amino acid sequence as set forth in SEQ ID NO: 24 or a variant thereof; or,
(3c) a heavy chain variable region (VH) comprising the following 3 CDRs: CDR-H1 having the amino acid sequence as set forth in SEQ ID NO: 41 or a variant thereof, CDR-H2 having the amino acid sequence as set forth in SEQ ID NO: 42 or a variant thereof, CDR-H3 having the amino acid sequence as set forth in SEQ ID NO: 43 or a variant thereof; and/or, a light chain variable region (VL) comprising the following 3 CDRs: CDR-L1 having the amino acid sequence as set forth in SEQ ID NO: 44 or a variant thereof, CDR-L2 having the amino acid sequence as set forth in SEQ ID NO: 31 or a variant thereof, and CDR-L3 having the amino acid sequence as set forth in SEQ ID NO: 52 or a variant thereof;
wherein, the variant described in any item of (3a), (3b), and (3c) has the amino acid sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2 or 3 amino acids) as compared to the sequence from which it is derived; preferably, the substitution is a conservative substitution.

In some embodiments, the antibody or antigen-binding fragment thereof comprises:
(1a) a heavy chain variable region (VH) comprising the following 3 CDRs: CDR-H1 having the amino acid sequence as set forth in SEQ ID NO: 1 or a variant thereof, CDR-H2 having the amino acid sequence as set forth in SEQ ID NO: 2 or a variant thereof, CDR-H3 having the amino acid sequence as set forth in SEQ ID NO: 3 or a variant thereof; and/or, a light chain variable region (VL) comprising the following 3 CDRs: CDR-L1 having the amino acid sequence as set forth in SEQ ID NO: 4 or a variant thereof, CDR-L2 having the amino acid sequence as set forth in SEQ ID NO: 5 or a variant thereof, CDR-L3 having the amino acid sequence as set forth in SEQ ID NO: 6 or a variant thereof; or,
(1b) a heavy chain variable region (VH) comprising the following 3 CDRs: CDR-H1 having the amino acid sequence as set forth in SEQ ID NO: 19 or a variant thereof, CDR-H2 having the amino acid sequence as set forth in SEQ ID NO: 20 or a variant thereof, CDR-H3 having the amino acid sequence as set forth in SEQ ID NO: 21 or a variant thereof; and/or, a light chain variable region (VL) comprising the following 3 CDRs: CDR-L1 having the amino acid sequence as set forth in SEQ ID NO: 22 or a variant thereof, CDR-L2 having the amino acid sequence as set forth in SEQ ID NO: 23 or a variant thereof, CDR-L3 having the amino acid sequence as set forth in SEQ ID NO: 24 or a variant thereof; or,
(1c) a heavy chain variable region (VH) comprising the following 3 CDRs: CDR-H1 having the amino acid sequence as set forth in SEQ ID NO: 36 or a variant thereof, CDR-H2 having the amino acid sequence as set forth in SEQ ID NO: 37 or a variant thereof, CDR-H3 having the amino acid sequence as set forth in SEQ ID NO: 38 or a variant thereof; and/or, a light chain variable region (VL) comprising the following 3 CDRs: CDR-L1 having the amino acid sequence as set forth in SEQ ID NO: 45 or a variant thereof, CDR-L2 having the amino acid sequence as set forth in SEQ ID NO: 23 or a variant thereof, and CDR-L3 having the amino acid sequence as set forth in SEQ ID NO: 52 or a variant thereof;
wherein, the variant described in any item of (1a), (1b), and (1c) has the amino acid sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2 or 3 amino acids) as compared to the sequence from which it is derived; preferably, the substitution is a conservative substitution;
or,
(2a) a heavy chain variable region (VH) comprising the following 3 CDRs: CDR-H1 having the amino acid sequence as set forth in SEQ ID NO: 7 or a variant thereof, CDR-H2 having the amino acid sequence as set forth in SEQ ID NO: 8 or a variant thereof, CDR-H3 having the amino acid sequence as set forth in SEQ ID NO: 9 or a variant thereof; and/or, a light chain variable region (VL) comprising the following 3 CDRs: CDR-L1 having the amino acid sequence as set forth in SEQ ID NO: 4 or a variant thereof, CDR-L2 having the amino acid sequence as set forth in SEQ ID NO: 5 or a variant thereof, CDR-L3 having the amino acid sequence as set forth in SEQ ID NO: 6 or a variant thereof; or,
(2b) a heavy chain variable region (VH) comprising the following 3 CDRs: CDR-H1 having the amino acid sequence as set forth in SEQ ID NO: 25 or a variant thereof, CDR-H2 having the amino acid sequence as set forth in SEQ ID NO: 26 or a variant thereof, CDR-H3 having the amino acid sequence as set forth in SEQ ID NO: 21 or a variant thereof; and/or, a light chain variable region (VL) comprising the following 3 CDRs: CDR-L1 having the amino acid sequence as set forth in SEQ ID NO: 22 or a variant thereof, CDR-L2 having the amino acid sequence as set forth in SEQ ID NO: 23 or a variant thereof, CDR-L3 having the amino acid sequence as set forth in SEQ ID NO: 24 or a variant thereof; or,
(2c) a heavy chain variable region (VH) comprising the following 3 CDRs: CDR-H1 having the amino acid sequence as set forth in SEQ ID NO: 39 or a variant thereof, CDR-H2 having the amino acid sequence as set forth in SEQ ID NO: 40 or a variant thereof, CDR-H3 having the amino acid sequence as set forth in SEQ ID NO: 38 or a variant thereof; and/or, a light chain variable region (VL) comprising the following 3 CDRs: CDR-L1 having the amino acid sequence as set forth in SEQ ID NO: 45 or a variant thereof, CDR-L2 having the amino acid sequence as set forth in SEQ ID NO: 23 or a variant thereof, and CDR-L3 having the amino acid sequence as set forth in SEQ ID NO: 52 or a variant thereof;
wherein, the variant described in any item of (2a), (2b), and (2c) has the amino acid sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2 or 3 amino acids) as compared to the sequence from which it is derived; preferably, the substitution is a conservative substitution;
or,
(3a) a heavy chain variable region (VH) comprising the following 3 CDRs: CDR-H1 having the amino acid sequence as set forth in SEQ ID NO: 10 or a variant thereof, CDR-H2 having the amino acid sequence as set forth in SEQ ID NO: 11 or a variant thereof, CDR-H3 having the amino acid sequence as set forth in SEQ ID NO: 12 or a variant thereof; and/or, a light chain variable region (VL) comprising the following 3 CDRs: CDR-L1 having the amino acid sequence as set forth in SEQ ID NO: 13 or a variant thereof, CDR-L2 having the amino acid sequence as set forth in SEQ ID NO: 14 or a variant thereof, CDR-L3 having the amino acid sequence as set forth in SEQ ID NO: 6 or a variant thereof; or,
(3b) a heavy chain variable region (VH) comprising the following 3 CDRs: CDR-H1 having the amino acid sequence as set forth in SEQ ID NO: 27 or a variant thereof, CDR-H2 having the amino acid sequence as set forth in SEQ ID NO: 28 or a variant thereof, CDR-H3 having the amino acid sequence as set forth in SEQ ID NO: 29 or a variant thereof; and/or, a light chain variable region (VL) comprising the following 3 CDRs: CDR-L1 having the amino acid sequence as set forth in SEQ ID NO: 30 or a variant thereof, CDR-L2 having the amino acid sequence as set forth in SEQ ID NO: 31 or a variant thereof, CDR-L3 having the amino acid sequence as set forth in SEQ ID NO: 24 or a variant thereof; or,
(3c) a heavy chain variable region (VH) comprising the following 3 CDRs: CDR-H1 having the amino acid sequence as set forth in SEQ ID NO: 41 or a variant thereof, CDR-H2 having the amino acid sequence as set forth in SEQ ID NO: 42 or a variant thereof, CDR-H3 having the amino acid sequence as set forth in SEQ ID NO: 43 or a variant thereof; and/or, a light chain variable region (VL) comprising the following 3 CDRs: CDR-L1 having the amino acid sequence as set forth in SEQ ID NO: 44 or a variant thereof, CDR-L2 having the amino acid sequence as set forth in SEQ ID NO: 31 or a variant thereof, and CDR-L3 having the amino acid sequence as set forth in SEQ ID NO: 52 or a variant thereof;
wherein, the variant described in any item of (3a), (3b), and (3c) has the amino acid sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2 or 3 amino acids) as compared to the sequence from which it is derived; preferably, the substitution is a conservative substitution.

In some embodiments, the antibody or antigen-binding fragment thereof comprises:
(1a) a heavy chain variable region (VH) comprising the following 3 CDRs: CDR-H1 having the amino acid sequence as set forth in SEQ ID NO: 1, CDR-H2 having the amino acid sequence as set forth in SEQ ID NO: 2, CDR-H3 having the amino acid sequence as set forth in SEQ ID NO: 3; and/or, a light chain variable region (VL) comprising the following 3 CDRs: CDR-L1 having the amino acid sequence as set forth in SEQ ID NO: 4, CDR-L2 having the amino acid sequence as set forth in SEQ ID NO: 5, CDR-L3 having the amino acid sequence as set forth in SEQ ID NO: 6; or,
(1b) a heavy chain variable region (VH) comprising the following 3 CDRs: CDR-H1 having the amino acid sequence as set forth in SEQ ID NO: 19, CDR-H2 having the amino acid sequence as set forth in SEQ ID NO: 20, CDR-H3 having the amino acid sequence as set forth in SEQ ID NO: 21; and/or, a light chain variable region (VL) comprising the following 3 CDRs: CDR-L1 having the amino acid sequence as set forth in SEQ ID NO: 22, CDR-L2 having the amino acid sequence as set forth in SEQ ID NO: 23, CDR-L3 having the amino acid sequence as set forth in SEQ ID NO: 24; or,
(1c) a heavy chain variable region (VH) comprising the following 3 CDRs: CDR-H1 having the amino acid sequence as set forth in SEQ ID NO: 36, CDR-H2 having the amino acid sequence as set forth in SEQ ID NO: 37, CDR-H3 having the amino acid sequence as set forth in SEQ ID NO: 38; and/or, a light chain variable region (VL) comprising the following 3 CDRs: CDR-L1 having the amino acid sequence as set forth in SEQ ID NO: 45, CDR-L2 having the amino acid sequence as set forth in SEQ ID NO: 23, and CDR-L3 having the amino acid sequence as set forth in SEQ ID NO: 52;
or,
(2a) a heavy chain variable region (VH) comprising the following 3 CDRs: CDR-H1 having the amino acid sequence as set forth in SEQ ID NO: 7, CDR-H2 having the amino acid sequence as set forth in SEQ ID NO: 8, CDR-H3 having the amino acid sequence as set forth in SEQ ID NO: 9; and/or, a light chain variable region (VL) comprising the following 3 CDRs: CDR-L1 having the amino acid sequence as set forth in SEQ ID NO: 4, CDR-L2 having the amino acid sequence as set forth in SEQ ID NO: 5, CDR-L3 having the amino acid sequence as set forth in SEQ ID NO: 6; or,
(2b) a heavy chain variable region (VH) comprising the following 3 CDRs: CDR-H1 having the amino acid sequence as set forth in SEQ ID NO: 25, CDR-H2 having the amino acid sequence as set forth in SEQ ID NO: 26, CDR-H3 having the amino acid sequence as set forth in SEQ ID NO: 21; and/or, a light chain variable region (VL) comprising the following 3 CDRs: CDR-L1 having the amino acid sequence as set forth in SEQ ID NO: 22, CDR-L2 having the amino acid sequence as set forth in SEQ ID NO: 23, CDR-L3 having the amino acid sequence as set forth in SEQ ID NO: 24; or,
(2c) a heavy chain variable region (VH) comprising the following 3 CDRs: CDR-H1 having the amino acid sequence as set forth in SEQ ID NO: 39, CDR-H2 having the amino acid sequence as set forth in SEQ ID NO: 40, CDR-H3 having the amino acid sequence as set forth in SEQ ID NO: 38; and/or, a light chain variable region (VL) comprising the following 3 CDRs: CDR-L1 having the amino acid sequence as set forth in SEQ ID NO: 45, CDR-L2 having the amino acid sequence as set forth in SEQ ID NO: 23, and CDR-L3 having the amino acid sequence as set forth in SEQ ID NO: 52;
or,
(3a) a heavy chain variable region (VH) comprising the following 3 CDRs: CDR-H1 having the amino acid sequence as set forth in SEQ ID NO: 10, CDR-H2 having the amino acid sequence as set forth in SEQ ID NO: 11, CDR-H3 having the amino acid sequence as set forth in SEQ ID NO: 12; and/or, a light chain variable region (VL) comprising the following 3 CDRs: CDR-L1 having the amino acid sequence as set forth in SEQ ID NO: 13, CDR-L2 having the amino acid sequence as set forth in SEQ ID NO: 14, CDR-L3 having the amino acid sequence as set forth in SEQ ID NO: 6; or,
(3b) a heavy chain variable region (VH) comprising the following 3 CDRs: CDR-H1 having the amino acid sequence as set forth in SEQ ID NO: 27, CDR-H2 having the amino acid sequence as set forth in SEQ ID NO: 28, CDR-H3 having the amino acid sequence as set forth in SEQ ID NO: 29; and/or, a light chain variable region (VL) comprising the following 3 CDRs: CDR-L1 having the amino acid sequence as set forth in SEQ ID NO: 30, CDR-L2 having the amino acid sequence as set forth in SEQ ID NO: 31, CDR-L3 having the amino acid sequence as set forth in SEQ ID NO: 24; or,
(3c) a heavy chain variable region (VH) comprising the following 3 CDRs: CDR-H1 having the amino acid sequence as set forth in SEQ ID NO: 41, CDR-H2 having the amino acid sequence as set forth in SEQ ID NO: 42, CDR-H3 having the amino acid sequence as set forth in SEQ ID NO: 43; and/or, a light chain variable region (VL) comprising the following 3 CDRs: CDR-L1 having the amino acid sequence as set forth in SEQ ID NO: 44, CDR-L2 having the amino acid sequence as set forth in SEQ ID NO: 31, and CDR-L3 having the amino acid sequence as set forth in SEQ ID NO: 52.

In some embodiments, the antibody or antigen-binding fragment thereof comprises: (1a) a heavy chain variable region (VH) comprising the following 3 CDRs: CDR-H1 having the amino acid sequence as set forth in SEQ ID NO: 1, CDR-H2 having the amino acid sequence as set forth in SEQ ID NO: 2, CDR-H3 having the amino acid sequence as set forth in SEQ ID NO: 3; and/or, a light chain variable region (VL) comprising the following 3 CDRs: CDR-L1 having the amino acid sequence as set forth in SEQ ID NO: 4, CDR-L2 having the amino acid sequence as set forth in SEQ ID NO: 5, CDR-L3 having the amino acid sequence as set forth in SEQ ID NO: 6.

In some embodiments, the antibody or antigen-binding fragment thereof comprises: (1b) a heavy chain variable region (VH) comprising the following 3 CDRs: CDR-H1 having the amino acid sequence as set forth in SEQ ID NO: 19, CDR-H2 having the amino acid sequence as set forth in SEQ ID NO: 20, CDR-H3 having the amino acid sequence as set forth in SEQ ID NO: 21; and/or, a light chain variable region (VL) comprising the following 3 CDRs: CDR-L1 having the amino acid sequence as set forth in SEQ ID NO: 22, CDR-L2 having the amino acid sequence as set forth in SEQ ID NO: 23, CDR-L3 having the amino acid sequence as set forth in SEQ ID NO: 24.

In some embodiments, the antibody or antigen-binding fragment thereof comprises: (1c) a heavy chain variable region (VH) comprising the following 3 CDRs: CDR-H1 having the amino acid sequence as set forth in SEQ ID NO: 36, CDR-H2 having the amino acid sequence as set forth in SEQ ID NO: 37, CDR-H3 having the amino acid sequence as set forth in SEQ ID NO: 38; and/or, a light chain variable region (VL) comprising the following 3 CDRs: CDR-L1 having the amino acid sequence as set forth in SEQ ID NO: 45, CDR-L2 having the amino acid sequence as set forth in SEQ ID NO: 23, and CDR-L3 having the amino acid sequence as set forth in SEQ ID NO: 52.

In some embodiments, the antibody or antigen-binding fragment thereof comprises: (2a) a heavy chain variable region (VH) comprising the following 3 CDRs: CDR-H1 having the amino acid sequence as set forth in SEQ ID NO: 7, CDR-H2 having the amino acid sequence as set forth in SEQ ID NO: 8, CDR-H3 having the amino acid sequence as set forth in SEQ ID NO: 9; and/or, a light chain variable region (VL) comprising the following 3 CDRs: CDR-L1 having the amino acid sequence as set forth in SEQ ID NO: 4, CDR-L2 having the amino acid sequence as set forth in SEQ ID NO: 5, CDR-L3 having the amino acid sequence as set forth in SEQ ID NO: 6.

In some embodiments, the antibody or antigen-binding fragment thereof comprises: (2b) a heavy chain variable region (VH) comprising the following 3 CDRs: CDR-H1 having the amino acid sequence as set forth in SEQ ID NO: 25, CDR-H2 having the amino acid sequence as set forth in SEQ ID NO: 26, CDR-H3 having the amino acid sequence as set forth in SEQ ID NO: 21; and/or, a light chain variable region (VL) comprising the following 3 CDRs: CDR-L1 having the amino acid sequence as set forth in SEQ ID NO: 22, CDR-L2 having the amino acid sequence as set forth in SEQ ID NO: 23, CDR-L3 having the amino acid sequence as set forth in SEQ ID NO: 24.

In some embodiments, the antibody or antigen-binding fragment thereof comprises: (2c) a heavy chain variable region (VH) comprising the following 3 CDRs: CDR-H1 having the amino acid sequence as set forth in SEQ ID NO: 39, CDR-H2 having the amino acid sequence as set forth in SEQ ID NO: 40, CDR-H3 having the amino acid sequence as set forth in SEQ ID NO: 38; and/or, a light chain variable region (VL) comprising the following 3 CDRs: CDR-L1 having the amino acid sequence as set forth in SEQ ID NO: 45, CDR-L2 having the amino acid sequence as set forth in SEQ ID NO: 23, and CDR-L3 having the amino acid sequence as set forth in SEQ ID NO: 52.

In some embodiments, the antibody or antigen-binding fragment thereof comprises: (3a) a heavy chain variable region (VH) comprising the following 3 CDRs: CDR-H1 having the amino acid sequence as set forth in SEQ ID NO: 10, CDR-H2 having the amino acid sequence as set forth in SEQ ID NO: 11, CDR-H3 having the amino acid sequence as set forth in SEQ ID NO: 12; and/or, a light chain variable region (VL) comprising the following 3 CDRs: CDR-L1 having the amino acid sequence as set forth in SEQ ID NO: 13, CDR-L2 having the amino acid sequence as set forth in SEQ ID NO: 14, CDR-L3 having the amino acid sequence as set forth in SEQ ID NO: 6.

In some embodiments, the antibody or antigen-binding fragment thereof comprises: (3b) a heavy chain variable region (VH) comprising the following 3 CDRs: CDR-H1 having the amino acid sequence as set forth in SEQ ID NO: 27, CDR-H2 having the amino acid sequence as set forth in SEQ ID NO: 28, CDR-H3 having the amino acid sequence as set forth in SEQ ID NO: 29; and/or, a light chain variable region (VL) comprising the following 3 CDRs: CDR-L1 having the amino acid sequence as set forth in SEQ ID NO: 30, CDR-L2 having the amino acid sequence as set forth in SEQ ID NO: 31, CDR-L3 having the amino acid sequence as set forth in SEQ ID NO: 24.

In some embodiments, the antibody or antigen-binding fragment thereof comprises: (3c) a heavy chain variable region (VH) comprising the following 3 CDRs: CDR-H1 having the amino acid sequence as set forth in SEQ ID NO: 41, CDR-H2 having the amino acid sequence as set forth in SEQ ID NO: 42, CDR-H3 having the amino acid sequence as set forth in SEQ ID NO: 43; and/or, a light chain variable region (VL) comprising the following 3 CDRs: CDR-L1 having the amino acid sequence as set forth in SEQ ID NO: 44, CDR-L2 having the amino acid sequence as set forth in SEQ ID NO: 31, and CDR-L3 having the amino acid sequence as set forth in SEQ ID NO: 52.

In some embodiments, the antibody or antigen-binding fragment thereof comprises:
(a) a VH as set forth in SEQ ID NO: 15 or a variant thereof, and/or, a VL as set forth in SEQ ID NO: 16 or a variant thereof;
(b) a VH as set forth in SEQ ID NO: 32 or a variant thereof, and/or, a VL as set forth in SEQ ID NO: 33 or a variant thereof; or
(c) a VH as set forth in SEQ ID NO: 46 or a variant thereof, and/or, a VL as set forth in SEQ ID NO: 47 or a variant thereof;
wherein, the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2, 3, 4 or 5 amino acids) as compared to the sequence from which it is derived; preferably, the substitution is a conservative substitution with the proviso that the amino acid sequences of the CDRs of the variant have 100% sequence identity to the amino acid sequences of the respective CDRs of the VH and VL of (a), (b), and (c) and the variant binds HER3.

In some embodiments, the antibody or antigen-binding fragment thereof comprises:
(a) a VH comprising the amino acid sequence as set forth in SEQ ID NO: 15 or a variant thereof, and, a VL comprising the amino acid sequence as set forth in SEQ ID NO: 16 or a variant thereof;
(b) a VH comprising the amino acid sequence as set forth in SEQ ID NO: 32 or a variant thereof, and, a VL comprising the amino acid sequence as set forth in SEQ ID NO: 33 or a variant thereof; or
(c) a VH comprising the amino acid sequence as set forth in SEQ ID NO: 46 or a variant thereof, and, a VL comprising the amino acid sequence as set forth in SEQ ID NO: 47 or a variant thereof;
wherein, the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2, 3, 4 or 5 amino acids) as compared to the sequence from which it is derived; preferably, the substitution is a conservative substitution.

In some embodiments, the antibody or antigen-binding fragment thereof comprises:
(a) a VH comprising the amino acid sequence as set forth in SEQ ID NO: 15, and, a VL comprising the amino acid sequence as set forth in SEQ ID NO: 16;
(b) a VH comprising the amino acid sequence as set forth in SEQ ID NO: 32, and, a VL comprising the amino acid sequence as set forth in SEQ ID NO: 33; or
(c) a VH comprising the amino acid sequence as set forth in SEQ ID NO: 46, and, a VL comprising the amino acid sequence as set forth in SEQ ID NO: 47.

In some embodiments, the antibody or antigen-binding fragment thereof comprises:
(a) a VH comprising the amino acid sequence as set forth in SEQ ID NO: 15, and, a VL comprising the amino acid sequence as set forth in SEQ ID NO: 16.

In some embodiments, the antibody or antigen-binding fragment thereof comprises:
(b) a VH comprising the amino acid sequence as set forth in SEQ ID NO: 32, and, a VL comprising the amino acid sequence as set forth in SEQ ID NO: 33.

In some embodiments, the antibody or antigen-binding fragment thereof comprises:
(c) a VH comprising the amino acid sequence as set forth in SEQ ID NO: 46, and, a VL comprising the amino acid sequence as set forth in SEQ ID NO: 47.

In some embodiments, the antibody or antigen-binding fragment thereof comprises: a heavy chain variable region (VH) comprising a CDR-H1, a CDR-H2, and a CDR-H3 comprising amino acid sequences of the CDR-H1, the CDR-H2, and the CDR-H3 as set forth in the VH comprising the amino acid sequence of SEQ ID NO: 15, respectively; and a light chain variable region (VL) comprising a CDR-L1, a CDR-L2, and a CDR-L3 comprising amino acid sequences of the CDR-L1, the CDR-L2, and the CDR-L3 as set forth in the VL comprising the amino acid sequence of SEQ ID NO: 16, respectively.

In some embodiments, the antibody or antigen-binding fragment thereof comprises: a heavy chain variable region (VH) comprising a CDR-H1, a CDR-H2, and a CDR-H3 comprising amino acid sequences of the CDR-H1, the CDR-H2, and the CDR-H3 as set forth in the VH comprising the amino acid sequence of SEQ ID NO: 32, respectively; and a light chain variable region (VL) comprising a CDR-L1, a CDR-L2, and a CDR-L3 comprising amino acid sequences of the CDR-L1, the CDR-L2, and the CDR-L3 as set forth in the VL comprising the amino acid sequence of SEQ ID NO: 33, respectively.

In some embodiments, the antibody or antigen-binding fragment thereof comprises: a heavy chain variable region (VH) comprising a CDR-H1, a CDR-H2, and a CDR-H3 comprising amino acid sequences of the CDR-H1, the CDR-H2, and the CDR-H3 as set forth in the VH comprising the amino acid sequence of SEQ ID NO: 46, respectively; and a light chain variable region (VL) comprising a CDR-L1, a CDR-L2, and a CDR-L3 comprising amino acid sequences of the CDR-L1, the CDR-L2, and the CDR-L3 as set forth in the VL comprising the amino acid sequence of SEQ ID NO: 47, respectively.

In some embodiments, the antibody or antigen-binding fragment thereof further comprises:
(a) a human immunoglobulin heavy chain constant region (CH) or a variant thereof, wherein, the variant has a substitution, deletion or addition of one or more amino acids (e.g., a substitution, deletion or addition of up to 20, up to 15, up to 10, or up to 5 amino acids; for example, a substitution, deletion or addition of 1, 2, 3, 4 or 5 amino acids) as compared to the wild-type sequence from which it is derived; and
(b) a human immunoglobulin light chain constant region (CL) or a variant thereof, wherein the variant has a substitution, deletion or addition of one or more amino acids (e.g., a substitution, deletion or addition of up to 20, up to 15, up to 10, or up to 5 amino acids; for example, a substitution, deletion or addition of 1, 2, 3, 4 or 5 amino acids) as compared to the wild-type sequence from which it is derived.

In some embodiments, the heavy chain constant region is an IgG heavy chain constant region, for example, an IgG1, IgG2, IgG3 or IgG4 heavy chain constant region, for example, a human IgG1 heavy chain constant region or a human IgG4 heavy chain constant region. In some embodiments, the antibody or antigen-binding fragment thereof comprises a heavy chain constant region (CH) as set forth in SEQ ID NO: 50 or a variant thereof, wherein the variant has a conservative substitution of up to 20 amino acids (e.g., a conservative substitution of up to 15, up to 10, or up to 5 amino acids; for example, a conservative substitution of 1, 2, 3, 4 or 5 amino acids) as compared to SEQ ID NO: 50.

In some embodiments, the light chain constant region is a k light chain constant region. In some embodiments, the antibody or antigen-binding fragment thereof comprises a light chain constant region (CL) as set forth in SEQ ID NO: 51 or a variant thereof, wherein the variant has a conservative substitution of up to 20 amino acids (e.g., a conservative substitution of up to 15, up to 10, or up to 5 amino acids; for example, a conservative substitution of 1, 2, 3, 4 or 5 amino acids) as compared to SEQ ID NO: 51.

In some embodiments, the antibody or antigen-binding fragment thereof comprises a heavy chain constant region (CH) as set forth in SEQ ID NO:50 and a light chain constant region (CL) as set forth in SEQ ID NO:51.

In some embodiments, the antibody or antigen-binding fragment thereof comprises:
(1) a heavy chain comprising a VH as set forth in SEQ ID NO: 15 and a heavy chain constant region (CH) as set forth in SEQ ID NO: 50, and, a light chain comprising a VL as set forth in SEQ ID NO: 16 and a light chain constant region (CL) as set forth in SEQ ID NO: 51;
(2) a heavy chain comprising a VH as set forth in SEQ ID NO: 32 and a heavy chain constant region (CH) as set forth in SEQ ID NO: 50, and, a light chain comprising a VL as set forth in SEQ ID NO: 33 and a light chain constant region (CL) as set forth in SEQ ID NO: 51; or
(3) a heavy chain comprising a VH as set forth in SEQ ID NO: 46 and a heavy chain constant region (CH) as set forth in SEQ ID NO: 50, and, a light chain comprising a VL as set forth in SEQ ID NO: 47 and a light chain constant region (CL) as set forth in SEQ ID NO: 51.

In some embodiments, the antibody or antigen-binding fragment thereof comprises: (1) a heavy chain comprising a VH comprising the amino acid sequence as set forth in SEQ ID NO: 15 and a heavy chain constant region (CH) comprising the amino acid sequence as set forth in SEQ ID NO: 50, and, a light chain comprising a VL comprising the amino acid sequence as set forth in SEQ ID NO: 16 and a light chain constant region (CL) comprising the amino acid sequence as set forth in SEQ ID NO: 51.

In some embodiments, the antibody or antigen-binding fragment thereof comprises: (2) a heavy chain comprising a VH comprising the amino acid sequence as set forth in SEQ ID NO: 32 and a heavy chain constant region (CH) comprising the amino acid sequence as set forth in SEQ ID NO: 50, and, a light chain comprising a VL comprising the amino acid sequence as set forth in SEQ ID NO: 33 and a light chain constant region (CL) comprising the amino acid sequence as set forth in SEQ ID NO: 51; or

In some embodiments, the antibody or antigen-binding fragment thereof comprises: (3) a heavy chain comprising a VH comprising the amino acid sequence as set forth in SEQ ID NO: 46 and a heavy chain constant region (CH) comprising the amino acid sequence as set forth in SEQ ID NO: 50, and, a light chain comprising a VL comprising the amino acid sequence as set forth in SEQ ID NO: 47 and a light chain constant region (CL) comprising the amino acid sequence as set forth in SEQ ID NO: 51.

In some embodiments, the antibody or antigen-binding fragment thereof comprises a heavy chain (HC) comprising the amino acid sequence as set forth in SEQ ID NO: 17, and a light chain (LC) comprising the amino acid sequence as set forth in SEQ ID NO: 18.

In some embodiments, the antibody or antigen-binding fragment thereof comprises a heavy chain (HC) comprising the amino acid sequence as set forth in SEQ ID NO: 34, and a light chain (LC) comprising the amino acid sequence as set forth in SEQ ID NO: 35.

In some embodiments, the antibody or antigen-binding fragment thereof comprises a heavy chain (HC) comprising the amino acid sequence as set forth in SEQ ID NO: 48, and a light chain (LC) comprising the amino acid sequence as set forth in SEQ ID NO: 49.

In particular embodiments of the antibody, the heavy chain constant domains as disclosed herein may comprise a C-terminal lysine or lack either a C-terminal lysine or a C-terminal glycine-lysine dipeptide. In some embodiments of the antibody or antigen binding fragment thereof, the N-terminal amino acid of the antibody or antigen binding fragment thereof variable domains may undergo cyclization to pyroglutamate. Thus, in a composition comprising a particular antibody disclosed herein, the composition may comprise a population of antibody species wherein each species may independently comprise a C-terminal lysine, lack a C-terminal lysine, lack a C-terminal glycine-lysine and/or comprise an N-terminal glutamine or glutamic acid or cyclization of the N-terminal amino acid to pyroglutamate.

In the antibody-drug conjugate, the cytotoxic drug can be conjugated to the antibody or antigen-binding fragment through a linker (e.g., the “M-L-E” fragment of the present application).

In some embodiments, M is

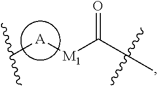

wherein Ring A is a 5- to 6-membered heteroaliphatic ring, or a 5- to 20-membered aromatic ring system, and the heteroaliphatic ring and aromatic ring system are optionally substituted with one or more groups selected from the group consisting of oxy (═O), halogen, cyano, amino, carboxyl, sulfhydryl group and C₁₋₆ alkyl; M₁ is selected from the group consisting of single bond, and C₁₋₂₀ alkylene, C₂₋₂₀ alkenylene or C₂₋₂₀ alkynylene.

In some embodiments, M is

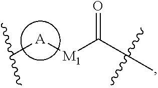

wherein Ring A is a 5-membered heteroaliphatic ring, 6-membered heteroaromatic ring, or a polycyclic ring formed by one or more 6-membered heteroaromatic rings and a benzene ring connected through a single bond, wherein the heteroaliphatic ring is optionally substituted with one or more groups selected from the group consisting of oxy (═O), halogen and C₁₋₄ alkyl; M₁ is selected from the group consisting of single bond, C₃₋₁₀ alkylene, C₃₋₁₀ alkenylene and C₃₋₁₀ alkynylene.

In some embodiments, M is

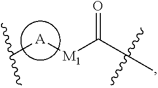

wherein Ring A is selected from from the group consisting of

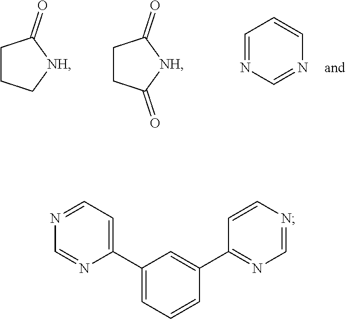 [SMILES: O=C1CCCN1 ; O=C1CCC(=O)N1 ; c1cncnc1 ; c1cc(-c2ccncn2)cc(-c2ccncn2)c1]

M₁ is selected from the group consisting of single bond, C₅₋₈ alkylene, C₅₋₈ alkenylene, and C₅₋₈ alkynylene.

In some embodiments, M is selected from the following structures:

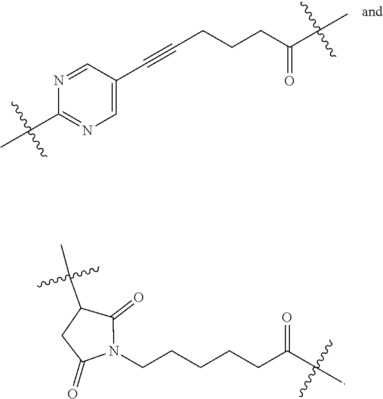

In some embodiments, M is selected from the following structure:

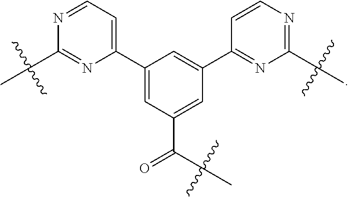

In some embodiments, L is a structure composed of one or more selected from the following: C₁₋₆ alkylene, —N(R′)—, carbonyl, —O—, Val, Cit, Phe, Lys, Lys (COCH₂CH₂ (OCH₂CH₂)ₛOCH₃), D-Val, Leu, Gly, Ala, Asn, Val-Cit, Val-Ala, Val-Lys, Val-Lys (Ac), Phe-Lys, Phe-Lys (Ac), D-Val-Leu-Lys, Gly-Gly-Arg, Ala-Ala-Asn, Ala-Ala-Ala, Val-Lys-Ala, Val-Lys-Gly, Gly-Gly-Gly, Gly-Gly-Phe-Gly, Gly-Gly-Gly-Gly-Gly,

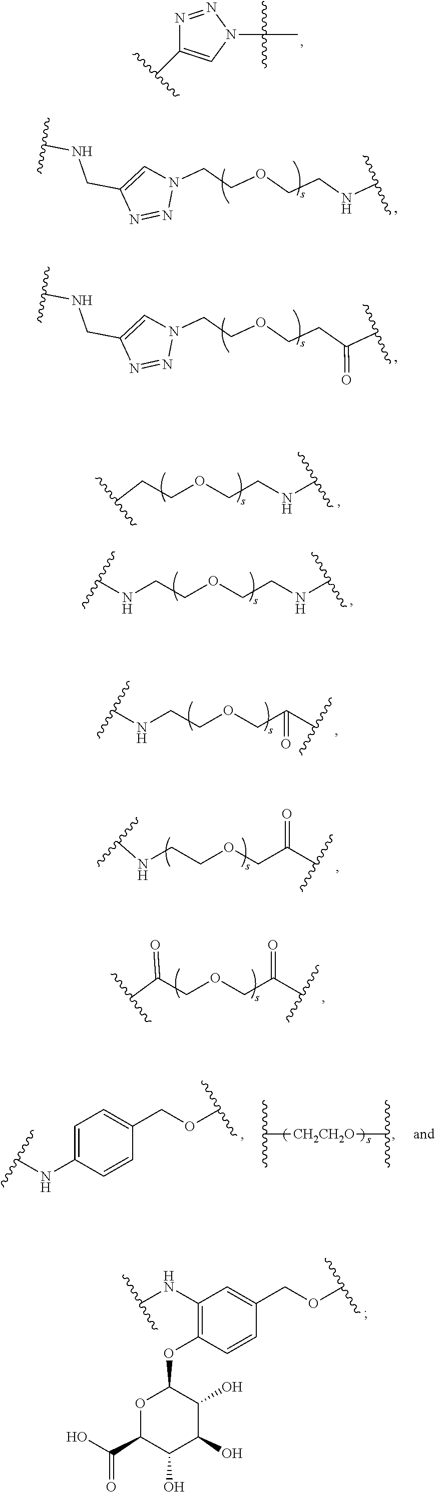

wherein R′ represents hydrogen, C₁₋₆ alkyl or alkyl containing —(CH₂CH₂O)ᵣ—; r is an integer selected from 1 to 10; s is an integer selected from 1 to 20.

In some embodiments, L is a structure composed of one or more selected from the following: C₁₋₆ alkylene, —NH—, Phe, Lys, Lys (COCH₂CH₂ (OCH₂CH₂)ₛOCH₃), Gly, Gly-Gly-Phe-Gly,

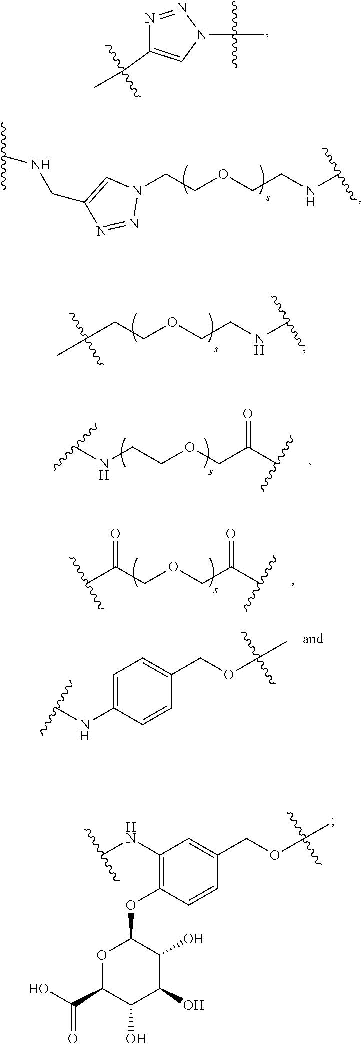

wherein s is an integer selected from 1 to 20.

In some embodiments, L is selected from the following structures:

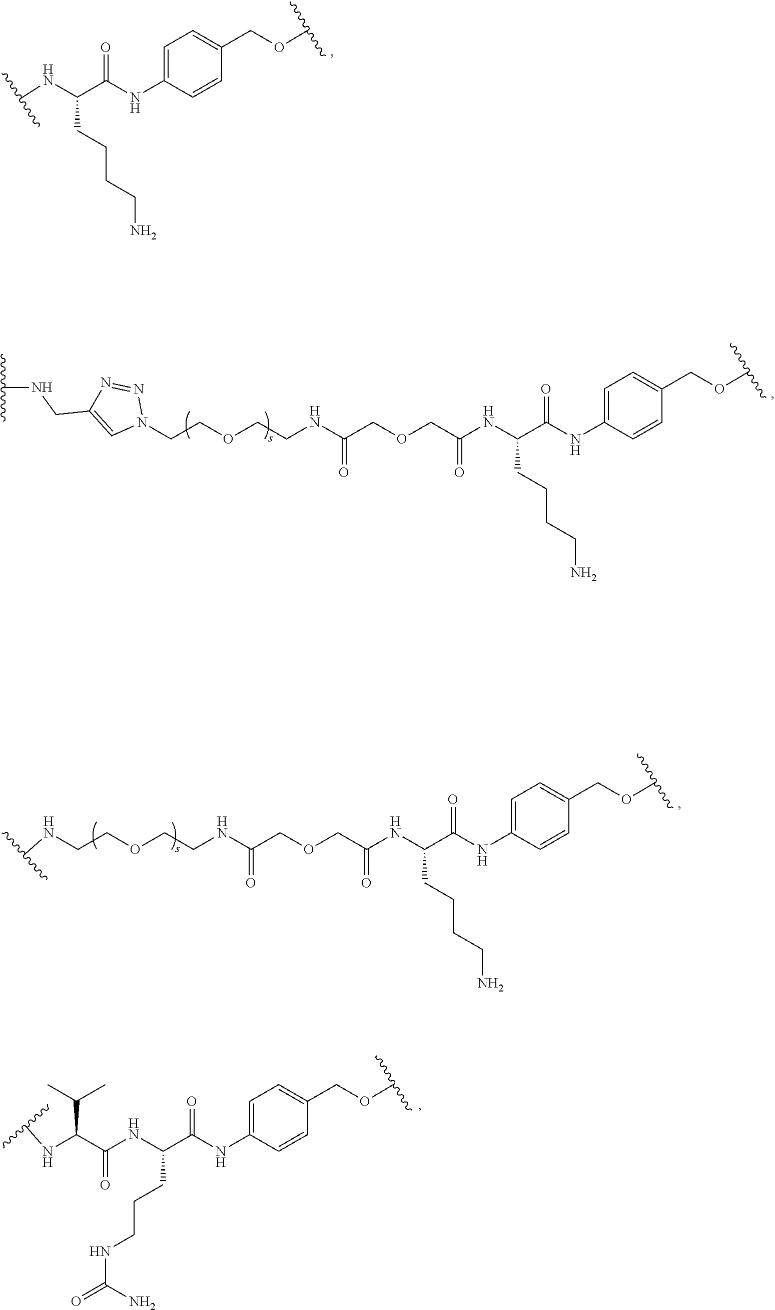

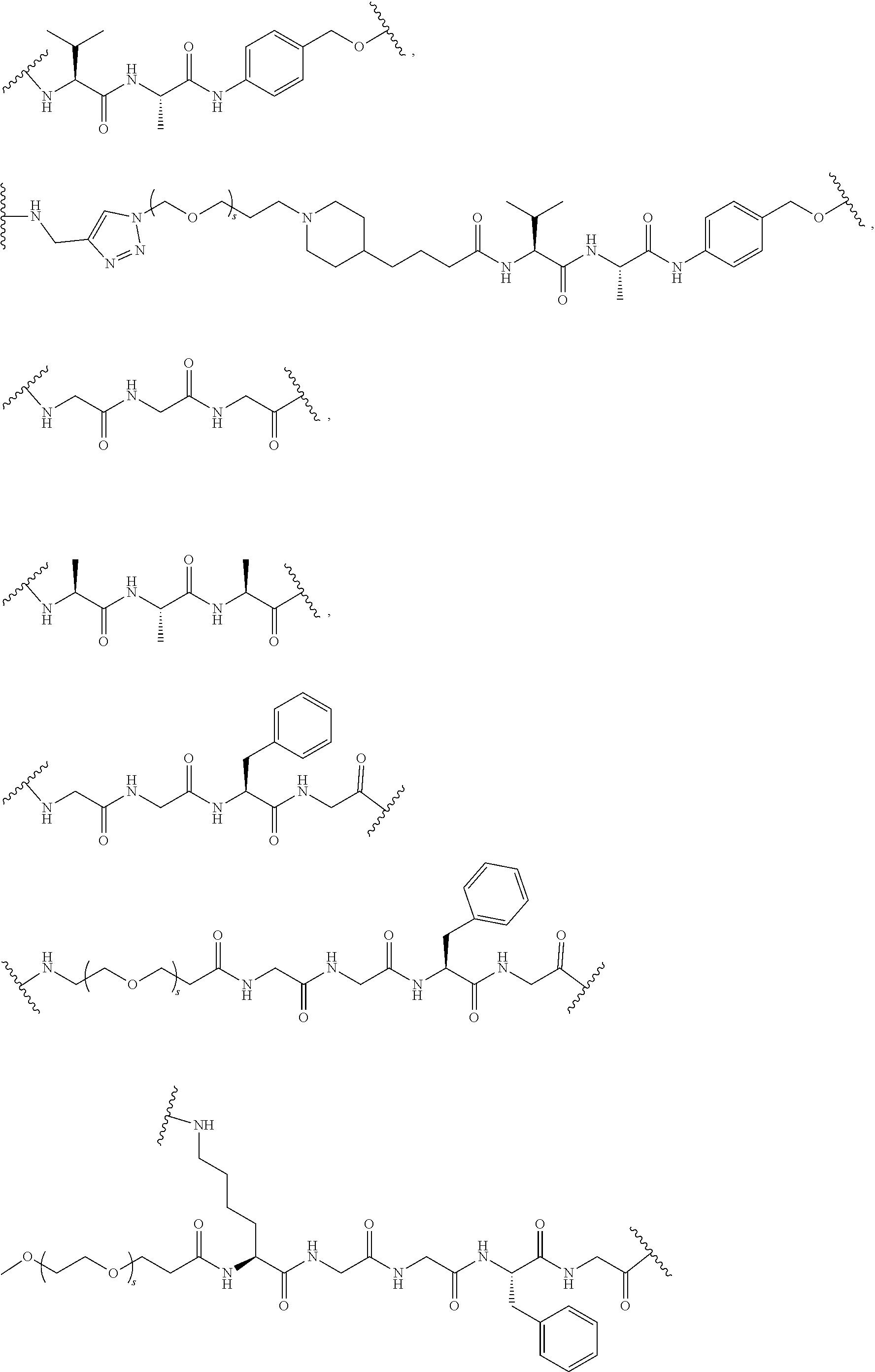

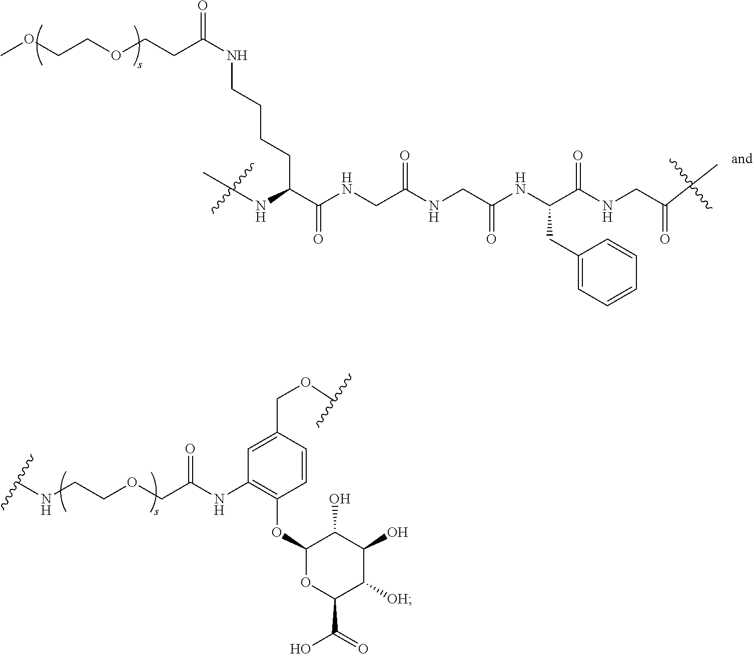

wherein s is an integer selected from 1 to 20.

In some embodiments, L is selected from the following structures:

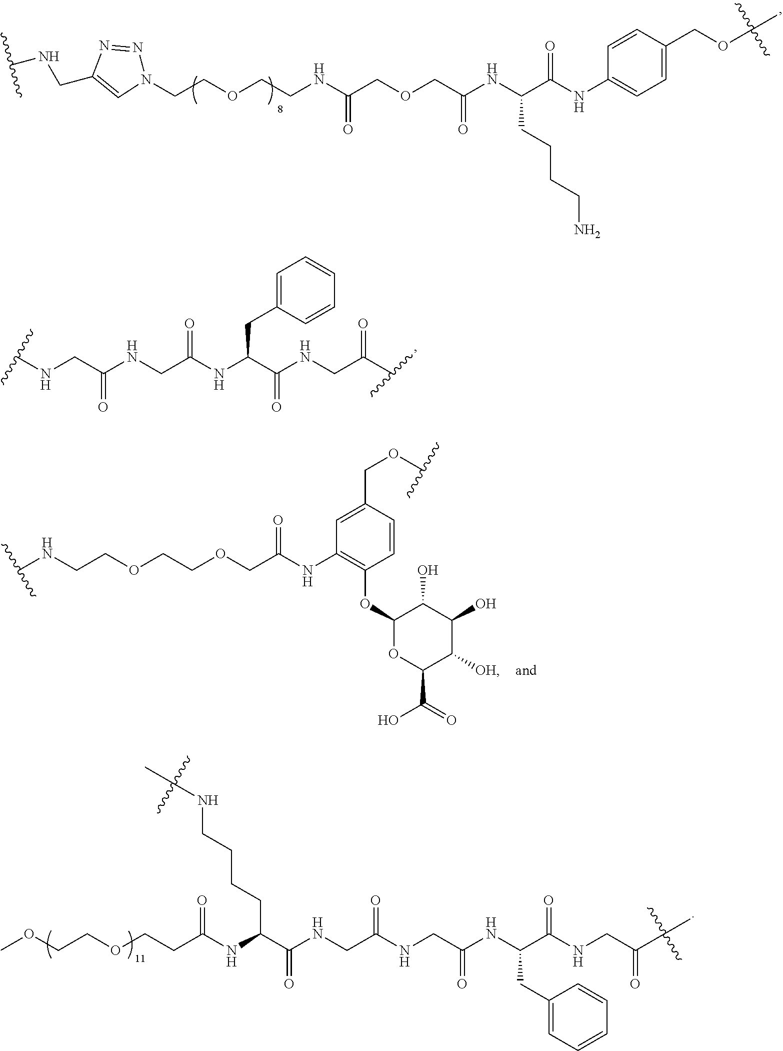

In some embodiments, L is selected from the following structures:

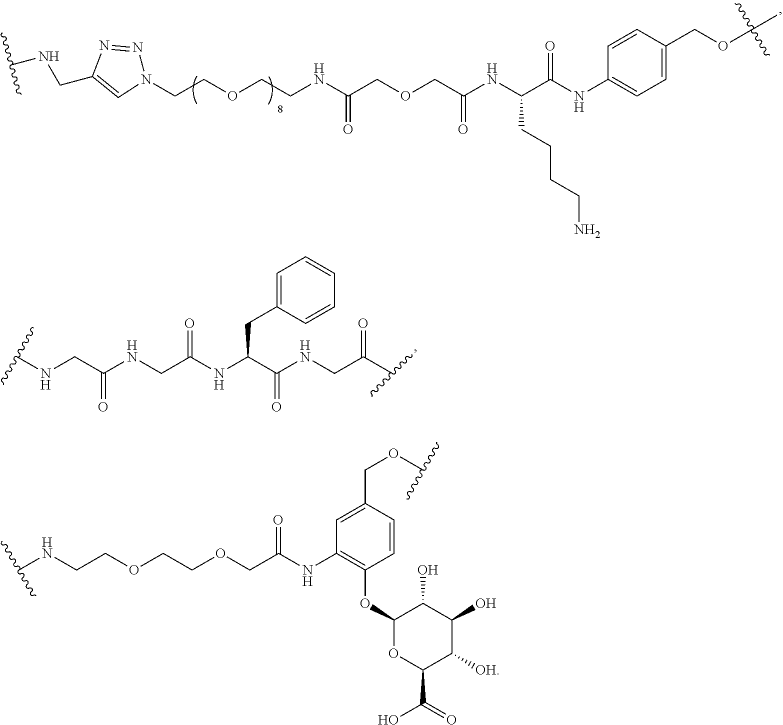

In some embodiments, E is a single bond, —NH—CH₂—, —NH—CH₂—O—CH₂—CO— or is selected from the following structures:

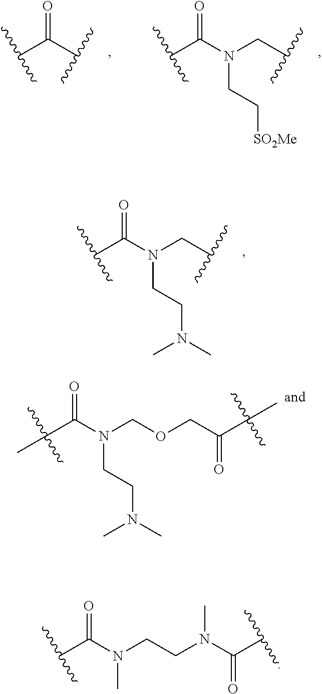

In some embodiments, E is a single bond, —NH—CH₂—, —NH—CH₂—O—CH₂—CO—,

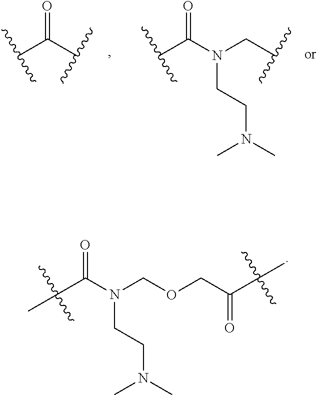

In some embodiments, E is-NH—CH₂—O—CH₂—CO—,

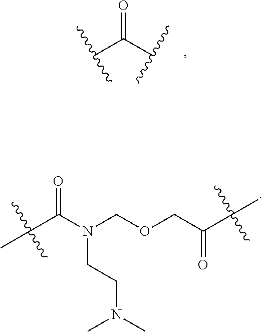

In some embodiments, E is-NH—CH₂—O—CH₂—CO— or

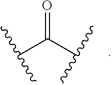 [SMILES: *C(*)=O ; "."]

In some embodiments, M is selected from the following structures:

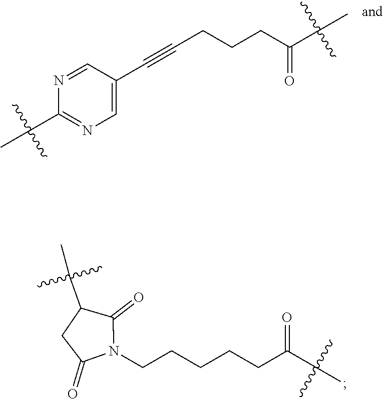

L is selected from the following structures:

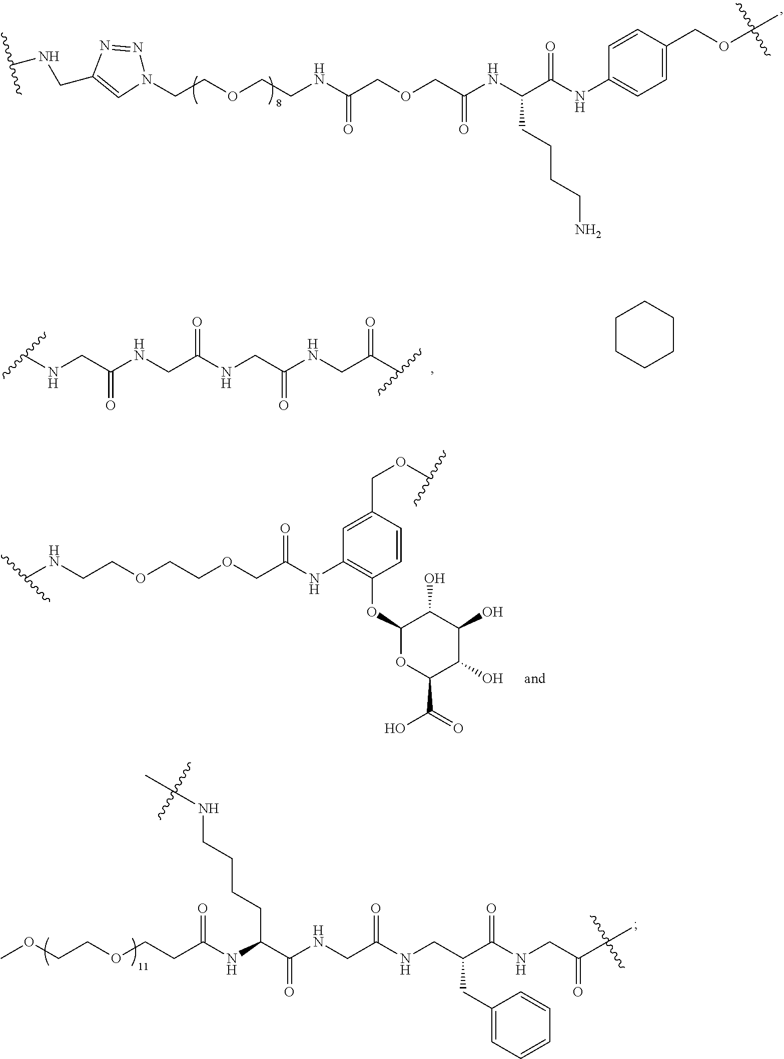

E is-NH—CH₂—O—CH₂—CO—,

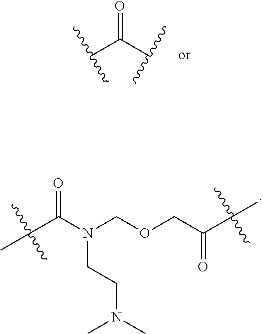

In some embodiments,

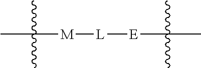

is selected from the following structures:

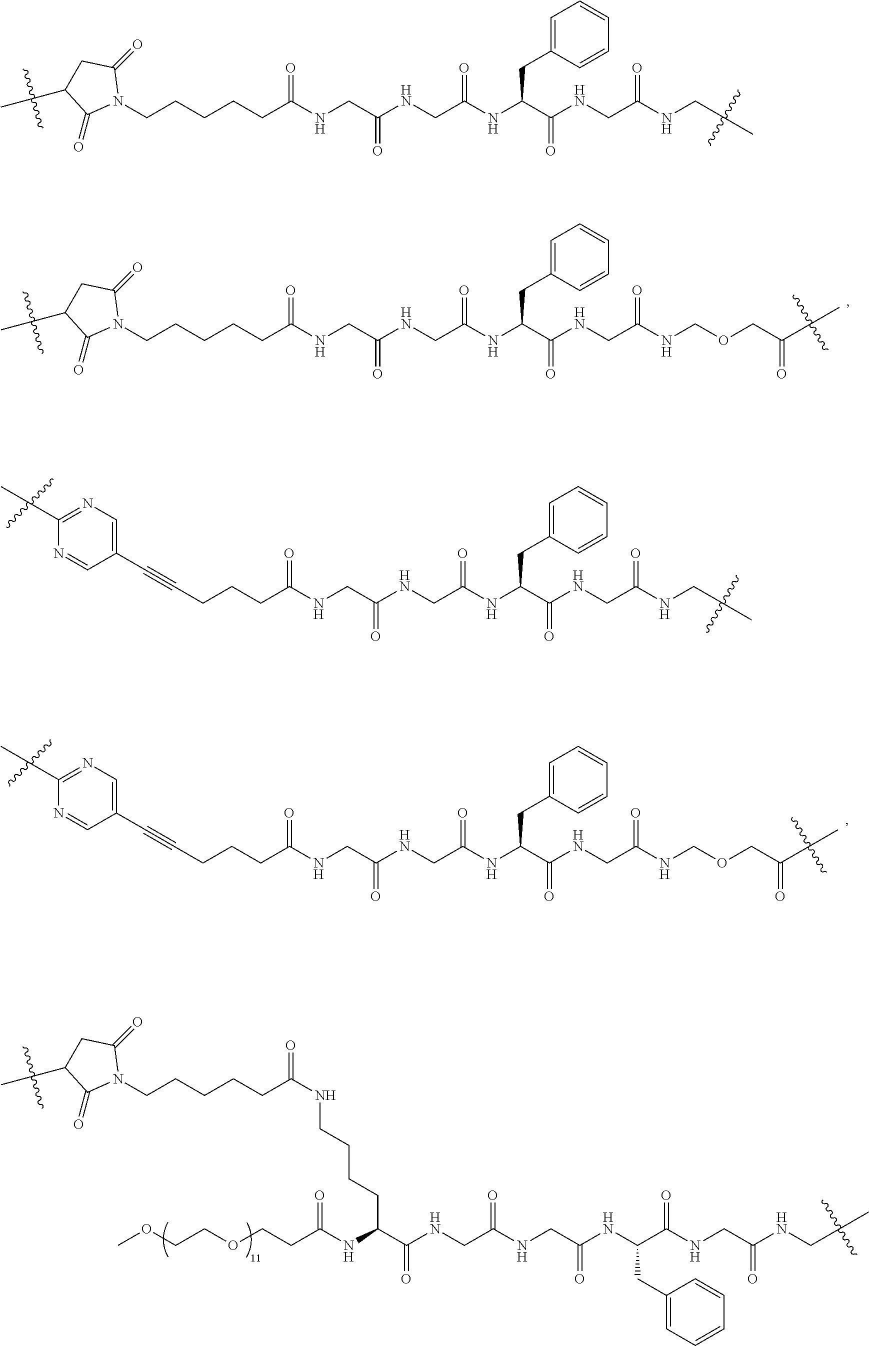

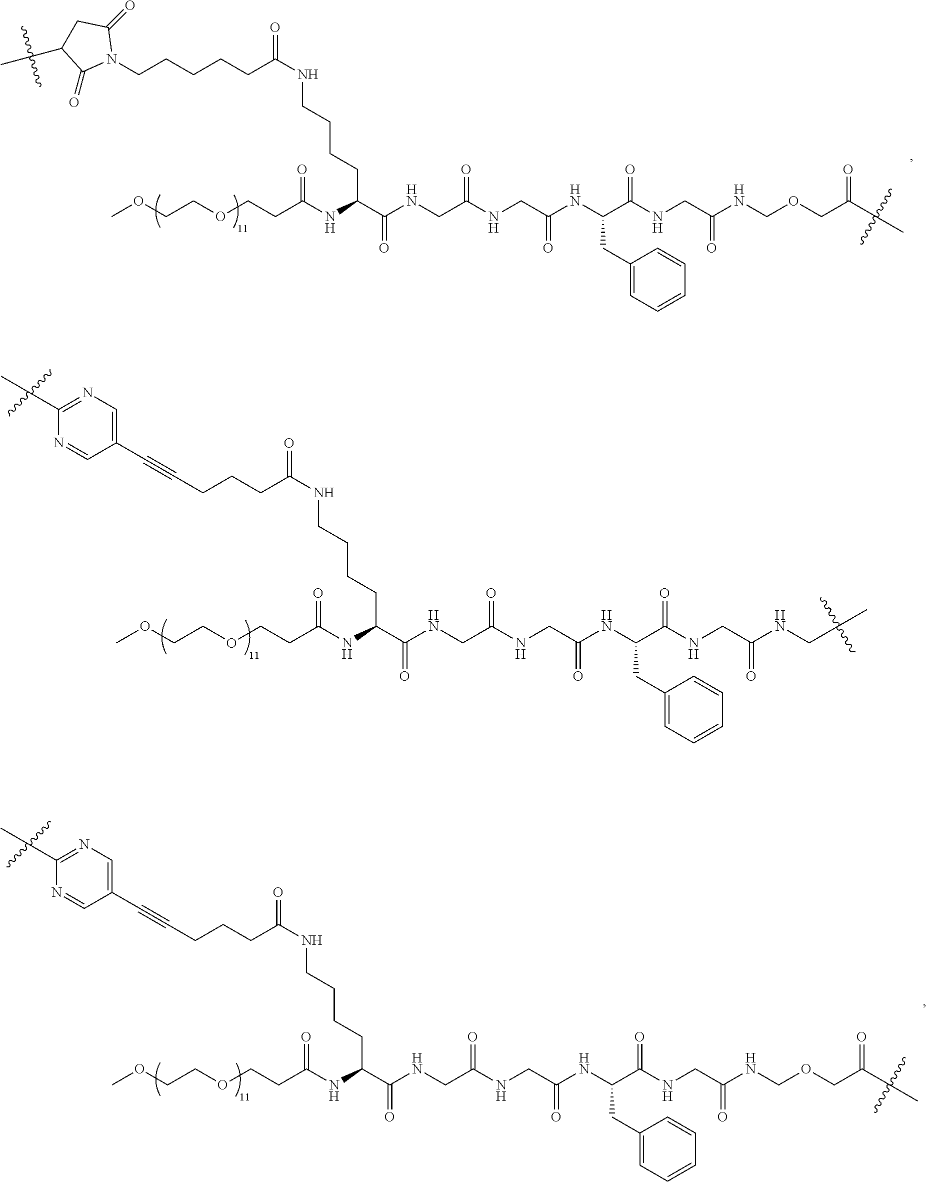

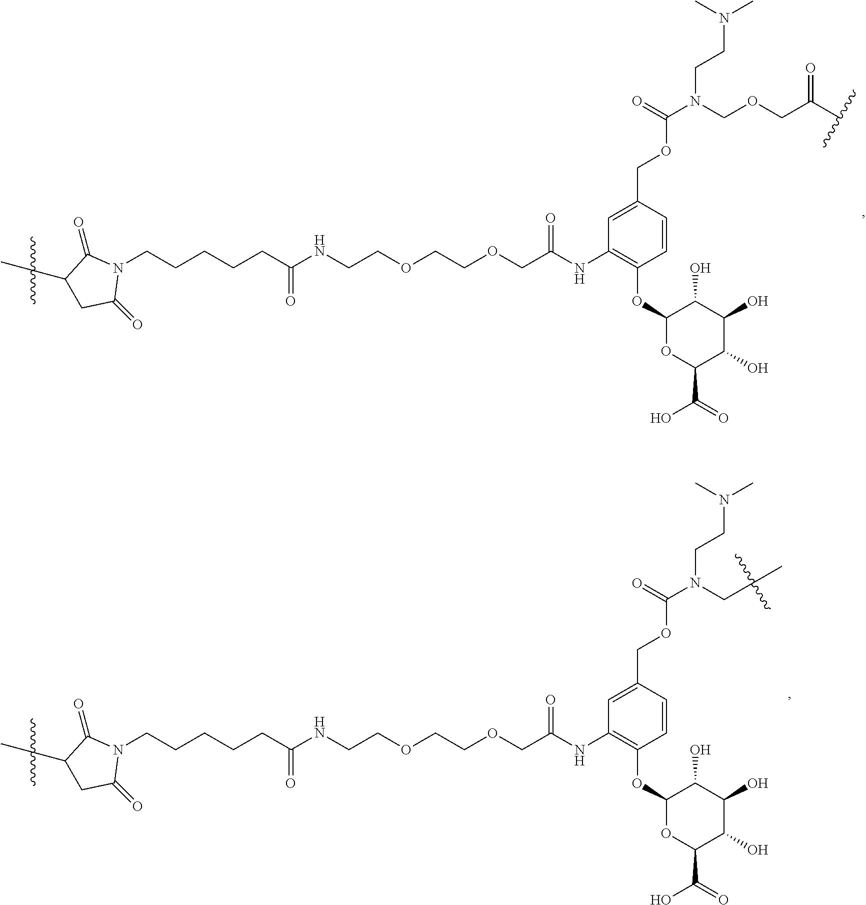 [SMILES: *C(=O)COCN(CCN(C)C)C(=O)OCc1ccc(O[C@@H]2O[C@H](C(=O)O)[C@@H](O)[C@H](O)[C@H]2O)c(NC(=O)COCCOCCNC(=O)CCCCCN2C(=O)CC(*)C2=O)c1 ; *CN(CCN(C)C)C(=O)OCc1ccc(O[C@@H]2O[C@H](C(=O)O)[C@@H](O)[C@H](O)[C@H]2O)c(NC(=O)COCCOCCNC(=O)CCCCCN2C(=O)CC(*)C2=O)c1]

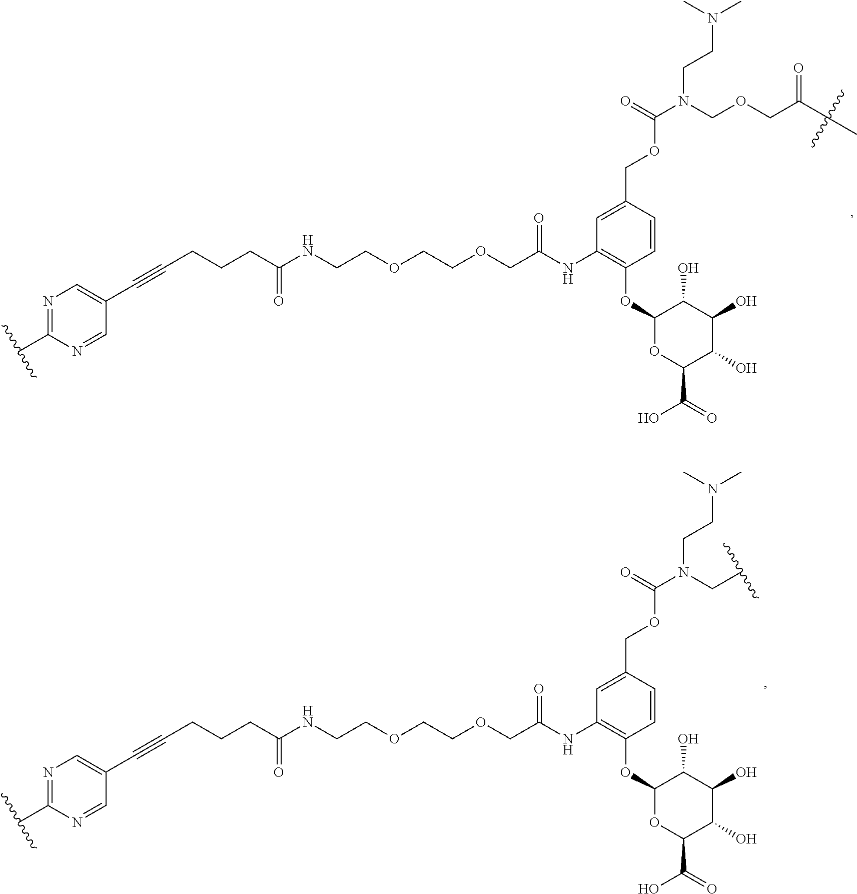 [SMILES: *C(=O)COCN(CCN(C)C)C(=O)OCc1ccc(O[C@@H]2O[C@H](C(=O)O)[C@@H](O)[C@H](O)[C@H]2O)c(NC(=O)COCCOCCNC(=O)CCCC#Cc2cnc(*)nc2)c1 ; *CN(CCN(C)C)C(=O)OCc1ccc(O[C@@H]2O[C@H](C(=O)O)[C@@H](O)[C@H](O)[C@H]2O)c(NC(=O)COCCOCCNC(=O)CCCC#Cc2cnc(*)nc2)c1]

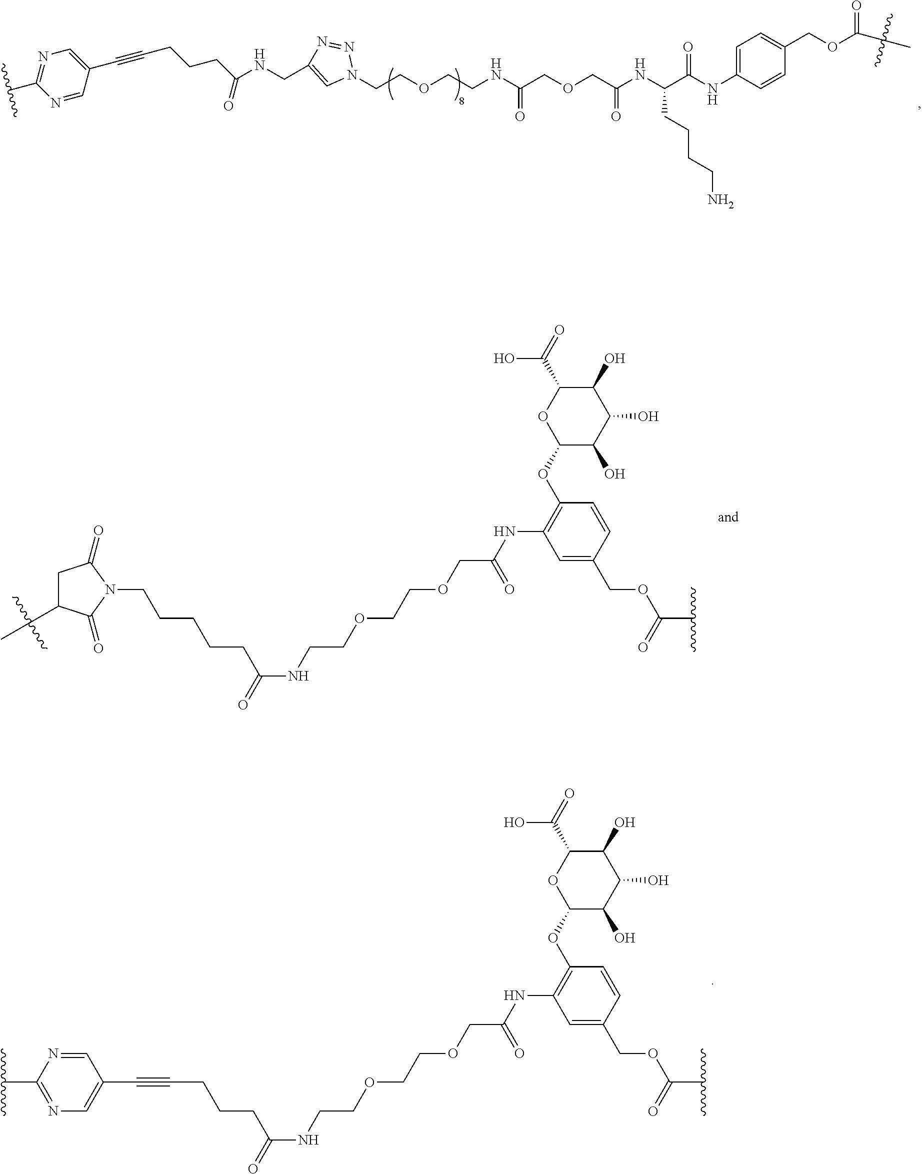

In some embodiments,

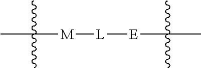

is selected from the following structures:

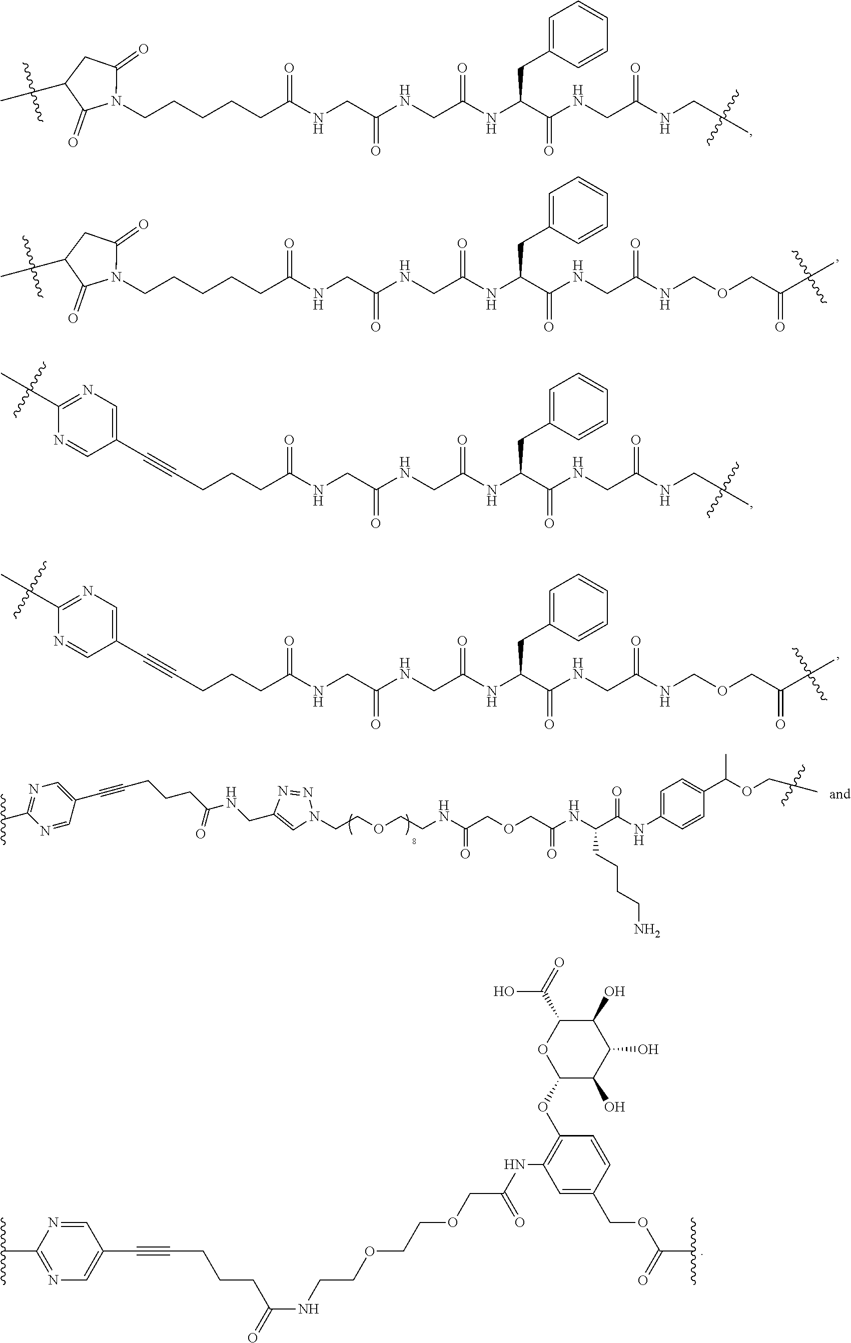

In some embodiments, the cytotoxic drug is selected from the group consisting of tubulin inhibitors, DNA intercalators, DNA topoisomerase inhibitors, and RNA polymerase inhibitors. In some embodiments, the tubulin inhibitor is an auristatin compound or a maytansine compound. In some embodiments, the DNA intercalator is pyrrolobenzodiazepine (PBD). In some embodiments, the DNA topoisomerase inhibitor is a topoisomerase I inhibitor (e.g., camptothecin, hydroxycamptothecin, 9-aminocamptothecin, SN-38, irinotecan, topotecan, belotecan, or rubitecan) and a topoisomerase II inhibitor (e.g., doxorubicin, PNU-159682, duocarmycin, daunorubicin, mitoxantrone, podophyllotoxin, or etoposide). In some embodiments, the RNA polymerase inhibitor is α-amanitin or a pharmaceutically acceptable salt, ester or analog thereof.

The cytotoxic drug disclosed in the present application usually contains a variety of functional groups, such as hydroxyl (—OH), carboxyl (—COOH), sulfhydryl (—SH), primary amino (—NH₂), secondary amine group (—NR_{A}H) or a tertiary amine group (—NR_{B}R_{C}), wherein R_{A}, R_{B}, and R_{C} herein only represent a non-hydrogen substituent on N, and the cytotoxic drug can be connected to the linker in the conjugate through these functional groups.

In some embodiments, the cytotoxic drug is linked to E in the antibody-drug conjugate through —OH, —SH, primary amino group, secondary amine group or tertiary amine group thereon.

In some embodiments, the cytotoxic drug is selected from the following Formulas I and II:

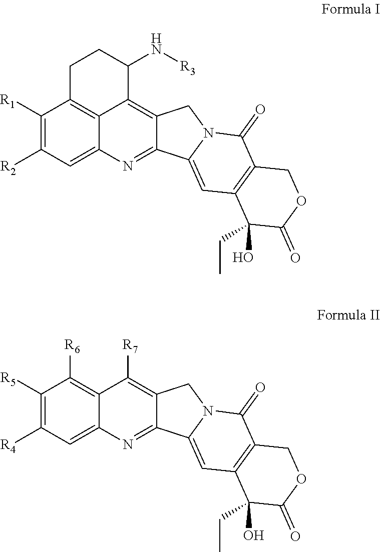

wherein, R₁ and R₂ are each independently selected from the group consisting of C₁₋₆ alkyl and halogen;
R₃ is selected from the group consisting of H and —CO—CH₂OH;
R₄ and R₅ are each independently selected from the group consisting of H, halogen and hydroxyl; or R₄ and R₅ together with the carbon atoms to which they are connected form a 5- to 6-membered oxygen-containing heterocycle;
R₆ is selected from the group consisting of hydrogen and —C₁₋₄ alkylene-NRₐR_{b};
R₇ is selected from the group consisting of C₁₋₆ alkyl and —C₁₋₄ alkylene-NRₐR_{b};
wherein, at each occurrence, Rₐ and R_{b} are each independently selected from the group consisting of H, C₁₋₆ alkyl, —SO₂—C₁₋₆ alkyl and —CO—C₁₋₆ alkyl.

In some embodiments, the cytotoxic drug is selected from the group consisting of the following compounds:

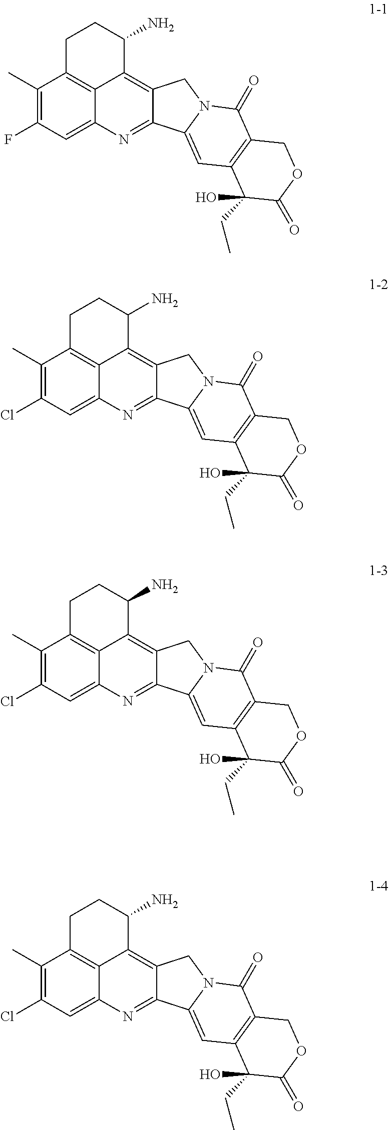 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(Cl)c(C)c3c1c2[C@H](N)CC3 ; CC[C@@]1(O)C(=O)OCc2c1cc1n(c2=O)Cc2c-1nc1cc(Cl)c(C)c3c1c2[C@@H](N)CC3]

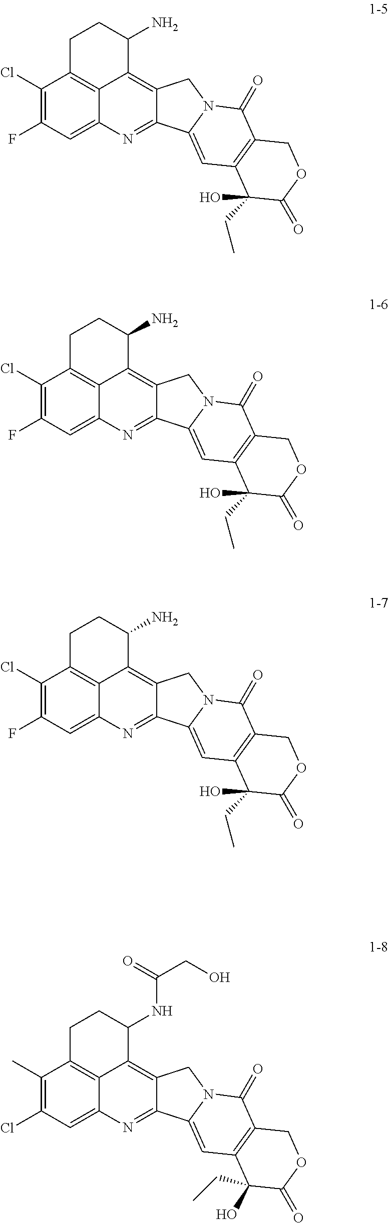 [SMILES: CC[C@@]1(O)C(=O)OCc2c1cc1n(c2=O)Cc2c-1nc1cc(F)c(Cl)c3c1c2C(N)CC3 ; CC[C@@]1(O)C(=O)OCc2c1cc1n(c2=O)Cc2c-1nc1cc(F)c(Cl)c3c1c2[C@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2C(NC(=O)CO)CC3]

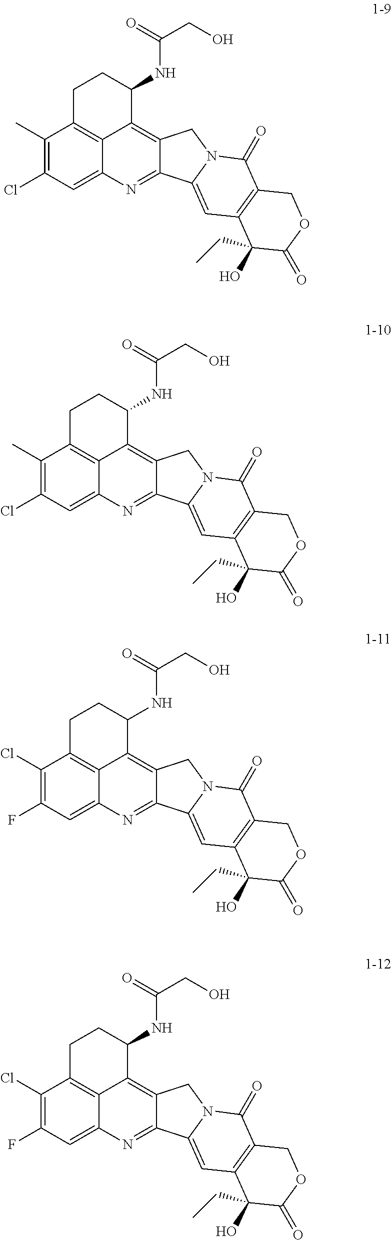 [SMILES: CC[C@@]1(O)C(=O)OCc2c1cc1n(c2=O)Cc2c-1nc1cc(Cl)c(C)c3c1c2[C@H](NC(=O)CO)CC3 ; CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2C(NC(=O)CO)CC3 ; CC[C@@]1(O)C(=O)OCc2c1cc1n(c2=O)Cc2c-1nc1cc(F)c(Cl)c3c1c2[C@H](NC(=O)CO)CC3]

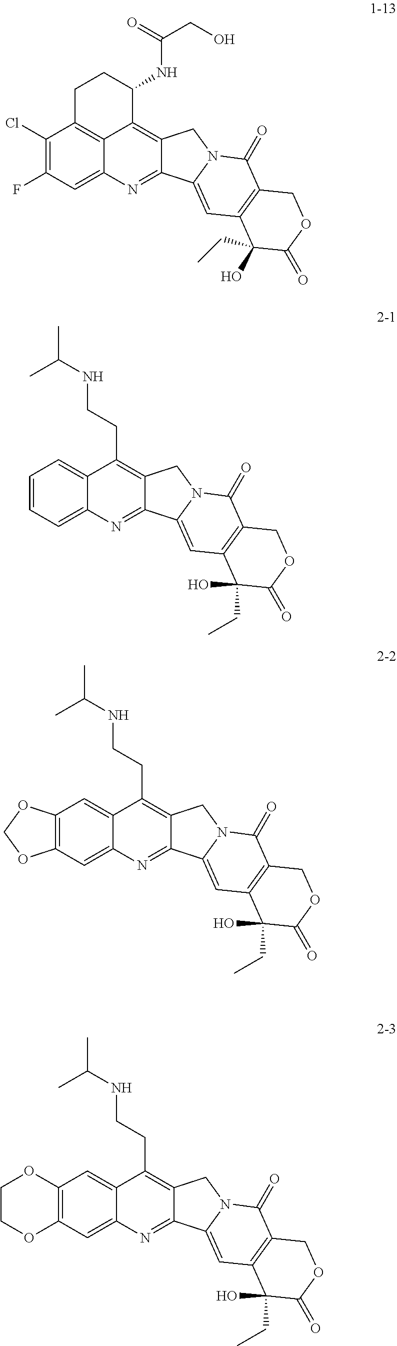 [SMILES: CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1ccccc1c2CCNC(C)C ; CC[C@@]1(O)C(=O)OCc2c1cc1n(c2=O)Cc2c-1nc1cc3c(cc1c2CCNC(C)C)OCO3 ; CC[C@@]1(O)C(=O)OCc2c1cc1n(c2=O)Cc2c-1nc1cc3c(cc1c2CCNC(C)C)OCCO3]

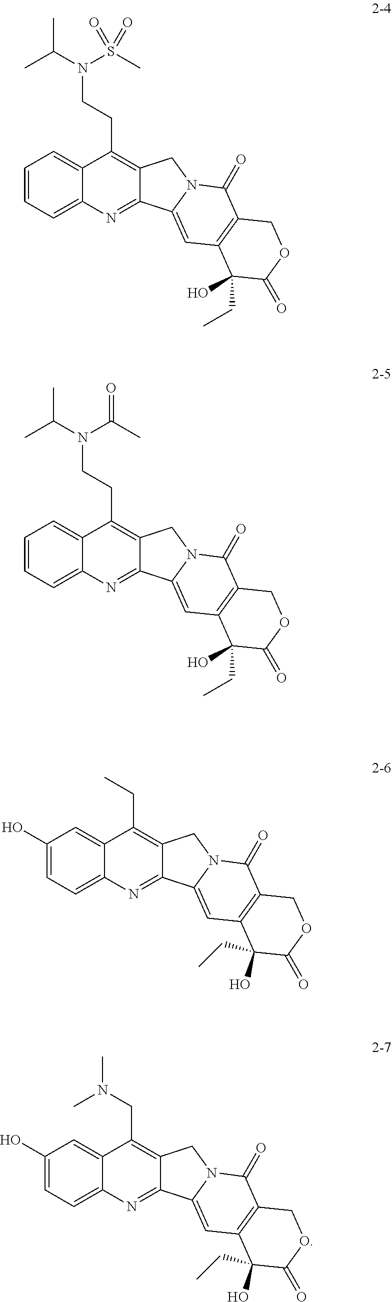 [SMILES: CC[C@@]1(O)C(=O)OCc2c1cc1n(c2=O)Cc2c-1nc1ccccc1c2CCN(C(C)C)S(C)(=O)=O ; CC[C@@]1(O)C(=O)OCc2c1cc1n(c2=O)Cc2c-1nc1ccccc1c2CCN(C(C)=O)C(C)C ; CCc1c2c(nc3ccc(O)cc13)-c1cc3c(c(=O)n1C2)COC(=O)[C@]3(O)CC ; CC[C@@]1(O)C(=O)OCc2c1cc1n(c2=O)Cc2c-1nc1ccc(O)cc1c2CN(C)C]

In some embodiments, the cytotoxic drug is selected from the group consisting of the following compounds:

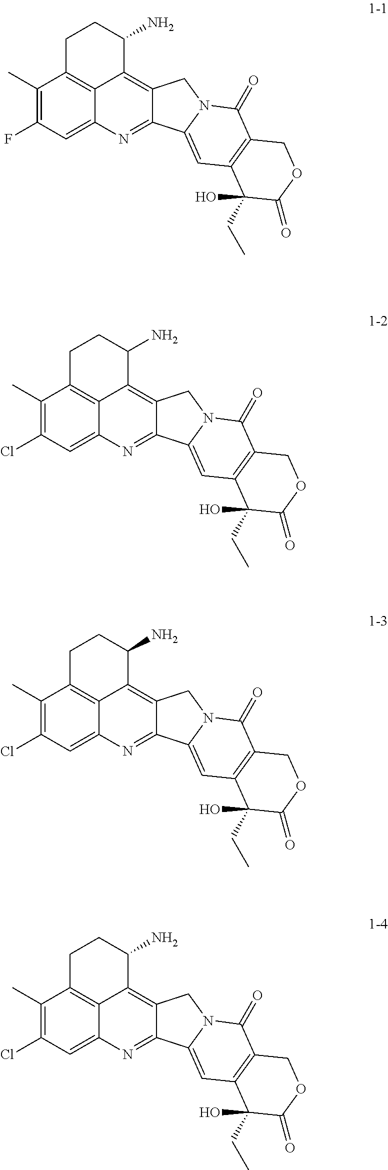 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(Cl)c(C)c3c1c2[C@H](N)CC3 ; CC[C@@]1(O)C(=O)OCc2c1cc1n(c2=O)Cc2c-1nc1cc(Cl)c(C)c3c1c2[C@@H](N)CC3]

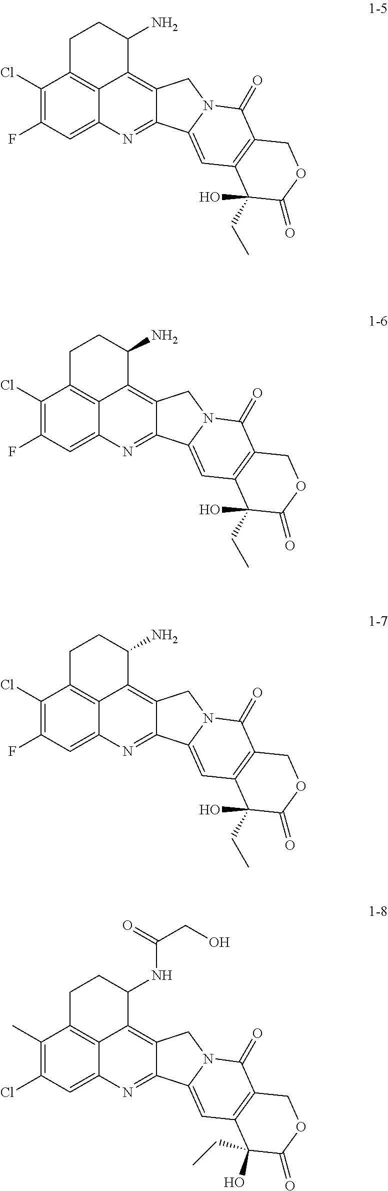 [SMILES: CC[C@@]1(O)C(=O)OCc2c1cc1n(c2=O)Cc2c-1nc1cc(F)c(Cl)c3c1c2C(N)CC3 ; CC[C@@]1(O)C(=O)OCc2c1cc1n(c2=O)Cc2c-1nc1cc(F)c(Cl)c3c1c2[C@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2C(NC(=O)CO)CC3]

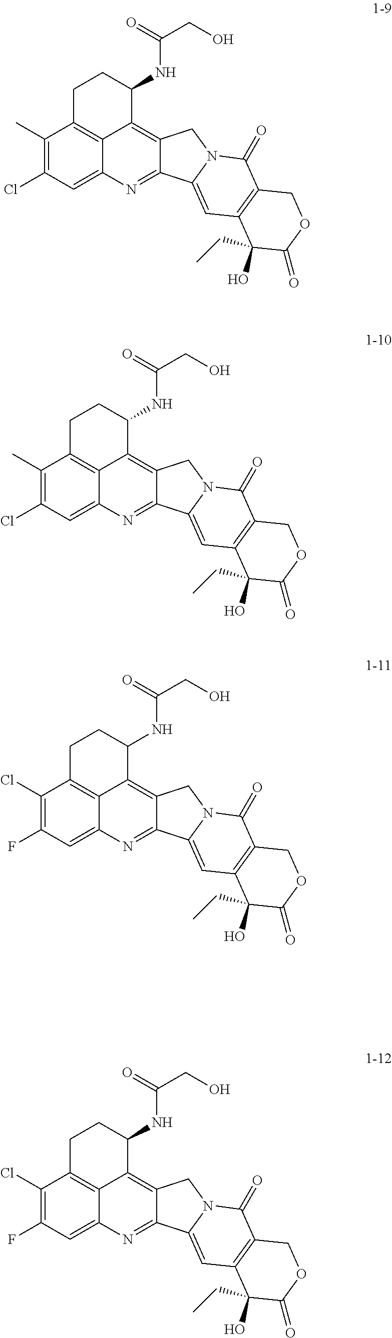 [SMILES: CC[C@@]1(O)C(=O)OCc2c1cc1n(c2=O)Cc2c-1nc1cc(Cl)c(C)c3c1c2[C@H](NC(=O)CO)CC3 ; CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2C(NC(=O)CO)CC3 ; CC[C@@]1(O)C(=O)OCc2c1cc1n(c2=O)Cc2c-1nc1cc(F)c(Cl)c3c1c2[C@H](NC(=O)CO)CC3]

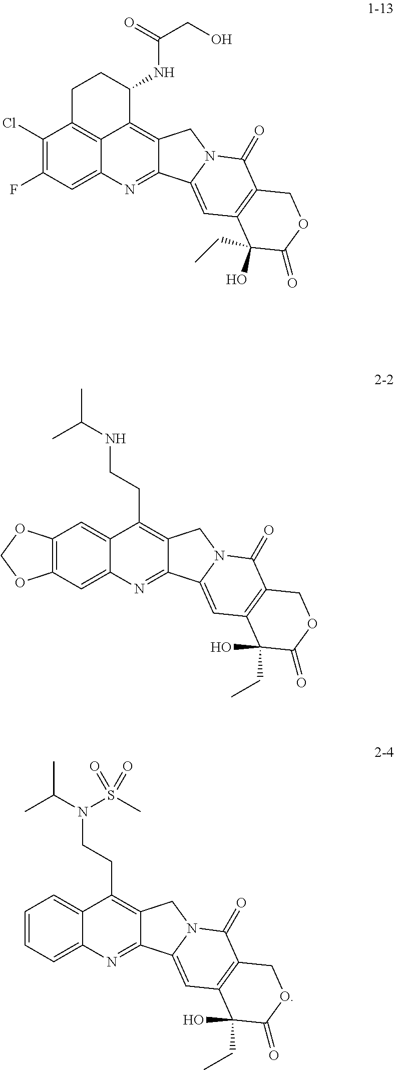 [SMILES: CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1cc3c(cc1c2CCNC(C)C)OCO3 ; CC[C@@]1(O)C(=O)OCc2c1cc1n(c2=O)Cc2c-1nc1ccccc1c2CCN(C(C)C)S(C)(=O)=O]

The corresponding fragment of the cytotoxic drug after being connected with the linker is D of the formula Ab-[M-L-E-D]ₓ. In some embodiments, D is a monovalent structure obtained by losing one H from —OH, —NH₂ or the secondary amine group on the cytotoxic drug.

In some embodiments, D is selected from the following structures:

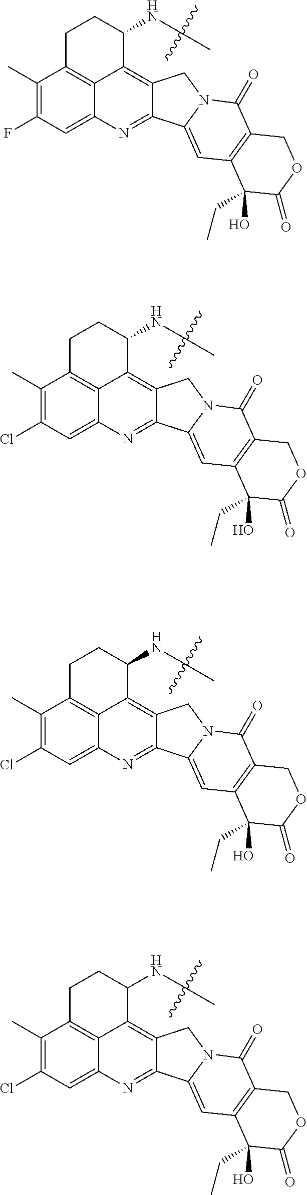 [SMILES: *N[C@H]1CCc2c(C)c(F)cc3nc4c(c1c23)Cn1c-4cc2c(c1=O)COC(=O)[C@]2(O)CC ; *N[C@H]1CCc2c(C)c(Cl)cc3nc4c(c1c23)Cn1c-4cc2c(c1=O)COC(=O)[C@]2(O)CC ; *N[C@@H]1CCc2c(C)c(Cl)cc3nc4c(c1c23)Cn1c-4cc2c(c1=O)COC(=O)[C@]2(O)CC ; *NC1CCc2c(C)c(Cl)cc3nc4c(c1c23)Cn1c-4cc2c(c1=O)COC(=O)[C@]2(O)CC]

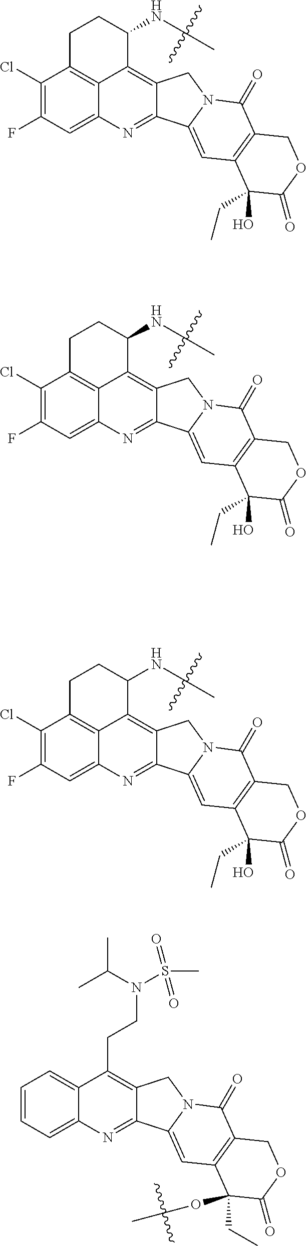 [SMILES: *N[C@H]1CCc2c(Cl)c(F)cc3nc4c(c1c23)Cn1c-4cc2c(c1=O)COC(=O)[C@]2(O)CC ; *N[C@@H]1CCc2c(Cl)c(F)cc3nc4c(c1c23)Cn1c-4cc2c(c1=O)COC(=O)[C@]2(O)CC ; *NC1CCc2c(Cl)c(F)cc3nc4c(c1c23)Cn1c-4cc2c(c1=O)COC(=O)[C@]2(O)CC ; *O[C@]1(CC)C(=O)OCc2c1cc1n(c2=O)Cc2c-1nc1ccccc1c2CCN(C(C)C)S(C)(=O)=O]

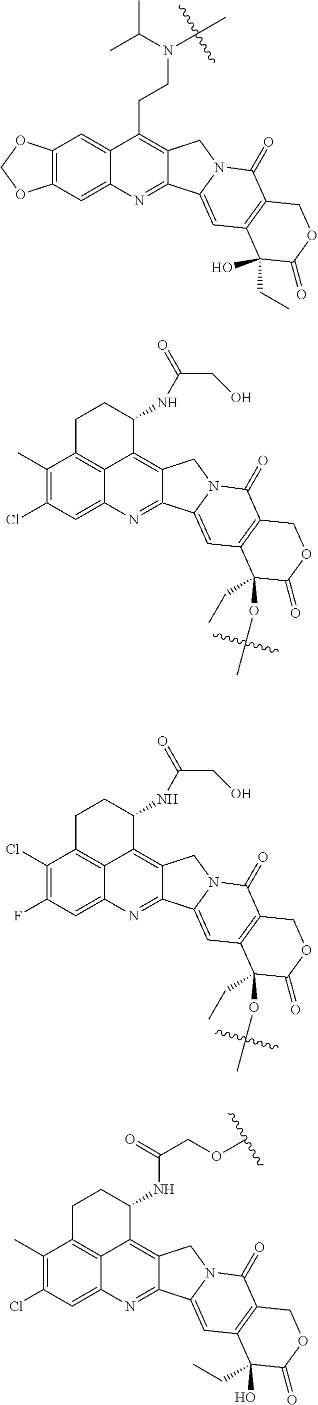 [SMILES: *N(CCc1c2c(nc3cc4c(cc13)OCO4)-c1cc3c(c(=O)n1C2)COC(=O)[C@]3(O)CC)C(C)C ; *O[C@]1(CC)C(=O)OCc2c1cc1n(c2=O)Cc2c-1nc1cc(Cl)c(C)c3c1c2[C@@H](NC(=O)CO)CC3 ; *O[C@]1(CC)C(=O)OCc2c1cc1n(c2=O)Cc2c-1nc1cc(F)c(Cl)c3c1c2[C@@H](NC(=O)CO)CC3 ; *OCC(=O)N[C@H]1CCc2c(C)c(Cl)cc3nc4c(c1c23)Cn1c-4cc2c(c1=O)COC(=O)[C@]2(O)CC]

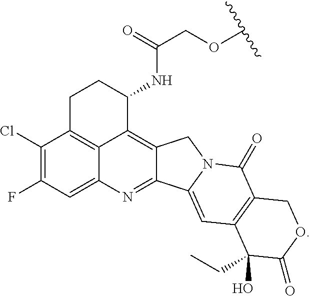 [SMILES: *OCC(=O)N[C@H]1CCc2c(Cl)c(F)cc3nc4c(c1c23)Cn1c-4cc2c(c1=O)COC(=O)[C@]2(O)CC]

In some embodiments, the antibody-drug conjugate is selected from the group consisting of ADC A-01 to ADC A-25, ADC B-01 to ADC B-05 as shown below:

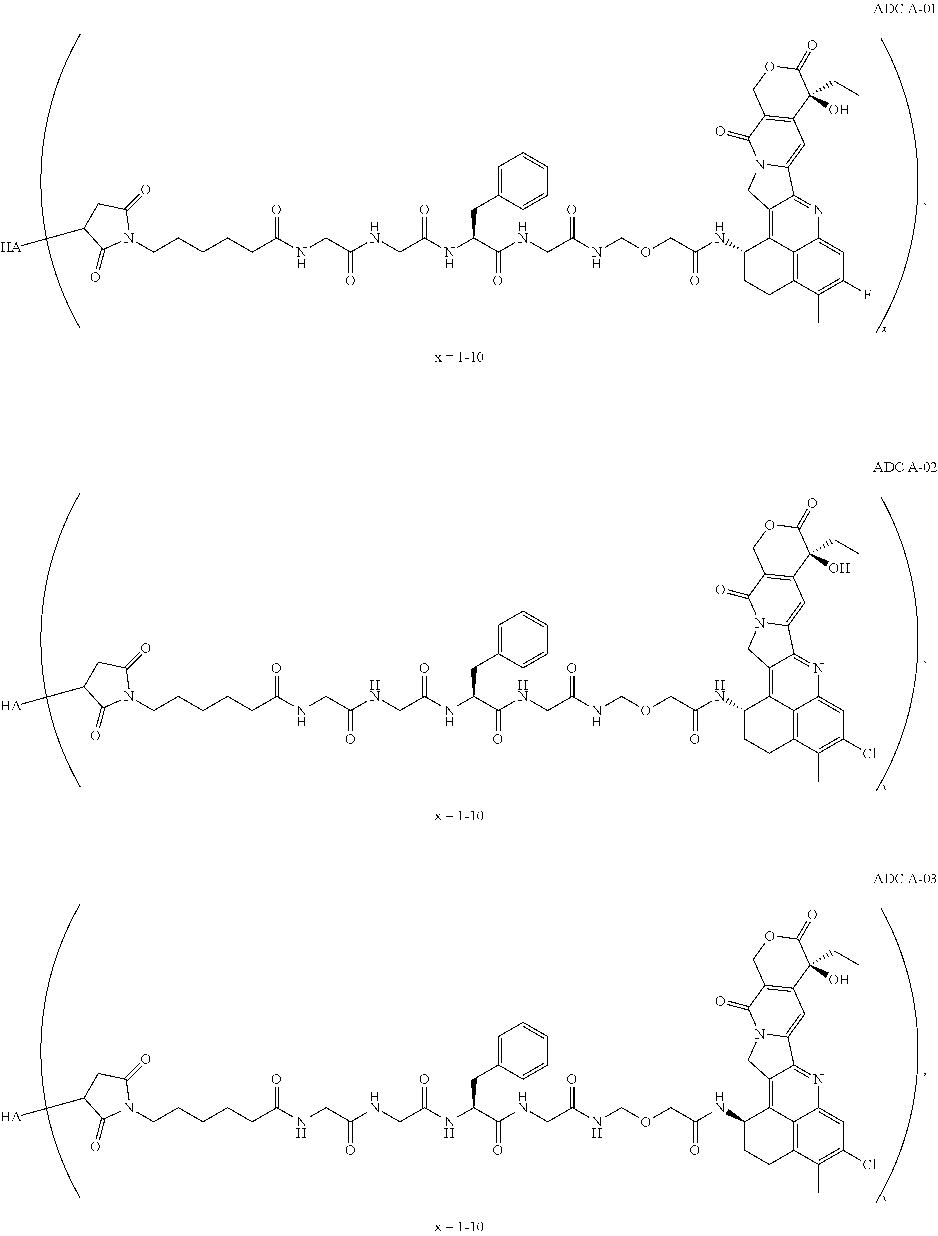

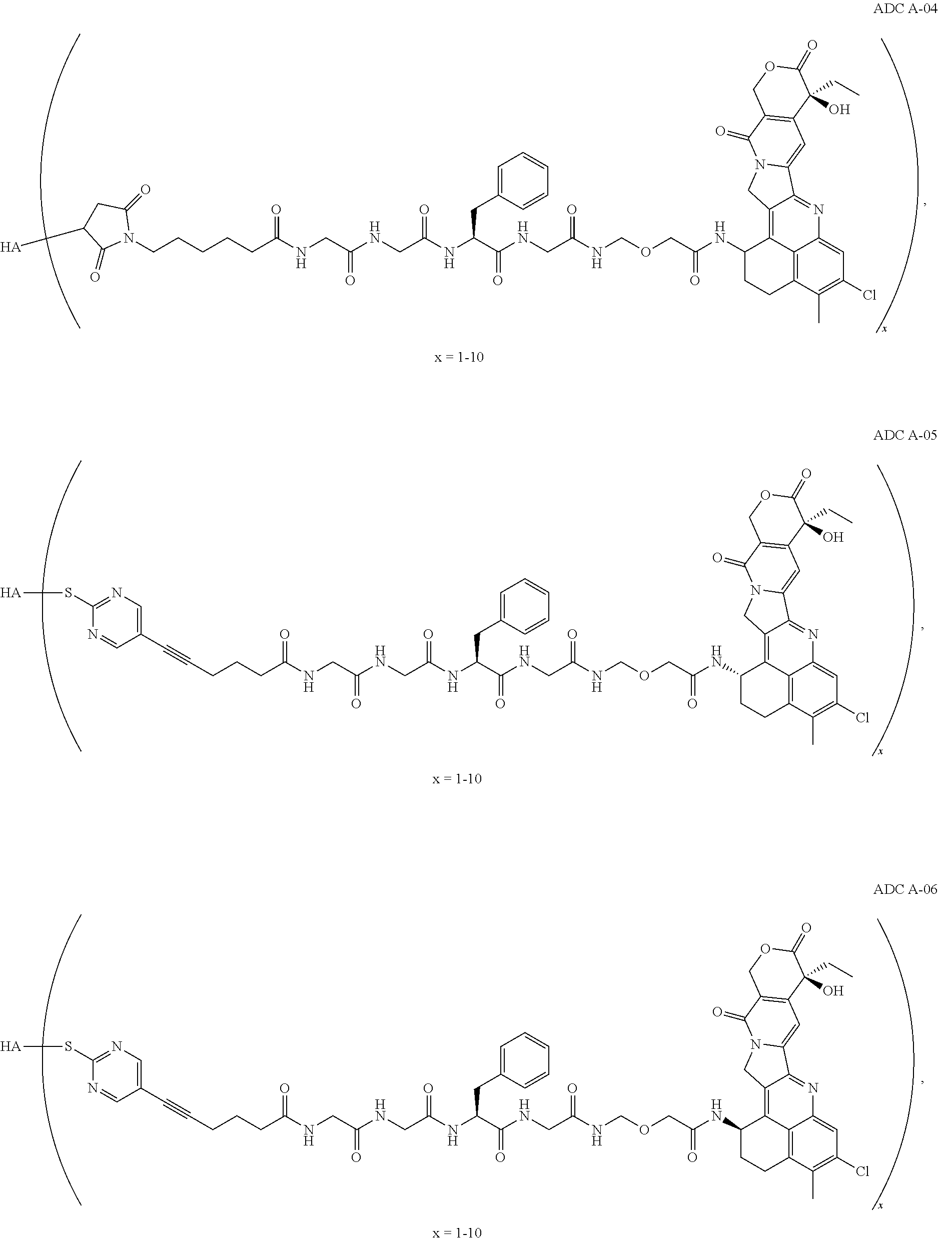

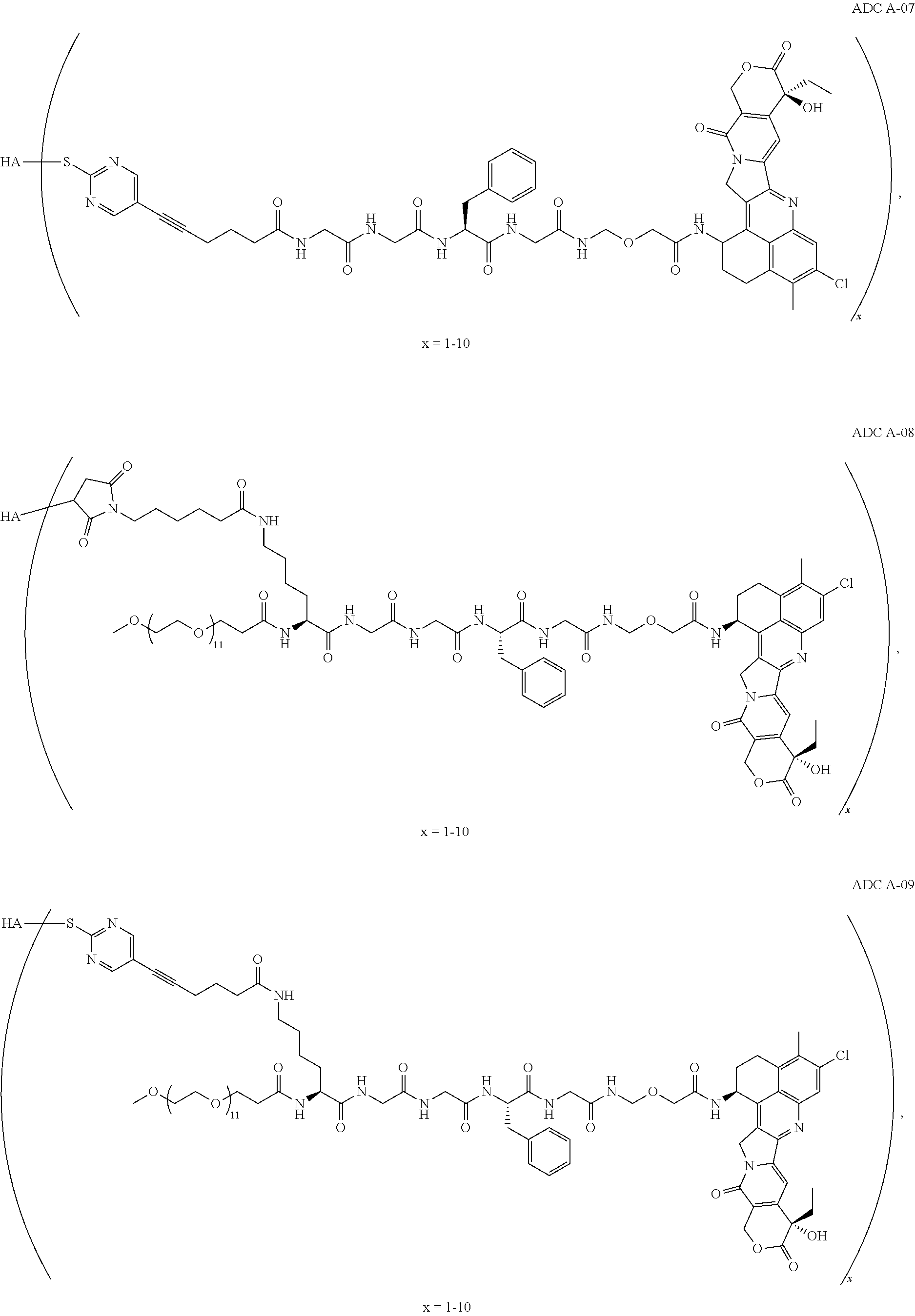

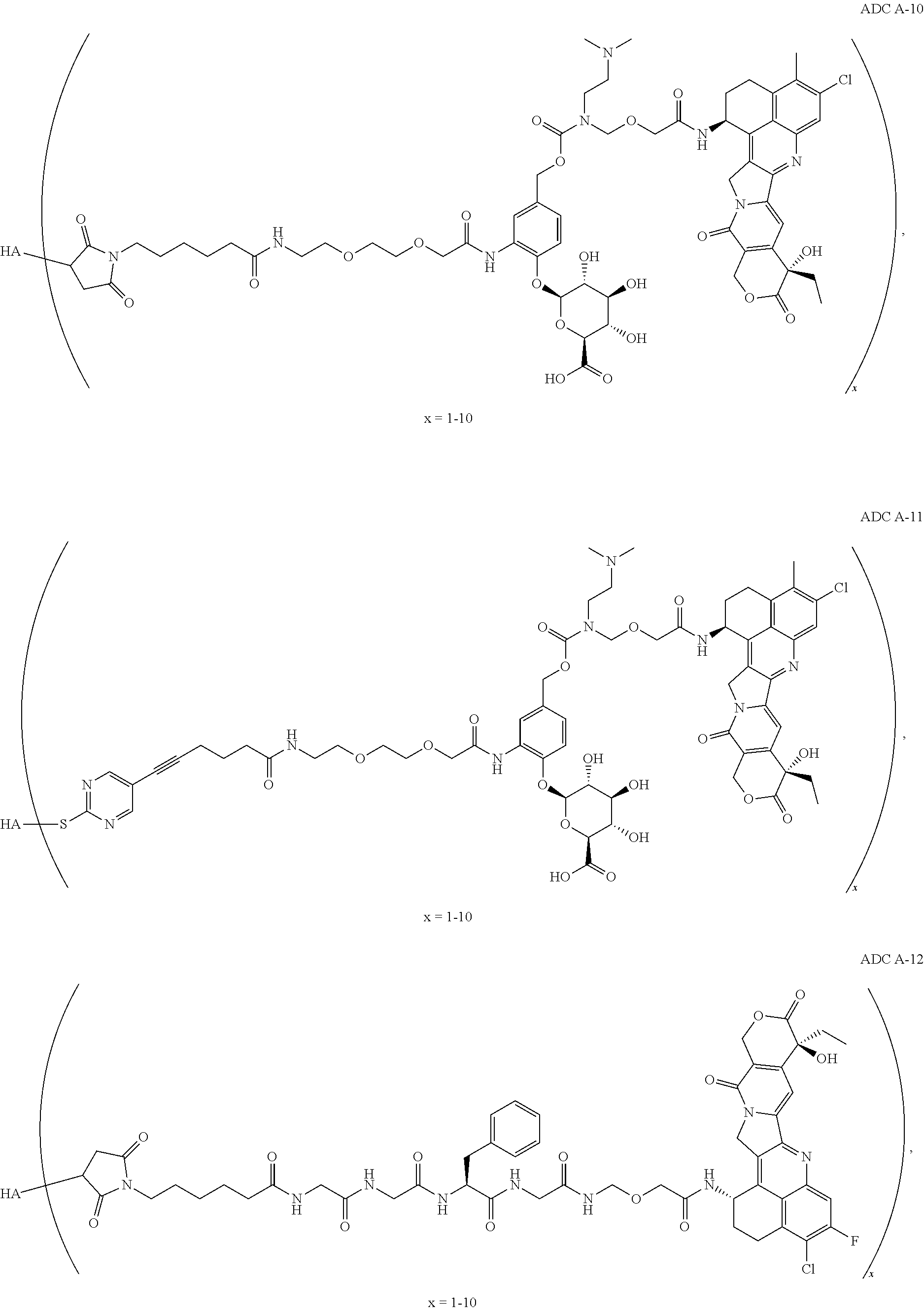

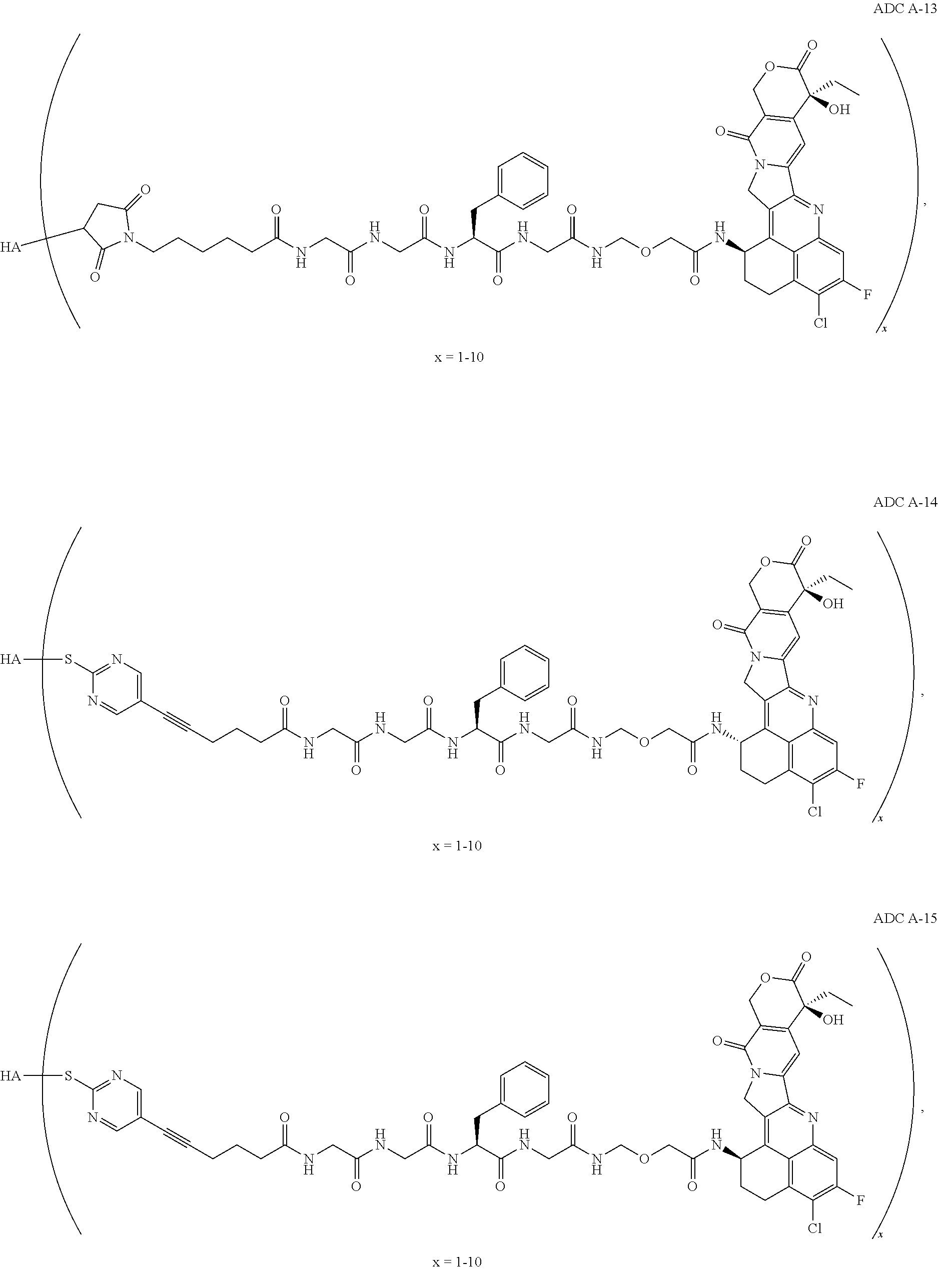

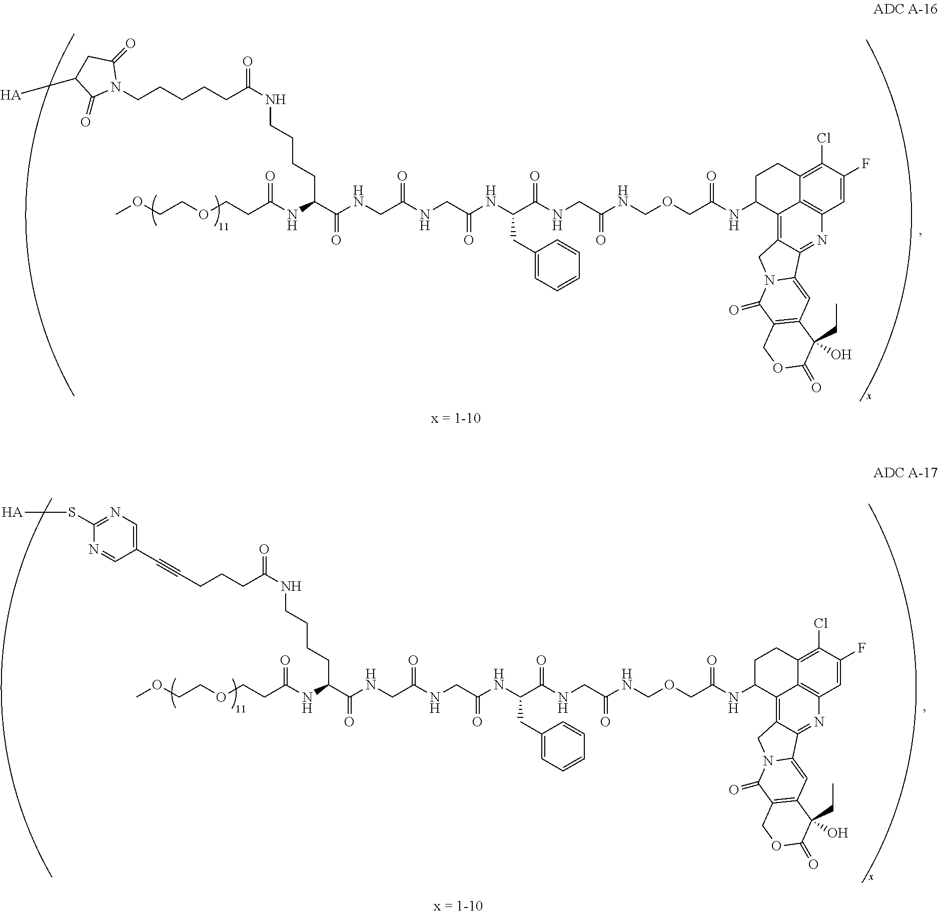

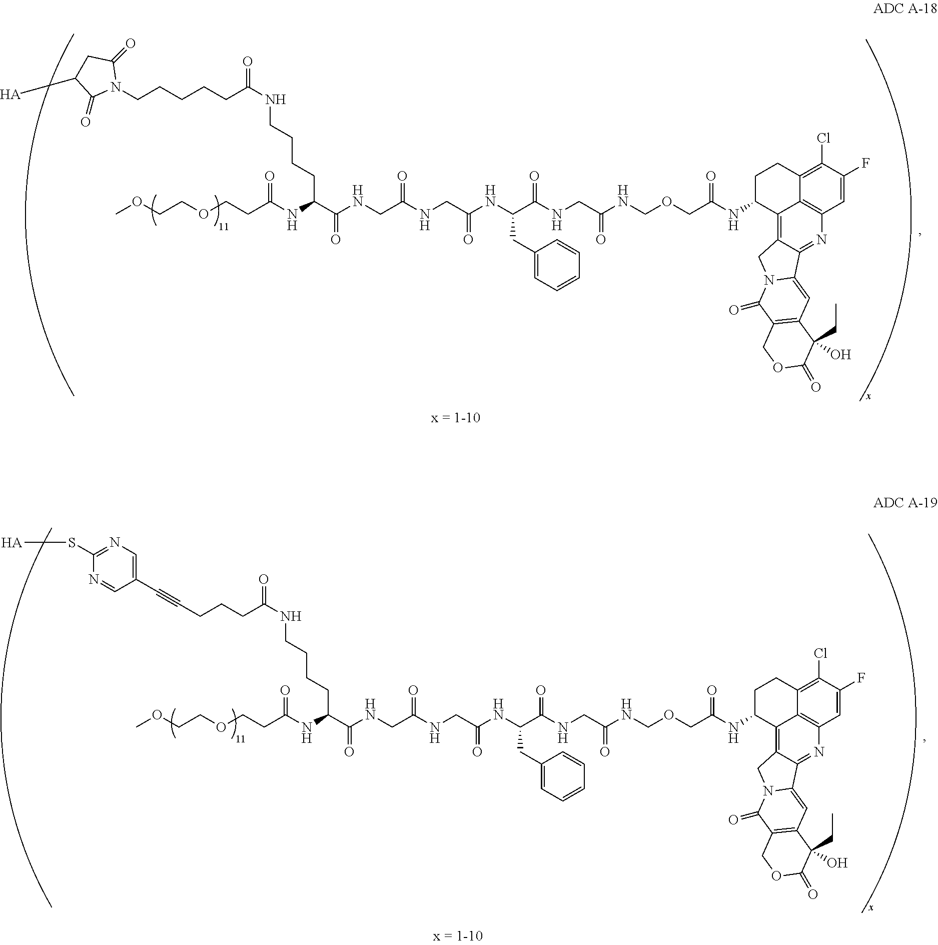

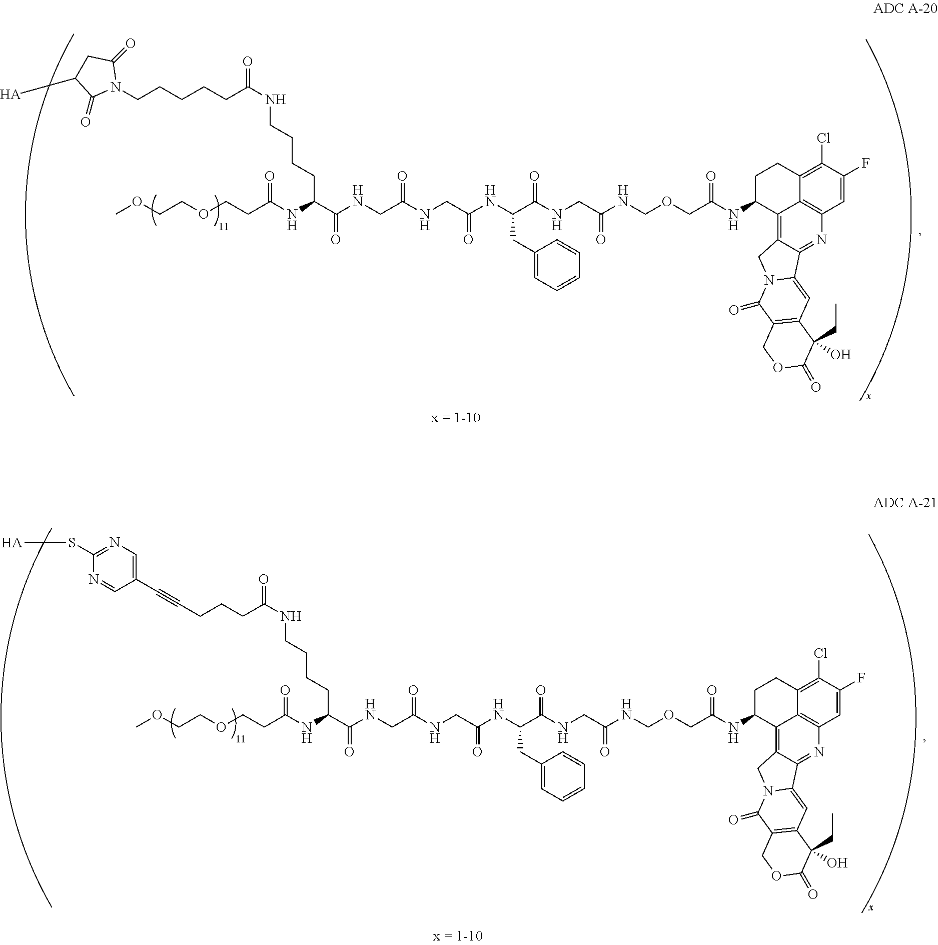

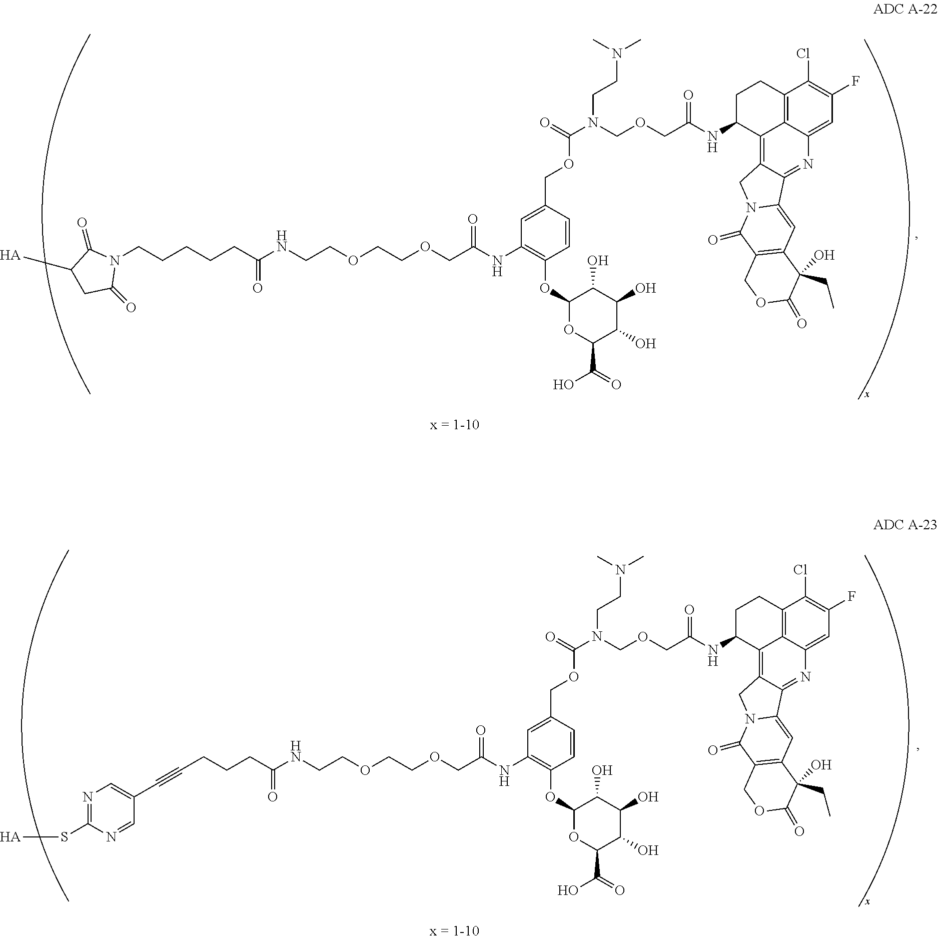

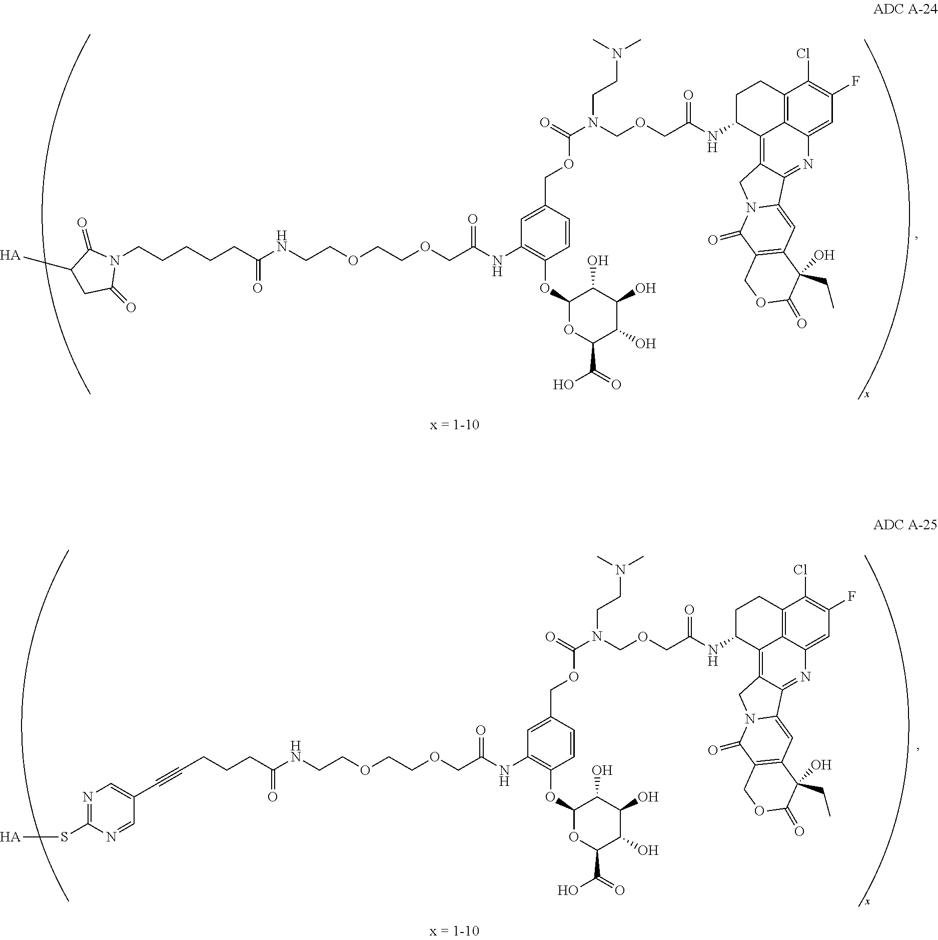

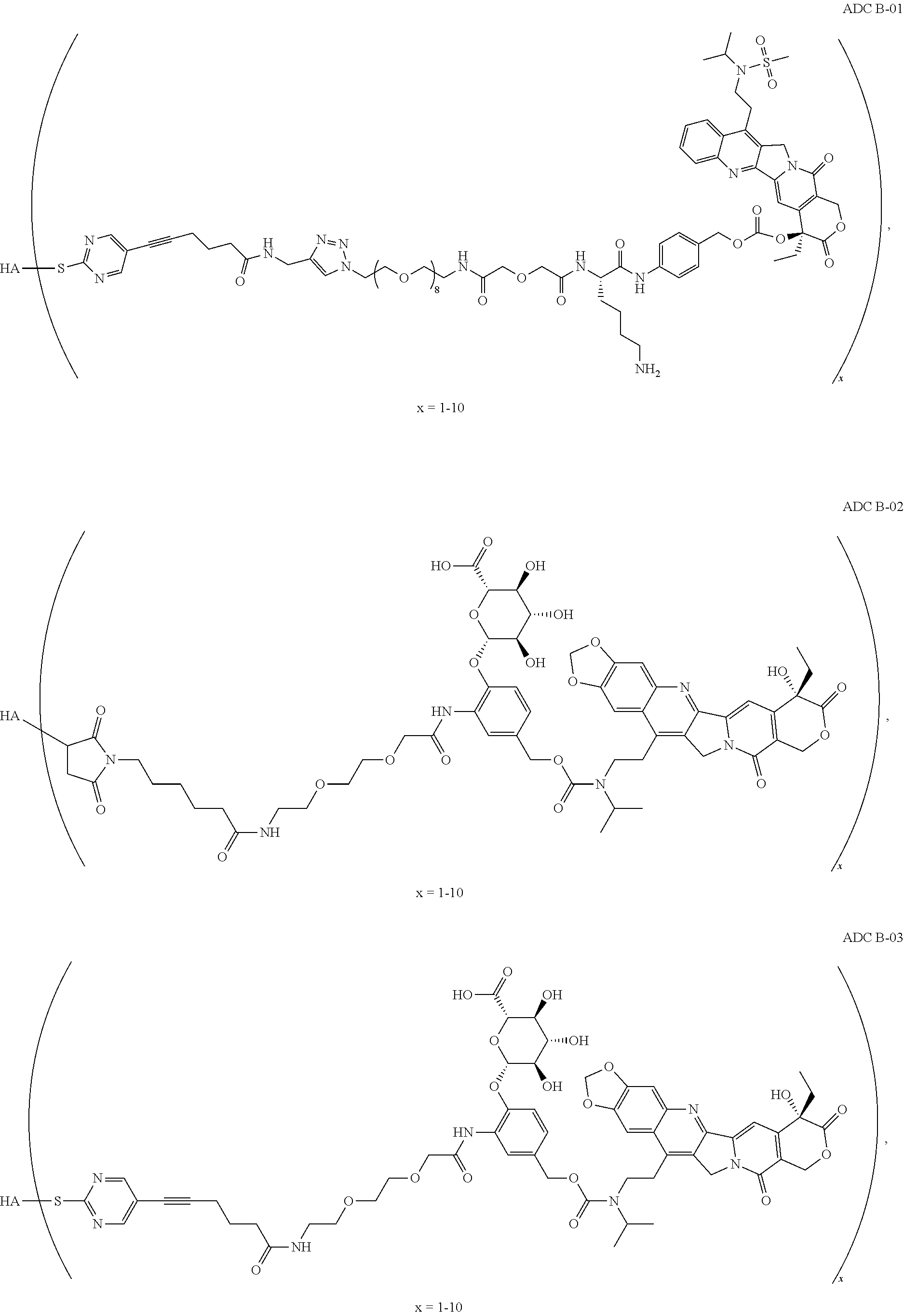

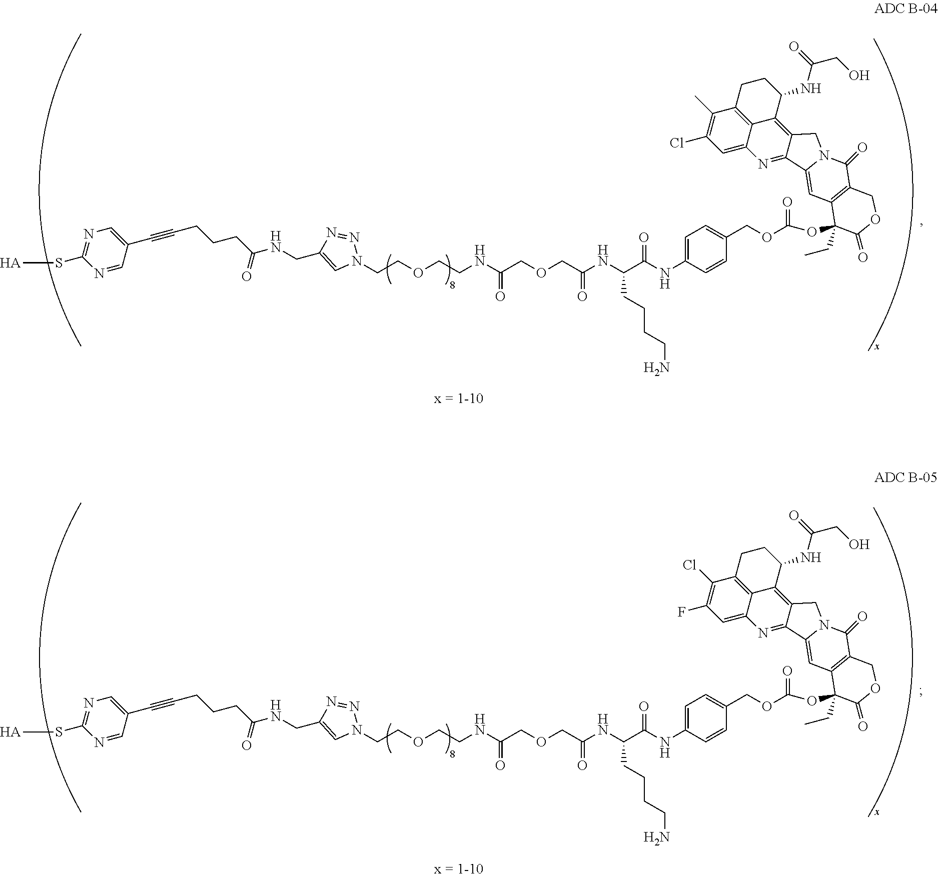

wherein, HA in each antibody-drug conjugate represents an antibody or antigen-binding fragment thereof comprising VH as set forth in SEQ ID NO: 15 and VL as set forth in SEQ ID NO: 16, for example, an antibody or antigen-binding fragment thereof comprising VH as set forth in SEQ ID NO: 15 and CH as set forth in SEQ ID NO: 50, as well as VL as set forth in SEQ ID NO: 16 and CL as set forth in SEQ ID NO: 51;
wherein,

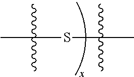 [SMILES: *S*]

represents a specific way that a sulfhydryl group in the antibody or antigen-binding fragment thereof connects with the linker.

In some embodiments, the antibody-drug conjugate is selected from:

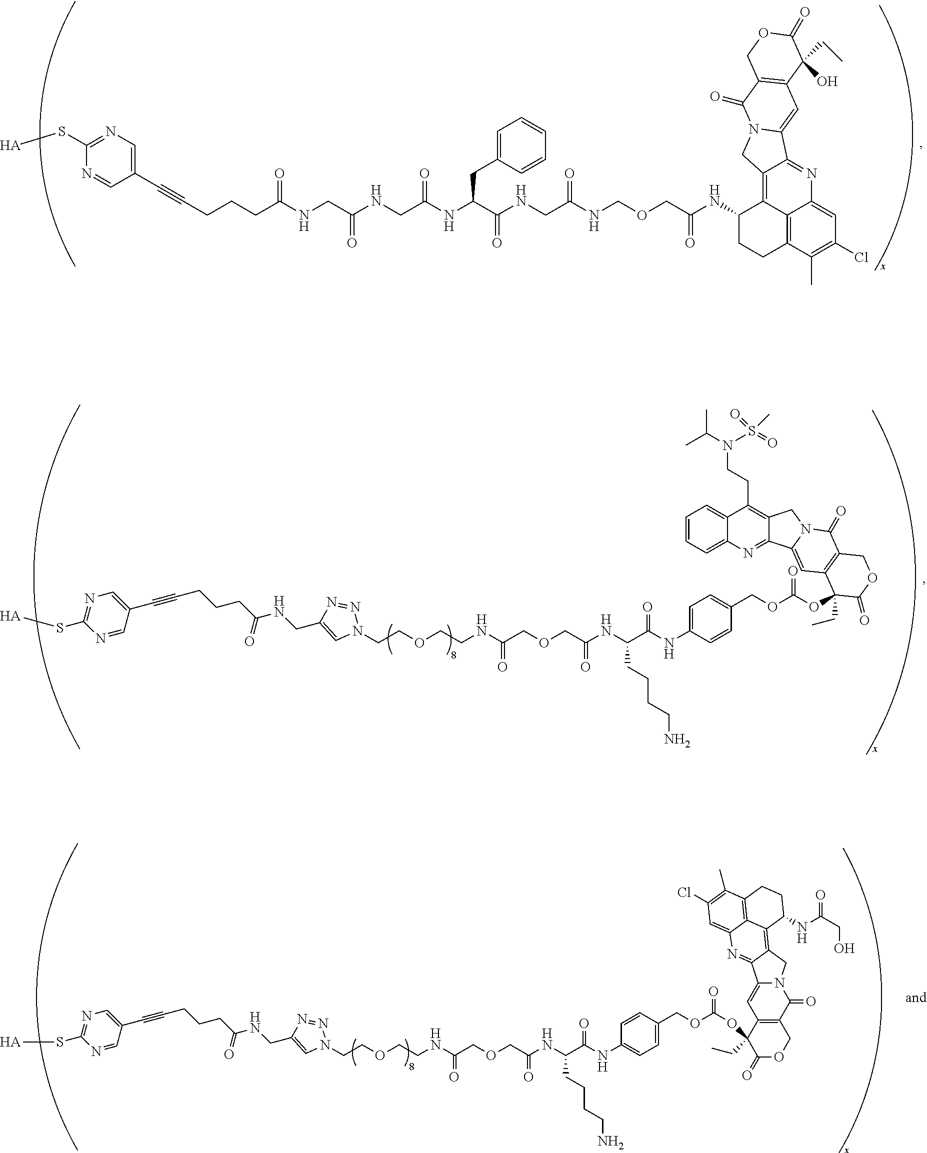

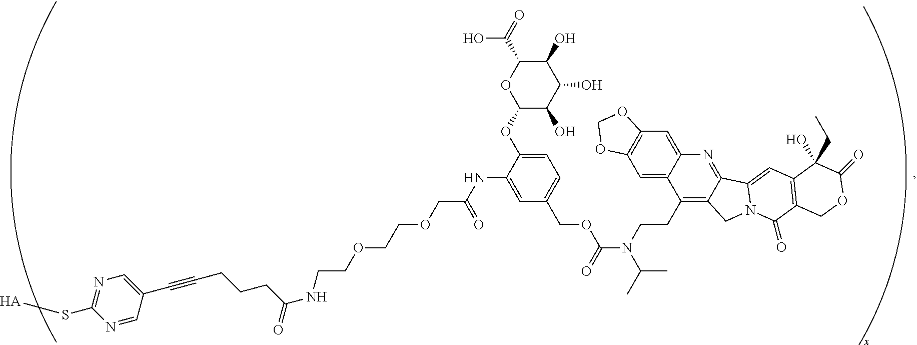

wherein, x is 3 to 8, or 3 to 4, or 7 to 8, and HA in each antibody-drug conjugate is selected from an antibody or antigen binding fragment comprising:
(1) a heavy chain variable region (VH) and/or a light chain variable region (VL) as follows, wherein the CDRs are defined by the Chothia numbering system:
(1a) a heavy chain variable region (VH) comprising the following 3 CDRs: CDR-H1 having the amino acid sequence as set forth in SEQ ID NO: 1 or a variant thereof, CDR-H2 having the amino acid sequence as set forth in SEQ ID NO: 2 or a variant thereof, CDR-H3 having the amino acid sequence as set forth in SEQ ID NO: 3 or a variant thereof; and/or, a light chain variable region (VL) comprising the following 3 CDRs: CDR-L1 having the amino acid sequence as set forth in SEQ ID NO: 4 or a variant thereof, CDR-L2 having the amino acid sequence as set forth in SEQ ID NO: 5 or a variant thereof, CDR-L3 having the amino acid sequence as set forth in SEQ ID NO: 6 or a variant thereof; or,
(1b) a heavy chain variable region (VH) comprising the following 3 CDRs: CDR-H1 having the amino acid sequence as set forth in SEQ ID NO: 19 or a variant thereof, CDR-H2 having the amino acid sequence as set forth in SEQ ID NO: 20 or a variant thereof, CDR-H3 having the amino acid sequence as set forth in SEQ ID NO: 21 or a variant thereof; and/or, a light chain variable region (VL) comprising the following 3 CDRs: CDR-L1 having the amino acid sequence as set forth in SEQ ID NO: 22 or a variant thereof, CDR-L2 having the amino acid sequence as set forth in SEQ ID NO: 23 or a variant thereof, CDR-L3 having the amino acid sequence as set forth in SEQ ID NO: 24 or a variant thereof; or,
(1c) a heavy chain variable region (VH) comprising the following 3 CDRs: CDR-H1 having the amino acid sequence as set forth in SEQ ID NO: 36 or a variant thereof, CDR-H2 having the amino acid sequence as set forth in SEQ ID NO: 37 or a variant thereof, CDR-H3 having the amino acid sequence as set forth in SEQ ID NO: 38 or a variant thereof; and/or, a light chain variable region (VL) comprising the following 3 CDRs: CDR-L1 having the amino acid sequence as set forth in SEQ ID NO: 45 or a variant thereof, CDR-L2 having the amino acid sequence as set forth in SEQ ID NO: 23 or a variant thereof, and CDR-L3 having the amino acid sequence as set forth in SEQ ID NO: 52 or a variant thereof;
wherein, the variant described in any item of (1a), (1b), and (1c) has the amino acid sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2 or 3 amino acids) as compared to the sequence from which it is derived; preferably, the substitution is a conservative substitution with the proviso that the amino acid sequences of the CDRs of the variant have 100% sequence identity to the amino acid sequences of the respective CDRs of the VH and VL of (1a), (1b), and (1c) and the variant binds HER3;
or,
(2) a heavy chain variable region (VH) and/or a light chain variable region (VL) as follows, wherein the CDRs are defined according to the Kabat numbering system:
(2a) a heavy chain variable region (VH) comprising the following 3 CDRs: CDR-H1 having the amino acid sequence as set forth in SEQ ID NO: 7 or a variant thereof, CDR-H2 having the amino acid sequence as set forth in SEQ ID NO: 8 or a variant thereof, CDR-H3 having the amino acid sequence as set forth in SEQ ID NO: 9 or a variant thereof; and/or, a light chain variable region (VL) comprising the following 3 CDRs: CDR-L1 having the amino acid sequence as set forth in SEQ ID NO: 4 or a variant thereof, CDR-L2 having the amino acid sequence as set forth in SEQ ID NO: 5 or a variant thereof, CDR-L3 having the amino acid sequence as set forth in SEQ ID NO: 6 or a variant thereof; or,
(2b) a heavy chain variable region (VH) comprising the following 3 CDRs: CDR-H1 having the amino acid sequence as set forth in SEQ ID NO: 25 or a variant thereof, CDR-H2 having the amino acid sequence as set forth in SEQ ID NO: 26 or a variant thereof, CDR-H3 having the amino acid sequence as set forth in SEQ ID NO: 21 or a variant thereof; and/or, a light chain variable region (VL) comprising the following 3 CDRs: CDR-L1 having the amino acid sequence as set forth in SEQ ID NO: 22 or a variant thereof, CDR-L2 having the amino acid sequence as set forth in SEQ ID NO: 23 or a variant thereof, CDR-L3 having the amino acid sequence as set forth in SEQ ID NO: 24 or a variant thereof; or,
(2c) a heavy chain variable region (VH) comprising the following 3 CDRs: CDR-H1 having the amino acid sequence as set forth in SEQ ID NO: 39 or a variant thereof, CDR-H2 having the amino acid sequence as set forth in SEQ ID NO: 40 or a variant thereof, CDR-H3 having the amino acid sequence as set forth in SEQ ID NO: 38 or a variant thereof; and/or, a light chain variable region (VL) comprising the following 3 CDRs: CDR-L1 having the amino acid sequence as set forth in SEQ ID NO: 45 or a variant thereof, CDR-L2 having the amino acid sequence as set forth in SEQ ID NO: 23 or a variant thereof, and CDR-L3 having the amino acid sequence as set forth in SEQ ID NO: 52 or a variant thereof;
wherein, the variant described in any item of (1a), (1b), and (1c) has the amino acid sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2 or 3 amino acids) as compared to the sequence from which it is derived; preferably, the substitution is a conservative substitution with the proviso that the amino acid sequences of the CDRs of the variant have 100% sequence identity to the amino acid sequences of the respective CDRs of the VH and VL of (2a), (2b), and (2c) and the variant binds HER3;
or,
(3) a heavy chain variable region (VH) and/or a light chain variable region (VL) as follows, wherein the CDRs are defined by the IMGT numbering system:
(3a) a heavy chain variable region (VH) comprising the following 3 CDRs: CDR-H1 having the amino acid sequence as set forth in SEQ ID NO: 10 or a variant thereof, CDR-H2 having the amino acid sequence as set forth in SEQ ID NO: 11 or a variant thereof, CDR-H3 having the amino acid sequence as set forth in SEQ ID NO: 12 or a variant thereof; and/or, a light chain variable region (VL) comprising the following 3 CDRs: CDR-L1 having the amino acid sequence as set forth in SEQ ID NO: 13 or a variant thereof, CDR-L2 having the amino acid sequence as set forth in SEQ ID NO: 14 or a variant thereof, CDR-L3 having the amino acid sequence as set forth in SEQ ID NO: 6 or a variant thereof; or,
(3b) a heavy chain variable region (VH) comprising the following 3 CDRs: CDR-H1 having the amino acid sequence as set forth in SEQ ID NO: 27 or a variant thereof, CDR-H2 having the amino acid sequence as set forth in SEQ ID NO: 28 or a variant thereof, CDR-H3 having the amino acid sequence as set forth in SEQ ID NO: 29 or a variant thereof; and/or, a light chain variable region (VL) comprising the following 3 CDRs: CDR-L1 having the amino acid sequence as set forth in SEQ ID NO: 30 or a variant thereof, CDR-L2 having the amino acid sequence as set forth in SEQ ID NO: 31 or a variant thereof, CDR-L3 having the amino acid sequence as set forth in SEQ ID NO: 24 or a variant thereof; or,
(3c) a heavy chain variable region (VH) comprising the following 3 CDRs: CDR-H1 having the amino acid sequence as set forth in SEQ ID NO: 41 or a variant thereof, CDR-H2 having the amino acid sequence as set forth in SEQ ID NO: 42 or a variant thereof, CDR-H3 having the amino acid sequence as set forth in SEQ ID NO: 43 or a variant thereof; and/or, a light chain variable region (VL) comprising the following 3 CDRs: CDR-L1 having the amino acid sequence as set forth in SEQ ID NO: 44 or a variant thereof, CDR-L2 having the amino acid sequence as set forth in SEQ ID NO: 31 or a variant thereof, and CDR-L3 having the amino acid sequence as set forth in SEQ ID NO: 52 or a variant thereof;
wherein, the variant described in any item of (1a), (1b), and (1c) has the amino acid sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2 or 3 amino acids) as compared to the sequence from which it is derived; preferably, the substitution is a conservative substitution with the proviso that the amino acid sequences of the CDRs of the variant have 100% sequence identity to the amino acid sequences of the respective CDRs of the VH and VL of (3a), (3b), and (3c) and the variant binds HER3.

In a further embodiment, the HA in each antibody-drug conjugate is selected from an antibody or antigen binding fragment comprising:
(a) a VH comprising the amino acid sequence as set forth in SEQ ID NO: 15 or a variant thereof, and/or, a VL comprising the amino acid sequence as set forth in SEQ ID NO: 16 or a variant thereof;
(b) a VH comprising the amino acid sequence as set forth in SEQ ID NO: 32 or a variant thereof, and/or, a VL comprising the amino acid sequence as set forth in SEQ ID NO: 33 or a variant thereof; or
(c) a VH comprising the amino acid sequence as set forth in SEQ ID NO: 46 or a variant thereof, and/or, a VL comprising the amino acid sequence as set forth in SEQ ID NO: 47 or a variant thereof;
wherein, the variant described in any item of (1a), (1b), and (1c) has the amino acid sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2 or 3 amino acids) as compared to the sequence from which it is derived; preferably, the substitution is a conservative substitution with the proviso that the amino acid sequences of the CDRs of the variant have 100% with the proviso that the amino acid sequences of the CDRs of the variant have 100% sequence identity to the amino acid sequences of the respective CDRs of the VH and VL of (a), (b), and (c) and the variant binds HER3.

In a further embodiment, the HA in each antibody-drug conjugate is selected from an antibody or antigen binding fragment comprising:
(a) a VH comprising the amino acid sequence set forth in SEQ ID NO: 15 and a VL comprising the amino acid sequence set forth in SEQ ID NO: 16;
(b) a VH comprising the amino acid sequence set forth in SEQ ID NO: 32 and a VL comprising the amino acid sequence set forth in SEQ ID NO: 33; or
(c) a VH comprising the amino acid sequence set forth in SEQ ID NO: 46 and a VL comprising the amino acid sequence set forth in SEQ ID NO: 47.

In a further embodiment, the antibody-drug conjugate is selected from:

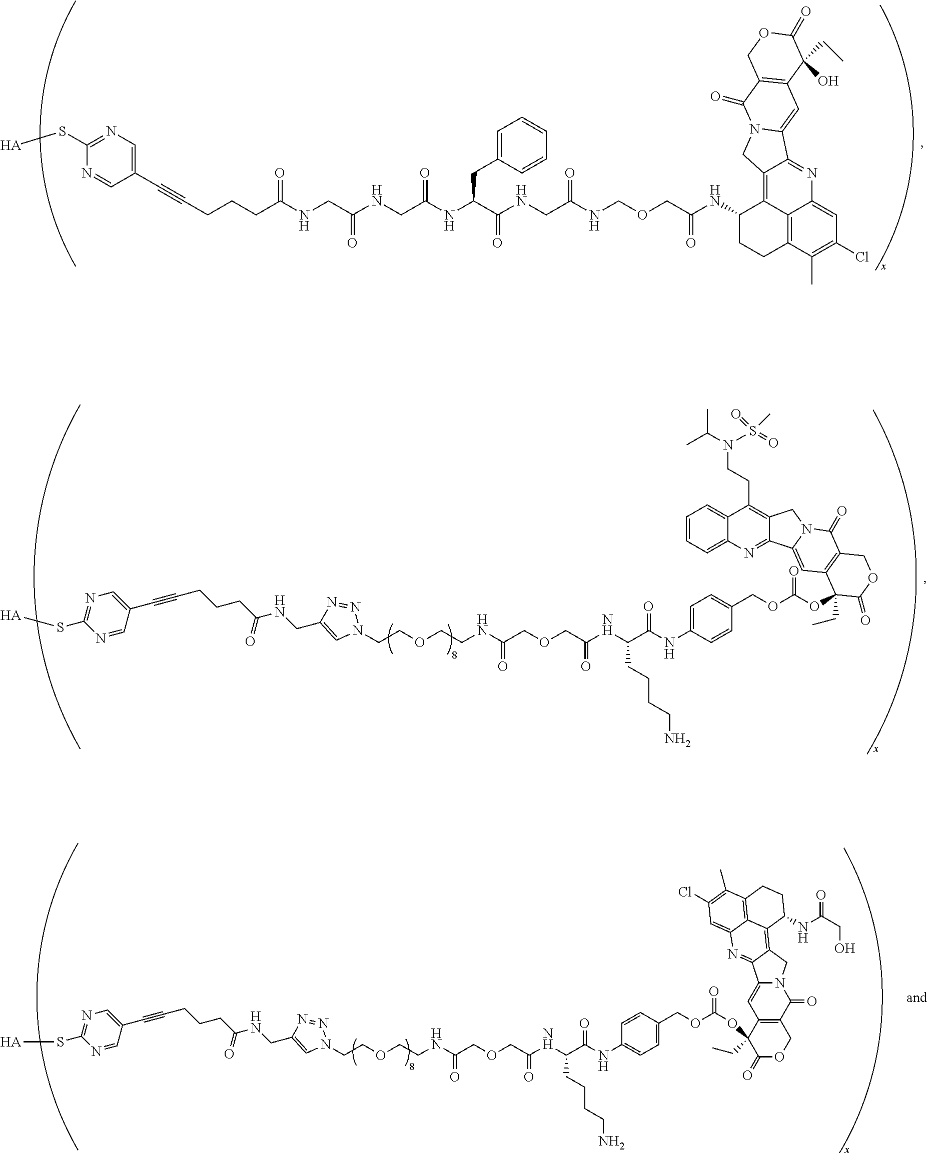

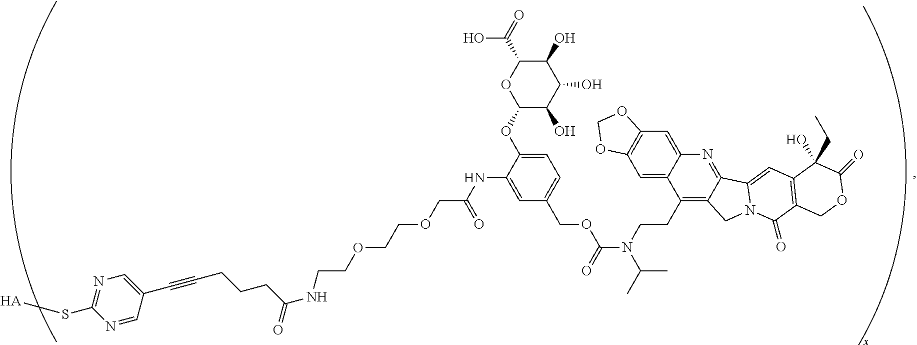

wherein the HA in each antibody-drug conjugate is selected from an antibody or antigen binding fragment comprising:
(1) an antibody or antigen-binding fragment thereof comprising a VH comprising the amino acid sequence as set forth in SEQ ID NO: 15 and a VL comprising the amino acid sequence as set forth in SEQ ID NO: 16, for example, an antibody or antigen-binding fragment thereof comprising a VH comprising the amino acid sequence as set forth in SEQ ID NO: 15 and CH as set forth in SEQ ID NO: 50, as well as a VL comprising the amino acid sequence as set forth in SEQ ID NO: 16 and a CL comprising the amino acid sequence as set forth in SEQ ID NO: 51;
(2) an antibody or antigen-binding fragment thereof comprising a VH comprising the amino acid sequence as set forth in SEQ ID NO: 32 and a VL comprising the amino acid sequence as set forth in SEQ ID NO: 33, for example, an antibody or antigen-binding fragment thereof comprising a VH comprising the amino acid sequence as set forth in SEQ ID NO: 32 and a CH comprising the amino acid sequence as set forth in SEQ ID NO: 50, as well as a VL comprising the amino acid sequence as set forth in SEQ ID NO: 33 and a CL comprising the amino acid sequence as set forth in SEQ ID NO: 51; and
(3) an antibody or antigen-binding fragment thereof comprising a VH comprising the amino acid sequence as set forth in SEQ ID NO: 46 and a VL comprising the amino acid sequence as set forth in SEQ ID NO: 47, for example, an antibody or antigen-binding fragment thereof comprising a VH comprising the amino acid sequence as set forth in SEQ ID NO: 46 and a CH comprising the amino acid sequence as set forth in SEQ ID NO: 50, as well as a VL comprising the amino acid sequence as set forth in SEQ ID NO: 47 and a CL comprising the amino acid sequence as set forth in SEQ ID NO: 51.

In some embodiments, the antibody-drug conjugate is:

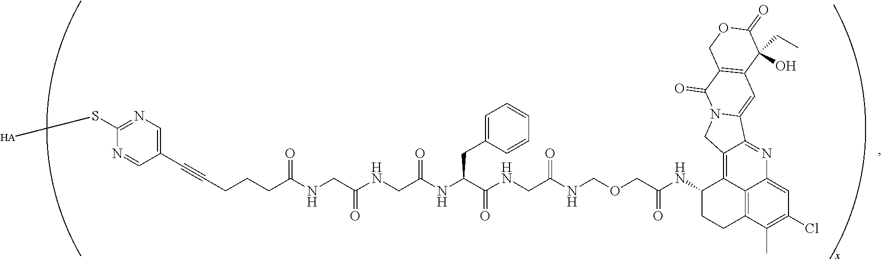

wherein the HA in each antibody-drug conjugate is selected from an antibody or antigen binding fragment comprising:
(1) an antibody or antigen-binding fragment thereof comprising a VH comprising the amino acid sequence as set forth in SEQ ID NO: 15 and a VL comprising the amino acid sequence as set forth in SEQ ID NO: 16, for example, an antibody or antigen-binding fragment thereof comprising a VH comprising the amino acid sequence as set forth in SEQ ID NO: 15 and a CH comprising the amino acid sequence as set forth in SEQ ID NO: 50, as well as a VL comprising the amino acid sequence as set forth in SEQ ID NO: 16 and a CL comprising the amino acid sequence as set forth in SEQ ID NO: 51;
(2) an antibody or antigen-binding fragment thereof comprising a VH comprising the amino acid sequence as set forth in SEQ ID NO: 32 and a VL comprising the amino acid sequence as set forth in SEQ ID NO: 33, for example, an antibody or antigen-binding fragment thereof comprising a VH comprising the amino acid sequence as set forth in SEQ ID NO: 32 and a CH comprising the amino acid sequence as set forth in SEQ ID NO: 50, as well as a VL comprising the amino acid sequence as set forth in SEQ ID NO: 33 and a CL comprising the amino acid sequence as set forth in SEQ ID NO: 51; and
(3) an antibody or antigen-binding fragment thereof comprising a VH comprising the amino acid sequence as set forth in SEQ ID NO: 46 and a VL comprising the amino acid sequence as set forth in SEQ ID NO: 47, for example, an antibody or antigen-binding fragment thereof comprising a VH comprising the amino acid sequence as set forth in SEQ ID NO: 46 and a CH comprising the amino acid sequence as set forth in SEQ ID NO: 50, as well as a VL comprising the amino acid sequence as set forth in SEQ ID NO: 47 and a CL comprising the amino acid sequence as set forth in SEQ ID NO: 51.

In some embodiments, the antibody-drug conjugate is:

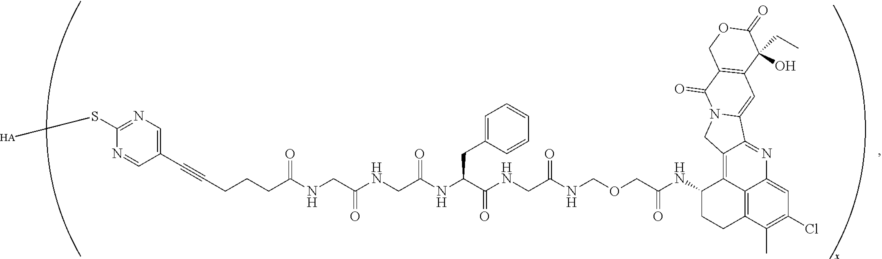

wherein the HA in each antibody-drug conjugate is selected from an antibody or antigen binding fragment comprising: (1) an antibody or antigen-binding fragment thereof comprising a VH comprising the amino acid sequence as set forth in SEQ ID NO: 15 and a VL comprising the amino acid sequence as set forth in SEQ ID NO: 16, for example, an antibody or antigen-binding fragment thereof comprising a VH comprising the amino acid sequence as set forth in SEQ ID NO: 15 and a CH comprising the amino acid sequence as set forth in SEQ ID NO: 50, as well as a VL comprising the amino acid sequence as set forth in SEQ ID NO: 16 and a CL comprising the amino acid sequence as set forth in SEQ ID NO: 51.

In some embodiments, the antibody-drug conjugate is:

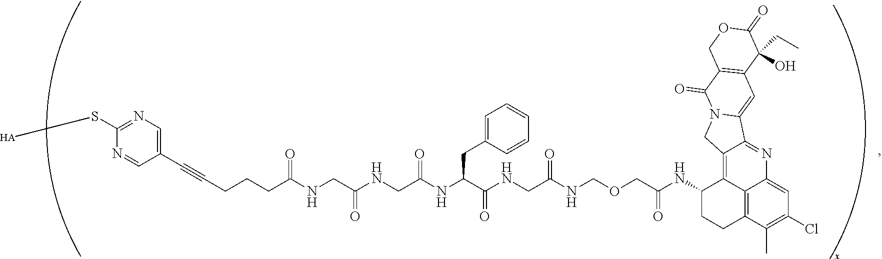

wherein the HA in each antibody-drug conjugate is selected from an antibody or antigen binding fragment comprising: (2) an antibody or antigen-binding fragment thereof comprising a VH comprising the amino acid sequence as set forth in SEQ ID NO: 32 and a VL comprising the amino acid sequence as set forth in SEQ ID NO: 33, for example, an antibody or antigen-binding fragment thereof comprising a VH comprising the amino acid sequence as set forth in SEQ ID NO: 32 and a CH comprising the amino acid sequence as set forth in SEQ ID NO: 50, as well as a VL comprising the amino acid sequence as set forth in SEQ ID NO: 33 and a CL comprising the amino acid sequence as set forth in SEQ ID NO: 51.

In some embodiments, the antibody-drug conjugate is:

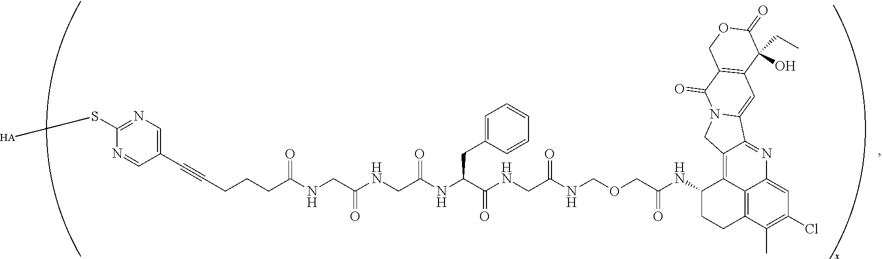

wherein the HA in each antibody-drug conjugate is selected from an antibody or antigen binding fragment comprising: (3) an antibody or antigen-binding fragment thereof comprising a VH comprising the amino acid sequence as set forth in SEQ ID NO: 46 and a VL comprising the amino acid sequence as set forth in SEQ ID NO: 47, for example, an antibody or antigen-binding fragment thereof comprising a VH comprising the amino acid sequence as set forth in SEQ ID NO: 46 and a CH comprising the amino acid sequence as set forth in SEQ ID NO: 50, as well as a VL comprising the amino acid sequence as set forth in SEQ ID NO: 47 and a CL comprising the amino acid sequence as set forth in SEQ ID NO: 51.

In some embodiments, the antibody-drug conjugate is:

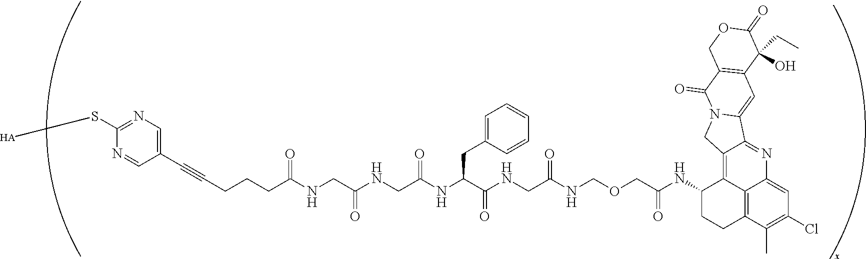

wherein, HA is an antibody or antigen-binding fragment thereof comprising a heavy chain (HC) comprising the amino acid sequence as set forth in SEQ ID NO: 17 and a light chain (LC) comprising the amino acid sequence as set forth in SEQ ID NO: 18; and x is 7 to 8.

In some embodiments, the antibody-drug conjugate is:

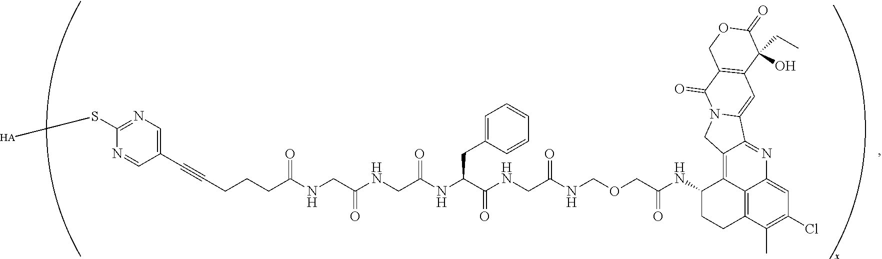

wherein, HA is an antibody or antigen-binding fragment thereof comprising a heavy chain (HC) comprising the amino acid sequence as set forth in SEQ ID NO: 34 and a light chain (LC) comprising the amino acid sequence as set forth in SEQ ID NO: 35; and x is 7 to 8.

In some embodiments, the antibody-drug conjugate is:

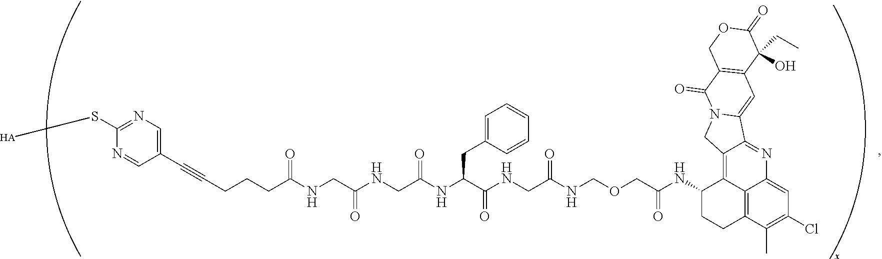

wherein, HA is an antibody or antigen-binding fragment thereof comprising a heavy chain (HC) comprising the amino acid sequence as set forth in SEQ ID NO: 48 and a light chain (LC) comprising the amino acid sequence as set forth in SEQ ID NO: 49; and x is 7 to 8.

In some embodiments, the antibody-drug conjugate is:

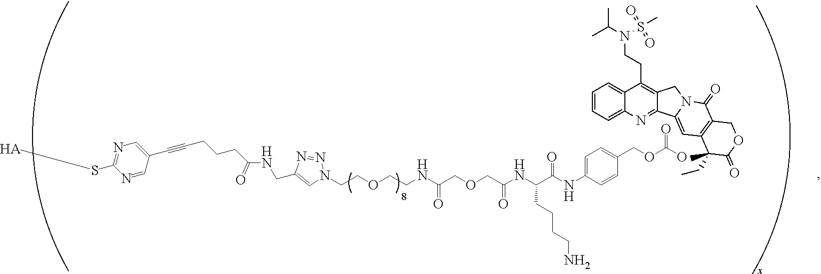

wherein the HA in each antibody-drug conjugate is selected from an antibody or antigen binding fragment comprising:
(1) an antibody or antigen-binding fragment thereof comprising a VH comprising the amino acid sequence as set forth in SEQ ID NO: 15 and a VL comprising the amino acid sequence as set forth in SEQ ID NO: 16, for example, an antibody or antigen-binding fragment thereof comprising a VH comprising the amino acid sequence as set forth in SEQ ID NO: 15 and a CH comprising the amino acid sequence as set forth in SEQ ID NO: 50, as well as a VL comprising the amino acid sequence as set forth in SEQ ID NO: 16 and a CL comprising the amino acid sequence as set forth in SEQ ID NO: 51;
(2) an antibody or antigen-binding fragment thereof comprising a VH comprising the amino acid sequence as set forth in SEQ ID NO: 32 and a VL comprising the amino acid sequence as set forth in SEQ ID NO: 33, for example, an antibody or antigen-binding fragment thereof comprising a VH comprising the amino acid sequence as set forth in SEQ ID NO: 32 and a CH comprising the amino acid sequence as set forth in SEQ ID NO: 50, as well as a VL comprising the amino acid sequence as set forth in SEQ ID NO: 33 and a CL comprising the amino acid sequence as set forth in SEQ ID NO: 51; and
(3) an antibody or antigen-binding fragment thereof comprising a VH comprising the amino acid sequence as set forth in SEQ ID NO: 46 and a VL comprising the amino acid sequence as set forth in SEQ ID NO: 47, for example, an antibody or antigen-binding fragment thereof comprising a VH comprising the amino acid sequence as set forth in SEQ ID NO: 46 and a CH comprising the amino acid sequence as set forth in SEQ ID NO: 50, as well as a VL comprising the amino acid sequence as set forth in SEQ ID NO: 47 and a CL comprising the amino acid sequence as set forth in SEQ ID NO: 51.

In some embodiments, the antibody-drug conjugate is:

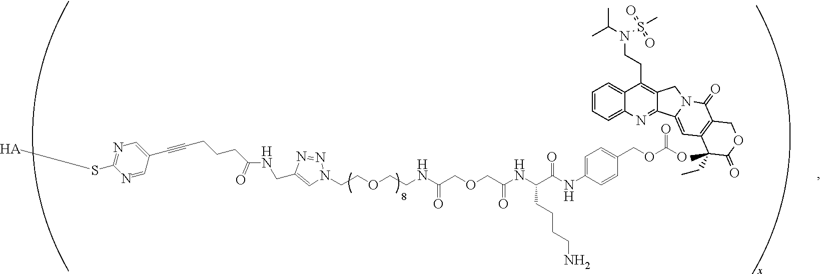

wherein the HA in each antibody-drug conjugate is selected from an antibody or antigen binding fragment comprising: (1) an antibody or antigen-binding fragment thereof comprising a VH comprising the amino acid sequence as set forth in SEQ ID NO: 15 and a VL comprising the amino acid sequence as set forth in SEQ ID NO: 16, for example, an antibody or antigen-binding fragment thereof comprising a VH comprising the amino acid sequence as set forth in SEQ ID NO: 15 and a CH comprising the amino acid sequence as set forth in SEQ ID NO: 50, as well as a VL comprising the amino acid sequence as set forth in SEQ ID NO: 16 and a CL comprising the amino acid sequence as set forth in SEQ ID NO: 51.

In some embodiments, the antibody-drug conjugate is:

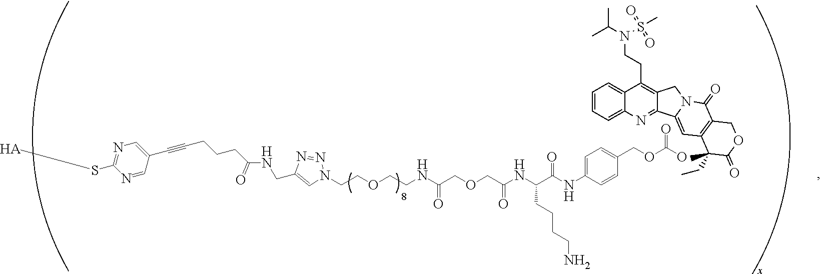

wherein the HA in each antibody-drug conjugate is selected from an antibody or antigen binding fragment comprising: (2) an antibody or antigen-binding fragment thereof comprising a VH comprising the amino acid sequence as set forth in SEQ ID NO: 32 and a VL comprising the amino acid sequence as set forth in SEQ ID NO: 33, for example, an antibody or antigen-binding fragment thereof comprising a VH comprising the amino acid sequence as set forth in SEQ ID NO: 32 and a CH comprising the amino acid sequence as set forth in SEQ ID NO: 50, as well as a VL comprising the amino acid sequence as set forth in SEQ ID NO: 33 and a CL comprising the amino acid sequence as set forth in SEQ ID NO: 51.

In some embodiments, the antibody-drug conjugate is:

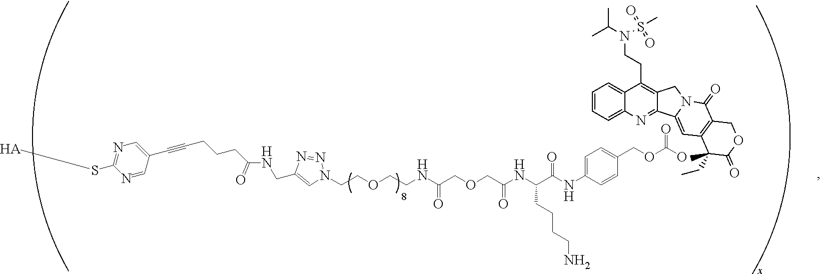

wherein the HA in each antibody-drug conjugate is selected from an antibody or antigen binding fragment comprising: (3) an antibody or antigen-binding fragment thereof comprising a VH comprising the amino acid sequence as set forth in SEQ ID NO: 46 and a VL comprising the amino acid sequence as set forth in SEQ ID NO: 47, for example, an antibody or antigen-binding fragment thereof comprising a VH comprising the amino acid sequence as set forth in SEQ ID NO: 46 and a CH comprising the amino acid sequence as set forth in SEQ ID NO: 50, as well as a VL comprising the amino acid sequence as set forth in SEQ ID NO: 47 and a CL comprising the amino acid sequence as set forth in SEQ ID NO: 51.

In some embodiments, the antibody-drug conjugate is:

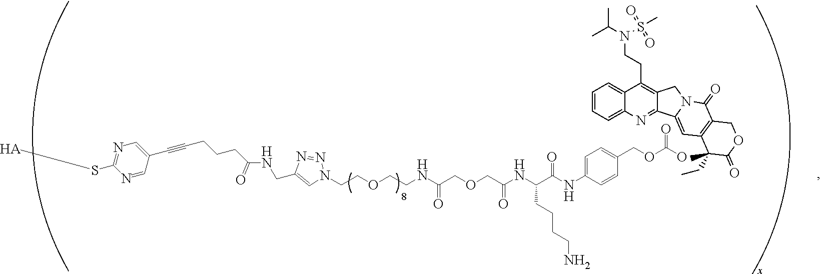

wherein, HA is an antibody or antigen-binding fragment thereof comprising a heavy chain (HC) comprising the amino acid sequence as set forth in SEQ ID NO: 17 and a light chain (LC) comprising the amino acid sequence as set forth in SEQ ID NO: 18; and x is 7 to 8.

In some embodiments, the antibody-drug conjugate is:

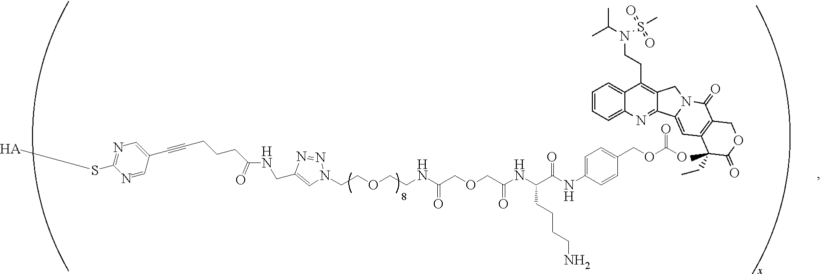

wherein, HA is an antibody or antigen-binding fragment thereof comprising a heavy chain (HC) comprising the amino acid sequence as set forth in SEQ ID NO: 34 and a light chain (LC) comprising the amino acid sequence as set forth in SEQ ID NO: 35; and x is 7 to 8.

In some embodiments, the antibody-drug conjugate is:

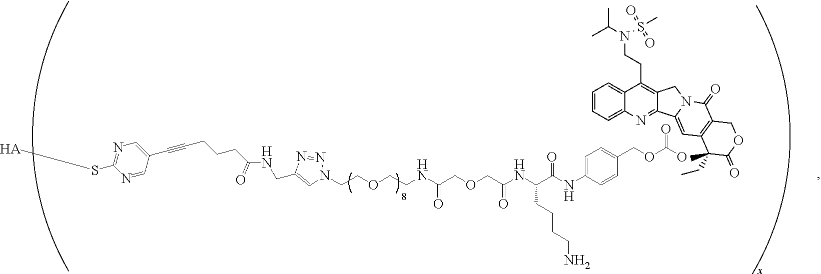

wherein, HA is an antibody or antigen-binding fragment thereof comprising a heavy chain (HC) comprising the amino acid sequence as set forth in SEQ ID NO: 48 and a light chain (LC) comprising the amino acid sequence as set forth in SEQ ID NO: 49; and x is 7 to 8.

In some embodiments, the antibody-drug conjugate is:

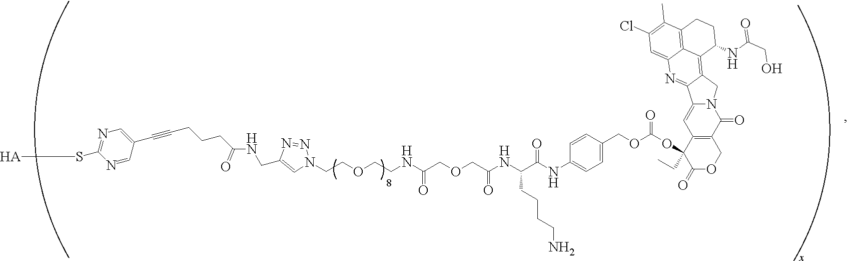

wherein the HA in each antibody-drug conjugate is selected from an antibody or antigen binding fragment comprising:
(1) an antibody or antigen-binding fragment thereof comprising a VH comprising the amino acid sequence as set forth in SEQ ID NO: 15 and a VL comprising the amino acid sequence as set forth in SEQ ID NO: 16, for example, an antibody or antigen-binding fragment thereof comprising a VH comprising the amino acid sequence as set forth in SEQ ID NO: 15 and a CH comprising the amino acid sequence as set forth in SEQ ID NO: 50, as well as a VL comprising the amino acid sequence as set forth in SEQ ID NO: 16 and a CL comprising the amino acid sequence as set forth in SEQ ID NO: 51;
(2) an antibody or antigen-binding fragment thereof comprising a VH comprising the amino acid sequence as set forth in SEQ ID NO: 32 and a VL comprising the amino acid sequence as set forth in SEQ ID NO: 33, for example, an antibody or antigen-binding fragment thereof comprising a VH comprising the amino acid sequence as set forth in SEQ ID NO: 32 and a CH comprising the amino acid sequence as set forth in SEQ ID NO: 50, as well as a VL comprising the amino acid sequence as set forth in SEQ ID NO: 33 and a CL comprising the amino acid sequence as set forth in SEQ ID NO: 51; and
(3) an antibody or antigen-binding fragment thereof comprising a VH comprising the amino acid sequence as set forth in SEQ ID NO: 46 and a VL comprising the amino acid sequence as set forth in SEQ ID NO: 47, for example, an antibody or antigen-binding fragment thereof comprising a VH comprising the amino acid sequence as set forth in SEQ ID NO: 46 and a CH comprising the amino acid sequence as set forth in SEQ ID NO: 50, as well as a VL comprising the amino acid sequence as set forth in SEQ ID NO: 47 and a CL comprising the amino acid sequence as set forth in SEQ ID NO: 51.

In some embodiments, the antibody-drug conjugate is:

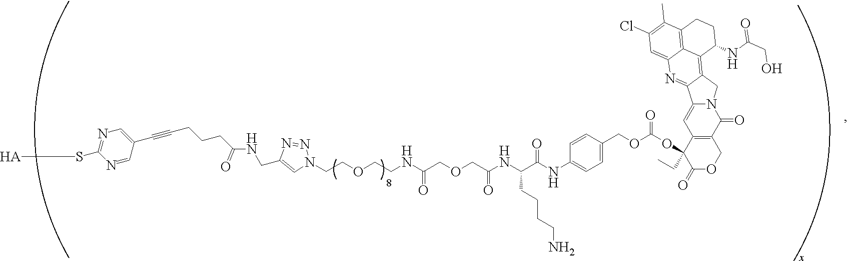

wherein the HA in each antibody-drug conjugate is selected from an antibody or antigen binding fragment comprising: (1) an antibody or antigen-binding fragment thereof comprising a VH comprising the amino acid sequence as set forth in SEQ ID NO: 15 and a VL comprising the amino acid sequence as set forth in SEQ ID NO: 16, for example, an antibody or antigen-binding fragment thereof comprising a VH comprising the amino acid sequence as set forth in SEQ ID NO: 15 and a CH comprising the amino acid sequence as set forth in SEQ ID NO: 50, as well as a VL comprising the amino acid sequence as set forth in SEQ ID NO: 16 and a CL comprising the amino acid sequence as set forth in SEQ ID NO: 51.

In some embodiments, the antibody-drug conjugate is:

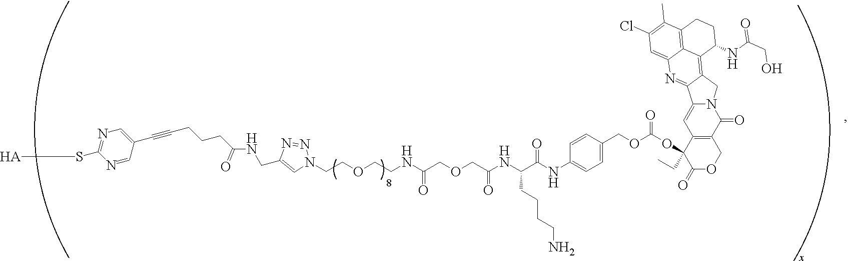

wherein the HA in each antibody-drug conjugate is selected from an antibody or antigen binding fragment comprising: (2) an antibody or antigen-binding fragment thereof comprising a VH comprising the amino acid sequence as set forth in SEQ ID NO: 32 and a VL comprising the amino acid sequence as set forth in SEQ ID NO: 33, for example, an antibody or antigen-binding fragment thereof comprising a VH comprising the amino acid sequence as set forth in SEQ ID NO: 32 and a CH comprising the amino acid sequence as set forth in SEQ ID NO: 50, as well as a VL comprising the amino acid sequence as set forth in SEQ ID NO: 33 and a CL comprising the amino acid sequence as set forth in SEQ ID NO: 51.

In some embodiments, the antibody-drug conjugate is:

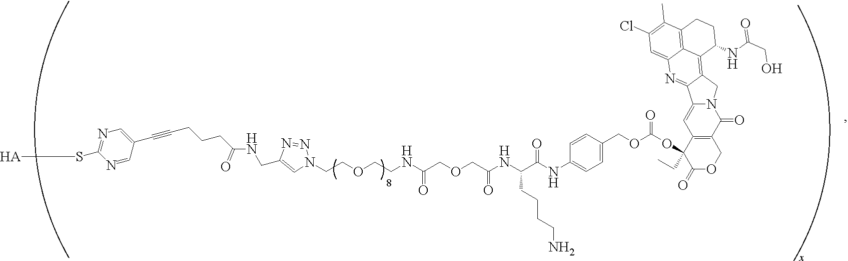

wherein the HA in each antibody-drug conjugate is selected from an antibody or antigen binding fragment comprising: (3) an antibody or antigen-binding fragment thereof comprising a VH comprising the amino acid sequence as set forth in SEQ ID NO: 46 and a VL comprising the amino acid sequence as set forth in SEQ ID NO: 47, for example, an antibody or antigen-binding fragment thereof comprising a VH comprising the amino acid sequence as set forth in SEQ ID NO: 46 and a CH comprising the amino acid sequence as set forth in SEQ ID NO: 50, as well as a VL comprising the amino acid sequence as set forth in SEQ ID NO: 47 and a CL comprising the amino acid sequence as set forth in SEQ ID NO: 51.

In some embodiments, the antibody-drug conjugate is:

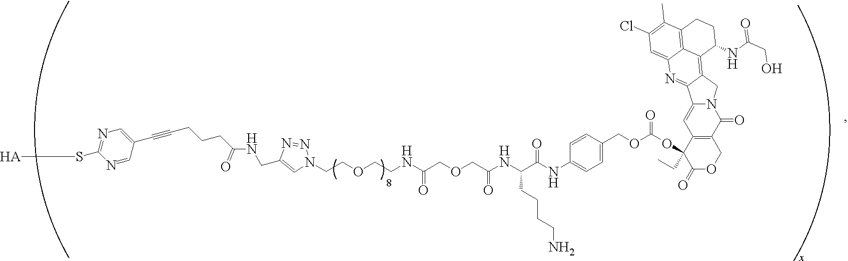

wherein, HA is an antibody or antigen-binding fragment thereof comprising a heavy chain (HC) comprising the amino acid sequence as set forth in SEQ ID NO: 17 and a light chain (LC) comprising the amino acid sequence as set forth in SEQ ID NO: 18; and x is 7 to 8.

In some embodiments, the antibody-drug conjugate is:

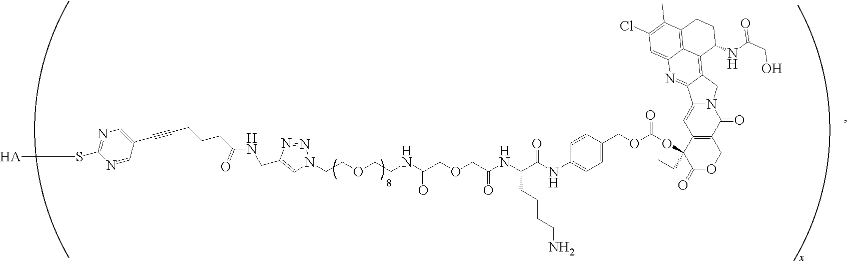

wherein, HA is an antibody or antigen-binding fragment thereof comprising a heavy chain (HC) comprising the amino acid sequence as set forth in SEQ ID NO: 34 and a light chain (LC) comprising the amino acid sequence as set forth in SEQ ID NO: 35; and x is 7 to 8.

In some embodiments, the antibody-drug conjugate is:

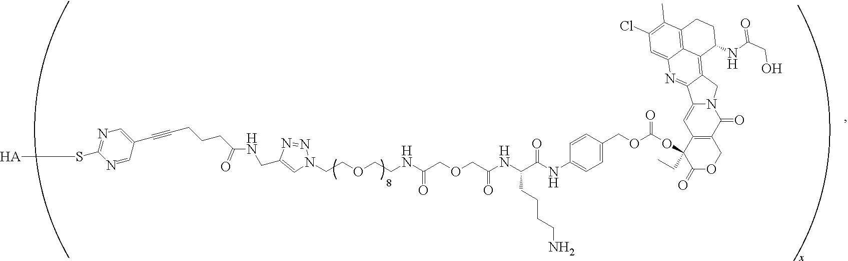

wherein, HA is an antibody or antigen-binding fragment thereof comprising a heavy chain (HC) comprising the amino acid sequence as set forth in SEQ ID NO: 48 and a light chain (LC) comprising the amino acid sequence as set forth in SEQ ID NO: 49; and x is 7 to 8.

In some embodiments, the antibody-drug conjugate is:

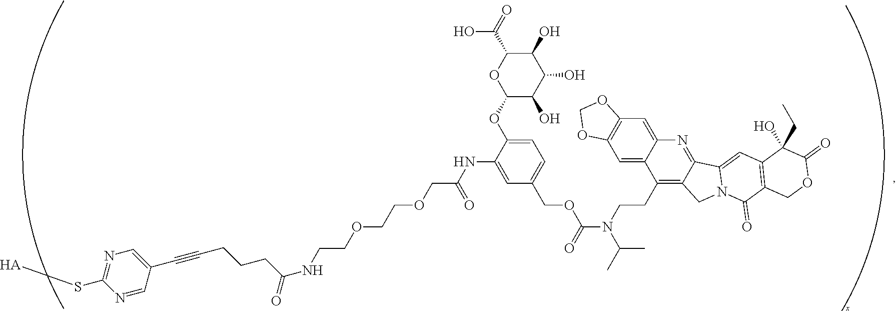

wherein the HA in each antibody-drug conjugate is selected from an antibody or antigen binding fragment comprising:
(1) an antibody or antigen-binding fragment thereof comprising a VH comprising the amino acid sequence as set forth in SEQ ID NO: 15 and a VL comprising the amino acid sequence as set forth in SEQ ID NO: 16, for example, an antibody or antigen-binding fragment thereof comprising a VH comprising the amino acid sequence as set forth in SEQ ID NO: 15 and a CH comprising the amino acid sequence as set forth in SEQ ID NO: 50, as well as a VL comprising the amino acid sequence as set forth in SEQ ID NO: 16 and a CL comprising the amino acid sequence as set forth in SEQ ID NO: 51;
(2) an antibody or antigen-binding fragment thereof comprising a VH comprising the amino acid sequence as set forth in SEQ ID NO: 32 and a VL comprising the amino acid sequence as set forth in SEQ ID NO: 33, for example, an antibody or antigen-binding fragment thereof comprising a VH comprising the amino acid sequence as set forth in SEQ ID NO: 32 and a CH comprising the amino acid sequence as set forth in SEQ ID NO: 50, as well as a VL comprising the amino acid sequence as set forth in SEQ ID NO: 33 and a CL comprising the amino acid sequence as set forth in SEQ ID NO: 51; and
(3) an antibody or antigen-binding fragment thereof comprising a VH comprising the amino acid sequence as set forth in SEQ ID NO: 46 and a VL comprising the amino acid sequence as set forth in SEQ ID NO: 47, for example, an antibody or antigen-binding fragment thereof comprising a VH comprising the amino acid sequence as set forth in SEQ ID NO: 46 and a CH comprising the amino acid sequence as set forth in SEQ ID NO: 50, as well as a VL comprising the amino acid sequence as set forth in SEQ ID NO: 47 and a CL comprising the amino acid sequence as set forth in SEQ ID NO: 51.

In some embodiments, the antibody-drug conjugate is:

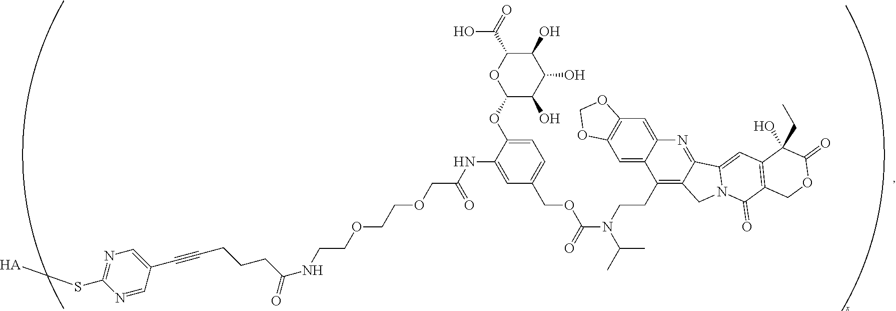

wherein the HA in each antibody-drug conjugate is selected from an antibody or antigen binding fragment comprising: (1) an antibody or antigen-binding fragment thereof comprising a VH comprising the amino acid sequence as set forth in SEQ ID NO: 15 and a VL comprising the amino acid sequence as set forth in SEQ ID NO: 16, for example, an antibody or antigen-binding fragment thereof comprising a VH comprising the amino acid sequence as set forth in SEQ ID NO: 15 and a CH comprising the amino acid sequence as set forth in SEQ ID NO: 50, as well as a VL comprising the amino acid sequence as set forth in SEQ ID NO: 16 and a CL comprising the amino acid sequence as set forth in SEQ ID NO: 51.

In some embodiments, the antibody-drug conjugate is:

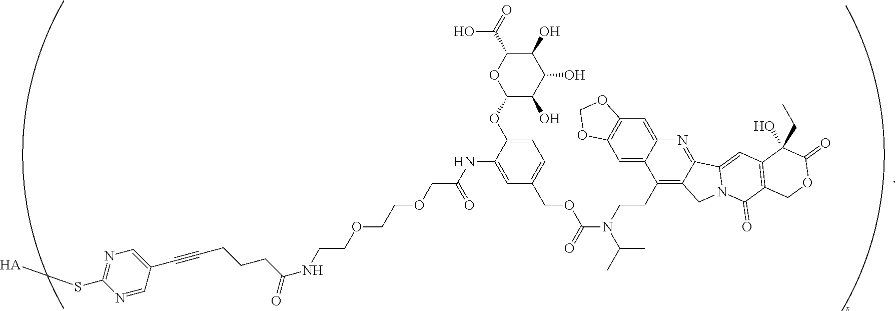

wherein the HA in each antibody-drug conjugate is selected from an antibody or antigen binding fragment comprising: (2) an antibody or antigen-binding fragment thereof comprising a VH comprising the amino acid sequence as set forth in SEQ ID NO: 32 and a VL comprising the amino acid sequence as set forth in SEQ ID NO: 33, for example, an antibody or antigen-binding fragment thereof comprising a VH comprising the amino acid sequence as set forth in SEQ ID NO: 32 and a CH comprising the amino acid sequence as set forth in SEQ ID NO: 50, as well as a VL comprising the amino acid sequence as set forth in SEQ ID NO: 33 and a CL comprising the amino acid sequence as set forth in SEQ ID NO: 51.

In some embodiments, the antibody-drug conjugate is:

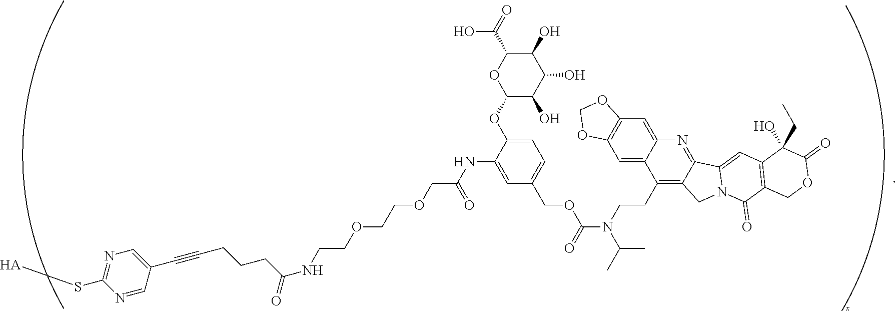

wherein the HA in each antibody-drug conjugate is selected from an antibody or antigen binding fragment comprising: (3) an antibody or antigen-binding fragment thereof comprising a VH comprising the amino acid sequence as set forth in SEQ ID NO: 46 and a VL comprising the amino acid sequence as set forth in SEQ ID NO: 47, for example, an antibody or antigen-binding fragment thereof comprising a VH comprising the amino acid sequence as set forth in SEQ ID NO: 46 and a CH comprising the amino acid sequence as set forth in SEQ ID NO: 50, as well as a VL comprising the amino acid sequence as set forth in SEQ ID NO: 47 and a CL comprising the amino acid sequence as set forth in SEQ ID NO: 51.

In some embodiments, the antibody-drug conjugate is:

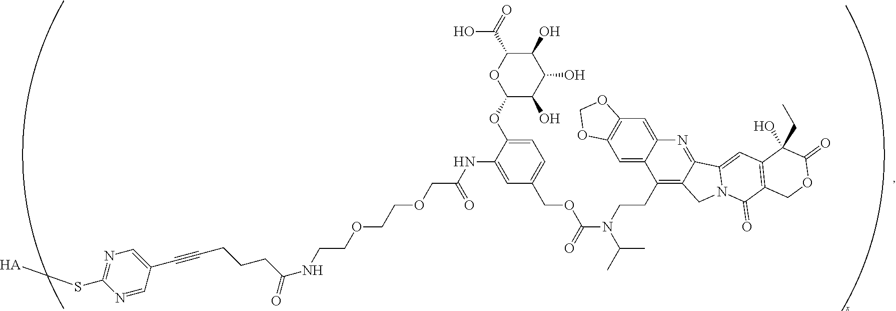

wherein, HA is an antibody or antigen-binding fragment thereof comprising a heavy chain (HC) comprising the amino acid sequence as set forth in SEQ ID NO: 17 and a light chain (LC) comprising the amino acid sequence as set forth in SEQ ID NO: 18; and x is 7 to 8.

In some embodiments, the antibody-drug conjugate is:

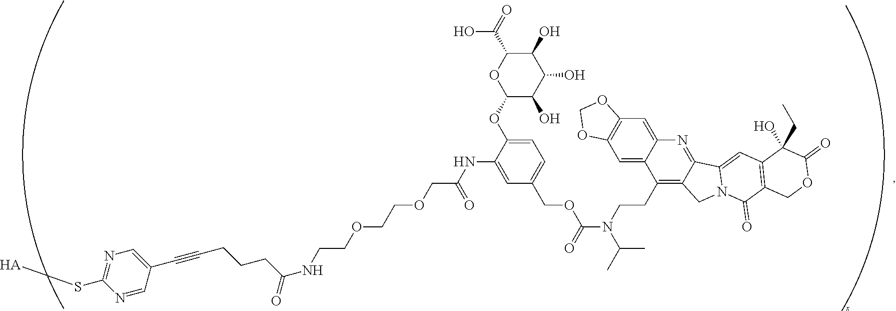

wherein, HA is an antibody or antigen-binding fragment thereof comprising a heavy chain (HC) comprising the amino acid sequence as set forth in SEQ ID NO: 34 and a light chain (LC) comprising the amino acid sequence as set forth in SEQ ID NO: 35; and x is 7 to 8.

In some embodiments, the antibody-drug conjugate is:

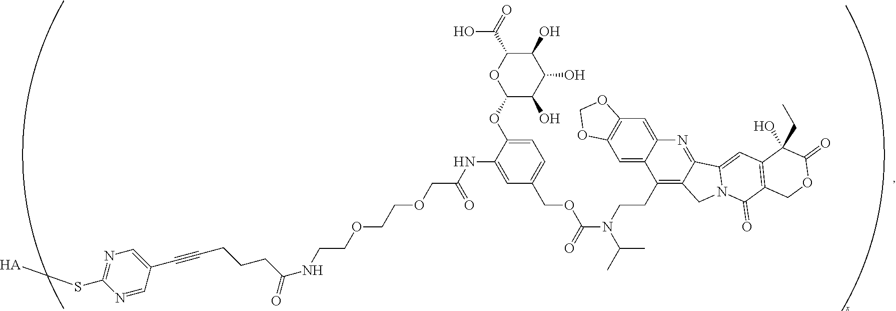

wherein, HA is an antibody or antigen-binding fragment thereof comprising a heavy chain (HC) comprising the amino acid sequence as set forth in SEQ ID NO: 48 and a light chain (LC) comprising the amino acid sequence as set forth in SEQ ID NO: 49; and x is 7 to 8.

In some embodiments, the antibody-drug conjugate (ADC) has an x of 1 to 10, for example, 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10.

In some embodiments, the antibody-drug conjugate (ADC) has an x of 1, 2, 3, 4, 5, 6, 7, 8, 9, or 10.

In some embodiments, the antibody disclosed herein is genetically engineered to comprise one or more cysteine or non-canonical amino acid substitutions of amino acids at defined locations within the antibody. These cysteine residues or non-canonical amino acid residues may then be conjugated to drug-linker via the sulfhydryl group of the cysteine residues or the reactive group of the non-canonical amino acid. Thus, the antibody-drug conjugate of the present invention may comprise one or more substitutions of an amino acid in the heavy chain or light chain of the antibody with a cysteine residue or non-canonical amino acid residue, which is then conjugated to a drug-linker disclosed herein. In particular embodiments, the amino acid positions that may be substituted are selected from positions 152, 153, 171, 172, 173, and 375 of the heavy chain constant domain (numbering according to Eu numbering scheme) and positions 165 and 168 of the light chain constant domain (numbering beginning with amino acid 1 at N-terminus). In particular embodiments, cysteine may be substituted for the amino acid at one or more of the positions 152, 153, 171, 172, 173, and 375 of the heavy chain constant domain (numbering according to Eu numbering scheme) and positions 165 and 168 of the light chain constant domain (numbering beginning with amino acid 1 at N-terminus). In particular embodiments, the antibody-drug conjugate comprises an S375C amino acid substitution that is conjugated to a drug-linker disclosed herein. In particular embodiments, the antibody comprises an S375C amino acid substitution and an E152C amino acid substitution, each conjugated to a drug-linker disclosed herein. In particular embodiments, the antibody comprises an S375C amino acid substitution and an S168C amino acid substitution, each conjugated to a drug-linker disclosed herein. In some embodiments, as explained herein, a composition of the ADC has a DAR value (drug-to-antibody ratio) of 1 to 10, for example: 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10, preferably 3 to 9, for example, 3.0 to 3.5, 3.0 to 4.0, 3.0 to 4.5, 3.0 to 5.0, 3.0 to 5.5, 3.0 to 6.0, 3.5 to 4.0, 3.5 to 4.5, 3.5 to 5.0, 3.5 to 5.5, 3.5 to 6.0, 3.5 to 6.5, 3.5 to 7.0, 3.5 to 7.5, 3.5 to 8.0, 4.0 to 4.5, 4.0 to 5.0, 4.0 to 5.5, 4.0 to 6.0, 4.0 to 6.5, 4.0 to 7.0, 4.0 to 7.5, 4.0 to 8.0, 4.5 to 5.0, 4.5 to 5.5, 4.5 to 6.0, 4.5 to 6.5, 4.5 to 7.0, 4.5 to 7.5, 4.5 to 8.0, 5.0 to 5.5, 5.0 to 6.0, 5.0 to 6.5, 5.0 to 7.0, 5.0 to 7.5, 5.0 to 8.0, 5.5 to 6.0, 5.5 to 6.5, 5.5 to 7.0, 5.5 to 7.5, 5.5 to 8.0, 6.0 to 6.5, 6.0 to 7.0, 6.0 to 7.5, 6.0 to 8.5, 6.5 to 7.0, 6.5 to 7.5, 6.5 to 8.5, 7.0 to 7.5, 7.0 to 9.0 or 7.5 to 9.0.

### Drug-Linkers

Those skilled in the art should understand that the antibody-drug conjugates (ADCs) described in the present application can be prepared modularly. For example, the free form of “drug-linker” (which can be understood as M′-L-E-D, where M′ is the structure of M before it is covalently connected to the antibody or antigen-binding fragment thereof) can be firstly obtained, and then it is covalently connected to the antibody or antigen-binding fragment thereof to obtain the antibody-drug conjugate of the present application. Correspondingly, one or more sulfhydryl (—SH), amino (—NH₂) or carboxyl (—COOH) groups on the antibody or antigen-binding fragment thereof are connected with M′ of the free form of the “drug-linker” through a substitution reaction (e.g., by cleavage of groups such as —SO₂Me or —Br of M′) or an addition reaction.

In another aspect, the present invention provides a drug-linker, which has a structure as shown in the Formula M′-L-E-D, wherein:
M′ is

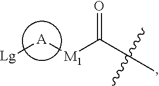

Lg is a leaving group (e.g., halogen, methylsulfonyl, fluorophenol or

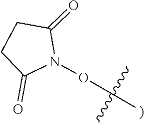

for a nucleophilic substitution reaction, or hydroxyl (—OH), sulfhydryl group (—SH) or amino (—NH₂); or, Lg together with an adjacent atom on Ring A forms an unsaturated double bond; Ring A is a 5- to 6-membered heteroaliphatic ring, or a 5- to 20-membered aromatic ring system, and the heteroaliphatic ring and aromatic ring system are optionally substituted with one or more groups selected from the group consisting of oxy (═O), halogen, cyano, amino, carboxyl, sulfhydryl group and C₁₋₆ alkyl; M₁ is selected from the group consisting of single bond, C₁₋₂₀ alkylene, C₂₋₂₀ alkenylene and C₂₋₂₀ alkynylene; the structures of L, E and D are as defined in any of the aforementioned embodiments of the antibody-drug conjugate.

In some embodiments, M′ is

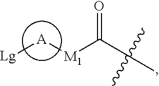

Lg is methylsulfonyl, or, Lg together with an adjacent atom on Ring A forms a carbon-carbon double bond; Ring A is a 5-membered heteroaliphatic ring, a 6-membered heteroaromatic ring, or a polycyclic ring formed by one or more 6-membered heteroaromatic rings and a benzene ring connected through a single bond, wherein the heteroaliphatic ring is optionally substituted with one or more groups selected from the group consisting of oxy (—O), halogen and C₁₋₄ alkyl; M₁ is selected from the group consisting of single bond, C₃₋₁₀ alkylene, C₃₋₁₀ alkenylene and C₃₋₁₀ alkynylene.

In some embodiments, M′ is

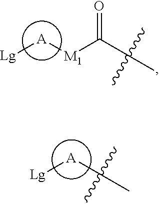

is selected from the group consisting of

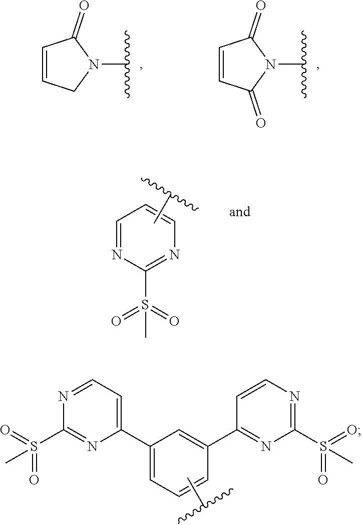

M₁ is selected from the group consisting of single bond, C₅₋₈ alkylene, C₅₋₈ alkenylene and C₅₋₈ alkynylene.

In some embodiments, M′ is selected from the group consisting of

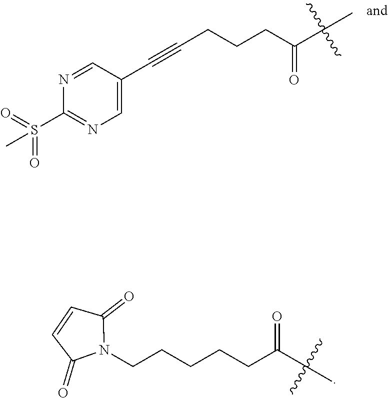

In some embodiments, M′ is

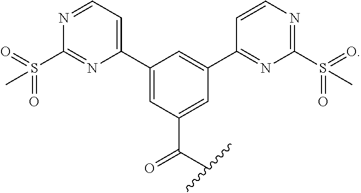 [SMILES: *C(=O)c1cc(-c2ccnc(S(C)(=O)=O)n2)cc(-c2ccnc(S(C)(=O)=O)n2)c1]

In some embodiments, M′ is selected from the group consisting of

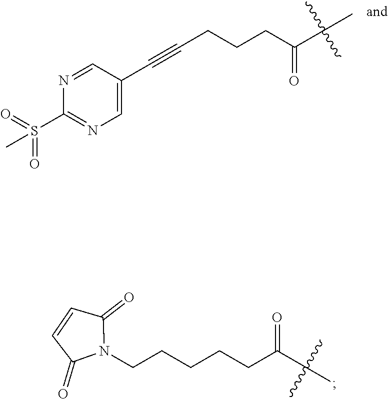

and, when M′ is

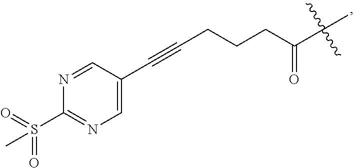

### E is

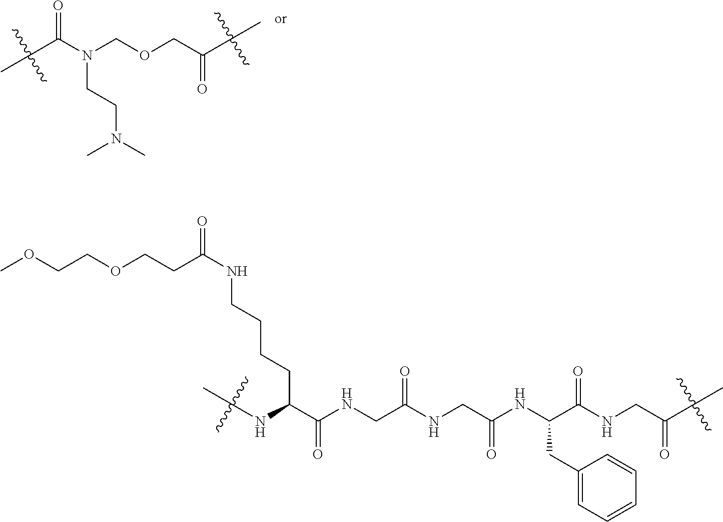

wherein s is an integer selected from 1 to 20.

In some embodiments, the “drug-linker” in free form is selected from the group consisting of A-01 to A-25, B-01 to B-05 as shown below:

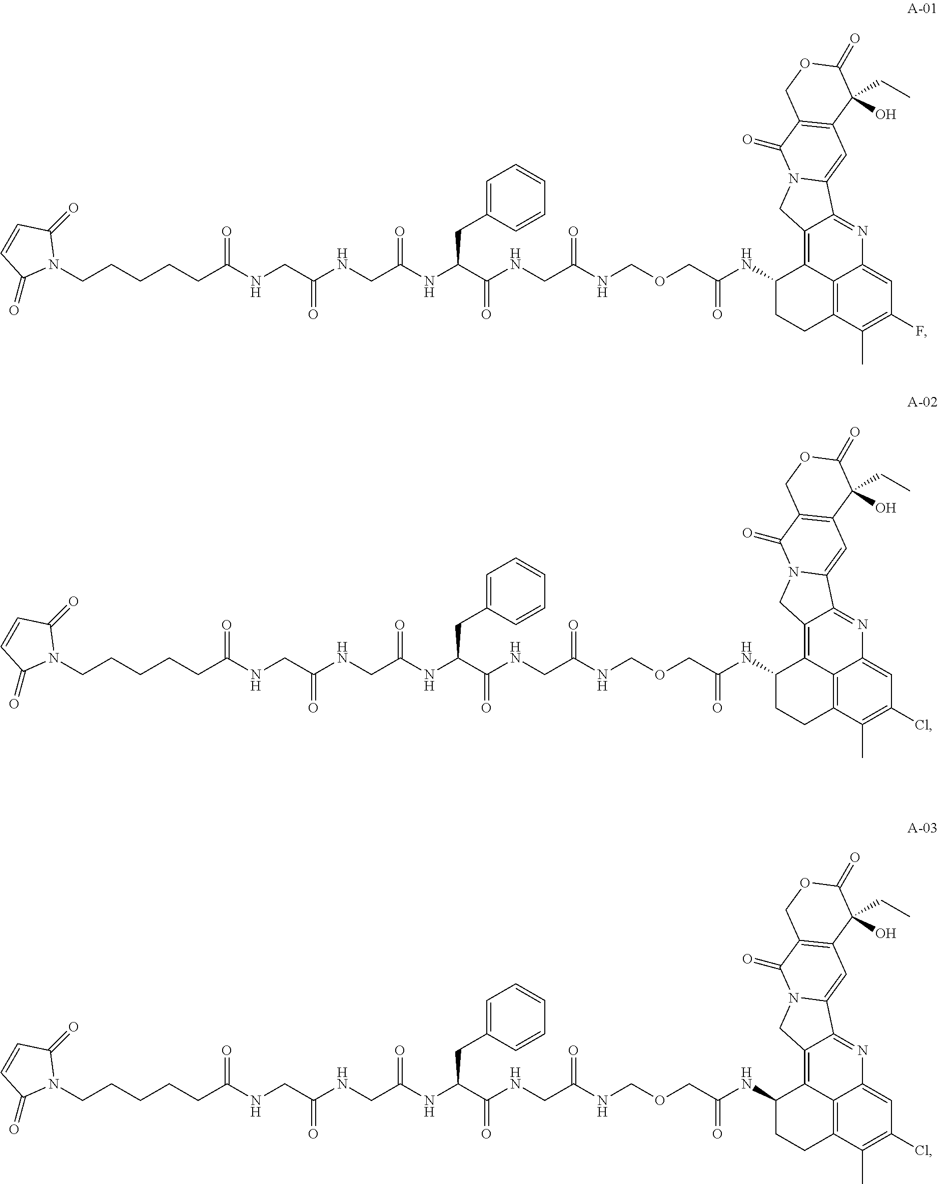 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3 ; CC[C@@]1(O)C(=O)OCc2c1cc1n(c2=O)Cc2c-1nc1cc(Cl)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3 ; CC[C@@]1(O)C(=O)OCc2c1cc1n(c2=O)Cc2c-1nc1cc(Cl)c(C)c3c1c2[C@H](NC(=O)COCNC(=O)CNC(=O)[C@H](Cc1ccccc1)NC(=O)CNC(=O)CNC(=O)CCCCCN1C(=O)C=CC1=O)CC3]

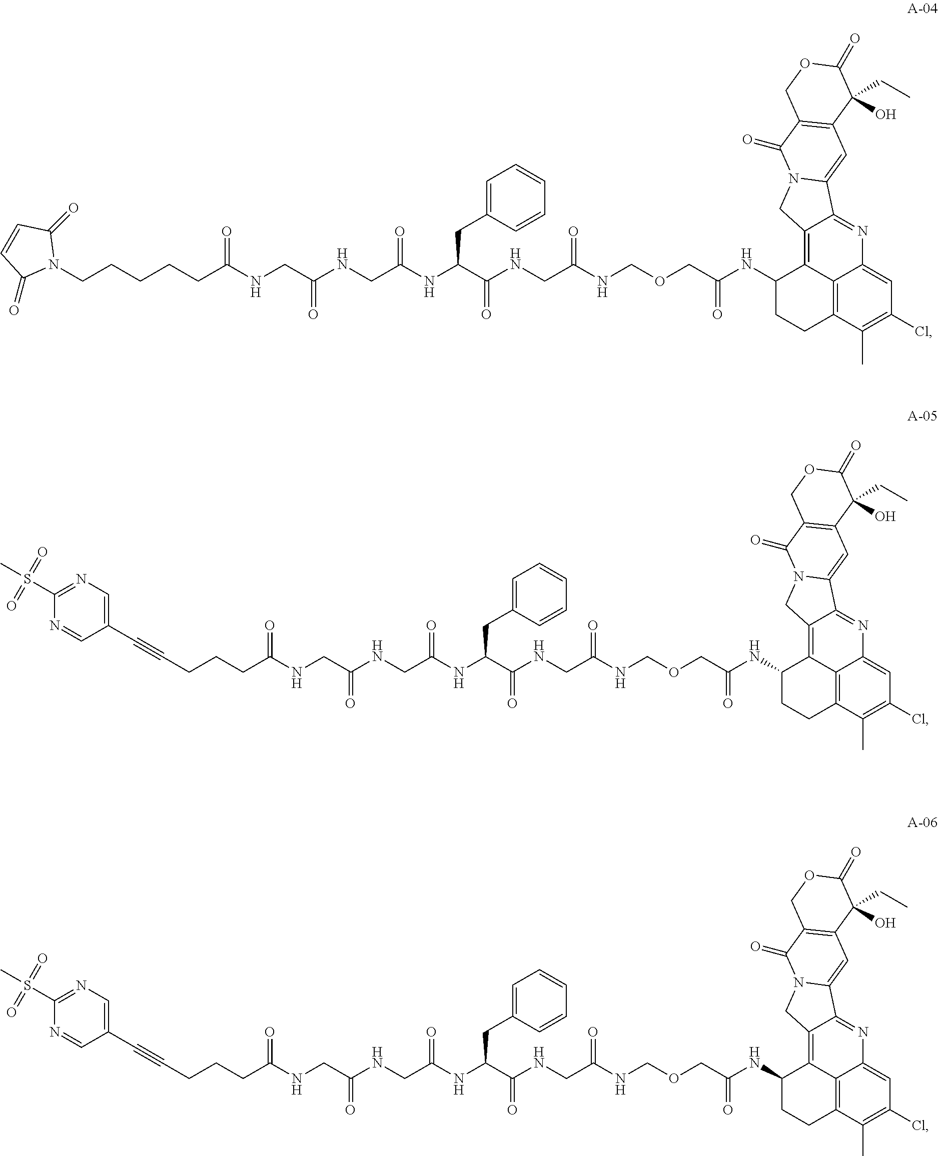 [SMILES: CC[C@@]1(O)C(=O)OCc2c1cc1n(c2=O)Cc2c-1nc1cc(Cl)c(C)c3c1c2C(NC(=O)COCNC(=O)CNC(=O)[C@H](Cc1ccccc1)NC(=O)CNC(=O)CNC(=O)CCCCCN1C(=O)C=CC1=O)CC3 ; CC[C@@]1(O)C(=O)OCc2c1cc1n(c2=O)Cc2c-1nc1cc(Cl)c(C)c3c1c2[C@@H](NC(=O)COCNC(=O)CNC(=O)[C@H](Cc1ccccc1)NC(=O)CNC(=O)CNC(=O)CCCC#Cc1cnc(S(C)(=O)=O)nc1)CC3 ; CC[C@@]1(O)C(=O)OCc2c1cc1n(c2=O)Cc2c-1nc1cc(Cl)c(C)c3c1c2[C@H](NC(=O)COCNC(=O)CNC(=O)[C@H](Cc1ccccc1)NC(=O)CNC(=O)CNC(=O)CCCC#Cc1cnc(S(C)(=O)=O)nc1)CC3]

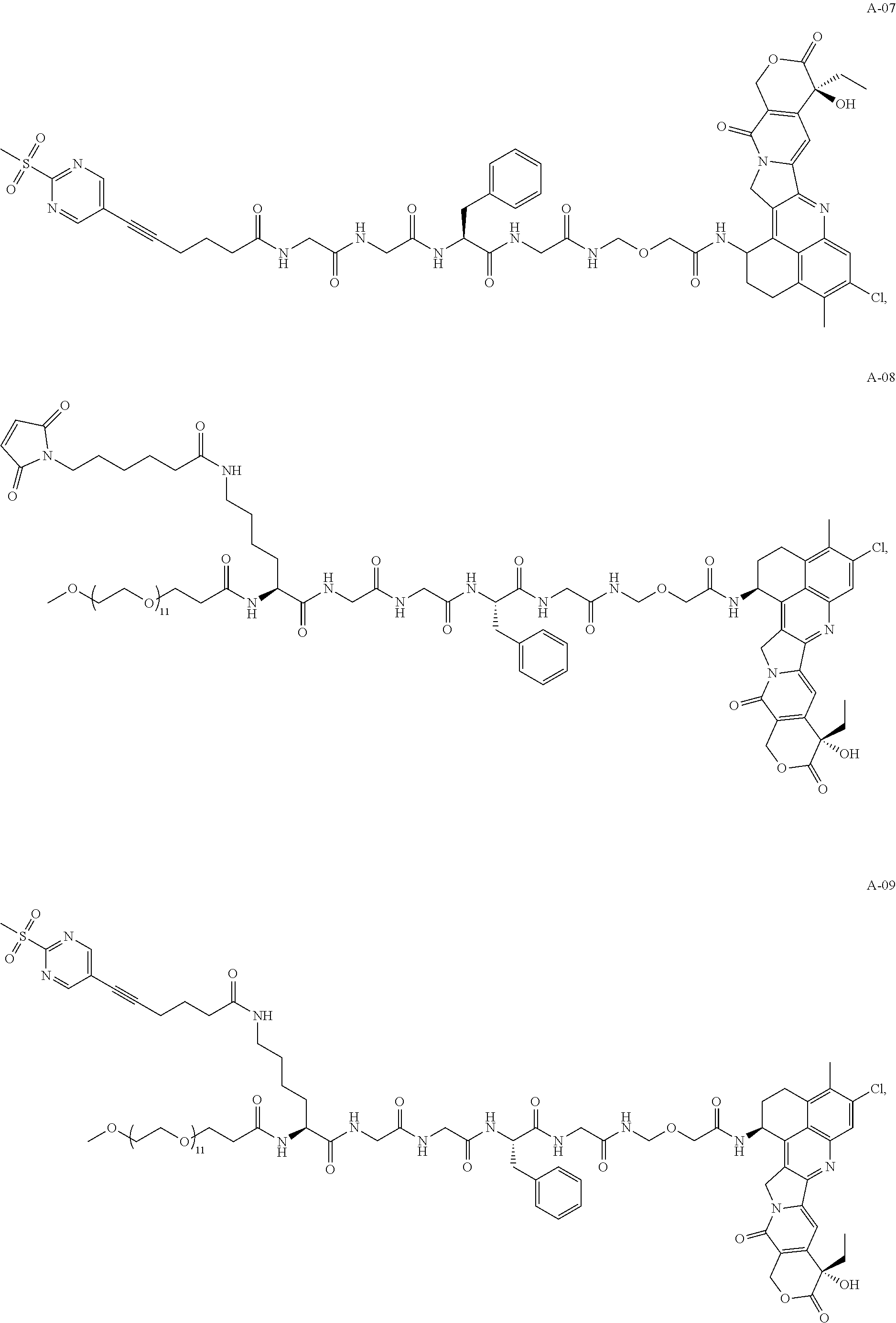

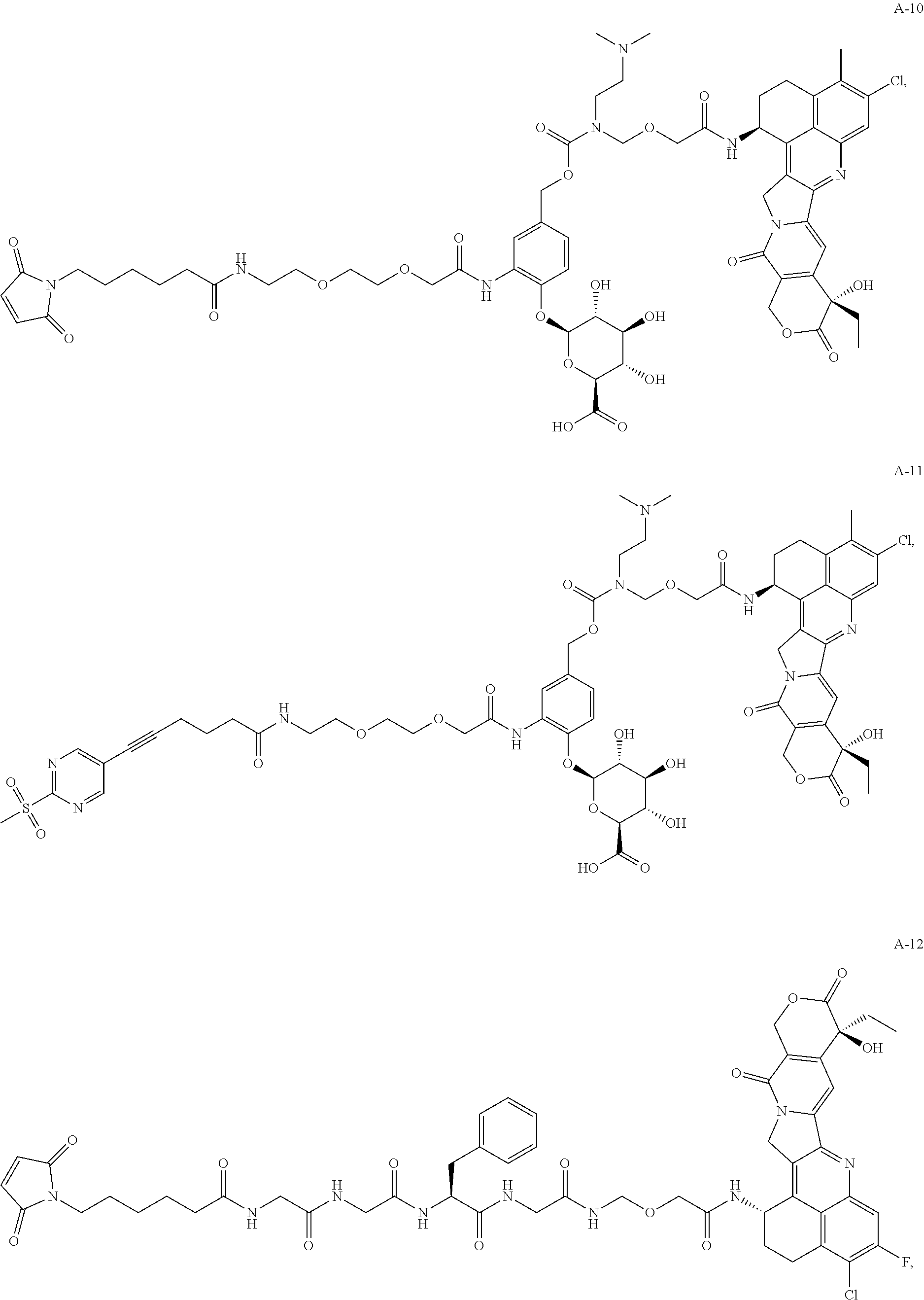

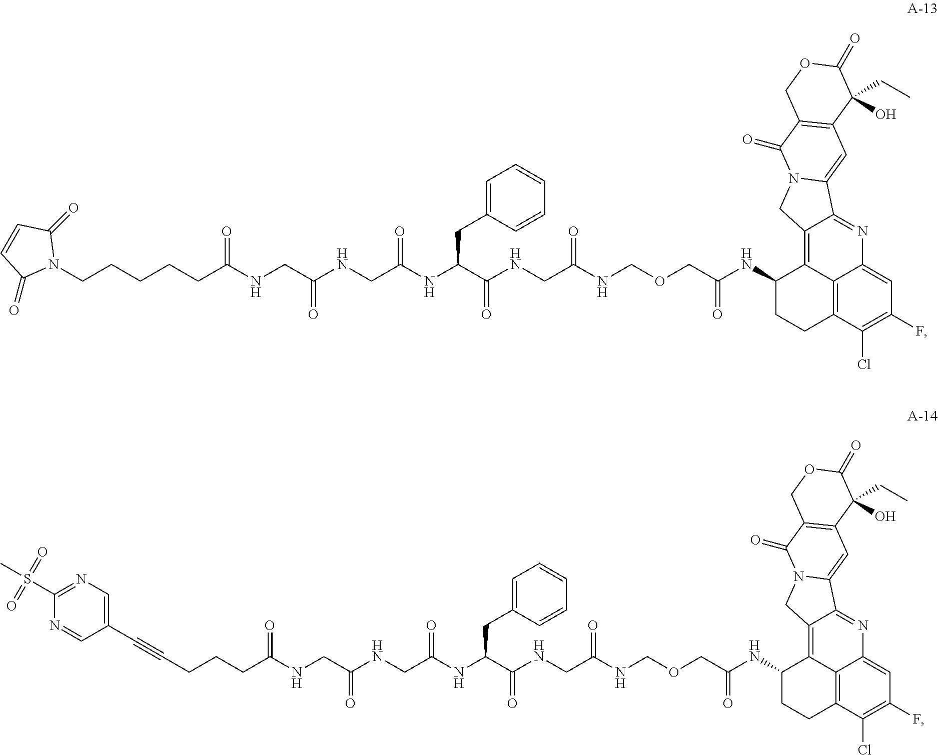 [SMILES: CC[C@@]1(O)C(=O)OCc2c1cc1n(c2=O)Cc2c-1nc1cc(F)c(Cl)c3c1c2[C@H](NC(=O)COCNC(=O)CNC(=O)[C@H](Cc1ccccc1)NC(=O)CNC(=O)CNC(=O)CCCCCN1C(=O)C=CC1=O)CC3 ; CC[C@@]1(O)C(=O)OCc2c1cc1n(c2=O)Cc2c-1nc1cc(F)c(Cl)c3c1c2[C@@H](NC(=O)COCNC(=O)CNC(=O)[C@H](Cc1ccccc1)NC(=O)CNC(=O)CNC(=O)CCCC#Cc1cnc(S(C)(=O)=O)nc1)CC3]

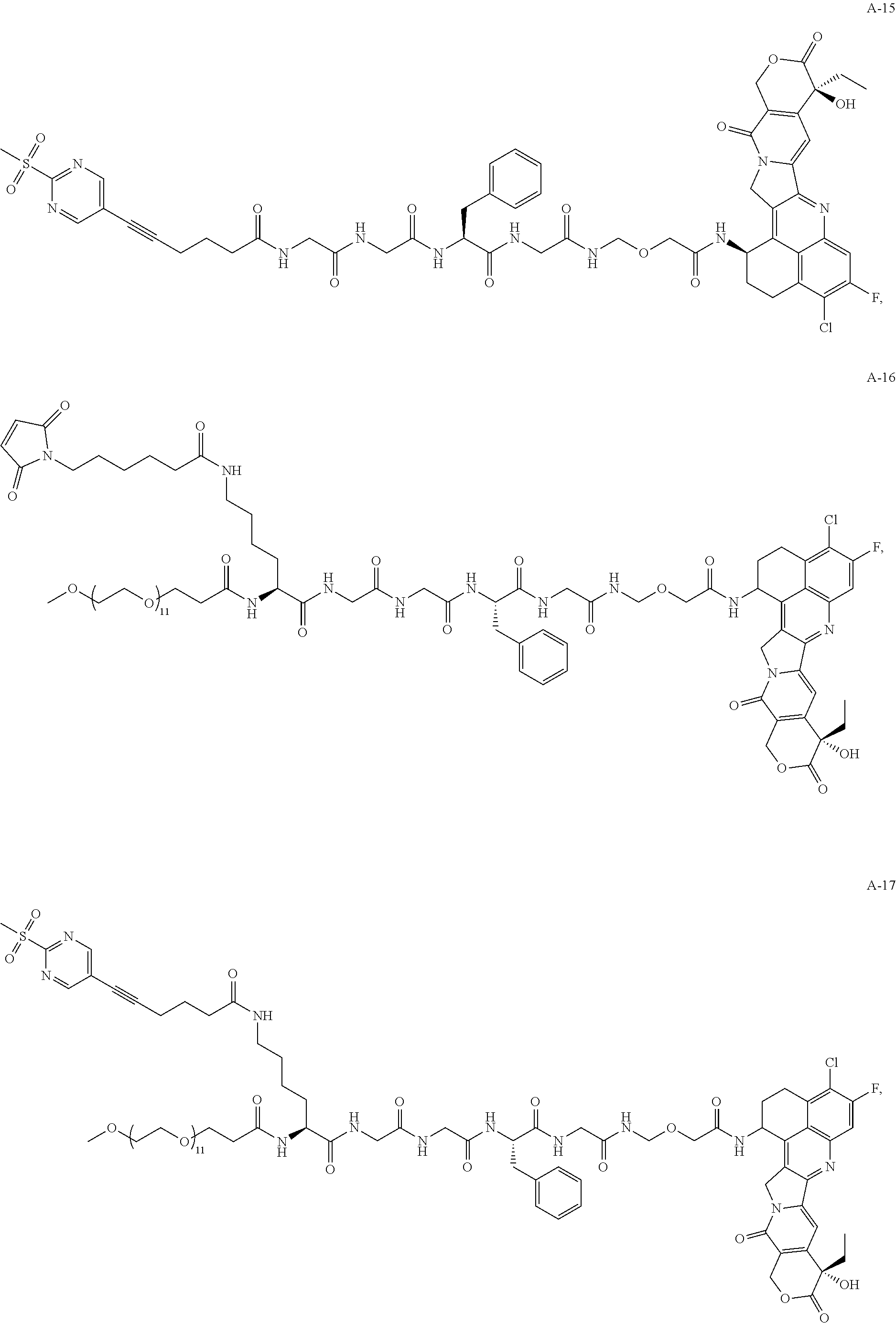

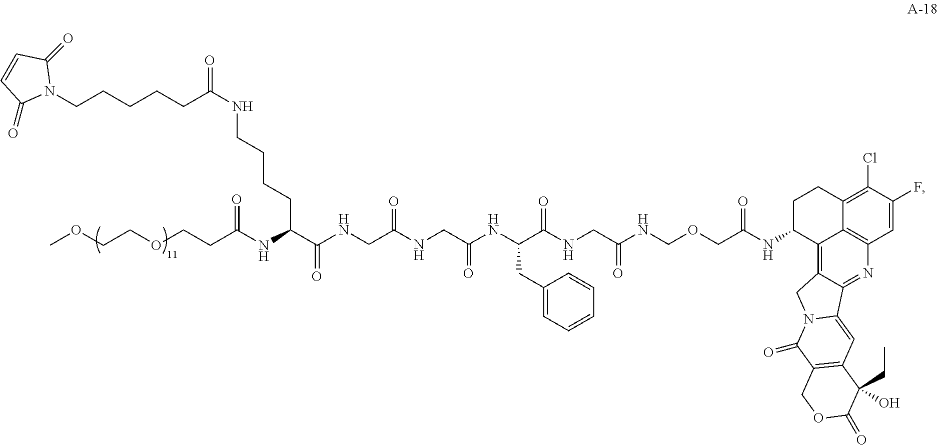

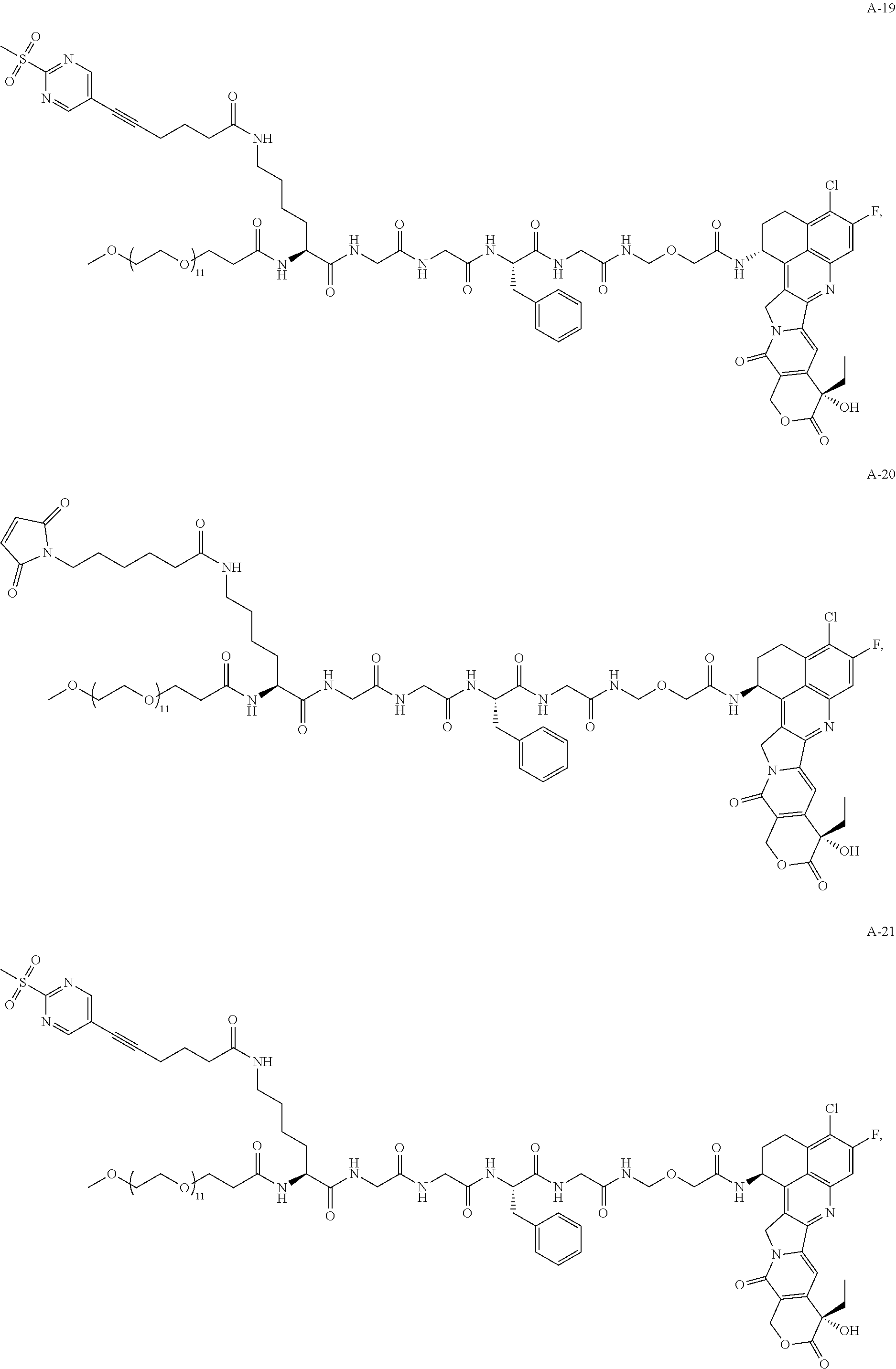

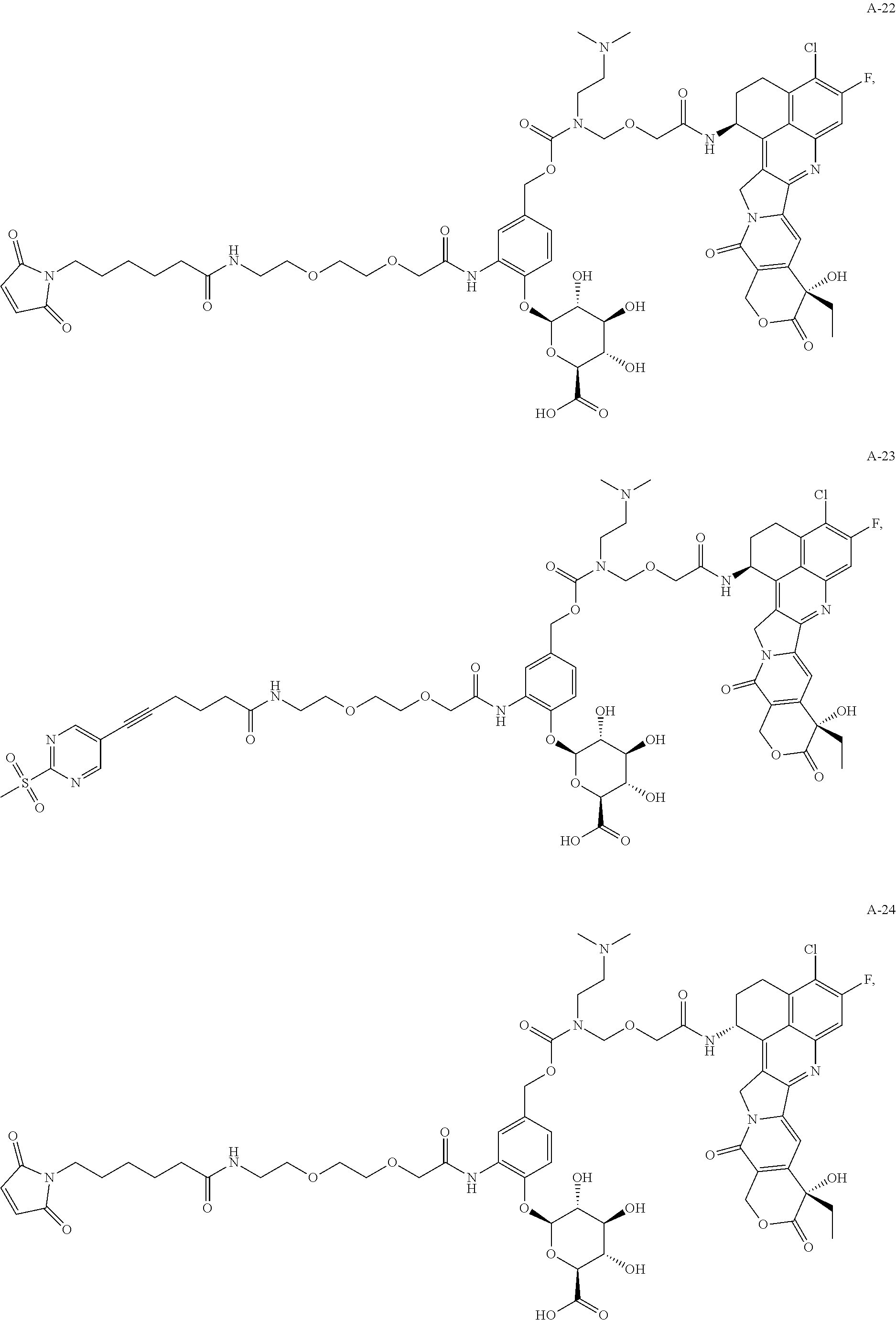

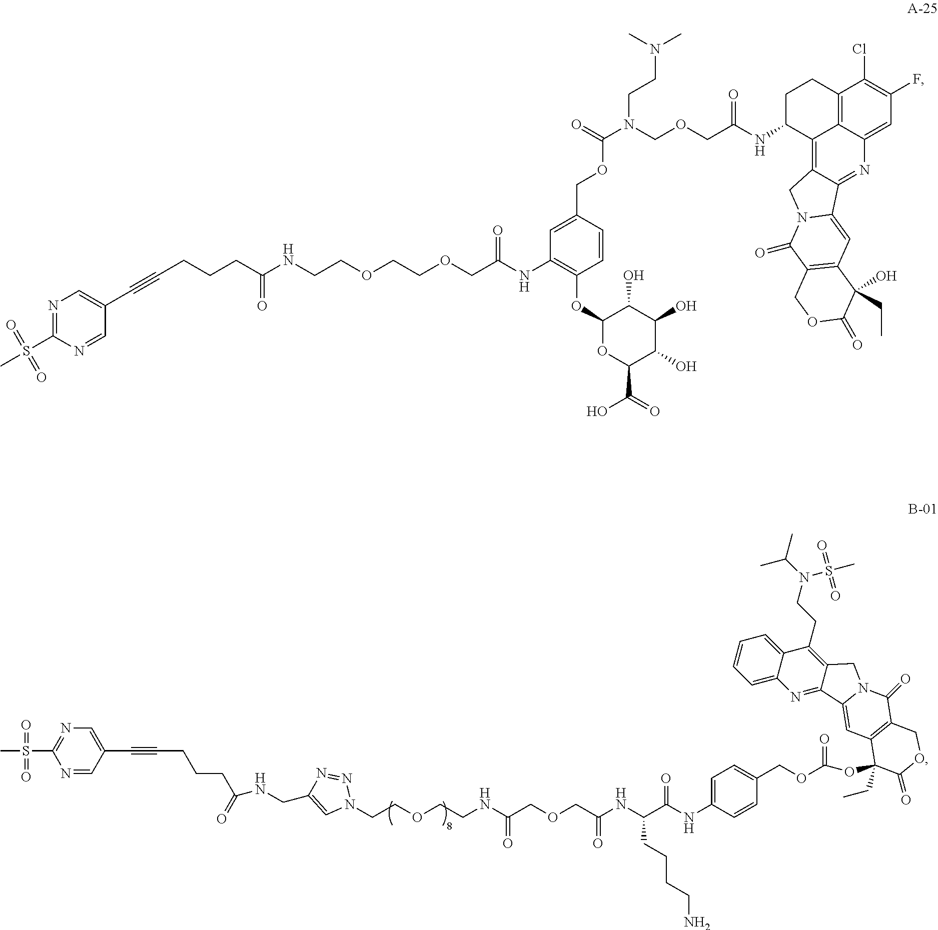

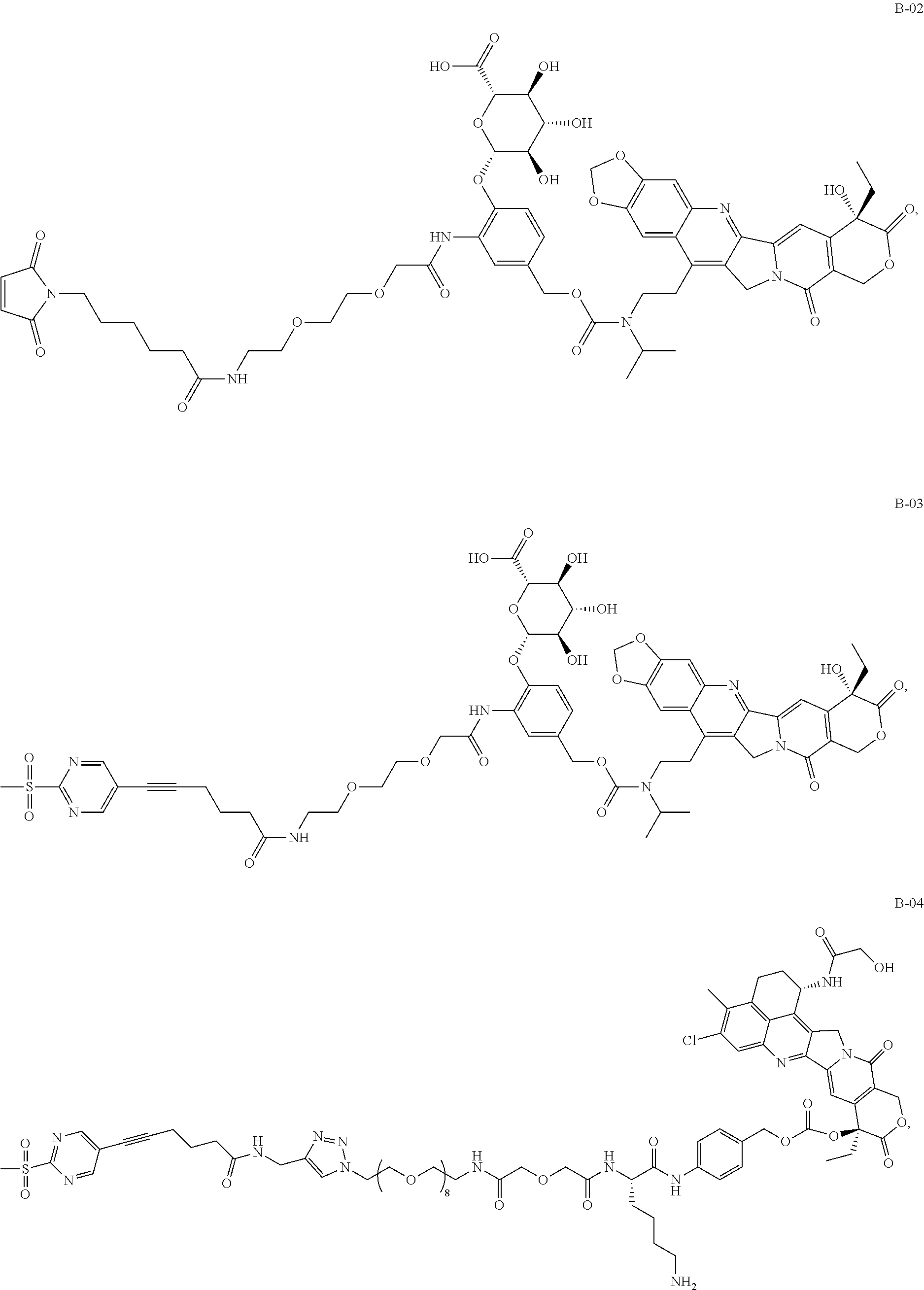

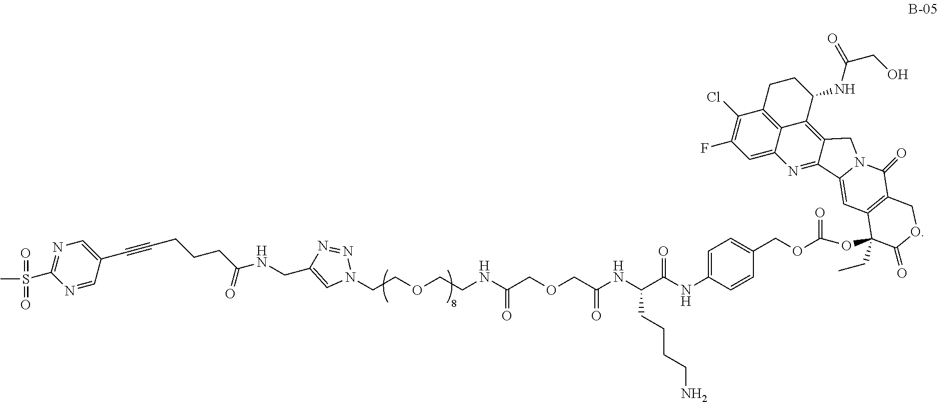

The present invention further provides an ADC comprising an antibody that binds HER3 conjugated via a cysteine residue to a drug-linker selected from the group consisting of A-01, A-02, A-03, A-04, A-05, A-06, A-07, A-08, A-09, A-10, A-12, A13, A-14, A15, A-16, A-17, A-18, A-19, A-20, A-21, A-22, A-23, A-24, A-25, B-01, B-02, B-03, B-04, and B-05.

In a further embodiment of the ADC, the antibody is an antibody or antigen binding fragment selected from the group consisting of:
(1) a heavy chain variable region (VH) and/or a light chain variable region (VL) as follows, wherein the CDRs are defined by the Chothia numbering system:
(1a) a heavy chain variable region (VH) comprising the following 3 CDRs: CDR-H1 having the amino acid as set forth in SEQ ID NO: 1 or a variant thereof, CDR-H2 having the amino acid sequence as set forth in SEQ ID NO: 2 or a variant thereof, CDR-H3 having the amino acid sequence as set forth in SEQ ID NO: 3 or a variant thereof; and/or, a light chain variable region (VL) comprising the following 3 CDRs: CDR-L1 having the amino acid sequence as set forth in SEQ ID NO: 4 or a variant thereof, CDR-L2 having the amino acid sequence as set forth in SEQ ID NO: 5 or a variant thereof, CDR-L3 having the amino acid sequence as set forth in SEQ ID NO: 6 or a variant thereof; or,
(1b) a heavy chain variable region (VH) comprising the following 3 CDRs: CDR-H1 having the amino acid sequence as set forth in SEQ ID NO: 19 or a variant thereof, CDR-H2 having the amino acid sequence as set forth in SEQ ID NO: 20 or a variant thereof, CDR-H3 having the amino acid sequence as set forth in SEQ ID NO: 21 or a variant thereof; and/or, a light chain variable region (VL) comprising the following 3 CDRs: CDR-L1 having the amino acid sequence as set forth in SEQ ID NO: 22 or a variant thereof, CDR-L2 having the amino acid sequence as set forth in SEQ ID NO: 23 or a variant thereof, CDR-L3 having the amino acid sequence as set forth in SEQ ID NO: 24 or a variant thereof; or,
(1c) a heavy chain variable region (VH) comprising the following 3 CDRs: CDR-H1 having the amino acid sequence as set forth in SEQ ID NO: 36 or a variant thereof, CDR-H2 having the amino acid sequence as set forth in SEQ ID NO: 37 or a variant thereof, CDR-H3 having the amino acid sequence as set forth in SEQ ID NO: 38 or a variant thereof; and/or, a light chain variable region (VL) comprising the following 3 CDRs: CDR-L1 having the amino acid sequence as set forth in SEQ ID NO: 45 or a variant thereof, CDR-L2 having the amino acid sequence as set forth in SEQ ID NO: 23 or a variant thereof, and CDR-L3 having the amino acid sequence as set forth in SEQ ID NO: 52 or a variant thereof;
or,
(2) a heavy chain variable region (VH) and/or a light chain variable region (VL) as follows, wherein the CDRs are defined according to the Kabat numbering system:
(2a) a heavy chain variable region (VH) comprising the following 3 CDRs: CDR-H1 having the amino acid sequence as set forth in SEQ ID NO: 7 or a variant thereof, CDR-H2 having the amino acid sequence as set forth in SEQ ID NO: 8 or a variant thereof, CDR-H3 having the amino acid sequence as set forth in SEQ ID NO: 9 or a variant thereof; and/or, a light chain variable region (VL) comprising the following 3 CDRs: CDR-L1 having the amino acid sequence as set forth in SEQ ID NO: 4 or a variant thereof, CDR-L2 having the amino acid sequence as set forth in SEQ ID NO: 5 or a variant thereof, CDR-L3 having the amino acid sequence as set forth in SEQ ID NO: 6 or a variant thereof; or,
(2b) a heavy chain variable region (VH) comprising the following 3 CDRs: CDR-H1 having the amino acid sequence as set forth in SEQ ID NO: 25 or a variant thereof, CDR-H2 having the amino acid sequence as set forth in SEQ ID NO: 26 or a variant thereof, CDR-H3 having the amino acid sequence as set forth in SEQ ID NO: 21 or a variant thereof; and/or, a light chain variable region (VL) comprising the following 3 CDRs: CDR-L1 having the amino acid sequence as set forth in SEQ ID NO: 22 or a variant thereof, CDR-L2 having the amino acid sequence as set forth in SEQ ID NO: 23 or a variant thereof, CDR-L3 having the amino acid sequence as set forth in SEQ ID NO: 24 or a variant thereof; or,
(2c) a heavy chain variable region (VH) comprising the following 3 CDRs: CDR-H1 having a sequence as set forth in SEQ ID NO: 39 or a variant thereof, CDR-H2 having the amino acid sequence as set forth in SEQ ID NO: 40 or a variant thereof, CDR-H3 having the amino acid sequence as set forth in SEQ ID NO: 38 or a variant thereof; and/or, a light chain variable region (VL) comprising the following 3 CDRs: CDR-L1 having the amino acid sequence as set forth in SEQ ID NO: 45 or a variant thereof, CDR-L2 having the amino acid sequence as set forth in SEQ ID NO: 23 or a variant thereof, and CDR-L3 having the amino acid sequence as set forth in SEQ ID NO: 52 or a variant thereof;
or,
(3) a heavy chain variable region (VH) and/or a light chain variable region (VL) as follows, wherein the CDRs are defined by the IMGT numbering system:
(3a) a heavy chain variable region (VH) comprising the following 3 CDRs: CDR-H1 having the amino acid sequence as set forth in SEQ ID NO: 10 or a variant thereof, CDR-H2 having the amino acid sequence as set forth in SEQ ID NO: 11 or a variant thereof, CDR-H3 having the amino acid sequence as set forth in SEQ ID NO: 12 or a variant thereof; and/or, a light chain variable region (VL) comprising the following 3 CDRs: CDR-L1 having the amino acid sequence as set forth in SEQ ID NO: 13 or a variant thereof, CDR-L2 having the amino acid sequence as set forth in SEQ ID NO: 14 or a variant thereof, CDR-L3 having the amino acid sequence as set forth in SEQ ID NO: 6 or a variant thereof; or,
(3b) a heavy chain variable region (VH) comprising the following 3 CDRs: CDR-H1 having the amino acid sequence as set forth in SEQ ID NO: 27 or a variant thereof, CDR-H2 having the amino acid sequence as set forth in SEQ ID NO: 28 or a variant thereof, CDR-H3 having the amino acid sequence as set forth in SEQ ID NO: 29 or a variant thereof; and/or, a light chain variable region (VL) comprising the following 3 CDRs: CDR-L1 having the amino acid sequence as set forth in SEQ ID NO: 30 or a variant thereof, CDR-L2 having the amino acid sequence as set forth in SEQ ID NO: 31 or a variant thereof, CDR-L3 having the amino acid sequence as set forth in SEQ ID NO: 24 or a variant thereof; or,
(3c) a heavy chain variable region (VH) comprising the following 3 CDRs: CDR-H1 having the amino acid sequence as set forth in SEQ ID NO: 41 or a variant thereof, CDR-H2 having the amino acid sequence as set forth in SEQ ID NO: 42 or a variant thereof, CDR-H3 having the amino acid sequence as set forth in SEQ ID NO: 43 or a variant thereof; and/or, a light chain variable region (VL) comprising the following 3 CDRs: CDR-L1 having the amino acid sequence as set forth in SEQ ID NO: 44 or a variant thereof, CDR-L2 having the amino acid sequence as set forth in SEQ ID NO: 31 or a variant thereof, and CDR-L3 having the amino acid sequence as set forth in SEQ ID NO: 52 or a variant thereof.

In a further embodiment of the ADC, the antibody or antigen binding fragment is selected from the group consisting of
(a) a VH comprising the amino acid sequence as set forth in SEQ ID NO: 15 or a variant thereof, and/or, a VL comprising the amino acid sequence as set forth in SEQ ID NO: 16 or a variant thereof;

(b) a VH comprising the amino acid sequence as set forth in SEQ ID NO: 32 or a variant thereof, and/or, a VL comprising the amino acid sequence as set forth in SEQ ID NO: 33 or a variant thereof; or
(c) a VH comprising the amino acid sequence as set forth in SEQ ID NO: 46 or a variant thereof, and/or, a VL comprising the amino acid sequence as set forth in SEQ ID NO: 47 or a variant thereof.

In a further embodiment of the ADC, the antibody or antigen binding fragment is selected from the group consisting of
(1) an antibody or antigen-binding fragment thereof comprising a VH comprising the amino acid sequence as set forth in SEQ ID NO: 15 and a VL comprising the amino acid sequence as set forth in SEQ ID NO: 16, for example, an antibody or antigen-binding fragment thereof comprising a VH comprising the amino acid sequence as set forth in SEQ ID NO: 15 and a CH comprising the amino acid sequence as set forth in SEQ ID NO: 50, as well as a VL comprising the amino acid sequence as set forth in SEQ ID NO: 16 and a CL comprising the amino acid sequence as set forth in SEQ ID NO: 51;
(2) an antibody or antigen-binding fragment thereof comprising a VH comprising the amino acid sequence as set forth in SEQ ID NO: 32 and a VL comprising the amino acid sequence as set forth in SEQ ID NO: 33, for example, an antibody or antigen-binding fragment thereof comprising a VH comprising the amino acid sequence as set forth in SEQ ID NO: 32 and a CH comprising the amino acid sequence as set forth in SEQ ID NO: 50, as well as a VL comprising the amino acid sequence as set forth in SEQ ID NO: 33 and a CL comprising the amino acid sequence as set forth in SEQ ID NO: 51; and
(3) an antibody or antigen-binding fragment thereof comprising a VH comprising the amino acid sequence as set forth in SEQ ID NO: 46 and a VL comprising the amino acid sequence as set forth in SEQ ID NO: 47, for example, an antibody or antigen-binding fragment thereof comprising a VH comprising the amino acid sequence as set forth in SEQ ID NO: 46 and a CH comprising the amino acid sequence as set forth in SEQ ID NO: 50, as well as a VL comprising the amino acid sequence as set forth in SEQ ID NO: 47 and a CL comprising the amino acid sequence as set forth in SEQ ID NO: 51.

In a further embodiment of the ADC, the antibody or antigen binding fragment comprises a VH comprising the amino acid sequence as set forth in SEQ ID NO: 15 and a VL comprising the amino acid sequence as set forth in SEQ ID NO: 16, for example, an antibody or antigen-binding fragment thereof comprising a VH comprising the amino acid sequence as set forth in SEQ ID NO: 15 and a CH comprising the amino acid sequence as set forth in SEQ ID NO: 50, as well as a VL comprising the amino acid sequence as set forth in SEQ ID NO: 16 and a CL comprising the amino acid sequence as set forth in SEQ ID NO: 51.

In a further embodiment of the ADC, the antibody or antigen binding fragment comprises a VH comprising the amino acid sequence as set forth in SEQ ID NO: 32 and a VL comprising the amino acid sequence as set forth in SEQ ID NO: 33, for example, an antibody or antigen-binding fragment thereof comprising a VH comprising the amino acid sequence as set forth in SEQ ID NO: 32 and a CH comprising the amino acid sequence as set forth in SEQ ID NO: 50, as well as a VL comprising the amino acid sequence as set forth in SEQ ID NO: 33 and a CL comprising the amino acid sequence as set forth in SEQ ID NO: 51.

In a further embodiment of the ADC, the antibody or antigen binding fragment comprises a VH comprising the amino acid sequence as set forth in SEQ ID NO: 46 and a VL comprising the amino acid sequence as set forth in SEQ ID NO: 47, for example, an antibody or antigen-binding fragment thereof comprising a VH comprising the amino acid sequence as set forth in SEQ ID NO: 46 and a CH comprising the amino acid sequence as set forth in SEQ ID NO: 50, as well as a VL comprising the amino acid sequence as set forth in SEQ ID NO: 47 and a CL comprising the amino acid sequence as set forth in SEQ ID NO: 51.

In a further embodiment, the antibody comprises a heavy chain (HC) comprising the amino acid sequence as set forth in SEQ ID NO: 17, and a light chain (LC) comprising the amino acid sequence as set forth in SEQ ID NO: 18.

In a further embodiment, the antibody comprises a heavy chain (HC) comprising the amino acid sequence as set forth in SEQ ID NO: 34, and a light chain (LC) comprising the amino acid sequence as set forth in SEQ ID NO: 35.

In a further embodiment, the antibody comprises a heavy chain (HC) comprising the amino acid sequence as set forth in SEQ ID NO: 48, and a light chain (LC) comprising the amino acid sequence as set forth in SEQ ID NO: 49.

### Antibody Preparation

The antibodies described herein can be prepared by various methods known in the art, for example, by genetic engineering and recombination techniques. For example, DNA molecules encoding the heavy and light chain genes of the antibodies of the present invention are obtained by chemical synthesis or PCR amplification. The resulting DNA molecule is inserted into an expression vector, which is then transfected into a host cell. Then, the transfected host cells are cultured under specific conditions, and express the antibody of the present invention.

### Conjugation

In another aspect, the present application provides a method for conjugating the drug-linkers described herein to the antibodies described herein to make the antibody-drug conjugates (ADCs) described herein.

In certain embodiments, the antibody described herein is conjugated to a drug-linker described herein via conjugation to a lysine in the antibody.

In certain embodiments, the antibody described herein is conjugated to a drug-linker described herein via conjugation to a cysteine in the antibody. In certain embodiments, the cysteines are from reduced intrachain disulfide bonds in the antibody. In certain embodiments, the cysteines are from reduced interchain disulfide bonds in the antibody.

In some embodiments, the antibody is conjugated to a drug-linker via conjugation to reduced interchain disulfide bonds in the antibody. For example, an IgG1 antibody consists of four polypeptide chains, two heavy chains comprising VH, CH1 and Fc (e.g., hinge, CH2 and CH3) domains, and two light chains comprising VL and CL domains, connected by interchain cysteine disulfide (—S—S—) bonds (e.g., two heavy chain-light chain interchain disulfide bonds and two hinge heavy chain-heavy chain interchain disulfide bonds). In certain embodiments, when these disulfide bonds are broken under reducing conditions, eight (8) reactive cysteine sulfhydryl moieties are produced. In certain embodiments, each of the eight reactive cysteine sulfhydryl moieties is a point of attachment for a linker-drug, such that a maximum of eight (x=8) drug-linkers may be attached to the reduced antibody. In certain embodiments, any one of the four disulfide bonds is broken under reducing conditions, two (2) reactive cysteine sulfhydryl moieties are produced. In further embodiments, each of the two reactive cysteine sulfhydryl moieties is a point of attachment for a linker-drug, such that two (x=2) linker-drugs may be attached to the reduced antibody. In certain embodiments, any two of the four disulfide bonds are broken under reducing conditions, four (4) reactive cysteine sulfhydryl moieties are produced. In further embodiments, each of the four reactive cysteine sulfhydryl moieties is a point of attachment for a linker-drug, such that four (x=4) linker-drugs may be attached to the reduced antibody. In certain embodiments, any three of the four disulfide bonds are broken under reducing conditions, six (6) reactive cysteine sulfhydryl moieties are produced. In further embodiments, each of the six reactive cysteine sulfhydryl moieties is a point of attachment for a linker-drug, such that six (x=6) linker-drugs may be attached to the reduced antibody.

In some embodiments, the interchain disulfide bond is between two cysteine residues, which are broken under reducing conditions, resulting in two reactive cysteine sulfhydryl moieties. In further embodiments, the interchain disulfide bridge in the antibody is between a heavy chain and a light chain, such as between C220 of a heavy chain and C214 of a kappa light chain according to the EU numbering, or between C220 of a heavy chain according to the EU numbering and C214 of a lambda light chain according to the Kabat numbering. Additionally, or alternatively, the interchain disulfide bridge in the antibody is between two heavy chains, such as between C226 and/or C229 of a first heavy chain and C226 and/or C229 of a second heavy chain according to the EU numbering. In some embodiments, the cysteine residues are in the hinge region of the antibody. In some embodiments, the cysteine residue is at any one or more of positions 220, 226, or 229 in the heavy chain according to EU numbering (also referred to herein as C220, C226 or C229, respectively). In some embodiments, the cysteine residue is at position 214 in the light chain according to EU and/or Kabat numbering (also referred to herein as C214, such as position 214 in the kappa light chain according to EU and Kabat numbering or position 214 in the lambda light chain according to the Kabat numbering). In one embodiment, the cysteine residues are at each of positions 220, 226, and 229 in the heavy chain according to the EU numbering and position 214 in the light chain, according to EU or Kabat numbering. In one embodiment, the cysteine residues are at each of positions 220, 226, and 229 in the heavy chain according to the EU numbering and position 214 in the kappa light chain, according to EU and Kabat numbering. In one embodiment, the cysteine residues are at each of positions 220, 226, and 229 in the heavy chain according to the EU numbering and position 214 in the lambda light chain, according to Kabat numbering. In one embodiment, the cysteine residues are at any one or more of the following positions:
(i) any one, or any two, or any three, or all four of positions 220, 226, and 229 in a first heavy chain according to the EU numbering;
(ii) any one, or any two, or any three, or all four of positions 220, 226, and 229 in a second heavy chain according to the EU numbering;
(iii) position 214 in a first light chain according to the Kabat numbering; and/or
(iv) position 214 in a second light chain according to the Kabat numbering.

As used herein, C220, C226, and C229 refer to amino acid residues (cysteine, Cys, C) of an immunoglobulin identified according to the EU numbering. As it would be understood by one of skill in the art, such numberings accordingly represent amino acid residues of a polypeptide aligned to those identified in an immunoglobulin, such as the one shown in www.imgt.org/IMGTScientificChart/Numbering/Hu_IGHGnber.html.

As used herein, the cysteine residue at position 214 of kappa light chain refer to amino acid residues (cysteine, Cys, C) of an immunoglobulin identified according to the Kabat numbering. As it would be understood by one of skill in the art, such numberings accordingly represent amino acid residues of a polypeptide aligned to those identified in an immunoglobulin, such as the one shown in www.imgt.org/IMGTScientificChart/Numbering/Hu_IGKCnber.html.

As used herein, the cysteine residue at position 214 of lambda light chain refer to amino acid residues (cysteine, Cys, C) of an immunoglobulin identified according to the Kabat numbering. As it would be understood by one of skill in the art, such numberings accordingly represent amino acid residues of a polypeptide aligned to those identified in an immunoglobulin, such as the one shown in www.imgt.org/IMGTScientificChart/Numbering/Hu_IGLCnber.html.

In certain embodiments, the antibodies described herein comprise four interchain disulfide bonds in the hinge region which may be reduced, thereby breaking the bond, and revealing a reactive sulfhydryl moiety that may be conjugated with a maleimide moiety on a drug-linker, such as the maleimide moiety on drug-linkers described herein.

In one embodiment, the present disclosure provides a method of making an ADC described herein, comprising the steps of:
a) providing a solution comprising the antibody;
b) contacting the solution of a) with a reducing agent;
c) contacting the solution of b) with a solution comprising a drug-linker or a salt thereof, as described herein,
to make the ADC.

In one embodiment, the reducing agent is tris(2 carboxyethyl) phosphine (TCEP).

### Compositions

In another aspect, the present application provides a composition of antibody-drug conjugates (ADCs) as described herein. Such composition may comprise a plurality of ADCs as described herein, wherein each ADC comprises a drug-linker as described herein, wherein x is independently 1, 2, 3, 4, 5, 6, 7, 8, 9 or 10. In other words, each antibody molecule in the composition may be conjugated to 1, 2, 3, 4, 5, 6, 7, 8, 9 or 10 drug-linkers. Therefore, a composition may be characterized by a “drug-to-antibody” ratio (DAR) ranging from about 1 to about 10. Methods to determine DAR are well known to the skilled person and include methods using Reverse Phase Chromatography, or HPLC-MS.

For example, in any embodiment, an ADC composition described herein has a DAR of about 1 to about 10 or any subrange there between, for example: about 1 to 2, about 1 to 3, about 1 to 4, about 1 to 5, about 1 to 6, about 1 to 7, about 1 to 8, about 1 to 9, about 1 to 10, about 2 to 3, about 2 to 4, about 2 to 5, about 2 to 6, about 2 to 7, about 2 to 8, about 2 to 9, about 2 to 10, about 3 to 4, about 3 to 5, about 3 to 6, about 3 to 7, about 3 to 8, about 3 to 9, about 3 to 10, about 4 to 5, about 4 to 6, about 4 to 7, about 4 to 8, about 4 to 9, about 4 to 10, about 5 to 6, about 5 to 7, about 5 to 8, about 5 to 9, about 5 to 10, about 6 to 7, about 6 to 8, about 6 to 9, about 6 to 10, about 7 to 8, about 7 to 9, about 7 to 10, about 8 to 9, about 8 to 10, or about 9 to 10.

In certain embodiments, an ADC composition described herein has a DAR of about 3 to 9, for example, about 3.0 to 3.5, about 3.0 to 4.0, about 3.0 to 4.5, about 3.0 to 5.0, about 3.0 to 5.5, about 3.0 to 6.0, about 3.5 to 4.0, about 3.5 to 4.5, about 3.5 to 5.0, about 3.5 to 5.5, about 3.5 to 6.0, about 3.5 to 6.5, about 3.5 to 7.0, about 3.5 to 7.5, about 3.5 to 8.0, about 4.0 to 4.5, about 4.0 to 5.0, about 4.0 to 5.5, about 4.0 to 6.0, about 4.0 to 6.5, about 4.0 to 7.0, about 4.0 to 7.5, about 4.0 to 8.0, about 4.5 to 5.0, about 4.5 to 5.5, about 4.5 to 6.0, about 4.5 to 6.5, about 4.5 to 7.0, about 4.5 to 7.5, about 4.5 to 8.0, about 5.0 to 5.5, about 5.0 to 6.0, about 5.0 to 6.5, about 5.0 to 7.0, about 5.0 to 7.5, about 5.0 to 8.0, about 5.5 to 6.0, about 5.5 to 6.5, about 5.5 to 7.0, about 5.5 to 7.5, about 5.5 to 8.0, about 6.0 to 6.5, about 6.0 to 7.0, about 6.0 to 7.5, about 6.0 to 8.5, about 6.5 to 7.0, about 6.5 to 7.5, about 6.5 to 8.5, about 7.0 to 7.5, about 7.0 to 9.0 or about 7.5 to 9.0.

### Pharmaceutical Compositions

In another aspect, the present application provides a pharmaceutical composition, which comprises the antibody-drug conjugate (ADC) described in any one of the foregoing embodiments and optionally the drug-linker described in any one of the foregoing embodiments, and one or more pharmaceutically acceptable auxiliaries.

Pharmaceutically acceptable auxiliaries include, for example, a pharmaceutically acceptable carrier and/or excipient. Pharmaceutically acceptable auxiliaries further includes salts and solvates.

The ADC described herein is typically formulated with a pharmaceutically acceptable parenteral vehicle to form a unit injectable form for parenteral application, such as bolus injection, intravenous injection, intratumoral injection, and the like. Optionally, the antibody-drug conjugate having the desired purity is mixed with a pharmaceutically acceptable diluents, carriers, excipients or stabilizers in the form of lyophilizate or solution (Remington's Pharmaceutical Sciences (1980) 16^{th} edition, Osol, A. Ed.). The antibody-drug conjugate described herein, or the pharmaceutical composition comprising the antibody-drug conjugate, can be administered via any route appropriate for the individual to be treated.

The ADC and pharmaceutical compositions described herein can be formulated into any dosage form known in the medical field, for example, tablets, pills, suspensions, emulsions, solutions, gels, capsules, powders, granules, elixirs, lozenges, suppositories, injections (including injections, sterile powders for injections and concentrated solutions for injections), inhalants, sprays, etc. The preferred dosage form depends on the intended mode of administration and therapeutic use. Pharmaceutical compositions of the invention should be sterile and stable under the conditions of manufacture and storage. A preferred dosage form is injection. Such injections can be sterile injectable solutions. For example, sterile injectable solutions can be prepared by: The necessary dose of the antibody of the present invention is incorporated in an appropriate solvent, and optionally, other desired ingredients (including but not limited to, pH adjusters, surfactants, adjuvants, ionic strength enhancers, etc. osmotic agent, preservative, diluent, or any combination thereof), followed by filter sterilization. In addition, sterile injectable solutions can be prepared as sterile lyophilized powder (e.g., by vacuum drying or freeze-drying) for ease of storage and use. Such sterile lyophilized powder can be dispersed in a suitable carrier, such as sterile pyrogen-free water, before use.

Furthermore, the ADCs described herein may be presented in pharmaceutical compositions in unit dosage form for ease of administration by any suitable method known in the art, including but not limited to, oral, oral, sublingual, ophthalmic, topical, parenteral, rectal, intrathecal, intracytoplasmic reticulum, inguinal, intravesical, topical (e.g., powder, ointment, or drops), or nasal routes. However, for many therapeutic uses, the preferred route/mode of administration is parenteral (e.g., intravenous, subcutaneous, intraperitoneal, intramuscular). The skilled artisan will understand that the route and/or manner of administration will vary depending on the intended purpose. In some preferred embodiments, the ADCs and pharmaceutical compositions described herein is administered by intravenous infusion or injection.

In certain embodiments, the pharmaceutical composition may also comprise additional pharmaceutically active agents. In certain embodiments, the additional pharmaceutically active agent is a drug having antineoplastic activity. In certain embodiments, the additional pharmaceutically active agent is selected from the group consisting of EGFR inhibitors, HER2 inhibitors, HER3 inhibitors, HER4 inhibitors, IGFR-1 inhibitors, mTOR inhibitors, PI3 kinase inhibitors, c-met or VEGF inhibitors, chemotherapy drugs, or any combination thereof. In certain embodiments, the ADCs described herein and the additional pharmaceutically active agent are provided as separate components or as admixed components. Accordingly, the antibody or antigen-binding fragment thereof of the invention and the additional pharmaceutically active agent may be administered simultaneously, separately or sequentially.

### Methods of Use

The antibody-drug conjugate described herein, drug-linker described herein, or pharmaceutical compositions thereof can be used for treating various diseases or conditions, such as cancers with high expression of HER3, including solid tumors or hematological malignancies, such as colon cancer, gastric cancer, breast cancer, lung cancer (e.g., non-small cell lung cancer, specifically lung adenocarcinoma), or lymphoma.

Therefore, the present application provides use of the antibody-drug conjugate (ADC), the drug-linker, or the pharmaceutical composition containing the same as described in any one of foregoing embodiments, in the manufacture of a medicament for the treatment of a cancer with high expression of HER3.

At the same time, the present application also provides a method for treating a cancer with high expression of HER3, which comprises a step of administering to a subject in need thereof a therapeutically effective amount of the antibody-drug conjugate (ADC), the drug-linker, or the pharmaceutical composition comprising the same as described in any one of foregoing embodiments.

In certain embodiments, the antibody-drug conjugate (ADC), drug-linker, or pharmaceutical composition is sufficient (e.g., in a subject) to:
(1) Inhibit the proliferation of cells (such as tumor cells);
(2) inhibit tumor growth;
(3) Induce and/or increase antibody-dependent cytotoxic activity;
(4) Inhibit HER3-mediated signal transduction;
(5) Prevention and/or treatment of HER3-mediated diseases/disorders; or
(6) Any combination of the above (1)-(5).

In certain embodiments, the HER3-mediated disease/disorder is a tumor, e.g., a HER3-expressing tumor. In certain embodiments, the tumor is selected from breast cancer, gastric cancer, lung cancer (such as non-small cell lung cancer), colorectal cancer, pancreatic cancer, squamous cell carcinoma of the head and neck, melanoma, ovarian cancer, prostate cancer, liver cancer, kidney cancer, bladder cancer, or any combination thereof.

### EXAMPLES

The present invention is further described by the following examples, which do not intend to limit the present invention. Those skilled in the art can make various modifications or improvements according to the teachings of the present invention without departing from the principles and scope of the present invention.

Information on the sequences involved in the present invention is described in the following Tables A and B:

TABLE A

| | | SEQ ID NO: | | | | | | | | | | | |
|---|---|---|---|---|---|---|---|---|---|---|---|---|---|
| | Numbering | CDR- | CDR- | CDR- | CDR- | CDR- | CDR- | | | | | | |
| Antibody | system | H1 | H2 | H3 | L1 | L2 | L3 | VH | VL | CH | CL | HC | LC |
| 22B6D2- | Chothia | 1 | 2 | 3 | 4 | 5 | 6 | 15 | 16 | 50 | 51 | 17 | 18 |
| hIgG1 | Kabat | 7 | 8 | 9 | 4 | 5 | 6 | | | | | | |
| | IMGT | 10 | 11 | 12 | 13 | 14 | 6 | | | | | | |
| 47A3E3- | Chothia | 19 | 20 | 21 | 22 | 23 | 24 | 32 | 33 | | | 34 | 35 |
| hIgG1 | Kabat | 25 | 26 | 21 | 22 | 23 | 24 | | | | | | |
| | IMGT | 27 | 28 | 29 | 30 | 31 | 24 | | | | | | |
| 100H7D3- | Chothia | 36 | 37 | 38 | 45 | 23 | 52 | 46 | 47 | | | 48 | 49 |
| hIgG1 | Kabat | 39 | 40 | 38 | 45 | 23 | 52 | | | | | | |
| | IMGT | 41 | 42 | 43 | 44 | 31 | 52 | | | | | | |

TABLE B

| Sequence No. | Sequence information | Sequence name |
|---|---|---|
| SEQ ID NO: 1 | GFTFRSY | 22B6D2-hIgG1 |
| | | Chothia CDR H1 |
| SEQ ID NO: 2 | WYDGSN | 22B6D2-hIgG1 |
| | | Chothia CDR H2 |
| SEQ ID NO: 3 | YYYDSNGYYDAFDI | 22B6D2-hIgG1 |
| | | Chothia CDR H3 |
| SEQ ID NO: 4 | RASQGISNYLA | 22B6D2-hIgG1 |
| | | Chothia/Kabat CDR L1 |
| SEQ ID NO: 5 | AASTLQS | 22B6D2-hIgG1 |
| | | Chothia/Kabat CDR L2 |
| SEQ ID NO: 6 | QQLNSYPIT | 22B6D2-hIgG1 |
| | | Chothia/Kabat/IMGT |
| | | CDR L3 |
| SEQ ID NO: 7 | SYGMH | 22B6D2-hIgG1 Kabat |
| | | CDR H1 |
| SEQ ID NO: 8 | VIWYDGSNKYYADSVKG | 22B6D2-hIgG1 Kabat |
| | | CDR H2 |
| SEQ ID NO: 9 | YYYDSNGYYDAFDI | 22B6D2-hIgG1 Kabat |
| | | CDR H3 |
| SEQ ID NO: 10 | GFTFRSYG | 22B6D2-hIgG1 IMGT |
| | | CDR H1 |
| SEQ ID NO: 11 | IWYDGSNK | 22B6D2-hIgG1 IMGT |
| | | CDR H2 |
| SEQ ID NO: 12 | ARYYYDSNGYYDAFDI | 22B6D2-hIgG1 IMGT |
| | | CDR H3 |
| SEQ ID NO: 13 | QGISNY | 22B6D2-hIgG1 IMGT |
| | | CDR L1 |
| SEQ ID NO: 14 | AAS | 22B6D2-hIgG1 IMGT |
| | | CDR L2 |
| SEQ ID NO: 15 | EVHLVESGGGVVQPGRSLRLSCATSGFTFRSYGM | 22B6D2-hIgG1 VH |
| | HWVRQAPGKGLEWVAVIWYDGSNKYYADSVKG | |
| | RFTISRDISKNTLYLQMNSLTAEDTAVYYCARYYY | |
| | DSNGYYDAFDIWGQGTMVTVSS | |
| SEQ ID NO: 16 | DIQLTQSPSFLSASVGDRVTITCRASQGISNYLAWY | 22B6D2-hIgG1 VL |
| | QQKPGKAPKLLIFAASTLQSGVPSRFSGSGSGAEFT | |
| | LTISSLEPEDFATYYCQQLNSYPITFGQGTRLEIK | |
| SEQ ID NO: 17 | EVHLVESGGGVVQPGRSLRLSCATSGFTFRSYGM | 22B6D2-hIgG1 heavy |
| | HWVRQAPGKGLEWVAVIWYDGSNKYYADSVKG | chain |
| | RFTISRDISKNTLYLQMNSLTAEDTAVYYCARYYY | |
| | DSNGYYDAFDIWGQGTMVTVSSASTKGPSVFPLA | |
| | PSSKSTSGGTAALGCLVKDYFPEPVTVSWNSGALT | |
| | SGVHTFPAVLQSSGLYSLSSVVTVPSSSLGTQTYIC | |
| | NVNHKPSNTKVDKRVEPKSCDKTHTCPPCPAPEL | |
| | LGGPSVFLFPPKPKDTLMISRTPEVTCVVVDVSHE | |
| | DPEVKFNWYVDGVEVHNAKTKPREEQYNSTYRV | |
| | VSVLTVLHQDWLNGKEYKCKVSNKALPAPIEKTI | |
| | SKAKGQPREPQVYTLPPSREEMTKNQVSLTCLVK | |
| | GFYPSDIAVEWESNGQPENNYKTTPPVLDSDGSFF | |
| | LYSKLTVDKSRWQQGNVFSCSVMHEALHNHYTQ | |
| | KSLSLSPGK | |
| SEQ ID NO: 18 | DIQLTQSPSFLSASVGDRVTITCRASQGISNYLAWY | 22B6D2-hIgG1 light |
| | QQKPGKAPKLLIFAASTLQSGVPSRFSGSGSGAEFT | chain |
| | LTISSLEPEDFATYYCQQLNSYPITFGQGTRLEIKRT | |
| | VAAPSVFIFPPSDEQLKSGTASVVCLLNNFYPREA | |
| | KVQWKVDNALQSGNSQESVTEQDSKDSTYSLSST | |
| | LTLSKADYEKHKVYACEVTHQGLSSPVTKSFNRG | |
| | EC | |
| SEQ ID NO: 19 | GDSVSSSQS | 47A3E3-hIgG1 Chothia |
| | | CDR H1 |
| SEQ ID NO: 20 | FYRSRWY | 47A3E3-hIgG1 Chothia |
| | | CDR H2 |
| SEQ ID NO: 21 | DTYYYGSGGFDY | 47A3E3-hIgG1 |
| | | Chothia/Kabat CDR H3 |
| SEQ ID NO: 22 | RASQSLSRWLA | 47A3E3-hIgG1 |
| | | Chothia/Kabat CDR L1 |
| SEQ ID NO: 23 | KASSLES | 47A3E3- |
| | | hIgG1/100H7D3- |
| | | hIgG1 Chothia/Kabat |
| | | CDR L2 |
| SEQ ID NO: 24 | QQYKNYYS | 47A3E3-hIgG1 |
| | | Chothia/Kabat/IMGT |
| | | CDR L3 |
| SEQ ID NO: 25 | SSQSTWN | 47A3E3-hIgG1 Kabat |
| | | CDR H1 |
| SEQ ID NO: 26 | RTFYRSRWYNDYALSMKS | 47A3E3-hIgG1 Kabat |
| | | CDR H2 |
| SEQ ID NO: 27 | GDSVSSSQST | 47A3E3-hIgG1 IMGT |
| | | CDR H1 |
| SEQ ID NO: 28 | TFYRSRWYN | 47A3E3-hIgG1 IMGT |
| | | CDR H2 |
| SEQ ID NO: 29 | ARDTYYYGSGGFDY | 47A3E3-hIgG1 IMGT |
| | | CDR H3 |
| SEQ ID NO: 30 | QSLSRW | 47A3E3-hIgG1 IMGT |
| | | CDR L1 |
| SEQ ID NO: 31 | KAS | 47A3E3- |
| | | hIgG1/100H7D3- |
| | | hIgG1 IMGT CDR L2 |
| SEQ ID NO: 32 | EVQLQESGPGLVKPSQTLSLTCAISGDSVSSSQST | 47A3E3-hIgG1 VH |
| | WNWIRQSPSRGLEWLGRTFYRSRWYNDYALSMK | |
| | SRITINPDTSKNQFSLQLNPVTPEDTAVYYCARDT | |
| | YYYGSGGFDYWGQGSRVTVSS | |
| SEQ ID NO: 33 | DIQMTQSPSTLSASVGDRVTITCRASQSLSRWLAW | 47A3E3-hIgG1 VL |
| | YQQKPEKAPKLLIYKASSLESGVPSRFSGSGSGTEF | |
| | TLTISTLQPDDFATYYCQQYKNYYSFGQGTKLEIK | |
| SEQ ID NO: 34 | EVQLQESGPGLVKPSQTLSLTCAISGDSVSSSQST | 47A3E3-hIgG1 heavy |
| | WNWIRQSPSRGLEWLGRTFYRSRWYNDYALSMK | chain |
| | SRITINPDTSKNQFSLQLNPVTPEDTAVYYCARDT | |
| | YYYGSGGFDYWGQGSRVTVSSASTKGPSVFPLAP | |
| | SSKSTSGGTAALGCLVKDYFPEPVTVSWNSGALTS | |
| | GVHTFPAVLQSSGLYSLSSVVTVPSSSLGTQTYICN | |
| | VNHKPSNTKVDKRVEPKSCDKTHTCPPCPAPELL | |
| | GGPSVFLFPPKPKDTLMISRTPEVTCVVVDVSHED | |
| | PEVKFNWYVDGVEVHNAKTKPREEQYNSTYRVV | |
| | SVLTVLHQDWLNGKEYKCKVSNKALPAPIEKTIS | |
| | KAKGQPREPQVYTLPPSREEMTKNQVSLTCLVKG | |
| | FYPSDIAVEWESNGQPENNYKTTPPVLDSDGSFFL | |
| | YSKLTVDKSRWQQGNVFSCSVMHEALHNHYTQK | |
| | SLSLSPGK | |
| SEQ ID NO: 35 | DIQMTQSPSTLSASVGDRVTITCRASQSLSRWLAW | 47A3E3-hIgG1 light |
| | YQQKPEKAPKLLIYKASSLESGVPSRFSGSGSGTEF | chain |
| | TLTISTLQPDDFATYYCQQYKNYYSFGQGTKLEIK | |
| | RTVAAPSVFIFPPSDEQLKSGTASVVCLLNNFYPRE | |
| | AKVQWKVDNALQSGNSQESVTEQDSKDSTYSLSS | |
| | TLTLSKADYEKHKVYACEVTHQGLSSPVTKSFNR | |
| | GEC | |
| SEQ ID NO: 36 | GFTFSRN | 100H7D3-hIgG1 |
| | | Chothia CDR H1 |
| SEQ ID NO: 37 | WHDGSN | 100H7D3-hIgG1 |
| | | Chothia CDR H2 |
| SEQ ID NO: 38 | DRGASDF | 100H7D3-hIgG1 |
| | | Chothia/Kabat CDR H3 |
| SEQ ID NO: 39 | RNAMH | 100H7D3-hIgG1 Kabat |
| | | CDR H1 |
| SEQ ID NO: 40 | VIWHDGSNKYYGDSVKG | 100H7D3-hIgG1 Kabat |
| | | CDR H2 |
| SEQ ID NO: 41 | GFTFSRNA | 100H7D3-hIgG1 IMGT |
| | | CDR H1 |
| SEQ ID NO: 42 | IWHDGSNK | 100H7D3-hIgG1 IMGT |
| | | CDR H2 |
| SEQ ID NO: 43 | ARDRGASDF | 100H7D3-hIgG1 IMGT |
| | | CDR H3 |
| SEQ ID NO: 44 | QSINNW | 100H7D3-hIgG1 IMGT |
| | | CDR L1 |
| SEQ ID NO: 45 | RASQSINNWLA | 100H7D3-hIgG1 |
| | | Chothia/Kabat CDR L1 |
| SEQ ID NO: 46 | EVQLVESGGGVVLPGRSLRLSCAASGFTFSRNAM | 100H7D3-hIgG1 VH |
| | HWVRQAPGKGLEWVAVIWHDGSNKYYGDSVKG | |
| | RFTISRDNSKNTLYLQMDSLTAEDTAVYYCARDR | |
| | GASDFRGQGTLVTVSS | |
| SEQ ID NO: 47 | DIQMTQSPSTLSASVGDRVTITCRASQSINNWLAW | 100H7D3-hIgG1 VL |
| | YQQKPEKAPKLLIYKASSLESGVPSRFSGSGSGTEF | |
| | TLTISSLQPDDFATYYCQQYNSYSLTFGGGTKVEI | |
| | K | |
| SEQ ID NO: 48 | EVQLVESGGGVVLPGRSLRLSCAASGFTFSRNAM | 100H7D3-hIgG1 heavy |
| | HWVRQAPGKGLEWVAVIWHDGSNKYYGDSVKG | chain |
| | RFTISRDNSKNTLYLQMDSLTAEDTAVYYCARDR | |
| | GASDFRGQGTLVTVSSASTKGPSVFPLAPSSKSTS | |
| | GGTAALGCLVKDYFPEPVTVSWNSGALTSGVHTF | |
| | PAVLQSSGLYSLSSVVTVPSSSLGTQTYICNVNHK | |
| | PSNTKVDKRVEPKSCDKTHTCPPCPAPELLGGPSV | |
| | FLFPPKPKDTLMISRTPEVTCVVVDVSHEDPEVKF | |
| | NWYVDGVEVHNAKTKPREEQYNSTYRVVSVLTV | |
| | LHQDWLNGKEYKCKVSNKALPAPIEKTISKAKGQ | |
| | PREPQVYTLPPSREEMTKNQVSLTCLVKGFYPSDI | |
| | AVEWESNGQPENNYKTTPPVLDSDGSFFLYSKLT | |
| | VDKSRWQQGNVFSCSVMHEALHNHYTQKSLSLS | |
| | PGK | |
| SEQ ID NO: 49 | DIQMTQSPSTLSASVGDRVTITCRASQSINNWLAW | 100H7D3-hIgG1 light |
| | YQQKPEKAPKLLIYKASSLESGVPSRFSGSGSGTEF | chain |
| | TLTISSLQPDDFATYYCQQYNSYSLTFGGGTKVEI | |
| | KRTVAAPSVFIFPPSDEQLKSGTASVVCLLNNFYP | |
| | REAKVQWKVDNALQSGNSQESVTEQDSKDSTYS | |
| | LSSTLTLSKADYEKHKVYACEVTHQGLSSPVTKSF | |
| | NRGEC | |
| SEQ ID NO: 50 | ASTKGPSVFPLAPSSKSTSGGTAALGCLVKDYFPE | Human IgG1 heavy |
| | PVTVSWNSGALTSGVHTFPAVLQSSGLYSLSSVVT | chain constant region |
| | VPSSSLGTQTYICNVNHKPSNTKVDKRVEPKSCDK | |
| | THTCPPCPAPELLGGPSVFLFPPKPKDTLMISRTPE | |
| | VTCVVVDVSHEDPEVKFNWYVDGVEVHNAKTKP | |
| | REEQYNSTYRVVSVLTVLHQDWLNGKEYKCKVS | |
| | NKALPAPIEKTISKAKGQPREPQVYTLPPSREEMT | |
| | KNQVSLTCLVKGFYPSDIAVEWESNGQPENNYKT | |
| | TPPVLDSDGSFFLYSKLTVDKSRWQQGNVFSCSV | |
| | MHEALHNHYTQKSLSLSPGK | |
| SEQ ID NO: 51 | RTVAAPSVFIFPPSDEQLKSGTASVVCLLNNFYPRE | Human IgG1 light chain |
| | AKVQWKVDNALQSGNSQESVTEQDSKDSTYSLSS | constant region |
| | TLTLSKADYEKHKVYACEVTHQGLSSPVTKSFNR | |
| | GEC | |
| SEQ ID NO: 52 | QQYNSYSLT | 100H7D3-hIgG1 |
| | | Chothia/Kabat/IMGT |
| | | CDR L3 |

Abbreviations used herein have the following meanings as shown in Table C below:

TABLE C

| Abbre- | | Abbre- | |
|---|---|---|---|
| viation | Meaning | viation | Meaning |
| HATU | N,N,N′,N′-tetramethyl- | NBS | N-bromosuccinimide |
| | O-(7-azabenzotriazol- | | |
| | 1-yl)urea | | |
| | hexafluorophosphate | | |
| EDCI | N-(3- | HOBt | 1- |
| | dimethylaminopropyl)- | | hydroxybenzotriazole |
| | N′-ethylcarbodiimide | | |
| | hydrochloride | | |
| DMTMM | 4-(4,6-dimethoxy- | DIPEA | Diisopropylethylamine |
| | 1,3,5-triazin-2-yl)- | | |
| | 4-methylmorpholinium | | |
| | chloride | | |
| DMAP | 4- | Triphosgene | Bis (trichloromethyl) |
| | dimethylaminopyridine | | carbonate |
| Et₃N | Triethylamine | Et₂NH | Diethylamine |
| TFA | Trifluoroacetic acid | TCEP | tris(2- |
| | | | carboxyethyl)phos- |
| | | | phine |
| Pd/C | Palladium on carbon | EDTA | Ethylenediamine- |
| | | | tetraacetic acid |
| Na₂HPO₄ | di-Sodium hydrogen | PB | Phosphate buffered |
| | phosphate | | saline |
| DCM | Dichloromethane | THF | Tetrahydrofuran |
| IPA | Isopropyl alcohol | DMSO | Dimethyl sulfoxide |
| MeOH | Methanol | DMF | N,N- |
| | | | Dimethylformamide |
| HPLC | High Performance | LC-MS | Liquid |
| | Liquid | | chromatography-mass |
| | Chromatography | | spectrometry |

The structures of the compounds described in the following Examples were confirmed by nuclear magnetic resonance (1H-NMR) or mass spectrometry (MS).

The determination of nuclear magnetic resonance (1H-NMR) was performed by using a Bruker 400 MHz nuclear magnetic resonance instrument; the deuterated reagent was hexadeuteriodimethylsulfoxide (DMSO-d6); and the internal standard substance was tetramethylsilane (TMS).

Abbreviations in the nuclear magnetic resonance (NMR) spectra used in Examples were shown below.
s: singlet, d: doublet, t: triplet, q: quartet, m: multiplet, br: broad, J: coupling constant, Hz: Hertz, DMSO-d6: deuterated dimethylsulfoxide. δ value was expressed in ppm.
Agilent (ESI) mass spectrometer Agilent 6120B was used to determine mass spectrum (MS).

### Example 1: N—((S)-10-benzyl-1-((1S,9S)-9-ethyl-5-fluoro-9-hydroxyl-4-methyl-10,13-dioxo-2,3,9,10,13,15-hexahydro-1H,12H-benzo[de]pyrano[3′,4′: 6,7]indolizino[1,2-b]quinolin-1-yl)amino-1,6,9,12,15-pentaoxo-3-oxa-5,8,11,14-tetraazahexadecan-16-yl)-6-(2,5-dioxo-2,5-dihydro-1-H-pyrrol-1-yl) hexanamide (A-01)

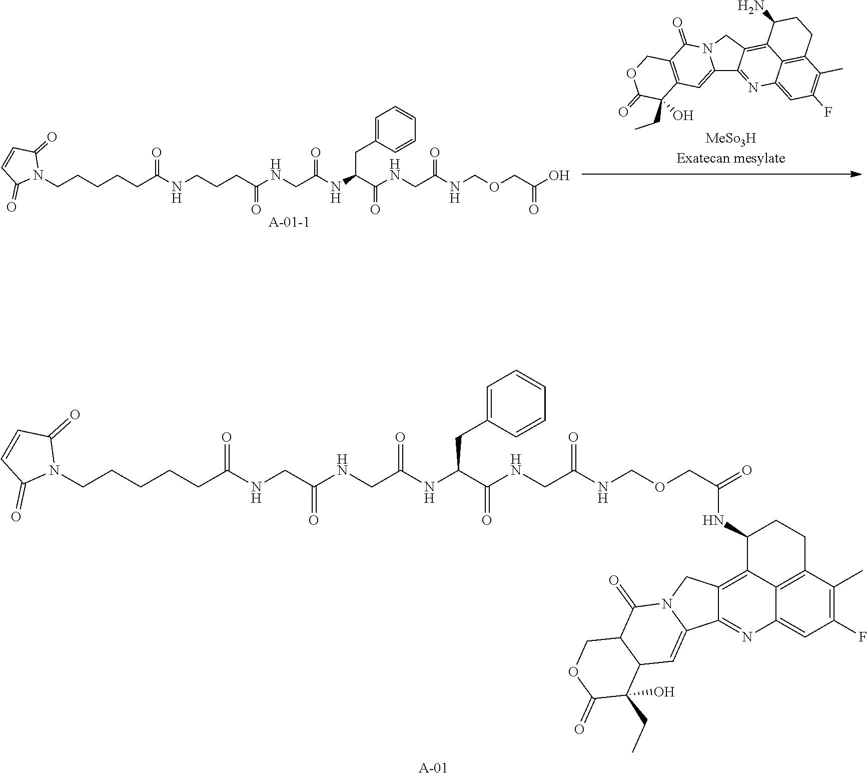 [SMILES: O=C(O)COCNC(=O)CNC(=O)[C@H](Cc1ccccc1)NC(=O)CNC(=O)CCCNC(=O)CCCCCN1C(=O)C=CC1=O ; CC[C@@]1(O)C(=O)OCc2c1cc1n(c2=O)Cc2c-1nc1cc(F)c(C)c3c1c2[C@@H](N)CC3 ; CC[C@@]1(O)C(=O)OCC2C(=O)N3Cc4c(nc5cc(F)c(C)c6c5c4[C@@H](NC(=O)COCNC(=O)CNC(=O)[C@H](Cc4ccccc4)NC(=O)CNC(=O)CNC(=O)CCCCCN4C(=O)C=CC4=O)CC6)C3=CC21 ; "A-01"]

Compound A-01-1 (0.40 g, 640.59 μmol, its synthesis could be referred to patent application publication No.: CN 111936169A) and exatecan mesylate (0.37 g, 704.65 μmol) were dissolved in DMF (8 mL), added with HATU (0.32 g, 832.77 μmol) and DIPEA (0.25 g, 1.92 mmol), reacted at 25° C. for 4 hours. DIPEA was removed under reduced pressure and most of DMF was removed by subjecting to lyophilization with water to obtain a crude product, and the crude product was purified by preparative high performance liquid chromatography under the following conditions to obtain 273 mg of the title compound.

Chromatographic column: Waters XBridge Prep C18 OBD 45 mm×450 mm×8.0 μm

Mobile phase A: acetonitrile; mobile phase B: water (0.05% trifluoroacetic acid)

| Time | | | |
|---|---|---|---|
| [min] | Mobile phase A [%] | Mobile phase B [%] | Flow rate [mL/min] |
| 0.00 | 30 | 70 | 70 |
| 5.00 | 30 | 70 | 70 |
| 60.00 | 70 | 30 | 70 |

The structural characterization data were as follows:
ESI-MS (m/z): 1034.4 [M+H]⁺.

### Example 2: N-((1S,9S)-5-chloro-9-ethyl-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2-hydroxyacetamide and N-((1R,9S)-5-chloro-9-ethyl-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2-hydroxyacetamide (1-8-A and 1-8-B)

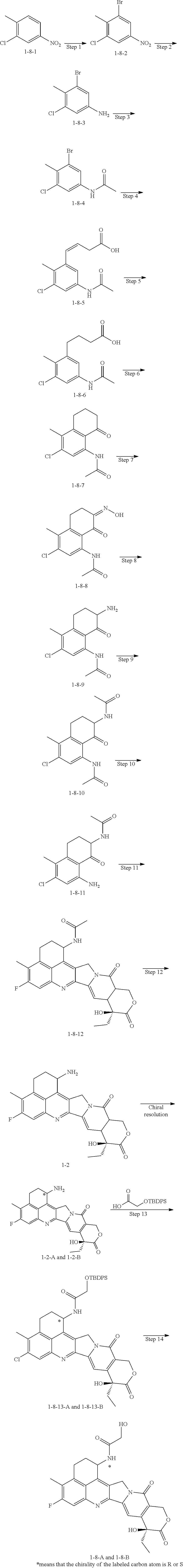

### Step 1: Synthesis of 1-chloro-3-bromo-2-methyl-5-nitrobenzene (1-8-2)

At 25° C., Compound 1-8-1 (5.00 g, 29.14 mmol) was dissolved in n-heptane (25 mL), added with concentrated sulfuric acid (25 mL), heated to 50° C., added with NBS in batches at 50° C. (6.22 g, 34.97 mmol), reacted at 50° C. for 2 hours, and the reaction was detected by thin-layer chromatography (ethyl acetate:petroleum ether=1:10). The reaction solution was cooled to room temperature, then added dropwise into ice water, extracted with toluene, and the organic phases were combined, washed with sodium sulfite solution, water, and saturated brine, dried over anhydrous sodium sulfate, concentrated under reduced pressure, and the crude product was purified by preparative high-performance liquid chromatography under the following conditions, the fractions were lyophilized to obtain 4.88 g of the title compound.

Chromatographic column: C18 ODS 45 mm×450 mm×8.0 μm

Mobile phase A: acetonitrile; Mobile phase B: water (0.05% formic acid)

| Time | | | |
|---|---|---|---|
| [min] | Mobile phase A [%] | Mobile phase B[%] | Flow rate [mL/min] |
| 0 | 60 | 40 | 60 |
| 10 | 60 | 40 | 60 |
| 40 | 100 | 0 | 60 |

### Step 2: Synthesis of 3-chloro-5-bromo-4-methylaniline (1-8-3)

At 25° C., Compound 1-8-2 (4.88 g, 19.48 mmol) was dissolved in ethyl acetate (100 mL), platinum on carbon (2.00 g, 19.48 mmol, content 5%) was added. Hydrogen replacement was performed, and the reaction was carried out at 60° C. for 4 hours under H₂ atmosphere, and the reaction was monitored by HPLC-MS. The reaction solution was filtered and concentrated to obtain 3.68 g of a crude product of the title compound, which was directly used in the next reaction without further purification.

### Step 3: Synthesis of N-(3-chloro-5-bromo-4-methylphenyl) acetamide (1-8-4)

At 20° C., Compound 1-8-3 (3.63 g, 14.82 mmol) was dissolved in ethyl acetate (70 mL), triethylamine (4.50 g, 44.45 mmol) and acetic anhydride (2.27 g, 22.23 mmol) were added, the reaction was carried out at 20° C. for 20 h, and the reaction was monitored by HPLC-MS. The reaction solution was added with water, extracted with ethyl acetate, the organic phases were combined, dried over anhydrous sodium sulfate, and concentrated under reduced pressure to obtain a crude product, which was slurried in a mixed solvent of ethyl acetate:petroleum ether=1:5 to obtain 2.86 g of the title compound.

### Step 4: Synthesis of (Z)-4-(5-acetamido-3-chloro-2-methylphenyl) but-3-enoic acid (1-8-5)

At 20° C., Compound 1-8-4 (1.80 g, 6.86 mmol) was dissolved in THF (20 mL) and water (5 mL), and vinylacetic acid (708.31 mg, 8.23 mmol), DIPEA (1.95 g, 15.08 mmol) and tris(o-methylphenyl)phosphorus (62.60 mg, 0.20 mmol) were added, the reaction system was subjected to nitrogen replacement, and then heated to 70° C. and reacted for 5 h, and the reaction was monitored by HPLC-MS. 1N sodium hydroxide solution was added to the reaction solution to adjust pH=8, ethyl acetate was added for extraction, the aqueous phase was adjusted to pH=3 with 1N hydrochloric acid, extracted with ethyl acetate, the organic phases were combined, dried over anhydrous sodium sulfate, and concentrated under reduced pressure to obtain 0.82 g of the title compound, which was directly used in the next reaction.

### Step 5: Synthesis of 4-(5-acetamido-3-chloro-2-methylphenyl) butanoic acid (1-8-6)

At 20° C., Compound 1-8-5 (2.60 g, 9.71 mmol) was dissolved in THF (50 mL), added with Pd/C (0.52 g, content 10%). The reaction system was subjected to hydrogen replacement, and then reacted under the protection of a hydrogen balloon at 40° C. for 2 h, and the reaction was monitored by HPLC-MS. The reaction solution was filtered, and the filtrate was concentrated to obtain 2.43 g of the title compound, which was directly used in the next reaction without further purification.

### Step 6: Synthesis of N-(3-chloro-4-methyl-8-oxo-5,6,7,8-tetrahydronaphth-1-yl) acetamide (1-8-7)

Compound 1-8-6 (2.43 g, 9.01 mmol) was dissolved in trifluoroacetic acid (10 mL), cooled to 5° C., added dropwise with trifluoroacetic anhydride (3.78 g, 18.02 mmol, 2.50 mL), reacted at 5° C. for 4 h, and the reaction was monitored by HPLC-MS. The reaction solution was added to water, adjusted with 10N sodium hydroxide to pH=9, added with ethyl acetate for extraction, the organic phases were combined, dried over anhydrous sodium sulfate, concentrated under reduced pressure, and purified by silica gel column (ethyl acetate:petroleum ether=0˜20%) to obtain 1.53 g of the title compound.

### Step 7: Synthesis of (Z)—N-(3-chloro-7-(hydroxyimino)-4-methyl-8-oxo-5,6,7,8-tetrahydronaphth-1-yl) acetamide (1-8-8)

At 5° C., potassium tert-butoxide (1.50 g, 13.37 mmol) was dissolved in THF (16 mL) and tert-butanol (4 mL), added dropwise with Compound 1-8-7 (1.53 g, 6.08 mmol) in THF solution (16 mL), followed by an addition of amyl nitrite (1.14 g, 9.73 mmol) in a dropwise manner after 10 minutes, reacted at 5° C. for 1 h, and the reaction was monitored by HPLC-MS. The reaction solution was adjusted to pH=5 with 1N hydrochloric acid, extracted with ethyl acetate, the organic phases were combined and dried over anhydrous sodium sulfate, concentrated under reduced pressure, and the crude product was slurried with methyl tert-butyl ether to obtain 1.20 g of the title compound.

### Step 8: Synthesis of N-(7-amino-3-chloro-4-methyl-8-oxo-5,6,7,8-tetrahydronaphth-1-yl) acetamide (1-8-9)

At 20° C., Compound 1-8-8 (0.50 g, 1.78 mmol) was dissolved in methanol (8 mL) and 2N hydrochloric acid (8 mL), and added with Pd/C (0.15 g, content 10%), the system was subjected to hydrogen replacement, then the reaction was carried out at 5° C. for 2 h under the protection of a hydrogen balloon, and the reaction was monitored by HPLC-MS. The reaction solution was filtered and concentrated to obtain 0.52 g of a hydrochloride salt of the title compound, which was directly used in the next reaction without further purification.

### Step 9: Synthesis of N,N′-(3-chloro-4-methyl-8-oxo-5,6,7,8-tetrahydronaphth-1,7-diyl)diacetamide (1-8-10)

At 20° C., Compound 1-8-9 (0.52 g, 1.70 mmol) was dissolved in pyridine (5 mL), added with acetic anhydride (2 mL), reacted at 20° C. for 2 h, and the reaction was monitored by HPLC-MS. The reaction solution was added to water, extracted with ethyl acetate, the organic phases were washed with water, combined, dried over anhydrous sodium sulfate, concentrated under reduced pressure, and purified by silica gel column (ethyl acetate:petroleum ether=0˜30%) to obtain 0.22 g of the title compound.

### Step 10: Synthesis of N-(8-amino-6-chloro-5-methyl-1-oxo-1,2,3,4-tetrahydronaphth-2-yl) acetamide (1-8-11)

At 20° C., Compound 1-8-10 (450.97 mg, 1.46 mmol) was dissolved in methanol (16 mL), added with 2N hydrochloric acid (16 mL), heated to 60° C. and reacted for 2 h, and the reaction was monitored by HPLC-MS. The reaction solution was cooled and adjusted to pH=8 by adding saturated sodium bicarbonate solution, extracted with ethyl acetate, the organic phases were combined, dried over anhydrous sodium sulfate, and concentrated under reduced pressure to obtain 230.00 mg of the title compound, which was used directly in the next reaction without further purification. Step 11: Synthesis of N-((9S)-5-chloro-9-ethyl-9-hydroxy-4-methyl-10,13-dioxo-2,3,9,10,13,15-hexahydrogen-1H,12H-benzopyrano[3′,4′: 6,7]indolizino[1,2-b]quinolin-1-yl) acetamide (1-8-12)

Compound 1-8-11 (230.00 mg, 0.78 mmol) was dissolved in toluene (10 mL), added with(S)-4-ethyl-4-hydroxy-7,8-dihydro-1H-pyrano[3,4-f]indolizine-3,6,10 (4H)-trione (230.00 mg, 0.87 mmol), and p-toluenesulfonic acid (26.73 mg, 0.16 mmol), heated to 140° C. and reacted for 5 h, and the reaction was monitored by HPLC-MS. The reaction solution was concentrated, and the crude product was purified by silica gel column (methanol:dichloromethane=0˜10%) to obtain 150.00 mg of the title compound.

### Step 12: Synthesis of (9S)-1-amino-5-chloro-9-ethyl-9-hydroxy-4-methyl-1,2,3,9,12,15-hexahydro-10H,13H-benzopyrano[3′,4′: 6,7]indolizino[1,2-b]quinoline-10,13-dione (1-2)

Compound 1-8-12 (40.00 mg, 0.081 mmol) was added into concentrated hydrochloric acid (1 mL), heated to 100° C. and reacted for 5 h, and the reaction was monitored by HPLC-MS. The reaction solution was filtered, the filtrate was purified by preparative high performance liquid chromatography under following conditions, and the obtained solution was lyophilized to obtain a hydrochloride of the title Compound 1-2. The hydrochloride of Compound 1-2 was separated under the following purification conditions to obtain two isomers, which were named as 1-2-A (trifluoroacetate, 5.00 mg; retention time, 9.85 min) and 1-2-B (trifluoroacetate, 7.00 mg; retention time, 10.62 min) according to retention time.

Chromatographic column: SunFire Prep C18 OBD 19 mm×150 mm×5.0 μm

Mobile phase A: acetonitrile; Mobile phase B: water (0.05% trifluoroacetic acid)

| Time | | | |
|---|---|---|---|
| [min] | Mobile phase A [%] | Mobile phase B[%] | Flow rate [mL/min] |
| 0 | 5 | 95 | 28 |
| 2 | 5 | 95 | 28 |
| 18 | 50 | 50 | 28 |

The structural characterization data were as follows:

### 1-2-A:

¹H-NMR (400 MHZ, DMSO-d6) δ 8.42 (s, 3H), 8.27 (s, 1H), 7.36 (s, 1H), 6.59 (s, 1H), 5.78-5.63 (m, 1H), 5.50-5.36 (m, 3H), 5.10-5.06 (m, 1H), 3.20-3.04 (m, 2H), 2.56 (s, 3H), 2.26-2.13 (m, 2H), 1.93-1.79 (m, 2H), 0.88 (t, J=7.2 Hz, 3H).

ESI-MS (m/z): 452.1 [M+H]⁺.

### 1-2-B:

¹H-NMR (400 MHZ, DMSO-d6) δ 8.42 (s, 3H), 8.27 (s, 1H), 7.36 (s, 1H), 6.58 (s, 1H), 5.78-5.63 (m, 1H), 5.50-5.36 (m, 3H), 5.10-5.06 (m, 1H), 3.20-3.04 (m, 2H), 2.55 (s, 3H), 2.26-2.13 (m, 2H), 1.93-1.79 (m, 2H), 0.88 (t, J=7.2 Hz, 3H).

ESI-MS (m/z): 452.0 [M+H]⁺.

### Step 13: Synthesis of 2-((tert-butyldiphenylsilyl)oxy)-N-((1S,9S)-5-chloro-9-ethyl-9-hydroxyl-4-methyl-10,13-dioxo-2,3,9,10,13,15-hexahydro-1H, 12H-benzo[de]pyrano[3′,4′: 6,7]indolizino[1,2-b]quinolin-1-yl) acetamide and 2-((tert-butyldiphenylsilyl)oxy)-N-((1R,9S)-5-chloro-9-ethyl-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl) acetamide (1-8-13-A and 1-8-13-B)

At 25° C., the hydrochloride salt of Compound 1-2 (40.00 mg, 81.91 μmol) was dissolved in N,N-dimethylformamide (1 mL), added in sequence with 2-((tert-butyldiphenylsilyl)oxy) acetic acid (30.91 mg, 98.29 μmol), HATU (62.25 mg, 163.81 μmol) and N,N-diisopropylethylamine (42.34 mg, 327.63 μmol), reacted at 25° C. for 0.5 h, and the reaction was monitored by HPLC-MS. After the reaction was completed, the reaction solution was added with water, extracted with dichloromethane/methanol (v/v=10/1), the organic phases were combined, dried over anhydrous sodium sulfate and concentrated under reduced pressure, and the crude product was purified and separated by preparative thin-layer chromatography (dichloromethane:methanol=20:1) to obtain two isomers, which were named as Jan. 8, 2013-A (15.00 mg, Rf value was 0.3) and Jan. 8, 2013-B (12.00 mg, Rf value was 0.35) according to their Rf values.

### Step 14: Synthesis of N-((1S,9S)-5-chloro-9-ethyl-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2-hydroxyacetamide and N-((1R,9S)-5-chloro-9-ethyl-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2-hydroxyacetamide (1-8-A and 1-8-B)

At 25° C., Jan. 8, 2013-A (15.00 mg) and Jan. 8, 2013-B (12.00 mg) were dissolved in tetrahydrofuran (1 mL) in two reaction flasks, respectively, added dropwise with tetrabutylammonium fluoride in a mixed solution of tetrabutylammonium fluoride (1M tetrahydrofuran solution)/glacial acetic acid (v/v=13/1) (50 μL), reacted at 25° C. for 0.5 h, and the reaction was monitored by HPLC-MS. After the reaction was completed, the reaction solution was purified by preparative high-performance liquid chromatography, and the prepared solution was lyophilized to obtain the title compounds 1-8-A (6.94 mg) and 1-8-B (4.00 mg).

Chromatographic column: SunFire Prep C18 OBD 19 mm×150 mm×5.0 μm

Mobile phase A: acetonitrile; Mobile phase B: water (0.05% formic acid)

| Time | | | |
|---|---|---|---|
| [min] | Mobile phase A [%] | Mobile phase B[%] | Flow rate [mL/min] |
| 0 | 20 | 80 | 28 |
| 3 | 20 | 80 | 28 |
| 18 | 90 | 10 | 28 |

The structural characterization data of 1-8-A were as follows:

¹H-NMR (400 MHZ, DMSO-d6) δ 8.43 (d, J=8.8 Hz, 1H), 8.16 (s, 1H), 7.31 (s, 1H), 6.55 (s, 1H), 5.65-5.36 (m, 4H), 5.21 (q, J=19.0 Hz, 2H), 3.95 (d, J=5.7 Hz, 2H), 3.26-3.11 (m, 2H), 2.53 (s, 3H), 2.30-2.08 (m, 2H), 1.94-1.79 (m, 2H), 0.87 (t, J=7.3 Hz, 3H).

ESI-MS (m/z): 510.1 [M+H]⁺.

The structural characterization data of 1-8-B were as follows:

¹H-NMR (400 MHZ, DMSO-d6) δ 8.45 (d, J=8.9 Hz, 1H), 8.15 (s, 1H), 7.31 (s, 1H), 6.54 (s, 1H), 5.64-5.35 (m, 4H), 5.19 (q, J=19.0 Hz, 2H), 3.97 (d, J=5.2 Hz, 2H), 3.27-3.10 (m, 2H), 2.51 (s, 3H), 2.27-2.10 (m, 2H), 1.93-1.80 (m, 2H), 0.88 (t, J=7.3 Hz, 3H).

ESI-MS (m/z): 510.1 [M+H]⁺.

### Example 3: Synthesis of N—((S)-10-benzyl-1-(((1S,9S)-5-chloro-9-ethyl-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1,6,9,12,15-pentaoxo-3-oxa-5,8,11,14-tetraazahexadecan-16-yl)-6-(2-(methylsulfonyl)pyrimidin-5-yl) hex-5-ynamide and N—((S)-10-benzyl-1-(((1R,9S)-5-chloro-9-ethyl-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)amino)-1,6,9,12,15-pentaoxo-3-oxa-5,8,11,14-tetraazahexadecan-16-yl)-6-(2-(methylsulfonyl)pyrimidin-5-yl) hex-5-ynamide (A-07-A and A-07-B)

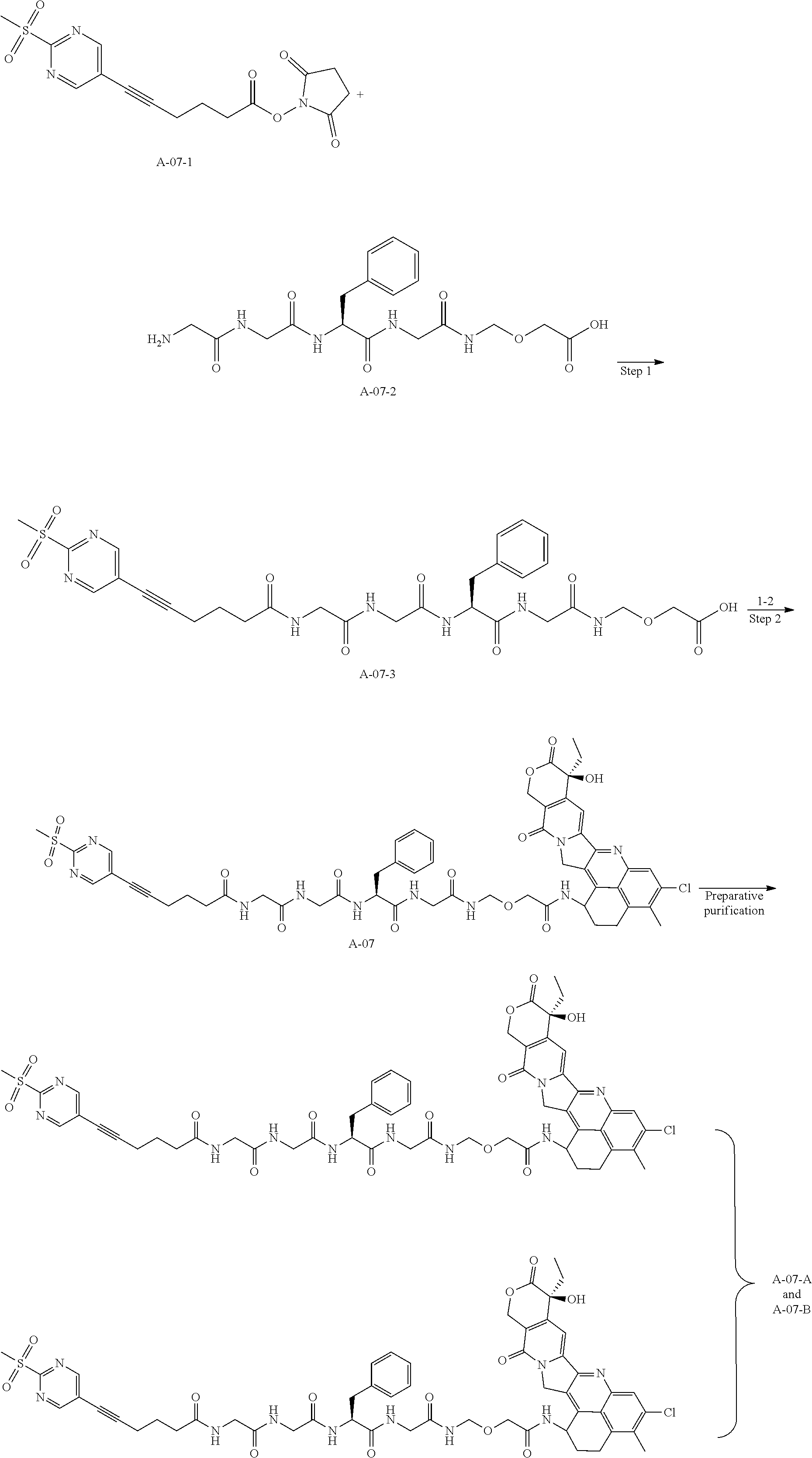

### Step 1: Synthesis of(S)-10-Benzyl-23-(2-(methylsulfonyl)pyrimidin-5-yl)-6,9,12,15,18-pentoxo-3-oxohetero-5,8,11,14,17-pentaazatricosane-22-yne carboxylic acid (A-07-3)

At 25° C., compound A-07-2 (30.00 mg, 0.07 mmol) was dissolved in DMF (0.2 mL), and 2,5-dioxypyrrolidin-1-yl-6-(2-(methylsulfonyl)pyrimidin-5-yl) hexyl-5-ynoate was added (A-07-1, 28.00 mg, 0.08 mmol), the mixture reacted at 30° C. for 1 h, and the reaction monitored by HPLC-MS. The reaction solution was directly purified by preparative high-performance liquid chromatography (conditions are as follows), and the preparative solution was lyophilized to obtain 20.00 mg of the title compound.

Chromatographic column: SunFire Prep C18 OBD 19 mm×150 mm×5.0 μm

Mobile phase A: acetonitrile; Mobile phase B: water (0.05% formic acid)

| Time | Mobile | Mobile | Flow rate |
|---|---|---|---|
| [min] | phase A [%] | phase B[%] | [mL/min] |
| 0.00 | 10 | 90 | 28 |
| 2.00 | 10 | 90 | 28 |
| 18.00 | 90 | 10 | 28 |

The structural characterization data were as follows:
ESI-MS (m/z): 691.0 [M+H₂O]⁺.

### Step 2: Synthesis of N—((S)-10-benzyl-1-(((1S,9S)-5-chloro-9-ethyl-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1,6,9,12,15-pentaoxo-3-oxa-5,8,11,14-tetraazahexadecan-16-yl)-6-(2-(methylsulfonyl)pyrimidin-5-yl) hex-5-ynamide and N—((S)-10-benzyl-1-(((1R,9S)-5-chloro-9-ethyl-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1,6,9,12,15-pentaoxo-3-oxa-5,8,11,14-tetraazahexadecan-16-yl)-6-(2-(methylsulfonyl)pyrimidin-5-yl) hex-5-ynamide (A-07-A and A-07-B)

At 25° C., the hydrochloride salt of 1-2 (30.00 mg, 61.43 μmol) was dissolved in N,N-dimethylformamide (1 mL), added in sequence with A-07-3 (49.66 mg, 73.72 μmol), HATU (35.01 mg, 92.14 μmol) and N,N-diisopropylethylamine (23.82 mg, 184.29 μmol), reacted at 25° C. for 0.5 hours, and the reaction was monitored by HPLC-MS. After the reaction was completed, the reaction solution was purified by preparative high-performance liquid chromatography under the following conditions, and the fractions were lyophilized to obtain the title compound A-07. A-07 was separated under the following purification conditions to obtain two isomers, which were named as A-07-A (11.04 mg, retention time was 7.5 min) and A-07-B (19.42 mg, retention time was 8.0 min), according to retention time.

Chromatographic column: SunFire Prep C18 OBD 19 mm×150 mm×5.0 μm

Mobile phase A: acetonitrile; Mobile phase B: water (0.05% formic acid)

| Time | Mobile | Mobile | Flow rate |
|---|---|---|---|
| [min] | phase A [%] | phase B[%] | [mL/min] |
| 0 | 30 | 70 | 28 |
| 3 | 30 | 70 | 28 |
| 18 | 90 | 10 | 28 |

The structural characterization data were as follows:

### A-07-A:

ESI-MS (m/z): 1107.3 [M+H]⁺.

### A-07-B:

ESI-MS (m/z): 1107.3 [M+H]⁺.

### Example 4: (S)-7-ethyl-7-hydroxy-14-(2-(isopropylamino)ethyl)-10,13-dihydro-11H-[1,3]dioxolo[4,5-g]pyrano[3′,4′: 6,7]indolizino[1,2-b]quinoline-8,11(7H)-dione (2-2)

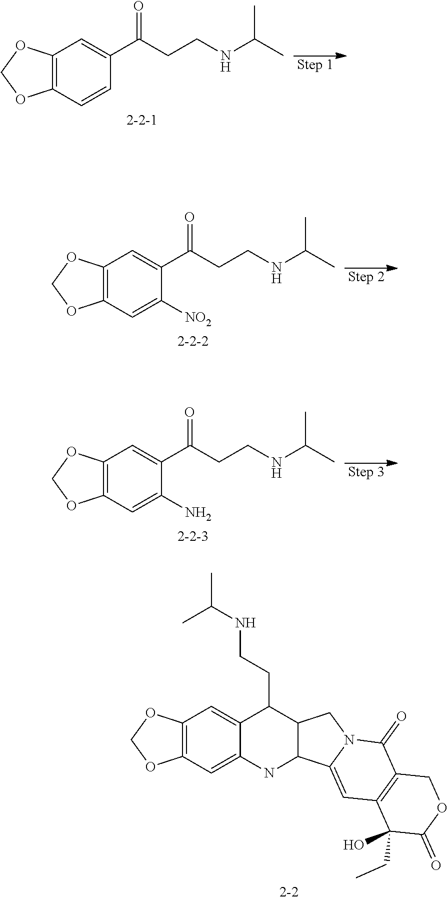 [SMILES: CC(C)NCCC(=O)c1ccc2c(c1)OCO2 ; CC(C)NCCC(=O)c1cc2c(cc1[N+](=O)[O-])OCO2 ; CC(C)NCCC(=O)c1cc2c(cc1N)OCO2 ; CC[C@@]1(O)C(=O)OCc2c1cc1n(c2=O)CC2C(CCNC(C)C)c3cc4c(cc3NC12)OCO4]

### Step 1: Synthesis of 3-(isopropylamino)-1-(6-nitrobenzo[d][1,3]dioxol-5-yl) propan-1-one (2-2-2)

Compound 2-2-1 (1.90 g, 8.08 mmol) was slowly added into nitric acid (8 mL) at 0° C., slowly heated to 25° C. and reacted for 1 hour. The reaction solution was directly purified by reverse-phase column chromatography (acetonitrile/0.5% aqueous formic acid) to obtain 1.50 g of a formate salt of the title compound.

The structural characterization data were as follows:
ESI-MS (m/z): 281.1 [M+H]⁺.

### Step 2: Synthesis of 1-(6-aminobenzo[d][1,3]dioxol-5-yl)-3-(isopropylamino) propan-1-one (2-2-3)

The formate salt of Compound 2-2-2 (1.25 g, 4.46 mmol) was added into tetrahydrofuran (20 mL), added with 10% palladium on carbon (125.00 mg), subjected to hydrogen replacement three times, and reacted at 25° C. for 16 hours. The reaction solution was filtered through celite, and the filtrate was concentrated to dryness under reduced pressure to obtain 895.00 mg of a crude product of the title compound, which was directly used in the next reaction without purification.

The structural characterization data were as follows:
ESI-MS (m/z): 251.1 [M+H]⁺.

### Step 3: Synthesis of(S)-7-ethyl-7-hydroxy-14-(2-(isopropylamino)ethyl)-10,13-dihydro-11H-[1,3]dioxolo[4,5-g]pyrano[3′,4′: 6,7]indolizino[1,2-b]quinoline-8,11 (7H)-dione (2-2)

Compound 2-2-3 (23.00 mg, 0.09 mmol) and (4S)-4-ethyl-4-hydroxy-7,8-dihydro-1H-pyrano[3,4-f]indolizino-3,6,10 (4H)-trione (21.00 mg, 0.09 mmol) were dissolved in toluene (4 mL), added with p-toluenesulfonic acid (1.40 mg, 0.009 mmol), and reacted at 140° C. for 12 h, dried under reduced pressure to remove the solvent and obtained a crude product of the title compound. The crude product was purified by HPLC (mobile phase A: acetonitrile, mobile phase B: 0.05% formic acid aqueous solution), the prepared solution was added with 3 drops of 3M hydrochloric acid and lyophilized to obtain 8.70 mg of a hydrochloride of the title compound.

The structural characterization data were as follows:
ESI-MS (m/z): 478.2 [M+H]⁺.

¹H-NMR (400 MHZ, DMSO-d6): δ 9.38 (brs, 2H), 7.85 (s, 1H), 7.52 (s, 1H), 7.25 (s, 1H), 6.30 (d, J=2.0 Hz, 2H), 5.43 (s, 2H), 5.30 (s, 2H), 3.57-3.45 (m, 2H), 3.40-3.25 (m, 1H), 3.22-3.06 (m, 2H), 1.95-1.77 (m, 2H), 1.27 (d, J=6.4 Hz, 6H), 0.87 (t, J=7.6 Hz, 3H).

### Example 5: Synthesis of 4-((S)-2-(4-aminobutyl)-35-(4-((6-(2-(methylsulfonyl)pyrimidin-5-yl) hex-5-ynamido)methyl)-1H-1,2,3-triazol-1-yl)-4,8-dioxo-6,12,15,18,21,24,27,30,33-nonaoxa-3,9-diazapentatriacontanamido)benzyl ((S)-4-ethyl-11-(2-(N-isopropylmethylsulfonamido)ethyl)-3,14-dioxo-3,4,12,14-tetrahydro-1H-pyrano[3′,4′: 6,7]indolizino[1,2-b]quinolin-4-yl) carbonate (B-01)

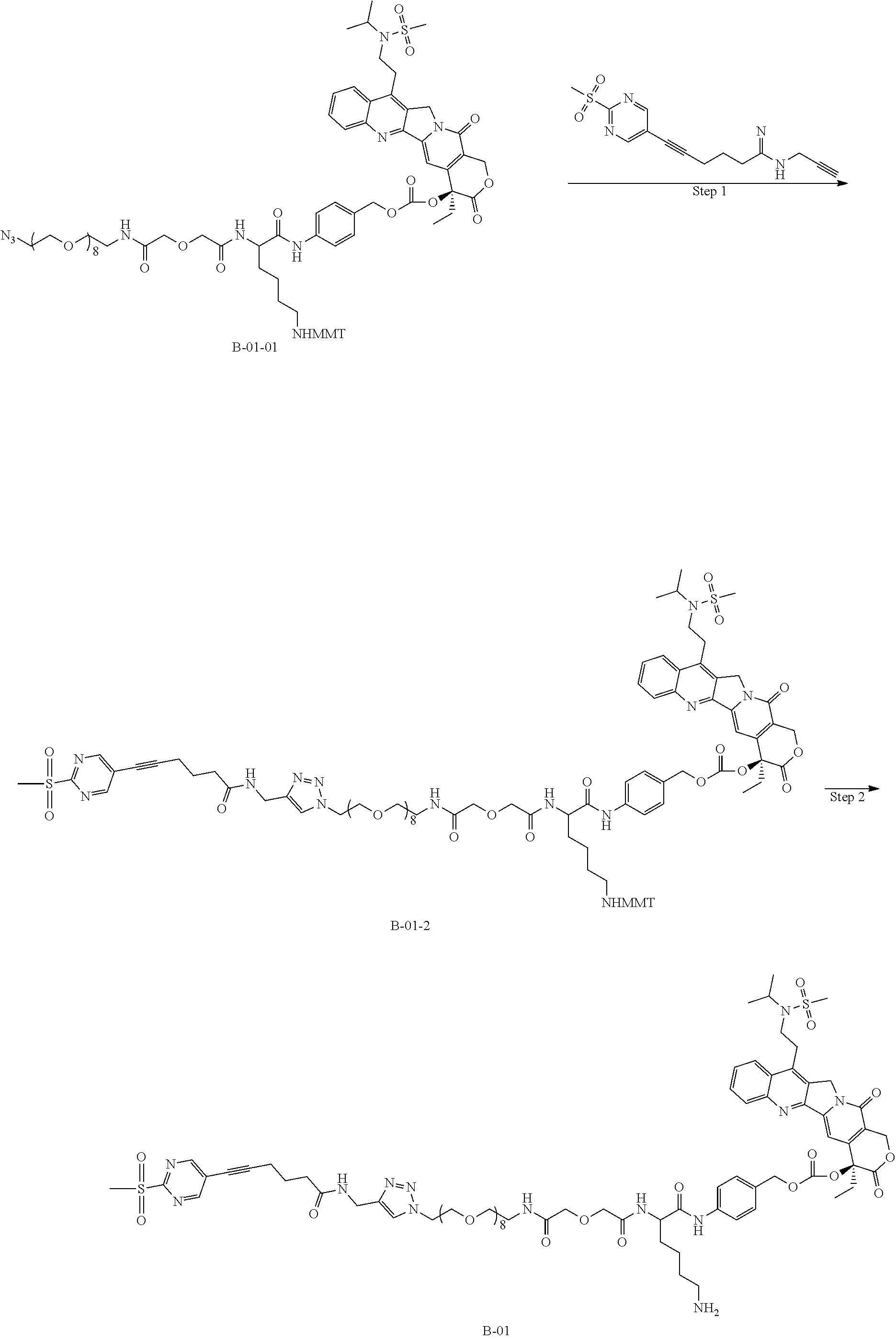

### Step 1: Synthesis of(S)-4-ethyl-11-(2-(N-isopropylmethylsulfonamido)ethyl)-3,14-dioxo-3,4,12,14-tetrahydro-1H-pyrano[3′,4′: 6,7]indolizino[1,2-b]quinolin-4-yl (4-((S)-2-(4-(((4-methoxyphenyl)diphenylmethyl)amino)butyl)-35-(4-((6-(2-(methylsulfonyl)pyrimidin-5-yl) hex-5-ynamido)methyl)-1H-1,2,3-triazol-1-yl)-4,8-dioxo-6,12,15,18,21,24,27,30,33-nonaoxa-3,9-diazapentatriacontanamido)benzyl) carbonate (B-01-2)

At room temperature, Compound B-01-1 (413.40 mg, 0.251 mmol, its synthesis was referred to patent No.: CN111295389B) was dissolved in dimethyl sulfoxide and water (2.0 mL: 0.5 mL), added with cuprous bromide (72.95 mg, 0.503 mmol) and 6-(2-(methylsulfonyl)pyrimidin-5-yl)-N-(prop-2-yn-1-yl)-hex-5-ynamide (95.10 mg, 0.302 mmol), stirred and reacted for 1 h, then filtered, and the filtrate was purified by preparative high performance liquid chromatography under the following conditions to obtain 30.00 mg of the title compound.

Chromatographic column: SunFire Prep C18 OBD 19 mm×150 mm×5.0 μm

Mobile phase A: acetonitrile; Mobile phase B: water

| Time | Mobile | Mobile | Flow rate |
|---|---|---|---|
| [min] | phase A [%] | phase B[%] | [mL/min] |
| 0.00 | 40 | 60 | 28 |
| 8.00 | 40 | 60 | 28 |
| 50.00 | 90 | 10 | 28 |

The structural characterization data were as follows:
ESI-MS (m/z): 815.9 [(M-273)/2+H]⁺.

### Step 2: Synthesis of 4-((S)-2-(4-aminobutyl)-35-(4-((6-(2-(methylsulfonyl)pyrimidin-5-yl) hex-5-ynamido)methyl)-1H-1,2,3-triazol-1-yl)-4,8-dioxo-6,12,15,18,21,24,27,30,33-nonaoxa-3,9-diazapentatriacontanamido)benzyl ((S)-4-ethyl-11-(2-(N-isopropylmethylsulfonamido)ethyl)-3,14-dioxo-3,4,12,14-tetrahydro-1H-pyrano[3′,4′: 6,7]indolizino[1,2-b]quinolin-4-yl) carbonate (B-01)

Compound B-01-2 (30.00 mg, 0.02 mmol) was dissolved in dichloromethane (1.0 mL), the reaction solution was added with trifluoroacetic acid (0.2 mL), and reacted at room temperature for 30 min. The reaction solution was concentrated under reduced pressure and purified by preparative high performance liquid chromatography under the following conditions to obtain 20.00 mg of a trifluoroacetate of the title compound.

Chromatographic column: SunFire Prep C18 OBD 19 mm×150 mm×5.0 μm

Mobile phase A: acetonitrile; Mobile phase B: water (0.05% trifluoroacetic acid)

| Time | Mobile | Mobile | Flow rate |
|---|---|---|---|
| [min] | phase A [%] | phase B[%] | [mL/min] |
| 0.00 | 15 | 85 | 28 |
| 8.00 | 15 | 85 | 28 |
| 50.00 | 60 | 40 | 28 |

The structural characterization data were as follows: ESI-MS (m/z):

816.0 [M/2+H]⁺

¹H NMR (400 MHZ, DMSO-d6) δ 10.18 (s, 1H), 9.10 (s, 2H), 8.38 (t, J=5.56 Hz, 1H), 8.32 (d, J=8.40 Hz, 1H), 8.22-8.20 (m, 2H), 8.09 (t, J=5.68 Hz, 1H), 7.91-7.87 (m, 2H), 7.82-7.78 (m, 1H), 7.69 (brs, 3H), 7.61 (d, J=8.56 Hz, 2H), 7.32 (d, J=8.56 Hz, 2H), 7.06 (s, 1H), 5.56 (d, J=16.96 Hz, 1H), 5.51 (d, J=16.96 Hz, 1H), 5.47 (d, J=19.28 Hz, 1H), 5.42 (d, J=19.28 Hz, 1H), 5.14 (d, J=12.20 Hz, 1H), 5.07 (d, J=12.16 Hz, 1H), 4.48 (t, J=5.24 Hz, 2H), 4.46-4.43 (m, 1H), 4.29 (d, J=5.60 Hz, 2H), 4.08-3.95 (m, 5H), 3.79 (t, J=5.28 Hz, 2H), 3.51-3.43 (m, 32H), 3.40 (s, 3H), 3.39-3.35 (m, 2H), 3.30-3.26 (m, 2H), 3.00 (s, 3H), 2.82-2.74 (m, 2H), 2.56 (t, J=7.08 Hz, 2H), 2.29 (t, J=7.36 Hz, 2H), 2.23-2.13 (m, 2H), 1.82 (p, J=7.24 Hz, 2H), 1.78-1.63 (m, 2H), 1.61-1.49 (m, 2H), 1.42-1.27 (m, 2H), 1.15 (d, J=6.80 Hz, 3H), 1.13 (d, J=6.76 Hz, 3H), 0.90 (t, J=7.32 Hz, 3H).

### Example 6: Synthesis of (2S,3S,4S,5R,6S)-6-(4-((((2-((S)-7-ethyl-7-hydroxy-8,11-dioxo-7,8,11,13-tetrahydro-10H-[1,3]dioxolo[4,5-g]pyrano[3′,4′: 6,7]indolizino[1,2-b]quinolin-14-yl)ethyl) (isopropyl) carbamoyl)oxy)methyl)-2-(2-(2-(2-(6-(2-(methylsulfonyl)pyrimidin-5-yl) hex-5-ynamido) ethoxy) ethoxy) acetamido) phenoxy)-3,4,5-trihydroxytetrahydro-2H-pyran-2-carboxylic acid (B-03)

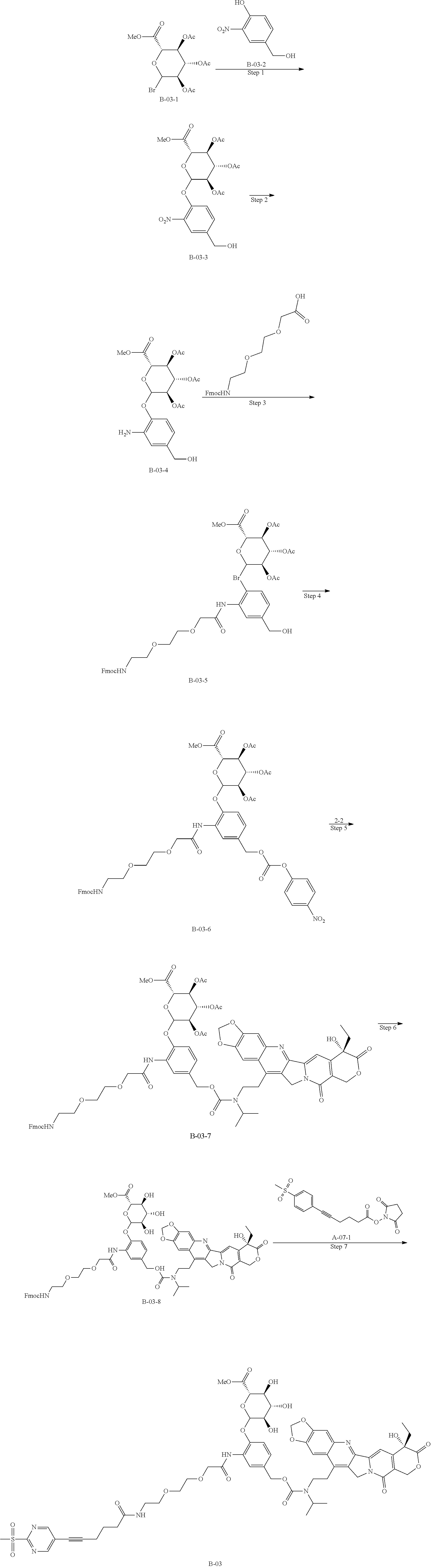

### Step 1: Synthesis of (2S,3R,4S,5S,6S)-2-(4-(hydroxymethyl)-2-nitrophenoxy)-6-(methoxycarbonyl)tetrahydro-2H-pyran-3,4,5-triyl triacetate (B-03-3)

Compounds (2R,3R,4S,5S,6S)-2-bromo-6-(methoxycarbonyl)tetrahydro-2H-pyran-3,4,5-triacetic acid triester (B-03-1, 12.32 g, 31.02 mmol) and 4-hydroxy-3-nitrobenzyl alcohol (Compound B-03-2, 5.00 g, 29.56 mmol) were dissolved in acetonitrile (200 mL), added under stirring with silver oxide (27.40 g, 118.25 mmol), subjected to nitrogen replacement and then reacted at room temperature for 12 hours in the dark. The reaction was monitored by HPLC-MS; the reaction solution was filtered through diatomite, and the filtrate was concentrated under reduced pressure and then purified by silica gel column chromatography (petroleum ether:ethyl acetate=1:3) to obtain 12.80 g of the title compound.

The structural characterization data were as follows:
ESI-MS (m/z): 503 [M+18]⁺.

### Step 2: Synthesis of (2S,3R,4S,5S,6S)-2-(2-amino-4-(hydroxymethyl) phenoxy)-6-(methoxycarbonyl)tetrahydro-2H-pyran-3,4,5-triyl triacetate (B-03-4)

Compound B-03-3 (2.20 g, 4.53 mmol) was dissolved in ethyl acetate and tetrahydrofuran (50 mL for each), added with PtO₂ (0.20 g), then the reaction system was replaced with a hydrogen balloon three times, and the reaction was carried out under hydrogen atmosphere for 2 hours. The reaction was monitored by HPLC-MS; the reaction solution was filtered directly, and the filter cake was rinsed with ethyl acetate, and the filtrate was evaporated to dryness under reduced pressure to obtain 2.02 g of a crude product of the title compound, which was directly used in the next reaction.

The structural characterization data were as follows:
ESI-MS (m/z): 456.1 [M+1]⁺.

### Step 3: Synthesis of (2S,3R,4S,5S,6S)-2-(2-(1-(9H-fluoren-9-yl)-3-oxo-2,7,10-trioxa-4-azadodecan-12-amido)-4-(hydroxymethyl)phenoxy)-6-(methoxycarbonyl)tetrahydro-2H-pyran-3,4,5-triyl triacetate (B-03-5)

Compound B-03-4 (456.00 mg, 1.00 mmol) and [2-[2-(Fmoc-amino) ethoxy]ethoxy]acetic acid (385.91 mg, 1.00 mmol) were dissolved in dichloromethane (10 mL), added under stirring with 2-ethoxy-1-ethoxycarbonyl-1,2-dihydroquinoline (495.22 mg, 2.00 mmol), and reacted under stirring for 2 hours. The reaction was monitored by HPLC-MS; the reaction solution was concentrated under reduced pressure and then purified by silica gel column chromatography (methanol:dichloromethane=1:20) to obtain 507.00 mg of the title compound.

The structural characterization data were as follows:
ESI-MS (m/z): 823.3 [M+1]⁺.

### Step 4: Synthesis of (2S,3R,4S,5S,6S)-2-(2-(1-(9H-fluoren-9-yl)-3-oxo-2,7,10-trioxa-4-azadodecan-12-amido)-4-((((4-nitrophenoxy)carbonyl)oxy)methyl) phenoxy)-6-(methoxycarbonyl)tetrahydro-2H-pyran-3,4,5-triyl triacetate (B-03-6)

Compound B-03-5 (507.00 mg, 616.18 μmol) and diisopropylethylamine (238.91 mg, 1.85 mmol) were dissolved in dichloromethane (20 mL), then p-nitrophenyl chloroformate (372.60 mg, 1.85 mmol) was dissolved in dichloromethane (1 mL) and the obtained solution was slowly dropped into the reaction solution, after the addition was completed, the reaction was carried out at room temperature for 15 h. The reaction was monitored by HPLC-MS; the reaction solution was concentrated under reduced pressure and then purified by silica gel column chromatography (methanol:dichloromethane=1:20) to obtain 496.00 mg of the title compound.

The structural characterization data were as follows:
ESI-MS (m/z): 988.5 [M+1]⁺.

### Step 5: Synthesis of (2S,3R,4S,5S,6S)-2-(2-(1-(9H-fluoren-9-yl)-3-oxo-2,7,10-trioxa-4-azadodecan-12-amido)-4-((((2-((S)-7-ethyl-7-hydroxy-8,11-dioxo-7,8,11,13-tetrahydro-10H-[1,3]dioxolo[4,5-g]pyrano[3′,4′: 6,7]indolizino[1,2-b]quinolin-14-yl)ethyl) (isopropyl) carbamoyl)oxy)methyl) phenoxy)-6-(methoxycarbonyl)tetrahydro-2H-pyran-3,4,5-triyl triacetate (B-03-7)

Compound B-03-6 (165.51 mg, 0.17 mmol), Compound 2-2 (40.00 mg, 0.084 mmol) and 1-hydroxybenzotriazole (33.96 mg, 0.25 mmol) were dissolved in DMF (4 mL), added dropwise with diisopropylethylamine (32.48 mg, 0.25 mmol), and reacted under stirring for 12 h, and the reaction was monitored by HPLC-MS. Water and ethyl acetate were added, stirred, and allowed to stand for liquid separation. The organic phase was washed with saturated brine, dried, and concentrated under reduced pressure to obtain 100.00 mg of a crude product of the title compound, which was directly used in the next reaction.

The structural characterization data were as follows:
ESI-MS (m/z): 1326.2 [M+1]⁺.

### Step 6: Synthesis of (2S,3S,4S,5R,6S)-6-(2-(2-(2-(2-aminoethoxy) ethoxy) acetamido)-4-((((2-((S)-7-ethyl-7-hydroxy-8,11-dioxo-7,8,11,13-tetrahydro-10H-[1,3]dioxolo[4,5-g]pyrano[3′,4′: 6,7]indolizino[1,2-b]quinolin-14-yl)ethyl) (isopropyl) carbamoyl)oxy)methyl) phenoxy)-3,4,5-trihydroxytetrahydro-2H-pyran-2-carboxylic acid (B-03-8)

Compound B-03-7 (100.00 mg, 0.08 mmol) was dissolved in MeOH (5 mL), added with 1 drop of dichloromethane, then added dropwise with lithium hydroxide monohydrate (15.82 mg, 0.377 mmol) in aqueous solution (1 mL), and reacted under stirring for 2 hours. The reaction was monitored by HPLC-MS; the reaction solution was added dropwise with 3N hydrochloric acid aqueous solution to adjust pH=4, concentrated under reduced pressure and then purified by preparative high-performance liquid chromatography under the following conditions, and the prepared solution was lyophilized to obtain 27.00 mg of the title compound.

Chromatographic column: SunFire Prep C18 OBD 19 mm×150 mm×5.0 μm Mobile phase A: acetonitrile; Mobile phase B: water (0.05% formic acid)

| Time | Mobile | Mobile | Flow rate |
|---|---|---|---|
| [min] | phase A [%] | phase B[%] | [mL/min] |
| 0.00 | 20 | 80 | 28 |
| 2.00 | 20 | 80 | 28 |
| 18.00 | 80 | 20 | 28 |

The structural characterization data were as follows:
ESI-MS (m/z): 964.2 [M+1]⁺.

### Step 7: Synthesis of (2S,3S,4S,5R,6S)-6-(4-((((2-((S)-7-ethyl-7-hydroxy-8,11-dioxo-7,8,11,13-tetrahydro-10H-[1,3]dioxolo[4,5-g]pyrano[3′,4′: 6,7]indolizino[1,2-b]quinolin-14-yl)ethyl) (isopropyl) carbamoyl)oxy)methyl)-2-(2-(2-(2-(6-(2-(methylsulfonyl)pyrimidin-5-yl) hex-5-ynamido) ethoxy) ethoxy) acetamido) phenoxy)-3,4,5-trihydroxytetrahydro-2H-pyran-2-carboxylic acid (B-03)

Compound B-03-8 (27.00 mg, 0.03 mmol) and 2,5-dioxopyrrolidin-1-yl 6-(2-(methylsulfonyl)pyrimidin-5-yl) hex-5-ynoate (A-07-1), 11.26 mg, 0.03 mmol) was dissolved in DMF (1 mL), and diisopropylethylamine (3.62 mg, 0.03 mmol) was added dropwise under stirring, and reacted at room temperature for 4 hours. The reaction was monitored by chromatography coupled with mass spectrometry. The reaction solution was purified by preparative high performance liquid chromatography (conditions are as follows), and the preparative solution was freeze-dried to obtain 11.70 mg of the title compound.

Chromatographic column: SunFire Prep C18 OBD 19 mm×150 mm×5.0 μm

Mobile phase A: acetonitrile; Mobile phase B: water (0.05% formic acid)

| Time | Mobile | Mobile | Flow rate |
|---|---|---|---|
| [min] | phase A [%] | phase B[%] | [mL/min] |
| 0.00 | 20 | 80 | 28 |
| 2.00 | 20 | 80 | 28 |
| 18.00 | 80 | 20 | 28 |

The structural characterization data were as follows:
ESI-MS (m/z): 1214.4 [M+1]⁺.

### Example 7: Synthesis of N-((1S,9S)-4-chloro-9-ethyl-5-fluoro-9-hydroxy-10,13-dioxo-2,3,9,10,13,15-hexahydro-1H, 12H-benzo[de]pyrano[3′,4′: 6,7]indolizino[1,2-b]quinolin-1-yl)-2-hydroxyacetamide and N-((1R,9S)-4-chloro-9-ethyl-5-fluoro-9-hydroxy-10,13-dioxo-2,3,9,10,13,15-hexahydro-1H,12H-benzo[de]pyrano[3′,4′: 6,7]indolizino[1,2-b]quinolin-1-yl)-2-hydroxyacetamide (1-11-A and 1-11-B)

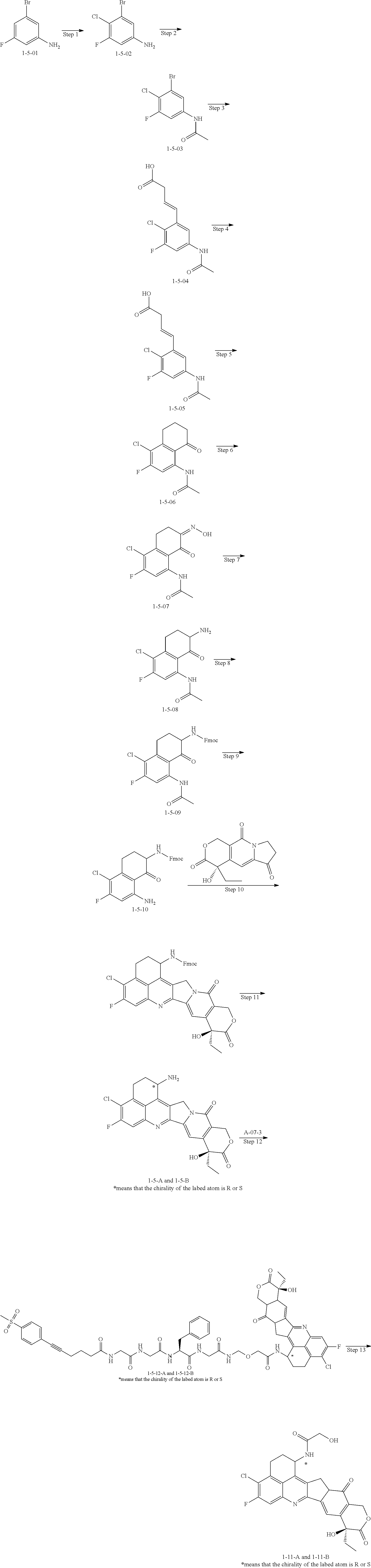

### Step 1: Synthesis of 3-bromo-4-chloro-5-fluoroaniline (1-5-02)

Compound 1-5-01 (2.00 g, 10.53 mmol) was dissolved in N,N-dimethylformamide (30 mL), then slowly added with N-chlorosuccinimide (1.69 g, 12.63 mmol), after the addition, the reaction was carried out at room temperature for 16 hours, and the reaction was monitored by HPLC-MS. The reaction solution was concentrated under reduced pressure to obtain a crude product, which was purified by flash silica gel column (ethyl acetate:petroleum ether=0-25%) to obtain 0.95 g of the title compound.

The structural characterization data were as follows:

¹H-NMR (400 MHZ, DMSO-d6) δ 6.77 (dd, J=2.5, 1.4 Hz, 1H), 6.51 (dd, J=11.7, 2.5 Hz, 1H), 5.84 (s, 2H).

### Step 2: Synthesis of N-(3-bromo-4-chloro-5-fluorophenyl) acetamide (1-5-03)

Compound 1-5-02 (0.95 g, 4.23 mmol) was dissolved in ethyl acetate (20 mL), added with acetic anhydride (648.13 mg, 6.35 mmol) under nitrogen protection; after the addition, the temperature was raised to 50° C., the reaction was carried out for 15 hours, and the reaction was monitored by HPLC-MS. The reaction solution was quenched with methanol (5 mL), then directly dried by reduced-pressure evaporation to obtain a crude product, and the crude product was purified by flash silica gel column (ethyl acetate:petroleum ether=0-40%) to obtain 1.01 g of the title compound.

The structural characterization data were as follows:
ESI-MS (m/z): 265.9 [M+H]⁺.

### Step 3: Synthesis of (E)-4-(5-acetamido-2-chloro-3-fluorophenyl) but-3-enoic acid (1-5-04)

Compound 1-5-03 and 3-butenoic acid (387.65 mg, 4.50 mmol) were dissolved in a mixed solvent of 1,4-dioxane (24 mL) and water (8 mL), then added with N,N-diisopropylethylamine (1.45 g, 11.26 mmol), tris(o-methylphenyl)phosphine (114.21 mg, 375.24 μmol) and palladium acetate (42.12 mg, 187.62 μmol), after the addition, the reaction system was subjected to nitrogen replacement three times, the temperature was raised to 100° C. under nitrogen atmosphere, and the reaction was carried out for 16 hours, and the reaction was monitored by HPLC-MS. The reaction solution was cooled to room temperature, then added with 1N sodium hydroxide aqueous solution (60 mL) and ethyl acetate (50 mL) and shaken for layering. The lower aqueous phase was separated and subjected to pH adjustment to about 3 with 4 mol/L hydrochloric acid aqueous solution, extraction was performed with ethyl acetate, the organic phases were combined, washed with saturated brine, dried over anhydrous sodium sulfate, filtered, and the filtrate was evaporated to dryness under reduced pressure to obtain 1.00 g of a crude product of the title compound.

The structural characterization data were as follows:
ESI-MS (m/z): 272.0 [M+H]⁺.

### Step 4: Synthesis of 4-(5-acetamido-2-chloro-3-fluorophenyl) butanoic acid (1-5-05)

The crude product of Compound 1-5-04 (1.00 g, 3.68 mmol) was dissolved in tetrahydrofuran (15 mL), then added with 10% palladium on carbon (0.10 g), after the addition, the atmosphere of the reaction system was replaced by a hydrogen balloon three times, the reaction was carried out under hydrogen atmosphere for 4 hours, and the reaction was monitored by HPLC-MS. The reaction solution was filtered, and the filtrate was concentrated to dryness under reduced pressure to obtain 1.00 g of a crude product of the title compound.

The structural characterization data were as follows:
ESI-MS (m/z): 274.0 [M+H]⁺.

### Step 5: Synthesis of N-(4-chloro-3-fluoro-8-oxo-5,6,7,8-tetrahydronaphthalen-1-yl) acetamide (1-5-06)

The crude product of Compound 1-5-05 (1.00 g, 3.65 mmol) was dissolved in trifluoroacetic acid (5 mL), cooled to 5° C., then added slowly with trifluoroacetic anhydride (3.84 g, 18.27 mmol, 2.54 mL), after the addition, the reaction was conducted at 5° C. for 2 hours, and the reaction was monitored by HPLC-MS. The reaction solution was slowly poured into water, then extracted with ethyl acetate, the organic phases were combined, washed with saturated brine, dried over anhydrous sodium sulfate, and then filtered, the filtrate was dried by reduced-pressure evaporation to obtain a crude product, and the crude product was purified by flash silica gel column to obtain 0.43 g of the title compound.

The structural characterization data were as follows:
ESI-MS (m/z): 256.1 [M+H]⁺.

### Step 6: Synthesis of N-(4-chloro-3-fluoro-7-(hydroxyimino)-8-oxo-5,6,7,8-tetrahydronaphth-1-yl) acetamide (1-5-07)

Added tetrahydrofuran (16 mL) and tert-butanol (4 mL) into the reaction flask, cooled to 5° C. in an ice bath, then added with potassium tert-butoxide (415.18 mg, 3.70 mmol), Compound 1-5-06 (0.43 mg, 1.68 mmol) was dissolved in tetrahydrofuran (1 mL), and slowly added dropwise to the reaction solution, after 10 minutes, isoamyl nitrite (315.24 mg, 2.69 mmol) was added, after the addition, the reaction was carried out at 5° C. for 1 hour, and the reaction was monitored by HPLC-MS. The reaction solution was quenched with saturated ammonium chloride aqueous solution, extracted with ethyl acetate, the organic phases were combined, washed with saturated brine, dried over anhydrous sodium sulfate, then filtered, and the filtrate was concentrated under reduced pressure to obtain 455.00 mg of a crude product of the title compound.

The structural characterization data were as follows:
ESI-MS (m/z): 285.0 [M+H]⁺.

### Step 7: Synthesis of N-(7-amino-4-chloro-3-fluoro-8-oxo-5,6,7,8-tetrahydronaphthalen-1-yl) acetamide (1-5-08)

The crude product of Compound 1-5-07 (0.40 g, 1.41 mmol) was dissolved in methanol (10 mL), then added with 3 mol/L aqueous hydrochloric acid (1 mL) and 10% palladium on carbon (40.00 mg), after the addition, the reaction system was subjected to hydrogen replacement three times, the reaction was carried out at room temperature under hydrogen atmosphere for 1 hour, and the reaction was monitored by HPLC-MS. The reaction solution was filtered, and the filtrate was concentrated to dryness under reduced pressure to obtain 0.43 g of a crude hydrochloride of the title compound.

The structural characterization data were as follows:
ESI-MS (m/z): 271.0 [M+H]⁺.

### Step 8: Synthesis of (9H-fluoren-9-yl)methyl) (8-acetamide-5-chloro-6-fluoro-1-oxo-1,2,3,4-tetrahydronaphthalen-2-yl) carbamate (1-5-09)

The crude hydrochloride of Compound 1-5-08 (0.43 g, 1.19 mmol) was dissolved in 1,4-dioxane (15 mL), then added with sodium bicarbonate (400.35 mg, 4.77 mmol), water (5 mL) and 9-fluorenylmethyl-N-succinimidyl carbonate (481.81 mg, 1.43 mmol); after the addition, the reaction was carried out under stirring at room temperature for 2 hours, and the reaction was monitored by HPLC-MS. The reaction solution was poured into water, then extracted with ethyl acetate, the organic phases were combined, washed with saturated brine, dried over anhydrous sodium sulfate, filtered, and the filtrate was concentrated under reduced pressure to obtain a crude product. The crude product was purified by C18 reverse-phase column (acetonitrile: 0.05% formic acid in water=20%-100%) to obtain 301.00 mg of the title compound.

The structural characterization data were as follows:
ESI-MS (m/z): 493.2 [M+H]⁺.

### Step 9: Synthesis of (9H-fluoren-9-yl)methyl) (8-amino-5-chloro-6-fluoro-1-oxo-1,2,3,4-tetrahydronaphthalen-2-yl) carbamate (1-5-10)

Compound 1-5-09 (300.00 mg, 608.61 μmol) was dissolved in dioxane (5 mL), added with 12 mol/L concentrated hydrochloric acid (1 mL); after the addition, the temperature was raised to 60° C. and the reaction was carried out for 2 hours, and the reaction was monitored by HPLC-MS. The reaction solution was poured into water, then extracted with ethyl acetate, the organic phases were combined, washed with saturated brine, dried over anhydrous sodium sulfate, filtered, and the filtrate was concentrated under reduced pressure to obtain a crude product. The crude product was purified by flash silica gel column (ethyl acetate:petroleum ether=0-50%) to obtain 198.00 mg of the title compound.

The structural characterization data were as follows:
ESI-MS (m/z): 451.1 [M+H]⁺.

### Step 10: Synthesis of (9H-fluoren-9-yl) methyl ((9S)-4-chloro-9-ethyl-5-fluoro-9-hydroxy-10,13-dioxo-2,3,9,10,13,15-hexahydro-1H,12H-benzo[de]pyrano[3′,4′: 6,7]indolizino[1,2-b]quinolin-1-yl) carbamate (1-5-11)

(S)-4-Ethyl-4-hydroxy-7,8-dihydro-1H-pyrano[3,4-f]indolezine-3,6,10 (4H)-trione (138.72 mg, 526.96 μmol) and Compound 1-5-10 (198.00 mg, 439.13 μmol) were added in toluene (10 mL), then added with p-toluenesulfonic acid (75.53 mg, 439.13 μmol); after the addition, the temperature was raised to 140° C. and the reaction was carried out for 4 hours, the reaction solution was directly dried by reduced-pressure evaporation at 140° C. to obtain a crude product, and the crude product was purified by flash silica gel column (methanol:dichloromethane=0-5%) to obtain 256.00 mg of the title compound.

The structural characterization data were as follows:
ESI-MS (m/z): 678.1 [M+H]⁺.

### Step 11: Synthesis of (1S,9S)-1-amino-4-chloro-9-ethyl-5-fluoro-9-hydroxy-1,2,3,9,12,15-hexahydro-10H,13H-benzo[de]pyrano[3′,4′: 6,7]indolizino[1,2-b]quinoline-10,13-dione and (1R,9S)-1-amino-4-chloro-9-ethyl-5-fluoro-9-hydroxy-1,2,3,9,12,15-hexahydro-10H, 13H-benzo[de]pyrano[3′,4′: 6,7]indolazino[1,2-b]quinoline-10,13-dione (1-5-A and 1-5-B)

Compound 1-5-11 (201.18 mg, 296.67 μmol) was dissolved in N,N-dimethylformamide (4 mL), then added with diethylamine (108.49 mg, 1.48 mmol); after the addition, the reaction was carried out at room temperature for 0.5 h, and the reaction was monitored by HPLC-MS. The reaction solution was evaporated under reduced pressure to remove ethylenediamine, 1 mol/L hydrochloric acid aqueous solution was used to adjust pH to 2-3, and the reaction solution was directly purified by preparative high performance liquid chromatography to obtain the title compounds, 1-5-A (44.00 mg) and 1-5-B (43.00 mg).

Chromatographic column: SunFire Prep C18 OBD 19 mm×150 mm×5.0 μm

Mobile phase A: acetonitrile; Mobile phase B: water (0.05% formic acid)

| Time | Mobile | Mobile | Flow rate |
|---|---|---|---|
| [min] | phase A [%] | phase B[%] | [mL/min] |
| 0 | 10 | 90 | 28 |
| 3 | 10 | 90 | 28 |
| 18 | 70 | 30 | 28 |

1-5-A (6 min LCMS peak appeared early, retention time: 1.276 min) The structural characterization data were as follows:

¹H-NMR (400 MHZ, DMSO-d6) δ 8.00 (d, J=10.3 Hz, 1H), 7.33 (s, 1H), 6.54 (s, 1H), 5.62 (d, J=19.3 Hz, 1H), 5.44 (s, 2H), 5.38 (d, J=19.3 Hz, 1H), 4.43-4.38 (m, 1H), 3.28-3.10 (m, 2H), 2.22-2.12 (m, 1H), 2.12-2.02 (m, 1H), 1.93-1.80 (m, 2H), 0.87 (t, J=7.3 Hz, 3H).

ESI-MS (m/z): 456.1 [M+H]⁺.

The structural characterization data of 1-5-B (6 min LCMS peak appeared later, retention time: 1.300 min) were as follows:

¹H-NMR (400 MHz, DMSO-d6) δ7.98 (d, J=10.3 Hz, 1H), 7.32 (s, 1H), 5.61 (d, J=19.4 Hz, 1H), 5.44 (s, 2H), 5.32 (d, J=19.4 Hz, 1H), 4.44-4.36 (m, 1H), 3.33-3.25 (m, 1H), 3.22-3.11 (m, 1H), 2.23-2.13 (m, 1H), 2.11-2.03 (m, 1H), 1.96-1.82 (m, 2H), 0.89 (t, J=7.3 Hz, 3H).

ESI-MS (m/z): 456.1 [M+H]⁺.

### 6 Min LCMS Conditions:

Chromatographic column: Waters SunFire C18 OBD 4.6 mm×50 mm×5.0 μm

Mobile phase A: 0.05% acetonitrile; Mobile phase B: water (0.05% formic acid)

| Time | Mobile | Mobile | Flow rate |
|---|---|---|---|
| [min] | phase A [%] | phase B[%] | [mL/min] |
| 0 | 90 | 10 | 2 |
| 4.2 | 10 | 90 | 2 |
| 5.7 | 10 | 90 | 2 |
| 5.71 | 90 | 10 | 2 |
| 6.70 | 90 | 10 | 2 |

### Step 12: Synthesis of N—((S)-10-benzyl-1-(((1S,9S)-4-chloro-9-ethyl-5-fluoro-9-hydroxy-10,13-dioxo-2,3,9,10,13,15-hexahydro-1H,12H-benzo[de]pyrano[3′,4′: 6,7]indolizino[1,2-b]quinolin-1-yl)amino)-1,6,9,12,15-pentaoxo-3-oxa-5,8,11,14-tetraazahexadecan-16-yl)-6-(2-(methylsulfonyl)pyrimidin-5-yl) hex-5-ynamide and N—((S)-10-benzyl-1-(((1R,9S)-4-chloro-9-ethyl-5-fluoro-9-hydroxy-10,13-dioxo-2,3,9,10,13,15-hexahydro-1H,12H-benzo[de]pyrano[3′,4′: 6,7]indolizino[1,2-b]quinolin-1-yl)amino)-1,6,9,12,15-pentaoxo-3-oxa-5,8,11,14-tetraazahexadecan-16-yl)-6-(2-(methylsulfonyl)pyrimidin-5-yl) hex-5-ynamide (1-5-12-A and Jan. 5, 2012-B)

Compound 1-5-A in single configuration (36.00 mg, 79.70 μmol) and Compound A-07-3 (64.43 mg, 95.64 μmol) were dissolved in N,N-dimethylformamide (2 mL), and then added with 4-(4,6-dimethoxytriazin-2-yl)-4-methylmorpholine hydrochloride (46.98 mg, 159.40 μmol) and triethylamine (24.19 mg, 239.10 μmol), after the addition, the reaction was carried out at room temperature for 1 hour, and the reaction was monitored by HPLC-MS. The reaction solution was directly purified by high performance liquid chromatography to obtain the title compound Jan. 5, 2012-A in single configuration (51.00 mg).

Chromatographic column: SunFire Prep C18 OBD 19 mm×150 mm×5.0 μm

Mobile phase A: acetonitrile; Mobile phase B: water (0.05% formic acid)

| Time | Mobile | Mobile | Flow rate |
|---|---|---|---|
| [min] | phase A [%] | phase B[%] | [mL/min] |
| 0 | 30 | 70 | 28 |
| 3 | 30 | 70 | 28 |
| 18 | 90 | 10 | 28 |

The structural characterization data were as follows:
ESI-MS (m/z): 1111.0 [M+H]⁺.

Compound 1-5-B in single configuration (36.00 mg, 79.70 μmol) and Compound A-07-3 (64.43 mg, 95.64 μmol) were dissolved in N,N-dimethylformamide (2 mL), then added with 4-(4,6-dimethoxytriazin-2-yl)-4-methylmorpholine hydrochloride (46.98 mg, 159.40 μmol) and triethylamine (24.19 mg, 239.10 μmol), after the addition, the reaction was carried out at room temperature for 1 hour, and the reaction was monitored by HPLC-MS. The reaction solution was directly purified by high performance liquid chromatography to obtain the title compound 1-5-12-B in single configuration (52.00 mg).

Chromatographic column: SunFire Prep C18 OBD 19 mm×150 mm×5.0 μm

Mobile phase A: acetonitrile; Mobile phase B: water (0.05% formic acid)

| Time | Mobile | Mobile | Flow rate |
|---|---|---|---|
| [min] | phase A [%] | phase B[%] | [mL/min] |
| 0 | 30 | 70 | 28 |
| 3 | 30 | 70 | 28 |
| 18 | 90 | 10 | 28 |

The structural characterization data were as follows:
ESI-MS (m/z): 1111.0 [M+H]⁺.

### Step 13: Synthesis of N-((1S,9S)-4-chloro-9-ethyl-5-fluoro-9-hydroxy-10,13-dioxo-2,3,9,10,13,15-hexahydro-1H,12H-benzo[de]pyrano[3′,4:6,7]indolizino[1,2-b]quinolin-1-yl)-2-hydroxyacetamide and N-((1R,9S)-4-chloro-9-ethyl-5-fluoro-9-hydroxy-10,13-dioxo-2,3,9,10,13,15-hexahydro-1H,12H-benzo[de]pyrano[3′,4:6,7]indolizino[1,2-b]quinolin-1-yl)-2-hydroxyacetamide (1-11-A and 1-11-B)

Compound 1-5-12-A (40.00 mg, 35.99 μmol) was weighed and dissolved in a mixed solvent of dichloromethane (2 mL) and methanol (1 mL), and then added with 4 mol/L hydrochloric acid in ethyl acetate (1 mL), after the addition, the reaction was carried out at room temperature for 0.5 hours, and the reaction was monitored by HPLC-MS. The reaction solution was directly concentrated to dryness under reduced pressure to obtain a crude product, and the crude product was purified by high performance liquid chromatography to obtain the title compound 1-11-A in single configuration (4.75 mg).

Chromatographic column: SunFire Prep C18 OBD 19 mm×150 mm×5.0 μm

Mobile phase A: acetonitrile; Mobile phase B: water (0.05% formic acid)

| Time | Mobile | Mobile | Flow rate |
|---|---|---|---|
| [min] | phase A [%] | phase B[%] | [mL/min] |
| 0 | 15 | 85 | 28 |
| 3 | 15 | 85 | 28 |
| 18 | 90 | 10 | 28 |

The structural characterization data were as follows:

¹H-NMR (400 MHZ, DMSO-d6) δ 8.50 (d, J=8.9 Hz, 1H), 8.05 (d, J=10.3 Hz, 1H), 7.33 (s, 1H), 6.55 (s, 1H), 5.67-5.60 (m, 1H), 5.49 (t, J=5.8 Hz, 1H), 5.43 (s, 2H), 5.21 (s, 2H), 3.96 (d, J=5.8 Hz, 2H), 3.32-3.22 (m, 2H), 2.28-2.15 (m, 2H), 1.93-1.80 (m, 2H), 0.87 (t, J=7.3 Hz, 3H).

ESI-MS (m/z): 514.0 [M+H]⁺.

Compound 1-5-12-B (40.00 mg, 35.99 μmol) was weighed and dissolved in a mixed solvent of dichloromethane (2 mL) and methanol (1 mL), then added with 4 mol/L hydrochloric acid in ethyl acetate (1 mL), after the addition, the reaction was carried out at room temperature for 0.5 hours, and the reaction was monitored by HPLC-MS. The reaction solution was directly concentrated to dryness under reduced pressure to obtain a crude product, and the crude product was purified by high performance liquid chromatography to obtain the title compound 1-11-B in single configuration (8.24 mg).

Chromatographic column: SunFire Prep C18 OBD 19 mm×150 mm×5.0 μm

Mobile phase A: acetonitrile; Mobile phase B: water (0.05% formic acid)

| Time | Mobile | Mobile | Flow rate |
|---|---|---|---|
| [min] | phase A [%] | phase B[%] | [mL/min] |
| 0 | 15 | 85 | 28 |
| 3 | 15 | 85 | 28 |
| 18 | 90 | 10 | 28 |

The structural characterization data were as follows:

¹H-NMR (400 MHZ, DMSO-d6) δ 8.52 (d, J=9.0 Hz, 1H), 8.05 (d, J=10.3 Hz, 1H), 7.34 (s, 1H), 6.55 (s, 1H), 5.68-5.58 (m, 1H), 5.53 (t, J=5.8 Hz, 1H), 5.43 (d, J=2.9 Hz, 2H), 5.20 (d, J=7.3 Hz, 2H), 3.97 (d, J=5.7 Hz, 2H), 3.31-3.21 (m, 2H), 2.26-2.15 (m, 2H), 1.92-1.82 (m, 2H), 0.87 (t, J=7.3 Hz, 3H).

ESI-MS (m/z): 514.0 [M+H]⁺.

### Example 8: Synthesis of N-((10S,19S)-10-benzyl-1-(((1R,9S)-4-chloro-9-ethyl-5-fluoro-9-hydroxy-10,13-dioxo-2,3,9,10,13,15-hexahydro-1H,12H-benzo[de]pyrano[3′,4′: 6,7]indolizino[1,2-b]quinolin-1-yl)amino)-30-(2-(methylsulfonyl)pyrimidin-5-yl)-1,6,9,12,15,18,25-heptaoxo-3-oxa-5,8,11,14,17,24-hexaazatriacont-29-yn-19-yl)-2,5,8,11,14,17,20,23,26,29,32,35-dodecaoxaoctatriacontan-38-amideor N-((10S,19S)-10-benzyl-1-(((1S,9S)-4-chloro-9-ethyl-5-fluoro-9-hydroxy-10,13-dioxo-2,3,9,10,13,15-hexahydro-1H,12H-benzo[de]pyrano[3′,4′: 6,7]indolizino[1,2-b]quinolin-1-yl)amino)-30-(2-(methylsulfonyl)pyrimidin-5-yl)-1,6,9,12,15,18,25-heptaoxo-3-oxa-5,8,11,14,17,24-hexaazatriacont-29-yn-19-yl)-2,5,8,11,14,17,20,23,26,29,32,35-dodecaoxaoctatriacontan-38-amide (A-17-A)

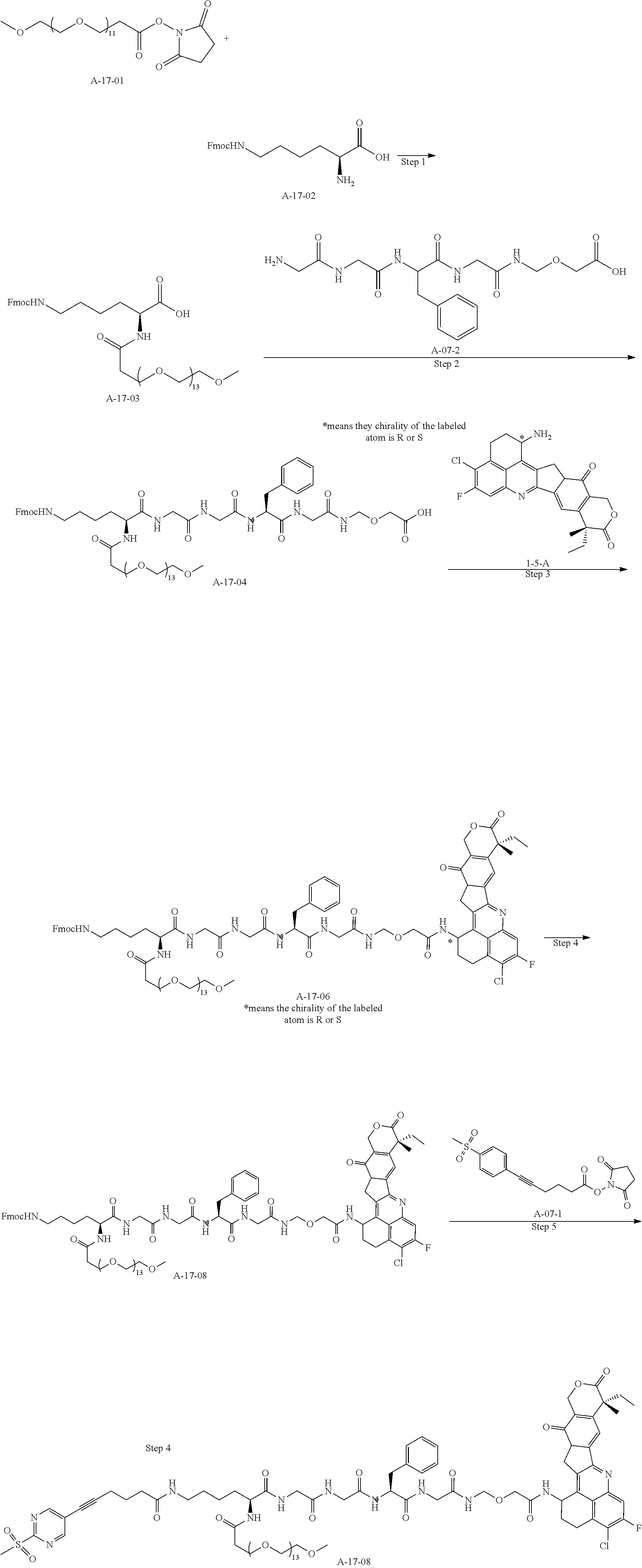

### Step 1: Synthesis of N⁶-(((9H-fluoren-9-yl) methoxy) carbonyl)-N2-(2,5,8,11,14,17,20,23,26,29,32,35-dodecaoxaoctatriacontan-38-oyl)-D-lysine (2S)-2-(2,5,8,11,14,17,20,23,26,29,32,35-dodeoxaoctadecane-38-amido)-6-({[(9H-fluoren-9-yl) methoxy]carbonyl}amino) hexanoic acid (A-17-03)

Hydrochloride salt of Compound A-17-02 (389.68 mg, 962.45 μmol) was dissolved in dichloromethane (8 mL), added with DIPEA (518.28 mg, 4.01 mmol, 713.88 μL) and Compound A-17-01 (550.00 mg, 802.04 μmol), and reacted at 25° C. for 1.5 hours. The pH of the reaction solution was adjusted to neutral with dilute hydrochloric acid, and the solvent was dried under reduced pressure. The concentrate was purified by preparative high performance liquid chromatography to obtain the title compound A-17-03 (450.00 mg).

Chromatographic column: SunFire Prep C18 OBD 19 mm×150 mm×5.0 μm

Mobile phase A: acetonitrile; Mobile phase B: water (0.05% formic acid)

| Time | Mobile | Mobile | Flow rate |
|---|---|---|---|
| [min] | phase A [%] | phase B[%] | [mL/min] |
| 0 | 10 | 90 | 28 |
| 2 | 10 | 90 | 28 |
| 18 | 80 | 20 | 28 |

ESI-MS (m/z): 939.3 [M+H]⁺.

### Step 2: Synthesis of (40S,49S)-40-(4-((((9H-fluoren-9-yl) methoxy) carbonyl)amino)butyl)-49-benzyl-38,41,44,47,50,53-hexaoxo-2,5,8,11,14,17,20,23,26,29,32,35,56-tridecaoxa-39,42,45,48,51,54-hexaazaoctapentacontan-58-oic acid (A-17-04)

Compound A-17-03 (50.00 mg, 118.09 μmol) was dissolved in DMF (2.5 mL), added with HATU (49.39 mg, 129.89 μmol), Compound A-07-2 (133.07 mg, 141.70 μmol) and DIPEA (45.78 mg, 354.26 μmol, 63.06 μL), and reacted at 25° C. for 1 hour. The solvent was dried under reduced pressure, and the concentrate was purified by preparative high performance liquid chromatography to obtain the title compound A-17-04 (40.00 mg).

Chromatographic column: SunFire Prep C18 OBD 19 mm×150 mm×5.0 μm

Mobile phase A: acetonitrile; Mobile phase B: water (0.05% formic acid)

| Time | Mobile | Mobile | Flow rate |
|---|---|---|---|
| [min] | phase A [%] | phase B[%] | [mL/min] |
| 0 | 10 | 90 | 28 |
| 2 | 10 | 90 | 28 |
| 18 | 80 | 20 | 28 |

ESI-MS (m/z): 1344.4 [M+H]⁺.

### Step 3: Synthesis of (9H-fluoren-9-yl) methyl ((40S)-40-(((10S)-10-benzyl-1-(((9S)-4-chloro-9-ethyl-5-fluoro-9-hydroxy-10,13-dioxo-2,3,9,10,13,15-hexahydro-1H,12H-benzo[de]pyrano[3′,4′: 6,7]indolizino[1,2-b]quinolin-1-yl)amino)-1,6,9,12,15-pentaoxo-3-oxa-5,8,11,14-tetraazahexadecan-16-yl) carbamoyl)-38-oxo-2,5,8,11,14,17,20,23,26,29,32,35-dodecaoxa-39-azatetratetracontan-44-yl) carbamate (A-17-05)

Compound A-17-04 (29.86 mg, 59.50 μmol) was dissolved in DMF (3 mL), added with HATU (27.15 mg, 71.40 μmol), Compound 1-5-A (80.00 mg, 59.50 μmol) and DIPEA (38.45 mg, 297.51 μmol, 52.96 μL), and reacted at 25° C. for 1 hour. The solvent was dried under reduced pressure, and the concentrate was purified by preparative high performance liquid chromatography to obtain the title compound A-17-05 (50.00 mg).

Chromatographic column: SunFire Prep C18 OBD 19 mm×150 mm×5.0 μm

Mobile phase A: acetonitrile; Mobile phase B: water (0.05% formic acid)

| Time | Mobile | Mobile | Flow rate |
|---|---|---|---|
| [min] | phase A [%] | phase B[%] | [mL/min] |
| 0 | 10 | 90 | 28 |
| 2 | 10 | 90 | 28 |
| 18 | 80 | 20 | 28 |

ESI-MS (m/z): 1781.6 [M+H]⁺.

### Step 4: Synthesis of N-((10S,19S)-23-amino-10-benzyl-1-(((9S)-4-chloro-9-ethyl-5-fluoro-9-hydroxy-10,13-dioxo-2,3,9,10,13,15-hexahydro-1H,12H-benzo[de]pyrano[3′,4′: 6,7]indolizino[1,2-b]quinolin-1-yl)amino)-1,6,9,12,15,18-hexaoxo-3-oxa-5,8,11,14,17-pentaazatricosan-19-yl)-2,5,8,11,14,17,20,23,26,29,32,35-dodecaoxaoctatriacontan-38-amide N-((10S,19S)-23-amino-10-benzyl-1-(((9S)-4-chloro-9-ethyl-5-fluoro-9-hydroxy-10,13-dioxo-2,3,9,10,13,15-Hexahydro-1H,12H-benzo[de]pyrano[3′,4]: 6,7]indoleazino[1,2-b]Quinolin-1-yl)amino)-1,6,9,12,15,18-hexaoxo-3-oxo-5,8,11,14,17-pentaaza230 Alk-19-yl)-2,5,8,11,14,17,20,23,26,29,32,35-Dodecyloxatrioctadecane-38-amide (A-17-06)

Compound A-17-05 (20.00 mg, 11.22 μmol) was dissolved in DMF (2.5 mL) and diethylamine (0.5 mL), and reacted at 25° C. for 2 hours. The solvent was dried under reduced pressure, and the concentrate was purified by preparative high performance liquid chromatography to obtain a formate of the title compound A-17-06 (10.00 mg).

Chromatographic column: SunFire Prep C18 OBD 19 mm×150 mm×5.0 μm

Mobile phase A: acetonitrile; Mobile phase B: water (0.05% formic acid)

| | Mobile | Mobile | Flow |
|---|---|---|---|
| Time [min] | phase A [%] | phase B[%] | rate [mL/min] |
| 0 | 15 | 85 | 28 |
| 2 | 15 | 85 | 28 |
| 18 | 80 | 20 | 28 |

ESI-MS (m/z): 1559.7 [M+H]⁺.

### Step 5: Synthesis of N-((10S,19S)-10-benzyl-1-(((1R,9S)-4-chloro-9-ethyl-5-fluoro-9-hydroxy-10,13-dioxo-2,3,9,10,13,15-hexahydro-1H,12H-benzo[de]pyrano[3′,4′: 6,7]indolizino[1,2-b]quinolin-1-yl)amino)-30-(2-(methylsulfonyl)pyrimidin-5-yl)-1,6,9,12,15,18,25-heptaoxo-3-oxa-5,8,11,14,17,24-hexaazatriacont-29-yn-19-yl)-2,5,8,11,14,17,20,23,26,29,32,35-dodecaoxaoctatriacontan-38-amideor N-((10S,19S)-10-benzyl-1-(((1S,9S)-4-chloro-9-ethyl-5-fluoro-9-hydroxy-10,13-dioxo-2,3,9,10,13,15-hexahydro-1H,12H-benzo[de]pyrano[3′,4′: 6,7]indolizino[1,2-b]quinolin-1-yl)amino)-30-(2-(methylsulfonyl)pyrimidin-5-yl)-1,6,9,12,15,18,25-heptaoxo-3-oxa-5,8,11,14,17,24-hexaazatriacont-29-yn-19-yl)-2,5,8,11,14,17,20,23,26,29,32,35-dodecaoxaoctatriacontan-38-amide (A-17-A)

The formate of Compound A-17-06 (7.00 mg, 4.49 μmol) and Compound A-07-1 (3.28 mg, 8.97 μmol) were dissolved in DMF (1 mL), added with DIPEA (1.74 mg, 13.46 μmol, 2.40 μL), and reacted at 25° C. for 2 hours. The solvent was dried under reduced pressure, and the concentrate was purified by preparative high performance liquid chromatography to obtain the title compound A-17-A (5.60 mg).

Chromatographic column: SunFire Prep C18 OBD 19 mm×150 mm×5.0 μm

Mobile phase A: acetonitrile; Mobile phase B: water (0.05% formic acid)

| | Mobile | Mobile | Flow |
|---|---|---|---|
| Time [min] | phase A [%] | phase B[%] | rate [mL/min] |
| 0 | 20 | 80 | 28 |
| 2 | 20 | 80 | 28 |
| 18 | 80 | 20 | 28 |

ESI-MS (m/z): 1809.8 [M+H]⁺.

### Example 9: Synthesis of N—((S)-10-benzyl-1-(((1S,9S)-5-chloro-9-ethyl-9-hydroxyl-4-methyl-10,13-dioxo-2,3,9,10,13,15-Hexahydro-1H,12H-benzo[de]pyran[3′,4′: 6,7]indolizine[1,2-b]quinoneLin-1-yl)amino)-1,6,9,12,15-pentoxo-3-oxyl-5,8,11,14-tetraazahexadecan-16-yl)-6-(2-(Methylsulfo)pyrimidin-5-yl) hexan-5-amide (A-05)

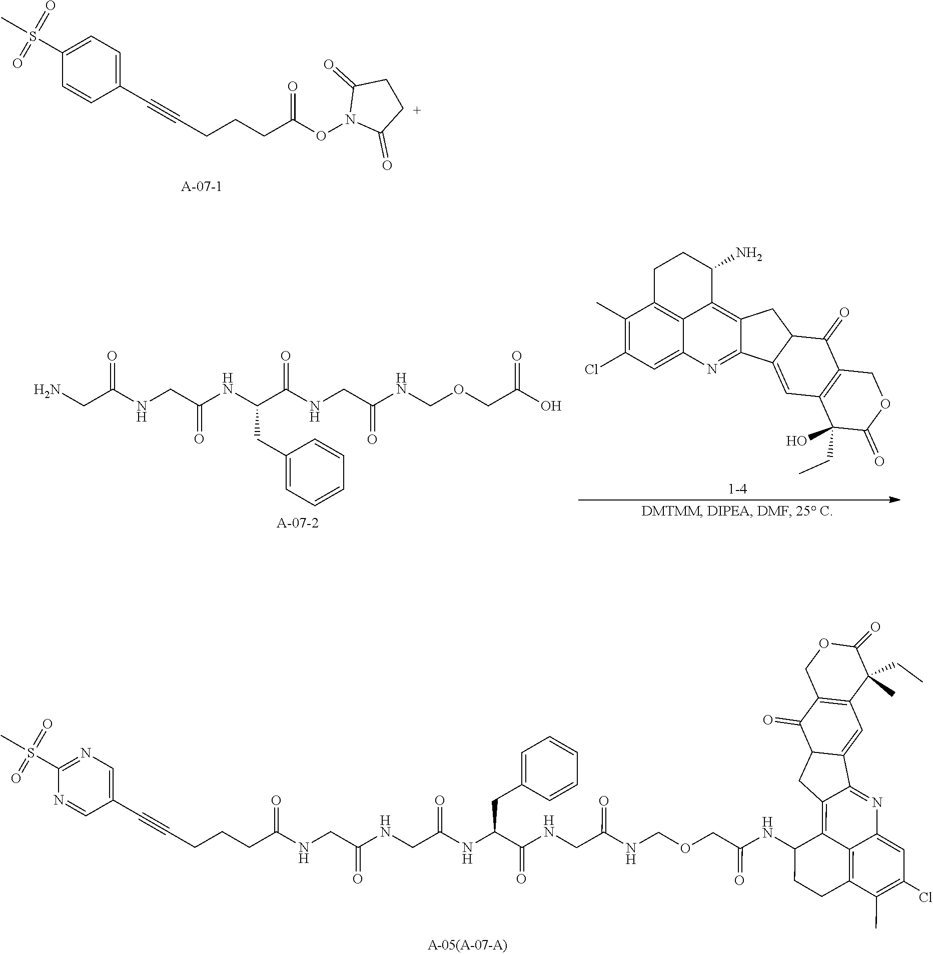

Under nitrogen protection, 2,5-dioxypyrrolidin-1-yl-6-(2-(methylsulfonyl)pyrimidin-5-yl) hexyl-5-ynoate (A-07-1, 0.66 g, 1.80 mmol) and A-07-2 (0.75 g, 1.77 mmol) were added to DMF (19 mL), heated to 35° C. for 16 hours, and then (1S,9S)-1-amino-5-chloro-9-ethyl-9-hydroxy-4-methyl-1,2,3,9,12,15-hexahydro-10H,13H-benzo[de]pyrano[3′,4′: 6,7]indolizino[1,2-b]quinoline-10,13-dione (1-4 (i.e. 1-2-A), 1.00 g, 1.77 mmol) was added and the mixture was cooled to 5˜15° C. in ice water. DMTMM (0.98 g, 3.53 mmol) was added, and DIPEA (1.14 g, 8.84 mmol) was added dropwise, and allowed to react at 25° C. for 16 hours. The reaction solution was poured into DCM (600 mL), IPA (60 mL), water (100 mL) and the mixture was stirred for 10 minutes, then the DCM phase was separated out and washed with brine (100 ml), concentrated to obtain crude product, and was purified by preparative high performance liquid chromatography. After purification, the product was lyophilized yielding 0.98 g of compound A-05 (that is, A-07-A in Example 3).

A-05 separation and purification method is as follows:

Column: Waters SunFire Prep C18 OBD (5 μm*19 mm*150 mm)

Mobile Phase A: Acetonitrile; Mobile Phase B: Water (0.05% Formic Acid)

| | Mobile | Mobile | Flow |
|---|---|---|---|
| Time [min] | Phase A [%] | Phase B [%] | Rate [mL/min] |
| 0.00 | 30 | 70 | 24 |
| 2.00 | 30 | 70 | 24 |
| 18.00 | 80 | 20 | 24 |

A-05 structural characterization data were as follows:

MS m/z (ESI): 1107.3 [M+H]⁺.

¹H NMR (400 MHZ, DMSO) δ 9.10 (s, 2H), 8.66-8.63 (m, 1H), 8.51 (d, J=8.8 Hz, 1H), 8.34-8.31 (m, 1H), 8.21-8.19 (m, 1H), 8.17-8.09 (m, 2H), 8.08-8.04 (m, 1H), 7.30 (s, 1H), 7.26-7.15 (m, 5H), 6.55 (s, 1H), 5.56-5.55 (m, 1H), 5.48-5.35 (m, 2H), 5.25-5.10 (m, 2H), 4.64 (d, J=6.4 Hz, 2H), 4.45-4.44 (m, 1H), 4.06-3.98 (m, 2H), 3.77-3.52 (m, 6H), 3.41 (s, 3H), 3.25-3.12 (m, 2H), 3.03-3.00 (m, 1H), 2.83-2.72 (m, 1H), 2.58-2.56 (m, 2H), 2.48 (s, 3H), 2.33-2.30 (m, 2H), 2.21-2.13 (m, 2H), 1.91-1.76 (m, 4H), 0.87 (t, J=7.2 Hz, 3H).

The following HPLC condition was used to detect the compounds A-07-A and A-07-B prepared in Example 3; and the compound A-07-A prepared in Example 3 and the compound A-05 prepared in Example 9.

Instrument: Agilent 1260 high-performance liquid chromatography VWD detector

Chromatographic column: Waters Xbridge C18 4.6*100 mm*3.5 μm

Mobile phase A: 0.01M ammonium phosphate aqueous solution/acetonitrile=90/10; Mobile phase B: acetonitrile

| | Mobile phase A | Mobile phase B |
|---|---|---|
| Time (min) | (%) | (%) |
| 0 | 100 | 0 |
| 6 | 22 | 78 |
| 11 | 22 | 78 |

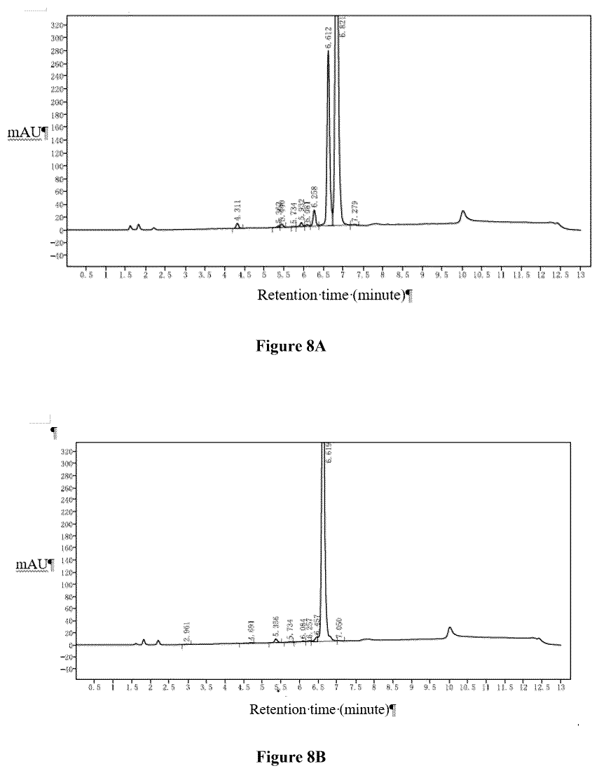

As shown in FIG. 8A, the retention times of compound A-07-A and compound A-07-B are 6.6 min and 6.8 min respectively, which illustrated that the above HPLC condition could separate isomers well.

As shown in FIG. 8B, there is a single peak for the compound A-07-A prepared in Example 3 and the compound A-05 prepared in Example 9, with the retention time of 6.6 min, which illustrated that the compound A-07-A and the compound A-05 are the same compound.

### Example 10: Synthesis of (1S,9S)-5-chloro-9-ethyl-1-(2-hydroxyacetamido)-4-methyl-10,13-dioxo-2,3,9,10,13,15-hexahydro-1H, 12H-benzo[de]pyrano[3′,4′: 6,7]indolizino[1,2-b]quinolin-9-yl 2-(4-((S)-2-(4-aminobutyl)-35-(4-((6-(2-(methylsulfonyl)pyrimidin-5-yl) hex-5-ynamido)methyl)-1H-1,2,3-triazol-1-yl)-4,8-dioxo-6,12,15,18,21,24,27,30,33-nonaoxa-3,9-diazapentatriacontanamido)phenyl) acetate (B-04)

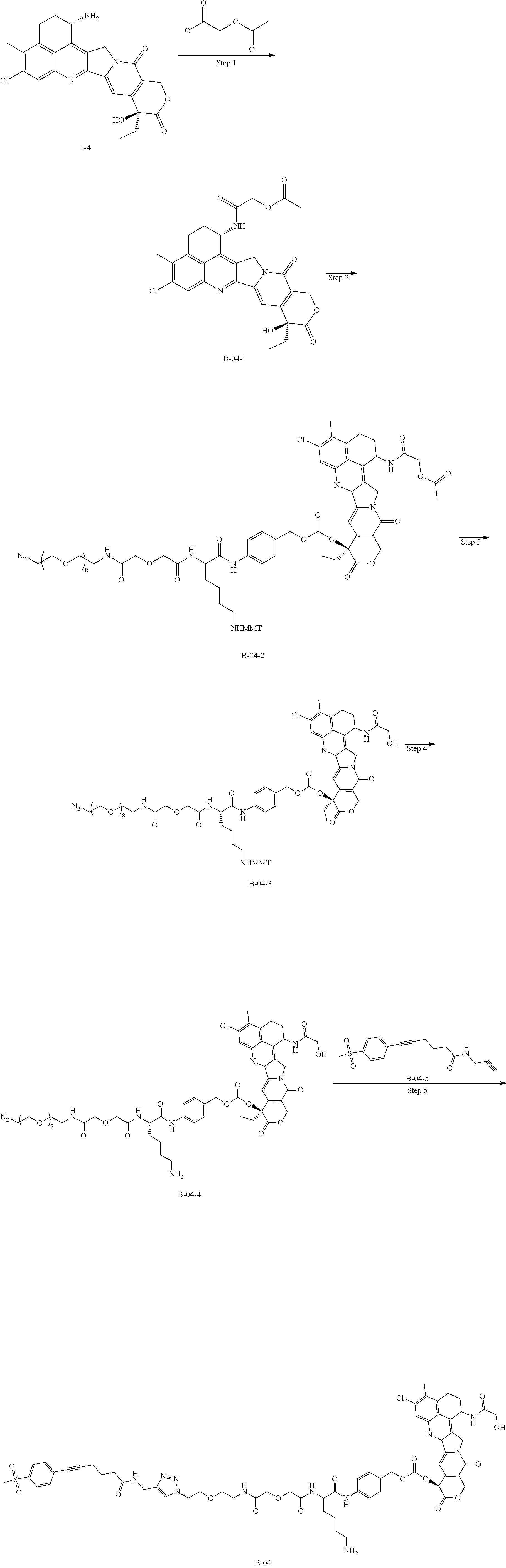

### Step 1: Preparation of 2-(((1S,9S)-5-chloro-9-ethyl-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-2-oxoethyl acetate (B-04-1)

(1S,9S)-1-amino-5-chloro-9-ethyl-9-hydroxy-4-methyl-1,2,3,9,12,15-hexahydro-10H, 13H-benzo[de]pyrano[3′,4′: 6,7]indolizino[1,2-b]quinoline-10,13-dione (2 g, 3.65 mmol) was dissolved in DMF (50 mL); DIPEA (1.18 g, 9.12 mmol, 1.59 mL) was added dropwise; acetoxyacetyl chloride (548.12 mg, 4.01 mmol, 431.59 μL) was added dropwise with stirring in an ice bath; and the stirring reaction was continued for 1 hour. The reaction solution was added to 0.1M dilute hydrochloric acid aqueous solution to precipitate a solid, which was filtered. The filter cake was dissolved in dichloromethane and methanol, dried over anhydrous sodium sulfate, filtered and concentrated to obtain the crude product, purified by silica gel column (methanol/dichloromethane=0%˜5%) and concentrated again to obtain the title compound (1.7 g, 3.077 mmol).

The structural characterization data were as follows:

SI-MS (m/z): 552.2 [M+1]⁺.

### Step 2: Preparation of (1S,9S)-1-(2-acetoxyacetamido)-5-chloro-9-ethyl-4-methyl-10,13-dioxo-2,3,9,10,13,15-hexahydro-1H,12H-benzo[de]pyrano[3′,4′: 6,7]indolizino[1,2-b]quinolin-9-yl 2-(4-((S)-35-azido-2-(4-(((4-methoxyphenyl)diphenylmethyl)amino)butyl)-4,8-dioxo-6,12,15,18,21,24,27,30,33-nonaoxa-3,9-diazapentatriacontanamido)phenyl) acetate (B-04-2)

2-(((1S,9S)-5-chloro-9-ethyl-9-hydroxy-4-methyl-10,13-dioxo-2,3,9,10,13,15-hexa Hydrogen-1H,12H-benzo[d]pyrano[3′,4′: 6,7]indeno[1,2-b]quinolin-1-yl)amino)-2-oxo Ethyl acetate (500 mg, 0.905 mmol) and DMAP (885.33 mg, 7.25 mmol) was dissolved in dry dichloromethane (5 mL), cooled to 0° C. under nitrogen protection, and triphosgene (268.81 mg, 0.905 mmol) was added dropwise. Dichloromethane solution (5 mL), keep stirring and reacting for 0.5 hours. Slowly added in(S)-2-(32-azido-5-oxo-3,9,12,15,18,21,24,27,30-nonyloxy-6-azatrinitro Dichloride of amino)-N-(4-(hydroxymethyl)phenyl)-6-(((4-methoxyphenyl)diphenylmethyl)amino) hexanamide (1.44 g, 1.36 mmol) Dichloromethane solution dropwise, naturally returned to room temperature and reacted for 4 hours. The reaction solution was quenched with water, extracted with dichloromethane 3 times (100 ml×3), combined the organic phases, washed with saturated brine, dried and concentrated, and purified bysilica gel column (MeOH/DCM=0%˜ 5%) the title compound (498 mg, 0.304 mmol). The structural characterization data were as follows:
ESI-MS (m/z): 1352.8 [M+1]⁺.

### Step 3: Synthesis of (1S,9S)-5-chloro-9-ethyl-1-(2-hydroxyacetamido)-4-methyl-10,13-dioxo-2,3,9,10,13,15-hexahydro-1H,12H-benzo[de]pyrano[3′,4′: 6,7]indolizino[1,2-b]quinolin-9-yl 2-(4-((S)-35-azido-2-(4-(((4-methoxyphenyl)diphenylmethyl)amino)butyl)-4,8-dioxo-6,12,15,18,21,24,27,30,33-nonaoxa-3,9-diazapentatriacontanamido)phenyl) acetate (B-04-3)

(1S,9S)-1-(2-acetoxyacetamido)-5-chloro-9-ethyl-4-methyl-10,13-dioxo-2,3,9,10,13,15-hexahydro-1H,12H-benzo[de]pyrano[3′,4′: 6,7]indolizino[1,2-b]quinolin-9-yl 2-(4-((S)-35-azido-2-(4-(((4-methoxyphenyl)diphenylmethyl)amino)butyl)-4,8-dioxo-6,12,15,18,21,24,27,30,33-nonaoxa-3,9-diazapentatriacontanamido)phenyl) acetate (B-04-2) (200 mg, 0.122 mmol) was dissolved in THF (3 mL) and MeOH (3 mL), and sodium carbonate (25.88 mg, 0.224 mmol) aqueous solution (1 mL) was added dropwise with stirring, and stirring was continued for 1 hour after the addition was complete. Add diluted hydrochloric acid to the reaction solution to neutralize the reaction, concentrated under reduced pressure and proceeded directly to the next step.

The structural characterization data were as follows:
ESI-MS (m/z): 1596.7 [M+1]⁺.

### Step 4: Synthesis of (1S,9S)-5-chloro-9-ethyl-1-(2-hydroxyacetamido)-4-methyl-10,13-dioxo-2,3,9,10,13,15-hexahydro-1H,12H-benzo[de]pyrano[3′,4′: 6,7]indolizino[1,2-b]quinolin-9-yl 2-(4-((S)-2-(4-aminobutyl)-35-azido-4,8-dioxo-6,12,15,18,21,24,27,30,33-nonaoxa-3,9-diazapentatriacontanamido)phenyl) acetate (B-04-4)

(1S,9S)-5-chloro-9-ethyl-1-(2-hydroxyacetamido)-4-methyl-10,13-dioxo-2,3,9,10,13,15-hexahydro-1H,12H-benzo[de]pyrano[3′,4′: 6,7]indolizino[1,2-b]quinolin-9-yl 2-(4-((S)-35-azido-2-(4-(((4-methoxyphenyl)diphenylmethyl)amino)butyl)-4,8-dioxo-6,12,15,18,21,24,27,30,33-nonaoxa-3,9-diazapentatriacontanamido)phenyl) acetate (B-04-3) (190 mg, 119.04 μmol) was dissolved in dichloromethane (5 mL), trifluoroacetic acid (0.5 mL) was added to continue the reaction for 1 hour. Added saturated aqueous sodium bicarbonate solution to the reaction liquid for neutralization, then separated the liquids, and concentrated the organic phase to obtain the crude product. The title compound (95 mg, 69.35 μmol) was obtained after purification by reverse-phase chromatography column (acetonitrile/1% aqueous formic acid=0%˜50%) and freeze-drying.

The structural characterization data were as follows:
ESI-MS (m/z): 1323.6 [M+1]⁺.

### Step 5: Synthesis of (1S,9S)-5-chloro-9-ethyl-1-(2-hydroxyacetamido)-4-methyl-10,13-dioxo-2,3,9,10,13,15-hexahydro-1H,12H-benzo[de]pyrano[3′,4′: 6,7]indolizino[1,2-b]quinolin-9-yl 2-(4-((S)-2-(4-aminobutyl)-35-(4-((6-(2-(methylsulfonyl)pyrimidin-5-yl) hex-5-ynamido)methyl)-1H-1,2,3-triazol-1-yl)-4,8-dioxo-6,12,15,18,21,24,27,30,33-nonaoxa-3,9-diazapentatriacontanamido)phenyl) acetate (B-04)

(1S,9S)-5-chloro-9-ethyl-1-(2-hydroxyacetamido)-4-methyl-10,13-dioxo-2,3,9,10,13,15-hexahydro-1H,12H-benzo[de]pyrano[3′,4′: 6,7]indolizino[1,2-b]quinolin-9-yl 2-(4-((S)-2-(4-aminobutyl)-35-azido-4,8-dioxo-6,12,15,18,21,24,27,30,33-nonaoxa-3,9-diazapentatriacontanamido)phenyl) acetate (B-04-4) (90 mg, 0.066 mmol) and N-ethynyl-6-(2-(methylsulfonyl)pyrimidin-5-yl) hex-5-ynamide (24.07 mg, 0.079 mmol) were dissolved in DMSO (2 mL) and water (0.2 mL), added cuprous bromide (9.42 mg, 0.066 mmol) and continued to stir for 2 hours. The reaction solution was directly filtered and the crude product was concentrated, which was purified by preparative high performance liquid chromatography and lyophilized to obtain the title compound (42.2 mg, 24.69 μmol).

The structural characterization data were as follows:
ESI-MS (m/z): 1628.7 [M+1]⁺.

Its preparation high performance liquid chromatography method is as follows:

Column: SunFire Prep C18 OBD 19 mm×150 mm×5.0 μm

Mobile phase A: acetonitrile; mobile phase B: water (0.05% formic acid)

| | Mobile | Mobile | Flow |
|---|---|---|---|
| Time [min] | PhaseA [%] | Phase B [%] | Rate [mL/min] |
| 0.00 | 10 | 90 | 28 |
| 2.00 | 10 | 90 | 28 |
| 18.00 | 90 | 10 | 28 |

### Example 11: Preparation of Antibody

Human-mouse chimeric antibodies, 22B6D2 (heavy chain variable region, SEQ ID NO: 15; light chain variable region, SEQ ID NO:16), 47A3E3 (heavy chain variable region, SEQ ID NO:32; light chain variable region, SEQ ID NO:33) and 100H7D3 (heavy chain variable region, SEQ ID NO:46; light chain variable region, SEQ ID NO:47), were obtained by immunizing H2L2 mice (provided by Harbour BioMed, and the antibodies produced by the mice were chimeric antibody composed of fully human variable region and murine constant region) through hybridoma screening, the above heavy chain variable region sequences were respectively combined with human IgG1 heavy chain constant region (SEQ ID NO:50), and the above light chain variable region sequences were respectively combined with human IgG1 light chain constant region (SEQ ID NO:51), thereby forming 3 complete fully human antibodies (see Table 1), which were subjected to codon optimization and then constructed into pTT5 plasmids, the pTT5 plasmid corresponding to each antibody heavy chain and light chain was simultaneously transfected into CHO-S cells, and protein A was used to purify the expressed antibodies in the supernatant to obtain the corresponding antibodies.

The HER3 control antibody was derived from U1-59 in the patent application No.: CN200680049887, which was subjected to codon optimization, then the nucleotide sequences of antibody heavy and light chains were synthesized and cloned into the pTT5 vector, then expressed and purified according to the above methods.

TABLE 1
Sequence information of 22B6D2-hIgG1, 47A3E3-hIgG1 and 100H7D3-hIgG1

| | Numbering | SEQ ID NO: | | | | | | | | | | | |
|---|---|---|---|---|---|---|---|---|---|---|---|---|---|
| Antibody | system | CDR-H1 | CDR-H2 | CDR-H3 | CDR-L1 | CDR-L2 | CDR-L3 | VH | VL | CH | CL | HC | LC |
| 22B6D2- | Chothia | 1 | 2 | 3 | 4 | 5 | 6 | 15 | 16 | 50 | 51 | 17 | 18 |
| hIgG1 | Kabat | 7 | 8 | 9 | 4 | 5 | 6 | | | | | | |
| | IMGT | 10 | 11 | 12 | 13 | 14 | 6 | | | | | | |
| 47A3E3- | Chothia | 19 | 20 | 21 | 22 | 23 | 24 | 32 | 33 | | | 34 | 35 |
| hIgG1 | Kabat | 25 | 26 | 21 | 22 | 23 | 24 | | | | | | |
| | IMGT | 27 | 28 | 29 | 30 | 31 | 24 | | | | | | |
| 100H7D3- | Chothia | 36 | 37 | 38 | 45 | 23 | 52 | 46 | 47 | | | 48 | 49 |
| hIgG1 | Kabat | 39 | 40 | 38 | 45 | 23 | 52 | | | | | | |
| | IMGT | 41 | 42 | 43 | 44 | 31 | 52 | | | | | | |

### Example 12: Conjugation of Compound Containing Cell Bioactive Molecule and Linker to Antibody

The antibodies 22B6, 47A3 and 100H7 involved in the antibody-drug conjugates prepared in the following examples were the antibodies 22B6D2-hIgG1, 47A3E3-hIgG1 and 100H7D3-hIgG1 as described in the second section, respectively.

The conjugation for the preparation of samples were described as follows:
0.46 ml of each of the antibodies 22B6, 47A3, 100H7, U1-59 and hIgG1 (concentration of each was adjusted to 11.0 mg/mL) was taken, diluted with 0.1M edetate disodium solution (pH 7.7), and then adjusted with 1M Na₂HPO₄ solution to pH 7.7, added with 10 mM TCEP (tris(2-carboxyethyl) phosphine) solution and mixed well, and allowed to stand at room temperature for 90 min. To the solution obtained as above, the “drug-linker” compound in 4.0 to 10 times the mass amount which was dissolved in dimethyl sulfoxide was added, mixed well, and allowed to stand at room temperature for 2 hours, then NAP-5 gel column (Cytiva) was used to replace buffer with 10 mM pH 6.0 histidine buffer solution, then sucrose and Tween 20 were added, and mixed well to obtain antibody-drug conjugates (i.e., ADC compounds), see Table 2.

The drug-to-antibody ratio (DAR value) of the conjugated samples was determined as follows:
LC-MS was used to determine the molecular weight of ADC samples, and the drug-to-antibody ratio (DAR value) was calculated;
The conjugated ADC samples were subjected to molecular weight analysis by LC-MS.

### Chromatographic Determination Conditions:

Liquid chromatography column: Thermo MAbPac RP 3.0*100 mm;
Mobile phase A: 0.1% FA/H₂O; Mobile phase B: 0.1% FA/ACN;
Flow rate: 0.25 ml/min; Sample chamber temperature: 8° C.; Column temperature: 60° C.; Injection volume: 2 μl;

| Time (minutes) | 2 | 20 | 22 | 25 | 26 | 30 |
|---|---|---|---|---|---|---|
| Mobile phase A (volume %) | 75 | 60 | 5 | 5 | 75 | 75 |
| Mobile phase B (volume %) | 25 | 40 | 95 | 95 | 25 | 25 |

### Mass Spectrometry Conditions:

Mass spectrometer model: AB Sciex Triple TOF 5600+; GS1 35; GS2 35; CUR 30; TEM 350; ISVF 5500; DP 250; CE 10; Accumulation time 0.5 s;
m/z 600-4000; Time bins to sum 40.

TABLE 2
ADC number and DAR

| | | Molar ratio of drug- | |
|---|---|---|---|
| Drug-linker No. | ADC No. | linker to antibody | DAR |
| A-07-A | 22B6-A-07-A-1 | 10:1 | 7.99 |
| A-07-A | 22B6-A-07-A-2 | 5:1 | 3.56 |
| A-07-A | 47A3-A-07-A | 10:1 | 6.27 |
| A-07-A | 100H7-A-07-A | 10:1 | 8.01 |
| A-07-A | U1-59-A-07-A | 10:1 | 8.02 |
| A-07-A | hIgG1-A-07-A | 10:1 | 8.03 |
| A-01 | 22B6-A-01 | 10:1 | 7.93 |
| A-01 | U1-59-A-01 | 10:1 | 7.89 |
| B-03 | 22B6-B-03-1 | 10:1 | 8.0 |
| B-03 | 22B6-B-03-2 | 5:1 | 3.14 |
| B-03 | hIgG1-B-03-1 | 10:1 | 7.90 |
| B-03 | hIgG1-B-03-2 | 5:1 | 4.20 |
| B-01 | 22B6- B-01 | 10:1 | 8.07 |
| B-01 | hIgG1-B-01 | 10:1 | 8.06 |
| A-01 | hIgG1-A-01 | 10:1 | 7.98 |

1. The molecular weight of ADC 22B6-A-07-A-1 was determined by LC-MS, and the drug-to-antibody ratio (DAR value) was calculated. The molecular weight analysis of the conjugated ADC 22B6-A-07-A-1 by LC-MS was shown in Table 3.

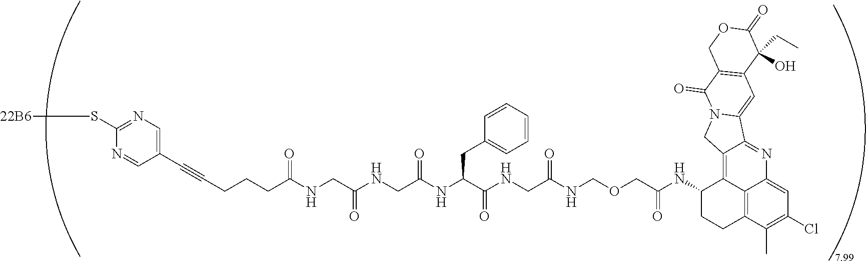

TABLE 3
Measured molecular weight of ADC 22B6-A-07-A-1

| Peptide chain | | mAb | DAR1 | DAR2 | DAR3 | DAR4 |
|---|---|---|---|---|---|---|
| LC | Measured value | 23333.66 | 24361.55 | N/A | N/A | N/A |
| HC | Measured value | 51311.72 | 52343.85 | 53371.29 | 54398.35 | 55424.24 |

TABLE 4
DAR value of ADC 22B6-A-07-A-1

| | Maximum signal strength | Ratio | |
|---|---|---|---|
| Name | * means that the chirality of the labeled atom is R or S | | DAR |
| LC-DAR0 | 17.76 | 0.002 | 7.99 |
| LC-DAR1 | 11714.79 | 0.998 | |
| HC-DAR0 | 2.19 | 0.001 | |
| HC-DAR1 | 5.33 | 0.002 | |
| HC-DAR2 | 38.61 | 0.014 | |
| HC-DAR3 | 2632.83 | 0.966 | |
| HC-DAR4 | 45.32 | 0.017 | |

The calculated drug-to-antibody ratio of ADC 22B6-A-07-A-1 was DAR=7.99, see Table 4.

2. The molecular weight of ADC 22B6-A-07-A-2 was determined by LC-MS, and the drug-to-antibody ratio (DAR value) was calculated. The molecular weight analysis of the conjugated ADC 22B6-A-07-A-2 by LC-MS was shown in Table 5.

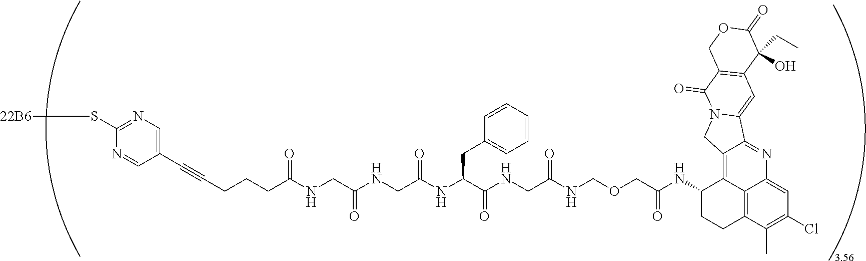

TABLE 5
Measured molecular weight of ADC 22B6-A-07-A-1

| Peptide chain | | mAb | DAR1 | DAR2 | DAR3 | DAR4 |
|---|---|---|---|---|---|---|
| LC | Measured value | 23333.90 | 24361.22 | N/A | N/A | N/A |
| HC | Measured value | 51315.83 | 52342.63 | 53369.65 | 54396.95 | N/A |

TABLE 6
DAR value of ADC 22B6-A-07-A-2

| Name | Maximum signal strength | Ratio | DAR |
|---|---|---|---|
| LC-DAR0 | 4913.02 | 0.548 | 3.56 |
| LC-DAR1 | 4053.08 | 0.452 | |
| HC-DAR0 | 621.45 | 0.239 | |
| HC-DAR1 | 885.02 | 0.341 | |
| HC-DAR2 | 707.16 | 0.272 | |
| HC-DAR3 | 382.56 | 0.147 | |
| HC-DAR4 | 0.00 | 0.000 | |

The calculated drug-to-antibody ratio of ADC 22B6-A-07-A-2 was DAR=3.56, see Table 6.

3. The molecular weight of ADC 47A3-A-07-A was determined by LC-MS, and the drug-to-antibody ratio (DAR value) was calculated. The molecular weight analysis of the conjugated ADC 47A3-A-07-A by LC-MS was shown in Table 7.

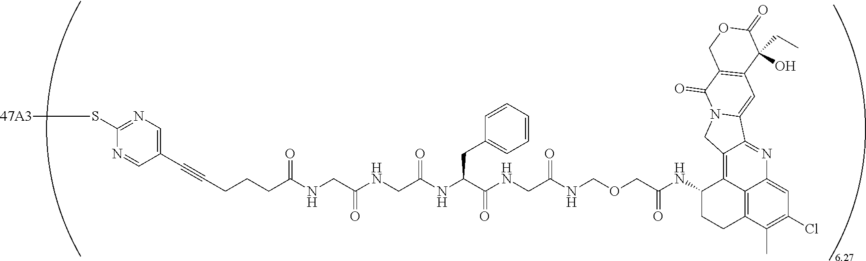

TABLE 7
Measured molecular weight of ADC 47A3-A-07-A

| Peptide chain | | mAb | DAR1 | DAR2 | DAR3 | DAR4 |
|---|---|---|---|---|---|---|
| LC | Measured value | 23564.32 | 24591.76 | N/A | N/A | N/A |
| HC | Measured value | 51443.47 | 52469.07 | 53498.19 | 54525.66 | 55555.34 |

TABLE 8
DAR value of ADC 47A3-A-07-A

| Name | Maximum signal strength | Ratio | DAR |
|---|---|---|---|
| LC-DAR0 | 777.53 | 0.180 | 6.27 |
| LC-DAR1 | 3542.16 | 0.820 | |
| HC-DAR0 | 336.77 | 0.222 | |
| HC-DAR1 | 1.70 | 0.001 | |
| HC-DAR2 | 25.97 | 0.017 | |
| HC-DAR3 | 1154.30 | 0.760 | |
| HC-DAR4 | 0.20 | 0.000 | |

The calculated drug-to-antibody ratio of ADC 47A3-A-07-A was DAR=6.27, see Table 8.

The molecular weight of ADC 100H7-A-07-A was determined by LC-MS, and the drug-to-antibody ratio (DAR value) was calculated. The molecular weight analysis of the conjugated ADC 100H7-A-07-A by LC-MS was shown in Table 9.

TABLE 9
Measured molecular weight of ADC 100H7-A-07-A

| Peptide chain | | mAb | DAR1 | DAR2 | DAR3 | DAR4 |
|---|---|---|---|---|---|---|
| LC | Measured value | 23459.14 | 24487.82 | N/A | N/A | N/A |
| HC | Measured value | 50140.37 | 51168.90 | 52193.09 | 53221.86 | 54248.08 |

TABLE 10
DAR value of ADC 100H7-A-07-A

| Name | Maximum signal strength | Ratio | DAR |
|---|---|---|---|
| LC-DAR0 | 0.22 | 0.000 | 8.01 |
| LC-DAR1 | 6088.44 | 1.000 | |
| HC-DAR0 | 1.94 | 0.001 | |
| HC-DAR1 | 0.00 | 0.000 | |
| HC-DAR2 | 0.98 | 0.000 | |
| HC-DAR3 | 1957.89 | 0.989 | |
| HC-DAR4 | 18.43 | 0.009 | |

The calculated drug-to-antibody ratio of ADC 100H7-A-07-A was DAR=8.01, see Table 10.

5. The molecular weight of ADC U1-59-A-07-A was determined by LC-MS, and the drug-to-antibody ratio (DAR value) was calculated. The molecular weight analysis of the coupled ADC U1-59-A-07-A by LC-MS was shown in Table 11.

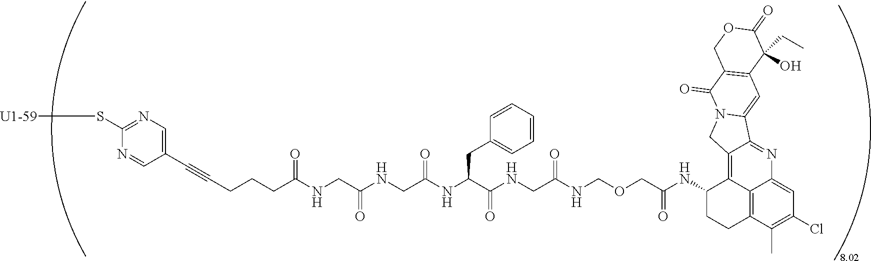

TABLE 11
Measured molecular weight of ADC U1-59-A-07-A

| Peptide chain | | mAb | DAR1 | DAR2 | DAR3 | DAR4 |
|---|---|---|---|---|---|---|
| LC | Measured value | 24290.64 | 25318.79 | N/A | N/A | N/A |
| HC | Measured value | N/A | 51531.74 | 52561.59 | 53587.44 | 54614.36 |

TABLE 12
DAR value of ADC U1-59-A-07-A

| Name | Maximum signal strength | Ratio | DAR |
|---|---|---|---|
| LC-DAR0 | 4.51 | 0.000 | 8.02 |
| LC-DAR1 | 9409.93 | 1.000 | |
| HC-DAR0 | 0.00 | 0.000 | |
| HC-DAR1 | 0.00 | 0.000 | |
| HC-DAR2 | 17.27 | 0.008 | |
| HC-DAR3 | 2247.34 | 0.977 | |
| HC-DAR4 | 36.06 | 0.016 | |

The calculated drug-to-antibody ratio of ADC U1-59-A-07-A was DAR=8.02, see Table 12.

6. The molecular weight of ADC hIgG1-A-07-A was determined by LC-MS, and the drug-to-antibody ratio (DAR value) was calculated. The molecular weight analysis of the conjugated ADC hIgG1-A-07-A by LC-MS was shown in Table 13.

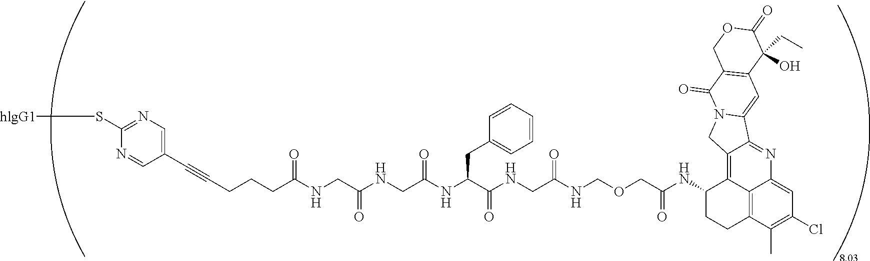

TABLE 13
Measured molecular weight of ADC hIgG1-A-07-A

| Peptide chain | | mAb | DAR1 | DAR2 | DAR3 | DAR4 |
|---|---|---|---|---|---|---|
| LC | Measured value | 23356.43 | 24384.30 | N/A | N/A | N/A |
| HC | Measured value | 50171.46 | 51198.30 | 52226.14 | 53254.03 | 54280.32 |

TABLE 14
DAR value of ADC hIgG1-A-07-A

| Name | Maximum signal strength | Ratio | DAR |
|---|---|---|---|
| LC-DAR0 | 0.01 | 0.000 | 8.03 |
| LC-DAR1 | 6859.36 | 1.000 | |
| HC-DAR0 | 0.64 | 0.000 | |
| HC-DAR1 | 0.15 | 0.000 | |
| HC-DAR2 | 0.81 | 0.000 | |
| HC-DAR3 | 2656.39 | 0.985 | |
| HC-DAR4 | 39.69 | 0.015 | |

The calculated drug-to-antibody ratio of ADC hIgG1-A-07-A (8) was DAR=8.03, see Table 14.

7 The molecular weight of ADC 22B6-A-01 was determined by LC-MS, and the drug-to-antibody ratio (DAR value) was calculated. The molecular weight analysis of the conjugated ADC 22B6-A-01 by LC-MS was shown in Table 15.

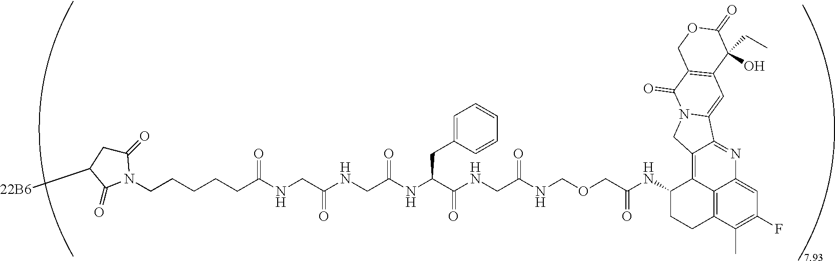

TABLE 15
Measured molecular weight of ADC 22B6-A-01

| Peptide chain | | mAb | DAR1 | DAR2 | DAR3 | DAR4 |
|---|---|---|---|---|---|---|
| LC | Measured value | 23333.84 | 24367.86 | N/A | N/A | N/A |
| HC | Measured value | 51315.60 | 52350.27 | 53383.32 | 54417.88 | 55452.52 |

TABLE 16
DAR value of ADC 22B6-A-01

| Name | Maximum signal strength | Ratio | DAR |
|---|---|---|---|
| LC-DAR0 | 63.87 | 0.010 | 7.93 |
| LC-DAR1 | 6265.77 | 0.990 | |
| HC-DAR0 | 3.26 | 0.002 | |
| HC-DAR1 | 7.83 | 0.005 | |
| HC-DAR2 | 16.62 | 0.010 | |
| HC-DAR3 | 1621.08 | 0.981 | |
| HC-DAR4 | 4.30 | | |

The calculated drug-to-antibody ratio of ADC 22B6-A-01 was DAR=7.93, see Table 16.

8. The molecular weight of ADC U1-59-A-01 was determined by LC-MS, and the drug-to-antibody ratio (DAR value) was calculated. The molecular weight analysis of the coupled ADC U1-59-A-01 by LC-MS was shown in Table 17.

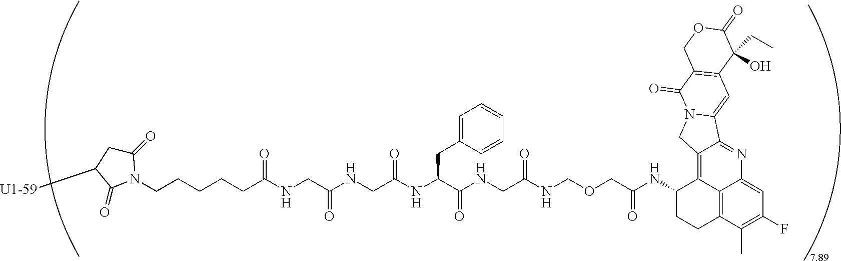

TABLE 17
Measured molecular weight of ADC U1-59-A-01

| Peptide chain | | mAb | DAR1 | DAR2 | DAR3 | DAR4 |
|---|---|---|---|---|---|---|
| LC | Measured value | 23881.57 | 25325.29 | N/A | N/A | N/A |
| HC | Measured value | N/A | 50720.49 | 52160.53 | 53606.48 | 55055.42 |

TABLE 18
DAR value of ADC U1-59-A-01

| Name | Maximum signal strength | Ratio | DAR |
|---|---|---|---|
| LC-DAR0 | 0.15 | 0.000 | 7.89 |
| LC-DAR1 | 5937.79 | 1.000 | |
| LC-DAR2 | 0.16 | 0.000 | |
| HC-DAR0 | 0.00 | 0.000 | |
| HC-DAR1 | 15.59 | 0.007 | |
| HC-DAR2 | 102.06 | 0.045 | |
| HC-DAR3 | 2139.31 | 0.946 | |
| HC-DAR4 | 4.81 | 0.002 | |

The calculated drug-to-antibody ratio of ADC U1-59-A-01 was DAR=7.89, see Table 18.

9. The molecular weight of ADC 22B6-B-03-1 was determined by LC-MS, and the drug-to-antibody ratio (DAR value) was calculated. The molecular weight analysis of the conjugated ADC 22B6-B-03-1 by LC-MS was shown in Table 19.

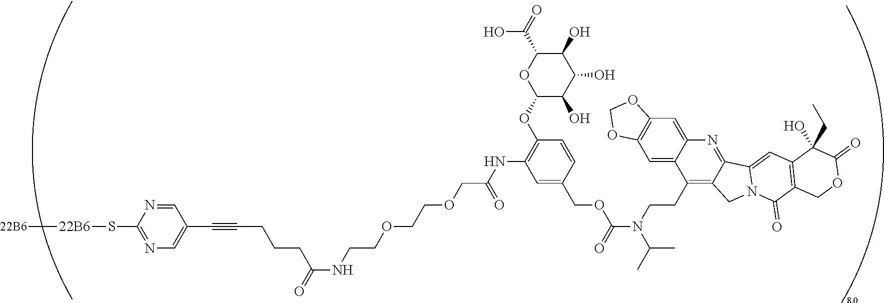

TABLE 19
Measured molecular weight of ADC 22B6-B-03-1

| Peptide chain | | mAb | DAR1 | DAR2 | DAR3 | DAR4 |
|---|---|---|---|---|---|---|
| LC | Measured value | 23333.71 | 24468.53 | N/A | N/A | N/A |
| HC | Measured value | N/A | 52452.66 | 53586.81 | 54719.16 | N/A |

TABLE 20
DAR value of ADC 22B6-B-03-1

| Name | Maximum signal strength | Ratio | DAR |
|---|---|---|---|
| LC-DAR0 | 0.55 | 0.000 | 8.0 |
| LC-DAR1 | 7491.47 | 1.000 | |
| HC-DAR0 | 0.00 | 0.000 | |
| HC-DAR1 | 0.16 | 0.000 | |
| HC-DAR2 | 1.77 | 0.002 | |
| HC-DAR3 | 986.79 | 0.998 | |
| HC-DAR4 | 0.00 | 0.000 | |

The calculated drug-to-antibody ratio of ADC 22B6-B-03-1 was DAR=8.0, see Table 20.

10. The molecular weight of ADC 22B6-B-03-2 was determined by LC-MS, and the drug-to-antibody ratio (DAR value) was calculated. The molecular weight analysis of the conjugated ADC 22B6-B-03-2 by LC-MS was shown in Table 21.

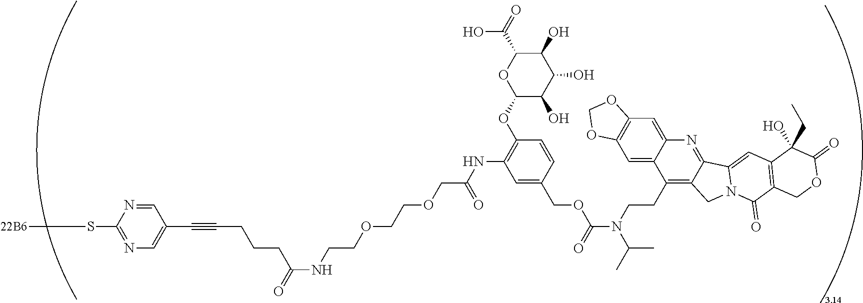

TABLE 21
Measured molecular weight of ADC 22B6-B-03-2

| Peptide chain | | mAb | DAR1 | DAR2 | DAR3 | DAR4 |
|---|---|---|---|---|---|---|
| LC | Measured value | 23333.86 | 24467.90 | N/A | N/A | N/A |
| HC | Measured value | 51315.63 | 52449.47 | 53583.27 | 54717.13 | N/A |

TABLE 22
DAR value of ADC 22B6-B-03-2

| Name | Maximum signal strength | Ratio | DAR |
|---|---|---|---|
| LC-DAR0 | 4458.58 | 0.594 | 3.14 |
| LC-DAR1 | 3053.31 | 0.406 | |
| HC-DAR0 | 564.55 | 0.296 | |
| HC-DAR1 | 684.51 | 0.359 | |
| HC-DAR2 | 446.79 | 0.234 | |
| HC-DAR3 | 212.83 | 0.112 | |
| HC-DAR4 | 0.00 | 0.000 | |

The calculated drug-to-antibody ratio of ADC 22B6-B-03-2 was DAR=3.14, see Table 22.

11. The molecular weight of ADC hIgG1-B-03-1 was determined by LC-MS, and the drug-to-antibody ratio (DAR value) was calculated. The molecular weight analysis of the conjugated ADC hIgG1-B-03-1 by LC-MS was shown in Table 23.

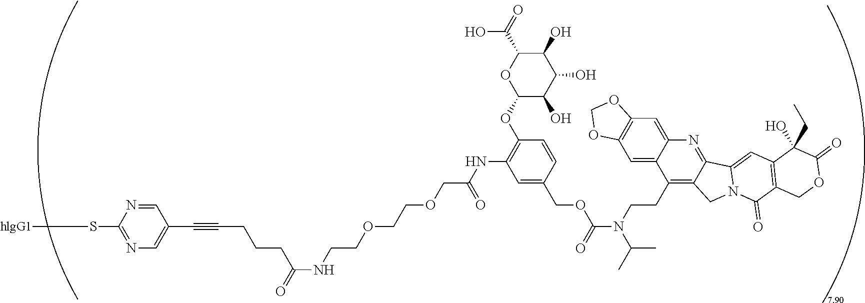

TABLE 23
Measured molecular weight of ADC hIgG1-B-03-1

| Peptide chain | | mAb | DAR1 | DAR2 | DAR3 | DAR4 |
|---|---|---|---|---|---|---|
| LC | Measured value | 23356.96 | 24491.30 | N/A | N/A | N/A |
| HC | Measured value | 50170.43 | 51302.49 | 52441.17 | 53574.55 | 54707.63 |

TABLE 24
DAR value of ADC hIgG1-B-03-1

| Name | Maximum signal strength | Ratio | DAR |
|---|---|---|---|
| LC-DAR0 | 221.82 | 0.027 | 7.90 |
| LC-DAR1 | 7905.94 | 0.973 | |
| HC-DAR0 | 13.64 | 0.008 | |
| HC-DAR1 | 0.55 | 0.000 | |
| HC-DAR2 | 1.35 | 0.001 | |
| HC-DAR3 | 1644.22 | 0.990 | |
| HC-DAR4 | 1.51 | 0.001 | |

The calculated drug-to-antibody ratio of ADC hIgG1-B-03-1 was DAR=7.90, see Table 24.

12. The molecular weight of ADC hIgG1-B-03-2 was determined by LC-MS, and the drug-to-antibody ratio (DAR value) was calculated. The molecular weight analysis of the conjugated ADC hIgG1-B-03-2 by LC-MS was shown in Table 25.

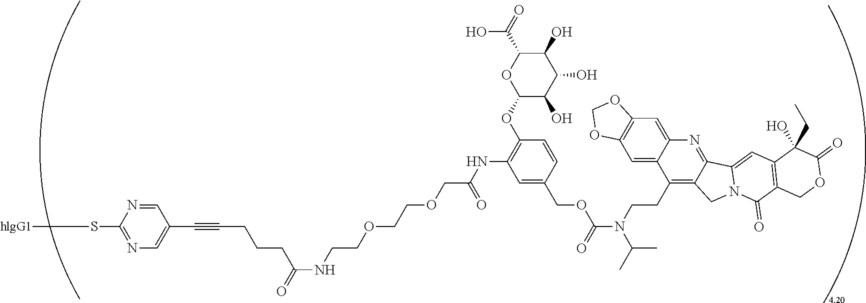

TABLE 25
Measured molecular weight of ADC hIgG1-B-03-2

| Peptide chain | | mAb | DAR1 | DAR2 | DAR3 | DAR4 |
|---|---|---|---|---|---|---|
| LC | Measured value | 23357.18 | 24491.24 | N/A | N/A | N/A |
| HC | Measured value | 50172.72 | 51306.68 | 52440.51 | 53574.02 | N/A |

TABLE 26
DAR value of ADC hIgG1-B-03-2

| Name | Maximum signal strength | Ratio | DAR |
|---|---|---|---|
| LC-DAR0 | 968.46 | 0.395 | 4.20 |
| LC-DAR1 | 1480.74 | 0.605 | |
| HC-DAR0 | 163.15 | 0.148 | |
| HC-DAR1 | 432.71 | 0.392 | |
| HC-DAR2 | 302.86 | 0.275 | |
| HC-DAR3 | 204.00 | 0.185 | |
| HC-DAR4 | 0.00 | 0.0 | |

The calculated drug-to-antibody ratio of ADC hIgG1-B-03-2 was DAR=4.20, see Table 26.

13. The molecular weight of ADC 22B6-B-01 was determined by LC-MS, and the drug-to-antibody ratio (DAR value) was calculated. The molecular weight analysis of the conjugated ADC 22B6-B-01 by LC-MS was shown in Table 27.

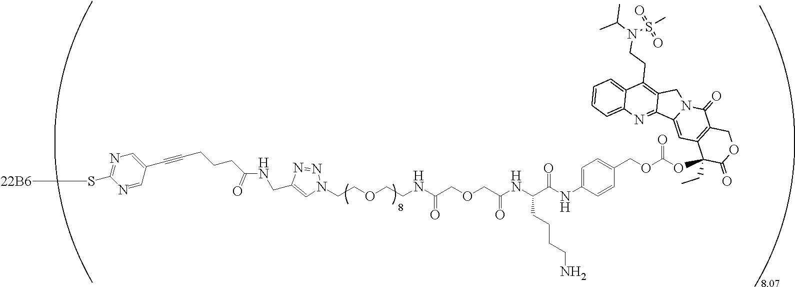

TABLE 27
Measured molecular weight of ADC 22B6-B-01

| Peptide chain | | mAb | DAR1 | DAR2 | DAR3 | DAR4 |
|---|---|---|---|---|---|---|
| LC | Measured value | N/A | 24884.55 | 26438.34 | N/A | N/A |
| HC | Measured value | N/A | N/A | 54418.55 | 55967.73 | 57516.65 |

TABLE 28
DAR value of ADC 22B6-B-01

| Name | Maximum signal strength | Ratio | DAR |
|---|---|---|---|
| LC-DAR0 | 0.00 | 0.000 | 8.07 |
| LC-DAR1 | 6812.74 | 0.999 | |
| LC-DAR2 | 4.37 | 0.001 | |
| HC-DAR0 | 0.00 | 0.000 | |
| HC-DAR1 | 0.00 | 0.000 | |
| HC-DAR2 | 30.50 | 0.017 | |
| HC-DAR3 | 1656.42 | 0.933 | |
| HC-DAR4 | 88.88 | 0.050 | |

The calculated drug-to-antibody ratio of ADC 22B6-B-01 was DAR=8.07, see Table 28.

14. The molecular weight of ADC hIgG1-B-01 was determined by LC-MS, and the drug-to-antibody ratio (DAR value) was calculated. The molecular weight analysis of the conjugated ADC hIgG1-B-01 by LC-MS was shown in Table 29.

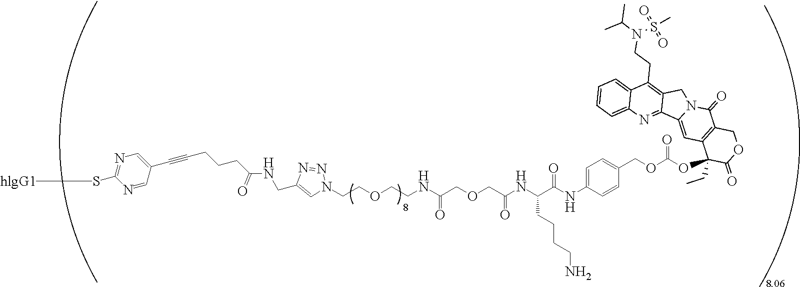

TABLE 29
Measured molecular weight of ADC hIgG1-B-01

| Peptide chain | | mAb | DARI1 | DAR2 | DAR3 | DAR4 |
|---|---|---|---|---|---|---|
| LC | Measured value | N/A | 24907.44 | 26456.26 | N/A | N/A |
| HC | Measured value | 50171.01 | N/A | 53272.32 | 54823.15 | 56372.21 |

TABLE 30
DAR value of ADC hIgG1-B-01

| Name | Maximum signal strength | Ratio | DAR |
|---|---|---|---|
| LC-DAR0 | 0.00 | 0.000 | 8.06 |
| LC-DAR1 | 7209.28 | 0.999 | |
| LC-DAR2 | 5.25 | 0.001 | |
| HC-DAR0 | 2.83 | 0.001 | |
| HC-DAR1 | 0.00 | 0.000 | |
| HC-DAR2 | 35.43 | 0.013 | |
| HC-DAR3 | 2610.46 | 0.940 | |
| HC-DAR4 | 127.17 | 0.046 | |

The calculated drug-to-antibody ratio of ADC hIgG1-B-01 was DAR=8.06, see Table 30.

15. The molecular weight of ADC hIgG1-A-01 was determined by LC-MS, and the drug-to-antibody ratio (DAR value) was calculated. The molecular weight analysis of the conjugated ADC hIgG1-A-01 by LC-MS was shown in the table below.

TABLE 31
DAR values of ADC hIgG1-A-01

| Name | Maximum signal strength | Ratio | DAR |
|---|---|---|---|
| LC-DAR0 | 0.16 | 0.000 | 7.96 |
| LC-DAR1 | 6021.53 | 1.000 | |
| LC-DAR2 | 0.18 | 0.000 | |
| HC-DAR0 | 0.00 | 0.000 | |
| HC-DAR1 | 15.60 | 0.007 | |
| HC-DAR2 | 102.06 | 0.045 | |
| HC-DAR3 | 2145.21 | 0.951 | |
| HC-DAR4 | 4.82 | 0.002 | |

### Example 13a: Conjugation of ADC 22B6-B-04

0.854 ml 22B6 antibody (23.43 mg/ml) was diluted with 42.68 μL of 20 mM PB+0.1M EDTA (pH 7.60), 1M Na₂HPO₄ solution was used to adjust pH to 7.57, then 20 mM TCEP (tris(2-carboxyethyl) phosphine, 73.68 μL, pH 7.60) was added and mixed well, and allowed to stand at room temperature for 1.5 hours. Then 12 times the mass amount of B-04 (164 μl, 10 mM), which was dissolved in dimethyl sulfoxide, was slowly added and mixed well, and allowed to stand at room temperature for 2 hours, then NAP-5 gel column (Cytiva) was used to replace buffer with 20 mM pH 6.0 histidine buffer solution, to obtain ADC 22B6-B-04-01, with DAR value of 8.62, which was measured by mass spectrometry.

### Example 13b: Conjugation of ADC 22B6-B-04 and ADC hIgG1-B-04

Alternatively, ADC 22B6-B-04 was prepared as follows: 2.667 ml 22B6 antibody (18.75 mg/ml) was diluted with 133.35 μL of 20 mM PB+0.1M EDTA (pH 7.60), 1M Na₂HPO₄ solution was used to adjust pH to 7.63, then 10 mM TCEP (tris(2-carboxyethyl) phosphine, 184.19 μL, pH 7.60) was added and mixed well, and allowed to stand at room temperature for 1.5 hours. Then 10 times the mass amount of B-04 (341.73 μl, 10 mM), which was dissolved in dimethyl sulfoxide, was slowly added and mixed well, and allowed to stand at room temperature for 2 hours, then NAP-5 gel column (Cytiva) was used to replace buffer with 20 mM pH 6.0 histidine buffer solution, to obtain ADC 22B6-B-04-02, with DAR value of 6.87, which was measured by mass spectrometry.

### Example 13c: Conjugation of ADC hIgG1-B-04

ADChIgG1-B-04 sample was prepared by using the same preparation method as in Example 13b and substituting the antibody with hIgG1 antibody. The mass spectrometry measured DAR value is 7.02.

### Example 14: Detection the Activity of the Antibody-Drug Conjugates

1. Detection of Cell Affinity of Anti-Human HER3 Antibodies and their Drug Conjugates

The affinity of anti-human HER3 ADC to MDA-MB-453, NCI-H358, KPL-4, and A431 cells was detected by flow cytometry (Beckman, model Cytoflex).

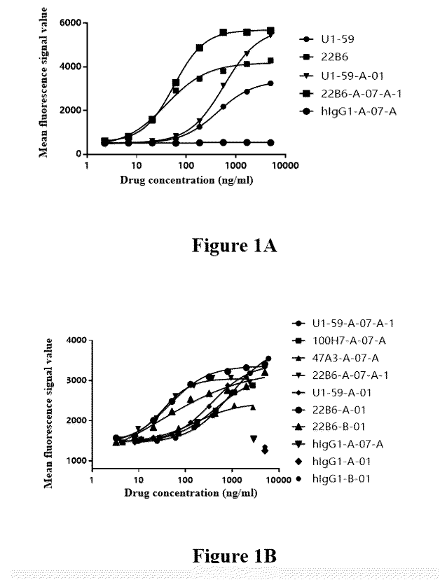

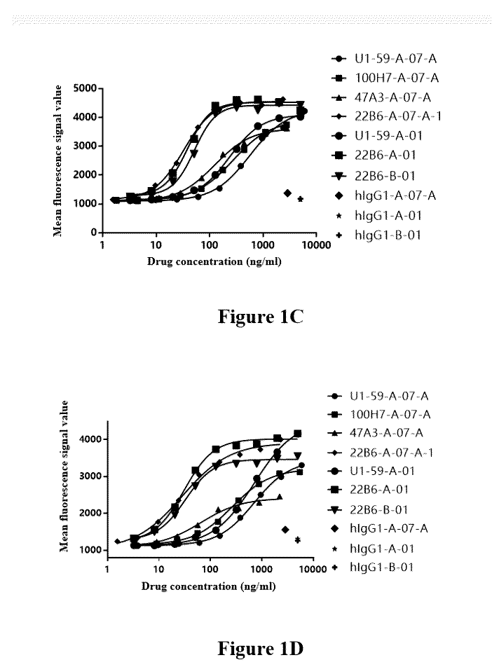

Adherent MDA-MB-453, NCI-H358, KPL-4, A431 cells were digested with Tryple (manufacturer: Gibco) solution, counted, and an appropriate amount of the cells were taken, washed twice with 1×PBS, resuspended in 1% BSA in PBS solution, and then transferred to a 96-well sharp-bottom plate, 50 μl per well; candidate antibodies and drug conjugates thereof were diluted with 1% BSA in PBS solution by 3-fold or 2.5-fold serial dilution starting from 5 μg/mL, and then 50 μl of the diluted antibody or antibody-drug conjugate was added to the sharp-bottom plate containing the cells, and incubation was carried out at 4° C. for 40 minutes; the cells were washed twice with PBS, then added with 50 μl of diluted secondary antibody to each well, mixed, and incubated at 4° C. for 30 minutes; the cells were washed with PBS twice, then resuspended in 400 μl of PBS and detected by flow cytometry. Data processing: the average fluorescence signal values were exported, and then input into GraphPad Prism 6 software to calculate EC50. The results were shown in Table 32 and FIGS. 1A, 1B, 1C, and 1D. The above results showed that, the affinity to cells of the ADCs obtained by conjugation of the antibodies of the present invention (e.g., 22B6, 47A3 and 100H7) was significantly higher than that of the ADCs obtained by conjugating control antibody U1-5; and the affinity to cells of the naked antibodies of the present invention (e.g., 22B6) was significantly higher than that of the naked control antibody U1-59. Moreover, when the naked antibody was formed into ADC through conjugation, the affinity to cells was less affected, and the affinity of 22B6 formed into ADC by conjugation was still the highest.

TABLE 32
Determination results of cell affinity
of anti-human HER3 antibody and ADC

| | EC50 (ng/ml) | | | |
|---|---|---|---|---|
| Drug | MDA-MB-453 | NCI-H358 | KPL-4 | A431 |
| U1-59-A-07-A | / | 938.8 | 567.8 | 710.6 |
| 100H7-A-07-A | / | 427.4 | 274.4 | 265.9 |
| 47A3-A-07-A | / | 157.6 | 124.6 | 65.6 |
| 22B6-A-07-A-1 | 55.98 | 32.91 | 32.51 | 28.36 |
| U1-59-A-01 | 572.7 | 356.5 | 236 | 716.1 |
| 22B6-A-01 | / | 44.55 | 38.68 | 30.74 |
| 22B6-B-01 | / | 29.79 | 51.9 | 32.78 |
| U1-59 | 426.4 | / | / | / |
| 22B6 | 39.19 | / | / | / |

Note:
“/” means not detected.

### 2. In Vitro Cytotoxicity of Anti-Human HER3 Antibody-Drug Conjugates

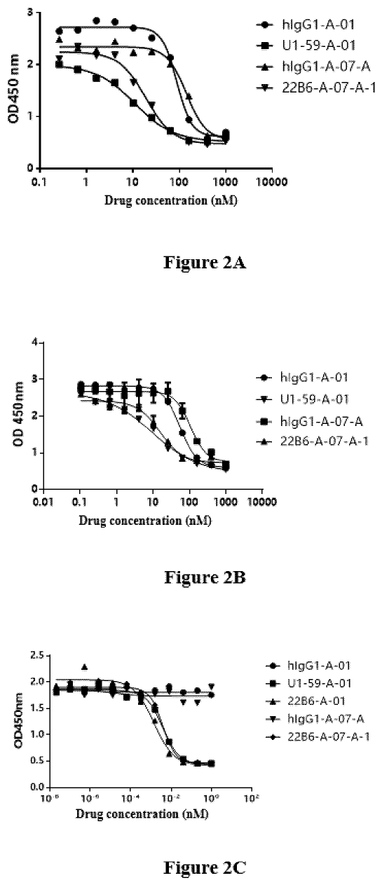

MDA-MB-453, HCC1569, and HEK293T-h HER3 cells were digested with TrypLE (manufacturer: Gibco) solution, counted, and an appropriate amount of the cells were taken, diluted with growth medium, plated on a 96-well plate, 5000 cells or 3000 cells (100 μl)/well, cultured overnight at 37° C., 5% CO₂; on the next day, ADC was diluted with the growth medium by 2.5-fold gradient dilution starting from 150 μg/ml, and then 100 μl of the diluted ADC was taken, added to the 96-well plate containing the cells, the plate was placed into an incubator and incubated at 37° C. and 5% CO₂ for 6 days, then 20 μl of CCK8 was added to each well, incubation was conducted for 2-5 h at 37° C., and the OD450 nm absorbance value was read with a microplate reader; the original data were input into Graph Prism 6 to calculate IC₅₀. As shown in Table 33 and FIGS. 2A, 2B and 2C, the ADC formed by the antibodies of the present invention (e.g., 22B6-A-01, 22B6-A-07-A-1) had target-specific killing effect, wherein the IC₅₀ of 22B6-A-07-A-1 against tumor cells differed from that of hIgG1-A-01 or hIgG1-A-07-A by 6 to 8 times, hIgG1-A-01 and hIgG1-A-07-A showed no obvious killing effect against the overexpression cells (HEK293T-h HER3), and the IC₅₀ of ADCs such as 22B6-A-01 and 22B6-A-07-A-1 differed more significantly from that of hIgG-A-01 or hIgG-A-07-A.

TABLE 33
In vitro cytotoxicity results of anti-human HER3 ADC

| | IC50 (nM) | | |
|---|---|---|---|
| Drug | MDA-MB-453 | HCC1569 | HEK293T-hHER3 |
| hIgG1-A-01 | 88.07 | 54.87 | >10000 |
| U1-59-A-01 | 10.95 | 9.74 | 0.0038 |
| 22B6-A-01 | / | / | 0.0014 |
| hIgG1-A-07-A | 141.6 | 106.9 | >10000 |
| 22B6-A-07-A-1 | 19.04 | 15.61 | 0.0038 |

Note:
“/” means not detected.

### 3. Bystander Effect of Anti-Human HER3 Antibody-Drug Conjugates

HEK293T and HEK293T-h HER3 cells were digested with 0.25% Trypsin-EDTA (manufacturer: Gibco), counted, and an appropriate amount of the cells were taken, washed twice with 1×PBS, the cell density was adjusted to 1×10⁶ cells/ml, and then Cell Trace Violet was added to reach a final concentration of 5 μM for labeling, mixed well and incubated in an incubator for 20 min, then the cells were washed with growth medium and counted, plated on 24-well plates (500 μl/well) according to the following ratios: HEK293T-labeled: HEK293T=4000:12000 cells/well, HEK293T-labeled: HEK293T-h HER3=4000:12000 cells/well, HEK293T-h HER3-labeled: HEK293T-h HER3=4000:12000 cells/well, and cultured overnight at 37° C., 5% CO₂;

On the next day, ADC was diluted with growth medium to reach concentrations of 20 nM, 4 nM, and 0.8 nM, then 500 μl of the diluted ADC was taken and added to the corresponding 24-well plates containing cells, and the plates were placed and cultured in an incubator at 37° C., 5% CO₂ for 4 days;

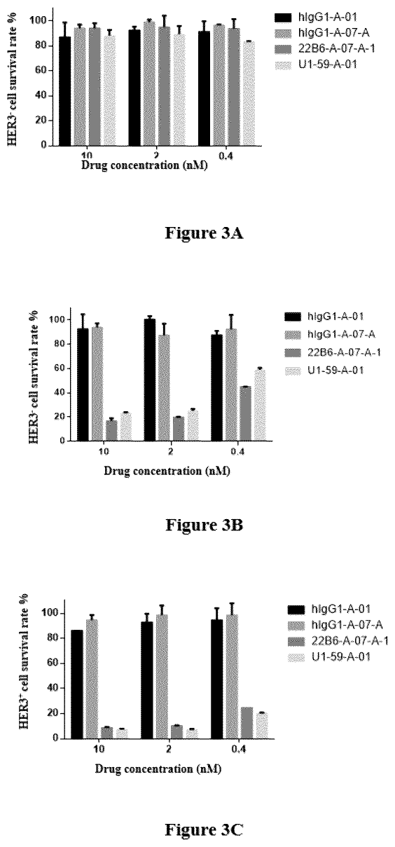

HEK293T and HEK293T-h HER3 cells were digested with 0.25% Trypsin-EDTA, added to a growth medium containing PI with a final concentration of 3 μM, mixed well, incubated at room temperature for 15 min, and then detected by flow cytometry (Thermo, model: Attune NxT). The results were shown in FIGS. 3A, 3B and 3C, wherein 22B6-A-07-A-1 and U1-59-A-01 basically did not kill HEK293T negative cells, but had significant specific killing effect on HEK293T-HER3; 22B6-A-07-A-1 and U1-59-A-01 had significant killing effect on the negative cells under the condition that negative cells HEK293T and positive cells HEK293T-HER3 coexisted, indicating that 22B6-A-07-A-1 and U1-59-A-01 had bystander killing activity.

### 4. Detection of Plasma Stability of Anti-Human HER3 Antibody-Drug Conjugate

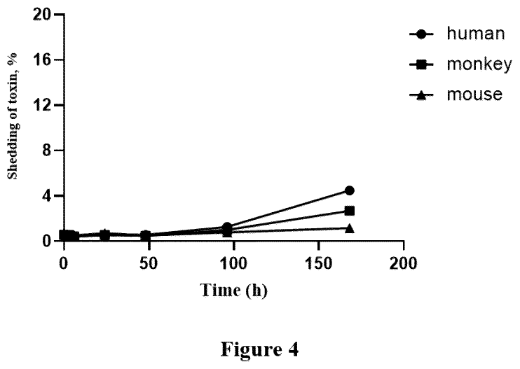

3.59 μl of 20.9 mg/ml 22B6-A-07-A-1 ADC sample was precisely weighted and transferred into EP tubes, added with 496 μl of human, cynomolgus monkey and mouse blank plasma respectively, and mixed well to obtain human, cynomolgus monkey and mouse plasma samples containing 22B6-A-07-A-1 ADC. The samples were placed in a constant temperature incubator and incubated at 37° C., and 50 μl of each sample was taken at 1, 3, 6, 24, 48, 96 and 168 hours, added with 200 μl of 0.5% formic acid-acetonitrile to precipitate protein, and centrifuged at 13,000 rpm at 4° C. for 10 min, then the supernatant was taken and stored at −80° C. After all samples were collected, the concentrations of the cytotoxic drugs were detected by LC-MS/MS. The results were shown in FIG. 4, and the shedding of the cytotoxic drugs did not exceed 5%, indicating that the anti-human HER3 antibody-drug conjugate of the present invention (e.g., 22B6-A-07-A-1) was stable in plasma of different species.

### 5. Detection of In Vivo Drug Efficacy of Anti-Human HER3 Antibody-Drug Conjugates in H358 Model

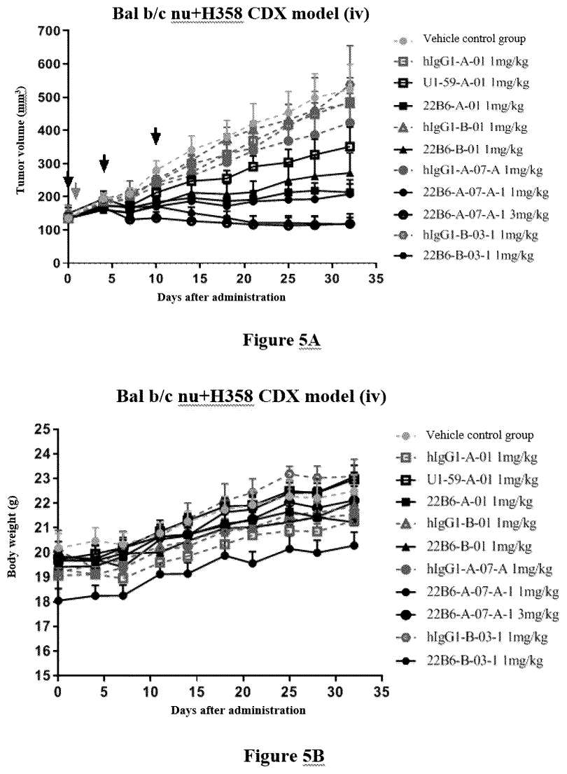

NCI-H358 cells were cultured at 37° C., 5% CO₂ in RPMI1640 medium containing 10% fetal bovine serum. The NCI-H358 cells in the exponential growth phase were collected, resuspended in PBS to an appropriate concentration, and inoculated subcutaneously in female BALB/c-nu mice to establish a non-small cell lung cancer model. When the average volume of the tumor was about 130 to 150 mm³, the animals were randomly divided into 11 groups according to tumor size, including: vehicle control group (i.e., negative control group), hIgG1-A-01 1 mg/kg group, U1-59-A-01 1 mg/kg group, ADC 22B6-A-01 1 mg/kg group, hIgG1-B-01 1 mg/kg group, ADC 22B6-B-01 1 mg/kg group, hIgG1-A-07-A 1 mg/kg group, ADC 22B6-A-07-A-1 1 mg/kg and 3 mg/kg groups, hIgG1-B-03-1 1 mg/kg group, ADC 22B6-B-03-1 1 mg/kg group. All animals were administered via injection through tail vein on Day 0, Day 1, Day 4 and Day 10, 3 times in total. After administration, the tumor diameter was measured twice a week with a vernier caliper, and the tumor volume was calculated according to the following formula: V=0.5 a×b², wherein a and b represented the long and short diameters of the tumor, respectively, and the death of animals was observed and recorded every day. The specific results were shown in Table 34, FIG. 5A, FIG. 5B.

The following formula was used to calculate the tumor growth inhibition rate TGI (%), which was used to evaluate the tumor inhibitory efficacy:

$$TGI ( \% ) = [ 1 - ( {VT}_{end} - {VT}_{start} ) / ( {VC}_{end} - {VC}_{start} ) ] * 100 \%$$

wherein,
VT_{end} represented a mean value of tumor volume at the end of the experiment in a treatment group
VTₛₜₐᵣₜ represented a mean value of tumor volume at the beginning of the administration in a treatment group
VC_{end} represented a mean value of tumor volume at the end of the experiment in the negative control group
VCₛₜₐᵣₜ represented a mean value of tumor volume at the beginning of the administration in the negative control group

It could be seen from the experimental results that the ADCs of the present invention had a significant tumor growth inhibitory effect on the NCI-H358 non-small cell lung cancer xenograft model. Compared with the vehicle control group, after 3 administrations, the Day 32 data showed that the tumor growth inhibition rate (TGI) of the ADC 22B6-A-01 1 mg/kg group and the 22B6-B-01 1 mg/kg group were 79.80% and 64.68%, the TGI of the 22B6-A-07-A-1 1 mg/kg group and 3 mg/kg group were 81.22%, 111.53% respectively, and the TGI of the 22B6-B-03-1 1 mg/kg group was 121.45%; the TGI of the positive control U1-59-A-01 1 mg/kg group was 44.51%. On Day 32, there was no animal death and significant animal body weight loss in each of the treatment groups, and no obvious drug toxicity was observed. During the treatment period, the mice were well tolerated to the ADCs of the present invention.

TABLE 34
Human non-small cell lung cancer cell NCI-H358 CDX model

| | | Day 32 | | | |
|---|---|---|---|---|---|
| | | | | | P value |
| | | Tumor volume | | | (vs. |
| Group | | (mm³) | TGI | T/C | Group |
| No. | Group name | (x̅ ± SEM) | (%) | (%) | 1) |
| 1 | Vehicle | 523.79 ± 75.33 | — | — | — |
| | control group | | | | |
| 2 | hIgG1-A-01 | 485.69 ± 72.91 | 9.68 | 93.02 | 0.724 |
| | 1 mg/kg | | | | |
| 3 | U1-59-A-01 | 350.75 ± 58.99 | 44.51 | 66.92 | 0.101 |
| | 1 mg/kg | | | | |
| 4 | 22B6-A-01 | 213.68 ± 24.79 | 79.80 | 40.70 | 0.003 |
| | 1 mg/kg | | | | |
| 5 | hIgG1-B-01 | 481.61 ± 30.05 | 10.89 | 91.83 | 0.614 |
| | 1 mg/kg | | | | |
| 6 | 22B6-B-01 | 271.94 ± 60.53 | 64.68 | 52.02 | 0.026 |
| | 1 mg/kg | | | | |
| 7 | hIgG1-A-07-A | 422.70 ± 66.85 | 26.04 | 80.57 | 0.339 |
| | 1 mg/kg | | | | |
| 8 | 22B6-A-07-A-1 | 208.35 ± 42.59 | 81.22 | 39.63 | 0.004 |
| | 1 mg/kg | | | | |
| 9 | 22B6-A-07-A-1 | 118.71 ± 29.81 | 111.53 | 22.77 | 0.003 |
| | 3 mg/kg | | | | |
| 10 | hIgG1-B-03-1 | 537.77 ± 117.56 | 0.05 | 92.90 | 0.920 |
| | 1 mg/kg | | | | |
| 11 | 22B6-B-03-1 | 117.40 ± 20.09 | 121.45 | 20.22 | 0.003 |
| | 1 mg/kg | | | | |

Note:
TGI represented tumor growth inhibition rate, T/C represented relative tumor proliferation rate, the same below. (In this experiment, Group 1 to Group 9 were grouped and administered on Day 0, and had a tumor volume (TV) ≈ 135 mm³; Group 10 and Group 11 were grouped and administered on Day 1, and had a tumor volume (TV) ≈ 149 mm³; after that, all groups were administered together on Day 4 and Day 10).

### 6. Detection of In Vivo Drug Efficacy of Anti-Human HER3 Antibody-Drug Conjugates in N87 Model

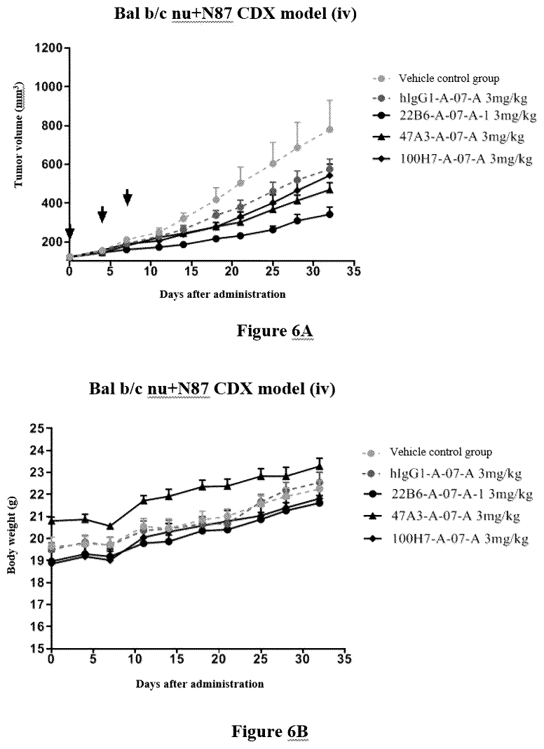

NCI-N87 cells were cultured at 37° C. with 5% CO₂ in RPMI1640 medium containing 10% fetal bovine serum. The NCI-N87 cells in the exponential growth phase were collected, resuspended in PBS to an appropriate concentration, and inoculated subcutaneously in female BALB/c-nu mice to establish a human gastric cancer model. When the average volume of tumor was about 120 mm³, the animals are randomly divided into groups according to the tumor size, including: vehicle control group, hIgG1-A-07-A 3 mg/kg group, ADC 22B6-A-07-A-1 3 mg/kg group, ADC 47A3-A-07-A 3 mg/kg group, and ADC 100H7-A-07-A 3 mg/kg group. All animals were administered via injection through tail vein on Day 0, Day 4 and Day 7, and 3 times in total. After administration, the tumor diameter was measured twice a week with a vernier caliper, and the tumor volume was calculated according to the following formula: V=0.5 a×b², wherein a and b represented the long and short diameters of tumor, respectively, and the death of animals was observed and recorded every day. The specific results were shown in Table 35, FIGS. 6A and 6B.

It could be seen from the experimental results that, as compared with the vehicle control group, after 3 administrations, the Day32 data showed that the ADC 22B6-A-07-A-1 3 mg/kg group, 47A3-A-07-A 3 mg/kg group, and 100H7-A-07-A 3 mg/kg group had a growth inhibition rate (TGI) of 66.59%, 47.24% and 36.04%, respectively; the ADC 22B6-A-07-A-1 3 mg/kg group and 47A3-A-07-A 3 mg/kg group showed a significant inhibitor effect on the tumor growth in the NCI-N87 gastric cancer xenograft model. On Day 32, there was no animal death and significant animal body weight loss in each of the treatment groups, and no obvious drug toxicity was observed. During the treatment period, the mice were well tolerated to the ADCs of the present invention.

TABLE 35
Human gastric cancer cell NCI-N87 CDX model

| | | Day 32 | | | |
|---|---|---|---|---|---|
| | | | | | P value |
| | | Tumor volume | | | (vs. |
| Group | | (mm³) | TGI | T/C | Group |
| No. | Group name | (x̅ ± SEM) | (%) | (%) | 1) |
| 1 | Vehicle | 779.52 ± 150.34 | — | — | — |
| | control group | | | | |
| 2 | hIgG1-A-07-A | 574.78 ± 52.79 | 31.21 | 73.40 | 0.228 |
| | 3 mg/kg | | | | |
| 3 | 22B6-A-07-A-1 | 342.37 ± 37.22 | 66.59 | 43.61 | 0.018 |
| | 3 mg/kg | | | | |
| 4 | 47A3-A-07-A | 468.82 ± 36.48 | 47.24 | 60.12 | 0.072 |
| | 3 mg/kg | | | | |
| 5 | 100H7-A-07-A | 542.62 ± 58.59 | 36.04 | 69.52 | 0.173 |
| | 3 mg/kg | | | | |

### 7. Detection of In Vivo Drug Efficacy of Anti-Human HER3 Antibody-Drug Conjugates in MDA-MB-453 Model

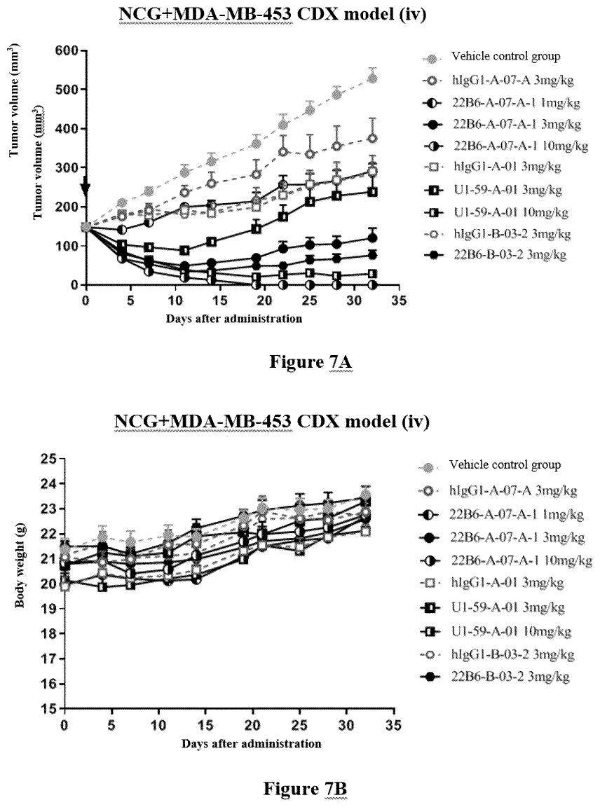

Human breast cancer cell line MDA-MB-453 was cultured in DMEM/F12 medium containing 10% fetal bovine serum at 37° C. and 5% CO₂. The MDA-MB-453 cells in the exponential growth phase were collected, resuspended to an appropriate concentration by adding PBS and Matrigel at 50% of the final concentration, and inoculated subcutaneously in female NCG immunodeficient mice to establish a human breast cancer xenograft tumor model. When the average volume of tumor was about 150 mm³, the animals were randomly divided into groups according to the tumor size, including: vehicle control group, hIgG1-A-07-A 3 mg/kg group, ADC 22B6-A-07-A-1 1 mg/kg group, 3 mg/kg group and 10 mg/kg group, hIgG1-A-01 3 mg/kg group, U1-59-A-01 3 mg/kg group and 10 mg/kg group, hIgG1-B-03-2 3 mg/kg group, ADC 22B6-B-03-2 3 mg/kg group. After the grouping, the animals were given a single dose via injection through tail vein on Day0. After administration, the tumor volume and body weight of the mice were observed and regularly measured, and the specific results were shown in Table 36, and FIGS. 7A, 7B.

All of the ADCs of the present invention had a significant inhibitory effect on the tumor growth of NCG mice implanted subcutaneously with human breast cancer MDA-MB-453. The data collected on Day 32 after giving a single dose showed that, as compared with the vehicle control group, the TGI values of the 22B6-A-07-A-1, 1 mg/kg group, 3 mg/kg group and 10 mg/kg group were 62.07%, 118.30% and 200.00%, respectively, wherein the tumors in the 10 mg/kg group were completely regressed from Day 19, and no growth was observed by Day32; the TGI value in the 22B6-B-03-2 3 mg/kg group was 148.58%; the TGI values of the positive control U1-59-A-01 3 mg/kg group and 10 mg/kg group were 76.11% and 180.65%, respectively. During the treatment period, the mice were well tolerated to the ADCs of the present invention.

TABLE 36
Human breast cancer cell MDA-MB-453 CDX model

| | | Day 32 | | | | |
|---|---|---|---|---|---|---|
| | | | | | Tumor | |
| | | Tumor volume | | | regression | |
| Group | | (mm³) | TGI | T/C | CR/PR | P value |
| No. | Group name | (x̅ ± SEM) | (%) | (%) | (n = 6) | (vs. Group 1) |
| 1 | Vehicle control | 528.12 ± 27.78 | — | — | 0/0 | — |
| | group | | | | | |
| 2 | hIgG1-A-07-A | 375.13 ± 51.92 | 0.13 | 71.44 | 0/0 | 0.027 |
| | 3 mg/kg | | | | | |
| 3 | 22B6-A-07-A-1 | 291.74 ± 18.54 | 62.07 | 55.64 | 0/0 | 0.000 |
| | 1 mg/kg | | | | | |
| 4 | 22B6-A-07-A-1 | 120.03 ± 25.22 | 118.30 | 23.05 | 0/3 | 0.000 |
| | 3 mg/kg | | | | | |
| 5 | 22B6-A-07-A-1 | 0.00 ± 0.00 | 200.00 | 0.00 | 6/0 | 0.000 |
| | 10 mg/kg | | | | | |
| 6 | hIgG1-A-01 | 290.74 ± 23.06 | 62.42 | 55.32 | 0/0 | 0.000 |
| | 3 mg/kg | | | | | |
| 7 | U1-59-A-01 | 238.41 ± 42.09 | 76.11 | 45.50 | 0/1 | 0.000 |
| | 3 mg/kg | | | | | |
| 8 | U1-59-A-01 | 28.59 ± 2.19 | 180.65 | 5.46 | 0/6 | 0.000 |
| | 10 mg/kg | | | | | |
| 9 | hIgG1-B-03-2 | 283.75 ± 47.85 | 64.12 | 54.18 | 0/1 | 0.001 |
| | 3 mg/kg | | | | | |
| 10 | 22B6-B-03-2 | 75.87 ± 13.04 | 148.58 | 14.51 | 0/6 | 0.000 |
| | 3 mg/kg | | | | | |

### 8. Detection of Cell Affinity of Anti-Human HER3 Antibody and its Drug Conjugates

The affinity of anti-human HER3 ADC to MDA-MB-453 cells was detected by flow cytometry (Beckman, model Cytoflex).

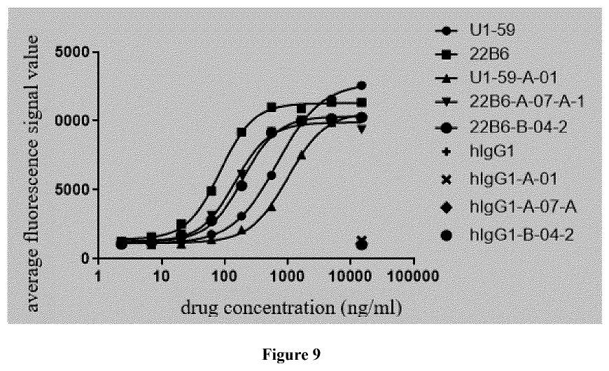

Adherent MDA-MB-453 cells were digested with Tryple (manufacturer: Gibco) solution, counted, and an appropriate amount of the cells were taken, washed twice with 1×PBS, resuspended in 1% BSA solution, and then transferred to a 96-well sharp-bottom plate, 50 μl per well; candidate antibodies and drug conjugates thereof were diluted with 1% BSA by 3-fold serial dilution starting from 15 μg/mL, and then 50 μl of the diluted antibody or antibody-drug conjugate was added to the sharp-bottom plate containing the cells, and incubation was carried out at 4° C. for 40 minutes; the cells were washed twice with PBS, then added with 50 μl of diluted secondary antibody to each well, mixed, and incubated at 4° C. for 30 minutes; the cells were washed with PBS twice, then resuspended in 200 μl of PBS and detected by flow cytometry. Data processing: the average fluorescence signal values were exported, and then input into GraphPad Prism 6 software to calculate EC50. The results were shown in Table 37 and FIG. 9. The above results showed that, the affinity to cells of the ADCs obtained by conjugation of the antibodies of the present invention (e.g., 22B6) was significantly higher than that of the ADCs obtained by conjugating control antibody U1-59.

TABLE 37
Determination results of cell affinity
of anti-human HER3 antibody and ADC

| | EC50(ng/ml) |
|---|---|
| Drug | MDA-MB-453 |
| 22B6-A-07-A-1 | 146.9 |
| 22B6-B-04-2 | 194.9 |
| U1-59-A-01 | 1014 |
| U1-59 | 684.2 |
| 22B6 | 85.39 |

### 9. Detection of Endocytosis Effects of Anti-Human HER3 Antibody Conjugates

The endocytosis effects of anti-human HER3 ADC in MDA-MB-453 cells were detected by flow cytometry (Beckman, model Cytoflex).

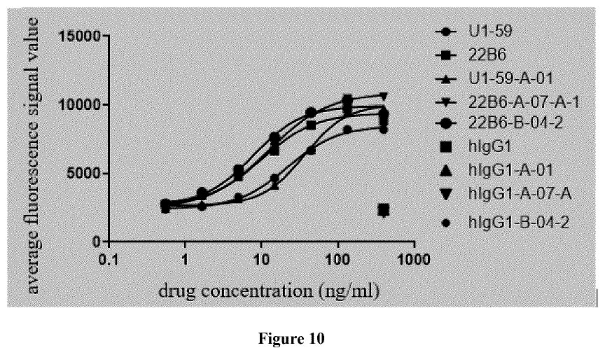

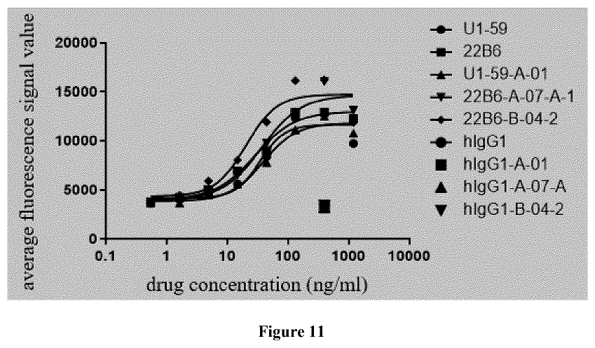

Adherent cells were digested with Trypsin-EDTA (0.25% Shanghai Basal Media Technologies) solution, counted, and adjusted the cell concentration to 1×105/ml with complete culture medium, then 100 μl of the cell suspension was added to the 96-well plate (the cell number is 1× 104/well), the 96-well plate was incubated in constant temperature CO₂ incubator at 37° C. for 24 hours. Then took out the 96-well plate, discarded the medium, and added 50 μl of fresh complete culture medium to each well, the bi-specific antibody and control antibody were diluted with complete culture medium by serial dilution, the final concentrations were 0.55, 1.64, 4.94, 14.81, 44.44, 133.33, 400, 1200 ng/ml, 8 concentrations in total; PHrodo solution (Thermo) was diluted with complete culture medium to 12 μg/ml (the final concentration of PHrodo is 3 μg/ml); the serial diluted antibody candidates and diluted PHrodo solution were 1:1 mixed (30 μl 30 μl), incubated at room temperature in dark for 30 minutes; add 50 μl antibody candidate and PHrodo solution mixture to 96-well bplace, cultured at 37° C., 5% CO₂ for 24 hours; took out the 96-well plate, discarded medium, washed once with sterile PBS, then cells were digested with 100 μl/well Trypsin-EDTA (0.25%), neutralized with 100 μl complete culture medium; then cells were separated by piping up and down and detected by flow cytometry. Data processing: the average fluorescence signal values were exported, and then input into GraphPad Prism 6 software to calculate EC50. The results were shown in Table 38 and FIGS. 10 and 11, which indicated that the endocytosis effects of 22B6 and its conjugated ADC were better or not worse than the control antibody U1-59 and its conjugated ADC.

TABLE 38
Determination results of endocytosis effects
of anti-human HER3 antibody and ADC

EC50(ng/ml)

| Drug | MDA-MB-453 | HCC1569 |
|---|---|---|
| 22B6-A-07-A-1 | 12.22 | 36.91 |
| 22B6-B-04-2 | 7.76 | 20.43 |
| U1-59-A-01 | 36.69 | 38.97 |
| U1-59 | 21.81 | 34.72 |
| 22B6 | 9.98 | 28.77 |

### 10. In Vitro Cytotoxicity of Anti-Human HER3 Antibody-Drug Conjugates

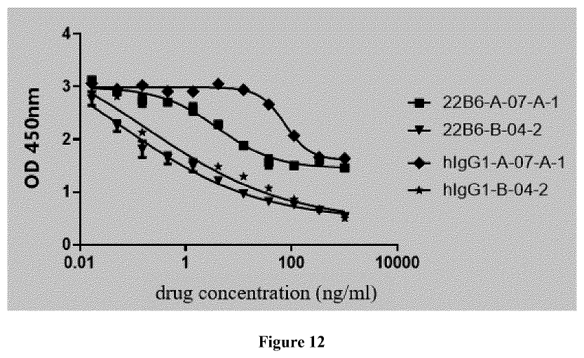

MDA-MB-453 cells were digested with TrypLE (manufacturer: Gibco) solution, counted, and an appropriate amount of the cells were taken, diluted with growth medium, plated on a 96-well plate, 5000 cells (100 μl)/well, cultured overnight at 37° C., 5% CO₂; on the next day, ADC was diluted with the growth medium by 2.5-fold gradient dilution starting from 150 μg/ml, and then 100 μl of the diluted ADC was taken, added to the 96-well plate containing the cells, the plate was placed into an incubator and incubated at 37° C. and 5% CO₂ for 6 days, then 20 μl of CCK8 was added to each well, incubation was conducted for 2-5 h at 37° C., and the OD450 nm absorbance value was read with a microplate reader; the original data were input into Graph Prism 6 to calculate IC50. As shown in Table 39 and FIG. 12, both of 22B6-A-07-A-1 and 22B6-B-04-2 have significant killing effects in MDA-MB-453 cells.

TABLE 39
Determination results of In vitro cytotoxicity
of anti-human HER3 antibody-drug conjugates

| | IC50(nM) |
|---|---|
| Drug | MDA-MB-453 |
| hIgG1-A-07-A | 29.4 |
| 22B6-A-07-A-1 | 4.0 |
| hIgG1-B-04-2 | 0.18 |
| 22B6-B-04-2 | 0.07 |

Note:
“/” stands for no detection.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

What is claimed is:

1. An antibody-drug conjugate, which has a structure as shown in formula
Ab-[M-L-E-D]ₓ, wherein:
Ab is an antibody or antigen-binding fragment thereof that specifically binds to human epidermal growth factor receptor 3;
M is a joint site to bind the antibody or antigen-binding fragment thereof;
L is a connector that connects the joint site M and E;
E is a fragment connecting L and D;
D is a fragment of a cytotoxic drug;
x is selected from 1 to 10.

2. The antibody-drug conjugate according to claim 1, wherein
the antibody or antigen-binding fragment thereof is characterized by any one of the following items (I) to (IV):
(I) the antibody or antigen-binding fragment thereof comprises:
(1) a heavy chain variable region (VH) and/or a light chain variable region (VL) as follows, wherein the CDRs are defined by the Chothia numbering system:
(1a) a heavy chain variable region (VH) comprising the following 3 CDRs: CDR-H1 having an amino acid sequence as set forth in SEQ ID NO: 1 or a variant thereof, CDR-H2 having an amino acid sequence as set forth in SEQ ID NO: 2 or a variant thereof, CDR-H3 having an amino acid sequence as set forth in SEQ ID NO: 3 or a variant thereof; and/or, a light chain variable region (VL) comprising the following 3 CDRs: CDR-L1 having an amino acid sequence as set forth in SEQ ID NO: 4 or a variant thereof, CDR-L2 having an amino acid sequence as set forth in SEQ ID NO: 5 or a variant thereof, CDR-L3 having an amino acid sequence as set forth in SEQ ID NO: 6 or a variant thereof, or,
(1b) a heavy chain variable region (VH) comprising the following 3 CDRs: CDR-H1 having an amino acid sequence as set forth in SEQ ID NO: 19 or a variant thereof, CDR-H2 having an amino acid sequence as set forth in SEQ ID NO: 20 or a variant thereof, CDR-H3 having an amino acid sequence as set forth in SEQ ID NO: 21 or a variant thereof; and/or, a light chain variable region (VL) comprising the following 3 CDRs: CDR-L1 having an amino acid sequence as set forth in SEQ ID NO: 22 or a variant thereof, CDR-L2 having an amino acid sequence as set forth in SEQ ID NO: 23 or a variant thereof, CDR-L3 having an amino acid sequence as set forth in SEQ ID NO: 24 or a variant thereof; or,
(1c) a heavy chain variable region (VH) comprising the following 3 CDRs: CDR-H1 having an amino acid sequence as set forth in SEQ ID NO: 36 or a variant thereof, CDR-H2 having an amino acid sequence as set forth in SEQ ID NO: 37 or a variant thereof, CDR-H3 having an amino acid sequence as set forth in SEQ ID NO: 38 or a variant thereof; and/or, a light chain variable region (VL) comprising the following 3 CDRs: CDR-L1 having an amino acid sequence as set forth in SEQ ID NO: 45 or a variant thereof, CDR-L2 having an amino acid sequence as set forth in SEQ ID NO: 23 or a variant thereof, and CDR-L3 having an amino acid sequence as set forth in SEQ ID NO: 52 or a variant thereof;
wherein, the variant described in any item of (1a), (1b), and (1c) has an amino acid sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the amino acid sequence from which it is derived, or the variant has a substitution, deletion or addition of one or several amino acids as compared to the amino acid sequence from which it is derived with the proviso that the amino acid sequences of the CDRs of the variant have 100% sequence identity to the amino acid sequences of the respective CDRs of the VH and VL of (1a), (1b), and (1c) and the variant binds HER3; or,
(2) a heavy chain variable region (VH) and/or a light chain variable region (VL) as follows, wherein the CDRs are defined according to the Kabat numbering system:
(2a) a heavy chain variable region (VH) comprising the following 3 CDRs: CDR-H1 having an amino acid sequence as set forth in SEQ ID NO: 7 or a variant thereof, CDR-H2 having an amino acid sequence as set forth in SEQ ID NO: 8 or a variant thereof, CDR-H3 having an amino acid sequence as set forth in SEQ ID NO: 9 or a variant thereof; and/or, a light chain variable region (VL) comprising the following 3 CDRs: CDR-L1 having an amino acid sequence as set forth in SEQ ID NO: 4 or a variant thereof, CDR-L2 having an amino acid sequence as set forth in SEQ ID NO: 5 or a variant thereof, CDR-L3 having an amino acid sequence as set forth in SEQ ID NO: 6 or a variant thereof; or,
(2b) a heavy chain variable region (VH) comprising the following 3 CDRs: CDR-H1 having an amino acid sequence as set forth in SEQ ID NO: 25 or a variant thereof, CDR-H2 having an amino acid sequence as set forth in SEQ ID NO: 26 or a variant thereof, CDR-H3 having an amino acid sequence as set forth in SEQ ID NO: 21 or a variant thereof; and/or, a light chain variable region (VL) comprising the following 3 CDRs: CDR-L1 having an amino acid sequence as set forth in SEQ ID NO: 22 or a variant thereof, CDR-L2 having an amino acid sequence as set forth in SEQ ID NO: 23 or a variant thereof, CDR-L3 having an amino acid sequence as set forth in SEQ ID NO: 24 or a variant thereof; or,
(2c) a heavy chain variable region (VH) comprising the following 3 CDRs: CDR-H1 having an amino acid sequence as set forth in SEQ ID NO: 39 or a variant thereof, CDR-H2 having an amino acid sequence as set forth in SEQ ID NO: 40 or a variant thereof, CDR-H3 having an amino acid sequence as set forth in SEQ ID NO: 38 or a variant thereof; and/or, a light chain variable region (VL) comprising the following 3 CDRs: CDR-L1 having an amino acid sequence as set forth in SEQ ID NO: 45 or a variant thereof, CDR-L2 having an amino acid sequence as set forth in SEQ ID NO: 23 or a variant thereof, and CDR-L3 having an amino acid sequence as set forth in SEQ ID NO: 52 or a variant thereof;
wherein, the variant described in any item of (2a), (2b), and (2c) has an amino acid sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the amino acid sequence from which it is derived, or the variant has a substitution, deletion or addition of one or several amino acids as compared to the amino acid sequence from which it is derived with the proviso that the amino acid sequences of the CDRs of the variant have 100% sequence identity to the amino acid sequences of the respective CDRs of the VH and VL of (2a), (2b), and (2c) and the variant binds HER3; or,
(3) a heavy chain variable region (VH) and/or a light chain variable region (VL) as follows, wherein the CDRs are defined by the IMGT numbering system:
(3a) a heavy chain variable region (VH) comprising the following 3 CDRs: CDR-H1 having an amino acid sequence as set forth in SEQ ID NO: 10 or a variant thereof, CDR-H2 having an amino acid sequence as set forth in SEQ ID NO: 11 or a variant thereof, CDR-H3 having an amino acid sequence as set forth in SEQ ID NO: 12 or a variant thereof; and/or, a light chain variable region (VL) comprising the following 3 CDRs: CDR-L1 having an amino acid sequence as set forth in SEQ ID NO: 13 or a variant thereof, CDR-L2 having an amino acid sequence as set forth in SEQ ID NO: 14 or a variant thereof, CDR-L3 having an amino acid sequence as set forth in SEQ ID NO: 6 or a variant thereof, or,
(3b) a heavy chain variable region (VH) comprising the following 3 CDRs: CDR-H1 having an amino acid sequence as set forth in SEQ ID NO: 27 or a variant thereof, CDR-H2 having an amino acid sequence as set forth in SEQ ID NO: 28 or a variant thereof, CDR-H3 having an amino acid sequence as set forth in SEQ ID NO: 29 or a variant thereof; and/or, a light chain variable region (VL) comprising the following 3 CDRs: CDR-L1 having an amino acid sequence as set forth in SEQ ID NO: 30 or a variant thereof, CDR-L2 having an amino acid sequence as set forth in SEQ ID NO: 31 or a variant thereof, CDR-L3 having an amino acid sequence as set forth in SEQ ID NO: 24 or a variant thereof; or,
(3c) a heavy chain variable region (VH) comprising the following 3 CDRs: CDR-H1 having an amino acid sequence as set forth in SEQ ID NO: 41 or a variant thereof, CDR-H2 having an amino acid sequence as set forth in SEQ ID NO: 42 or a variant thereof, CDR-H3 having an amino acid sequence as set forth in SEQ ID NO: 43 or a variant thereof; and/or, a light chain variable region (VL) comprising the following 3 CDRs: CDR-L1 having an amino acid sequence as set forth in SEQ ID NO: 44 or a variant thereof, CDR-L2 having an amino acid sequence as set forth in SEQ ID NO: 31 or a variant thereof, and CDR-L3 having an amino acid sequence as set forth in SEQ ID NO: 52 or a variant thereof;
wherein, the variant described in any item of (3a), (3b), and (3c) has an amino acid sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the amino acid sequence from which it is derived, or the variant has a substitution, deletion or addition of one or several amino acids as compared to the amino acid sequence from which it is derived with the proviso that the amino acid sequences of the CDRs of the variant have 100% sequence identity to the amino acid sequences of the respective CDRs of the VH and VL of (3a), (3b), and (3c) and the variant binds HER3;
(II) the antibody or antigen-binding fragment thereof comprises:
(a) a VH comprising the amino acid sequence as set forth in SEQ ID NO: 15 or a variant thereof, and/or, a VL comprising the amino acid sequence as set forth in SEQ ID NO: 16 or a variant thereof;
(b) a VH comprising the amino acid sequence as set forth in SEQ ID NO: 32 or a variant thereof, and/or, a VL comprising the amino acid sequence as set forth in SEQ ID NO: 33 or a variant thereof; or
(c) a VH comprising the amino acid sequence as set forth in SEQ ID NO: 46 or a variant thereof, and/or, a VL comprising the amino acid sequence as set forth in SEQ ID NO: 47 or a variant thereof;
wherein, the variant has an amino acid sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the amino acid sequence from which it is derived, or the variant has a substitution, deletion or addition of one or several amino acids as compared to the amino acid sequence from which it is derived with the proviso that the amino acid sequences of the CDRs of the variant have 100% sequence identity to the amino acid sequences of the respective CDRs of the VH and VL of (a), (b), and (c) and the variant binds HER3;
(III) the antibody or antigen-binding fragment thereof further comprises:
(a) a human immunoglobulin heavy chain constant region (CH) or a variant thereof, wherein, the variant has a substitution, deletion or addition of one or more amino acids as compared to the wild-type sequence from which it is derived; and
(b) a human immunoglobulin light chain constant region (CL) or a variant thereof, wherein the variant has a substitution, deletion or addition of one or more amino acids as compared to the wild-type sequence from which it is derived;
(IV) the antibody or antigen-binding fragment thereof comprises:
(1) a heavy chain comprising a VH comprising the amino acid sequence as set forth in SEQ ID NO: 15 and a heavy chain constant region (CH) comprising the amino acid sequence as set forth in SEQ ID NO: 50, and, a light chain comprising a VL comprising the amino acid sequence as set forth in SEQ ID NO: 16 and a light chain constant region (CL) comprising the amino acid sequence as set forth in SEQ ID NO: 51;
(2) a heavy chain comprising a VH comprising the amino acid sequence as set forth in SEQ ID NO: 32 and a heavy chain constant region (CH) comprising the amino acid sequence as set forth in SEQ ID NO: 50, and, a light chain comprising a VL comprising the amino acid sequence as set forth in SEQ ID NO: 33 and a light chain constant region (CL) comprising the amino acid sequence as set forth in SEQ ID NO: 51; or
(3) a heavy chain comprising a VH comprising the amino acid sequence as set forth in SEQ ID NO: 46 and a heavy chain constant region (CH) comprising the amino acid sequence as set forth in SEQ ID NO: 50, and, a light chain comprising a VL comprising the amino acid sequence as set forth in SEQ ID NO: 47 and a light chain constant region (CL) comprising the amino acid sequence as set forth in SEQ ID NO: 51.

3-5. (canceled)

6. The antibody-drug conjugate according to claim 1, wherein M is characterized by any one of the following items (1) to (5);
(1) M is 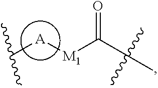
wherein, Ring A is a 5- to 6-membered heteroaliphatic ring, or a 5- to 20-membered aromatic ring system, and the heteroaliphatic ring and aromatic ring system are optionally substituted with one or more groups selected from the group consisting of oxy (═O), halogen, cyano, amino, carboxyl, sulfhydryl group and C₁₋₆ alkyl; M₁ is selected from the group consisting of single bond, C₁₋₂₀ alkylene, C₂₋₂₀ alkenylene, and C₂₋₂₀ alkynylene;
(2) M is 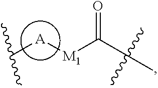
wherein Ring A is a 5-membered heteroaliphatic ring, 6-membered heteroaromatic ring, or a polycyclic ring formed by one or more 6-membered heteroaromatic rings and a benzene ring connected through a single bond, wherein the heteroaliphatic ring is optionally substituted with one or more groups selected from the group consisting of oxy (═O), halogen and C₁₋₄ alkyl; and M₁ is selected from the group consisting of single bond, C₃₋₁₀ alkylene, C₃₋₁₀ alkenylene and C₃₋₁₀ alkynylene;
(3) M is 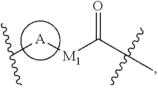
wherein Ring A is selected from the group consisting of 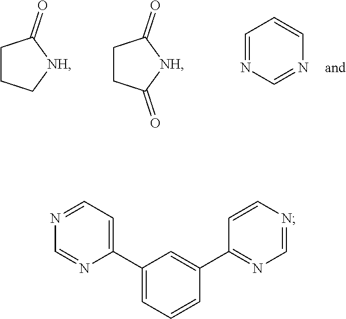 [SMILES: O=C1CCCN1 ; O=C1CCC(=O)N1 ; c1cncnc1 ; c1cc(-c2ccncn2)cc(-c2ccncn2)c1]
and M₁ is selected from the group consisting of single bond, C₅₋₈ alkylene, C₅₋₈ alkenylene, and C₅₋₈ alkynylene;
(4) M is 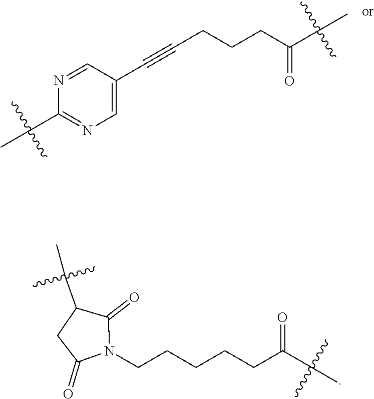
(5) M is

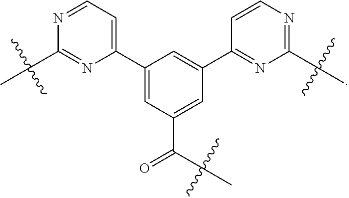

7-10. (canceled)

11. The antibody-drug conjugate according to claim 1, wherein L is characterized by any one of the following items (1) to (5);
(1) L is a structure composed of one or more selected from the following: C₁₋₆ alkylene, —N(R′)—, carbonyl, —O—, Val, Cit, Phe, Lys, Lys (COCH₂CH₂ (OCH₂CH₂)ₛOCH₃), D-Val, Leu, Gly, Ala, Asn, Val-Cit, Val-Ala, Val-Lys, Val-Lys (Ac), Phe-Lys, Phe-Lys (Ac), D-Val-Leu-Lys, Gly-Gly-Arg, Ala-Ala-Asn, Ala-Ala-Ala, Val-Lys-Ala, Val-Lys-Gly, Gly-Gly-Gly, Gly-Gly-Phe-Gly, Gly-Gly-Gly-Gly-Gly, 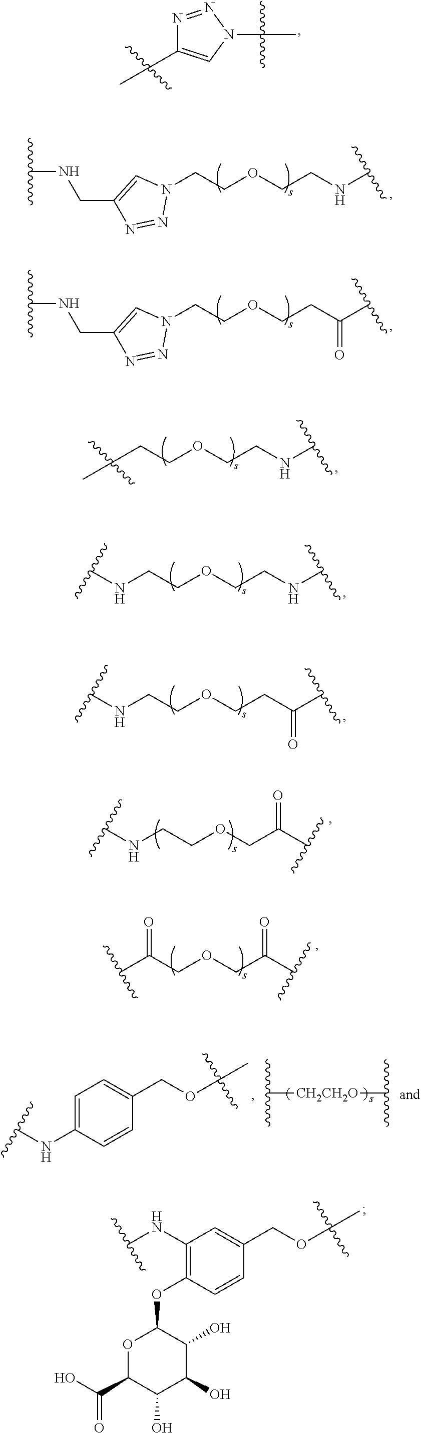
wherein R′ represents hydrogen, C₁₋₆ alkyl or alkyl containing —(CH₂CH₂O)ᵣ—; r is an integer selected from 1 to 10; and s is an integer selected from 1 to 20;
(2) L is a structure composed of one or more selected from the following: C₁₋₆ alkylene, —NH—, Phe, Lys, Lys (COCH₂CH₂ (OCH₂CH₂)ₛOCH₃), Gly, Gly-Gly-Phe-Gly, 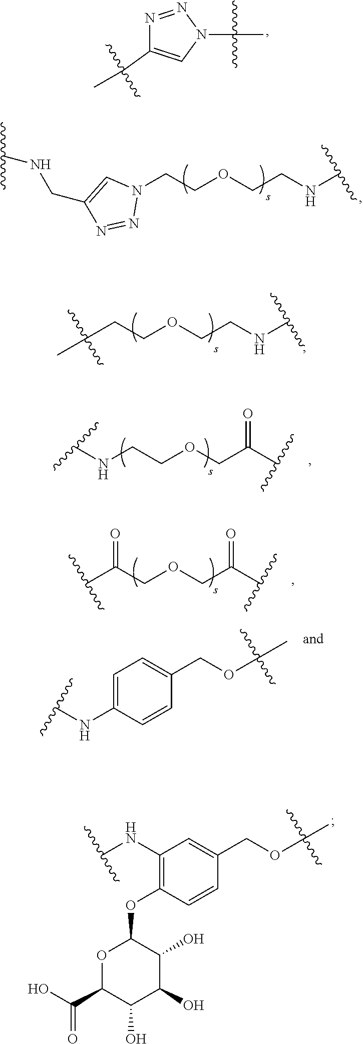
wherein s is an integer selected from 1 to 20;
(3) L is selected from the following structures: 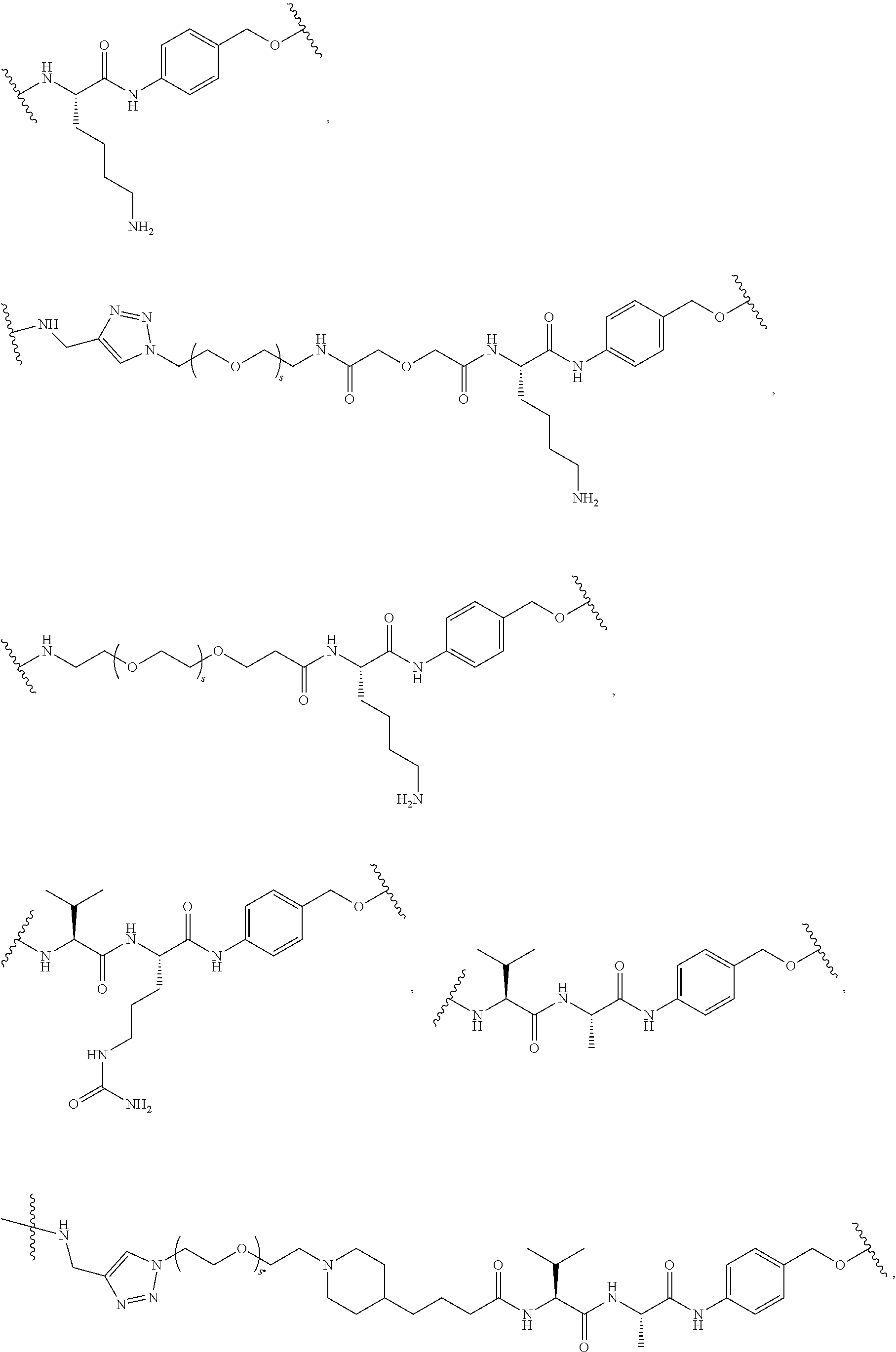 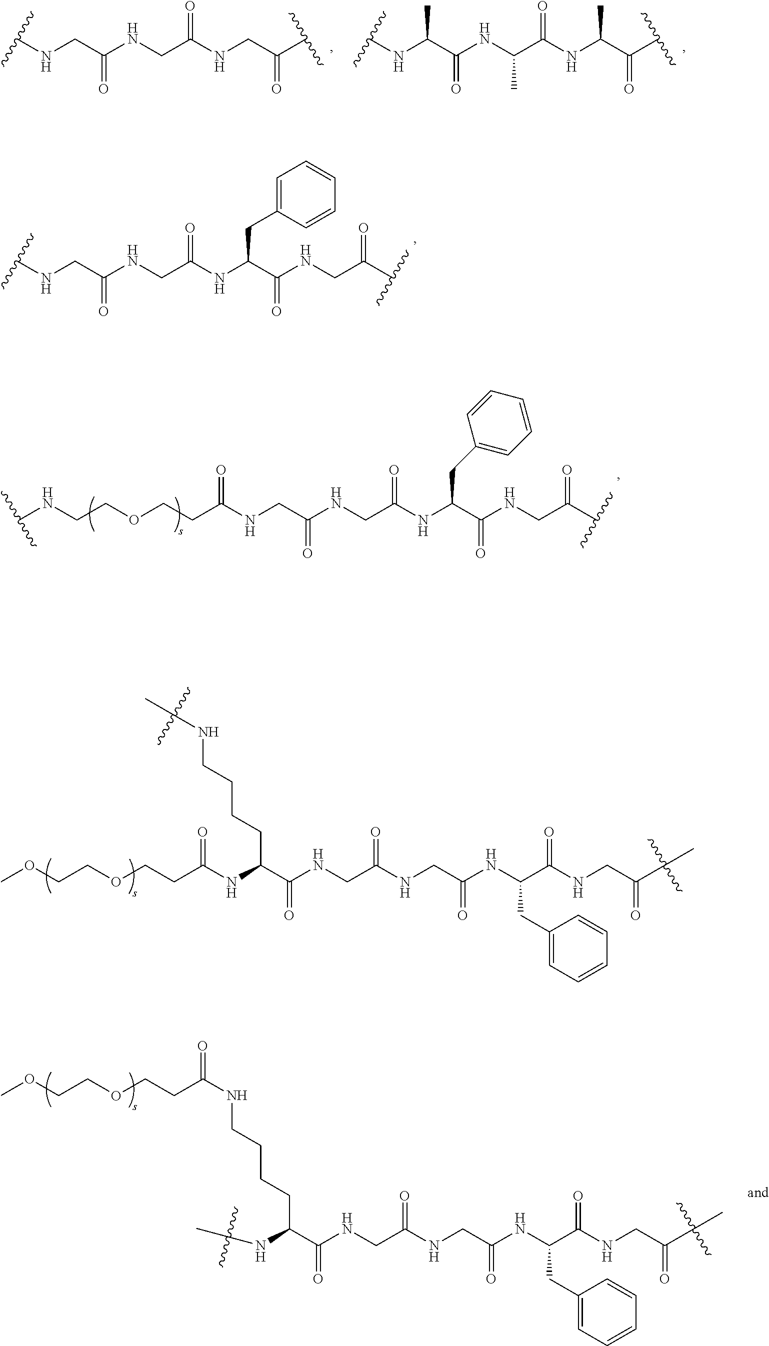 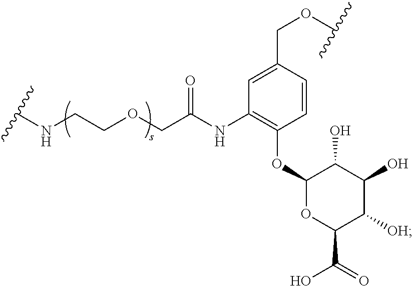
wherein s is an integer selected from 1 to 20;
(4) L is selected from the following structures: 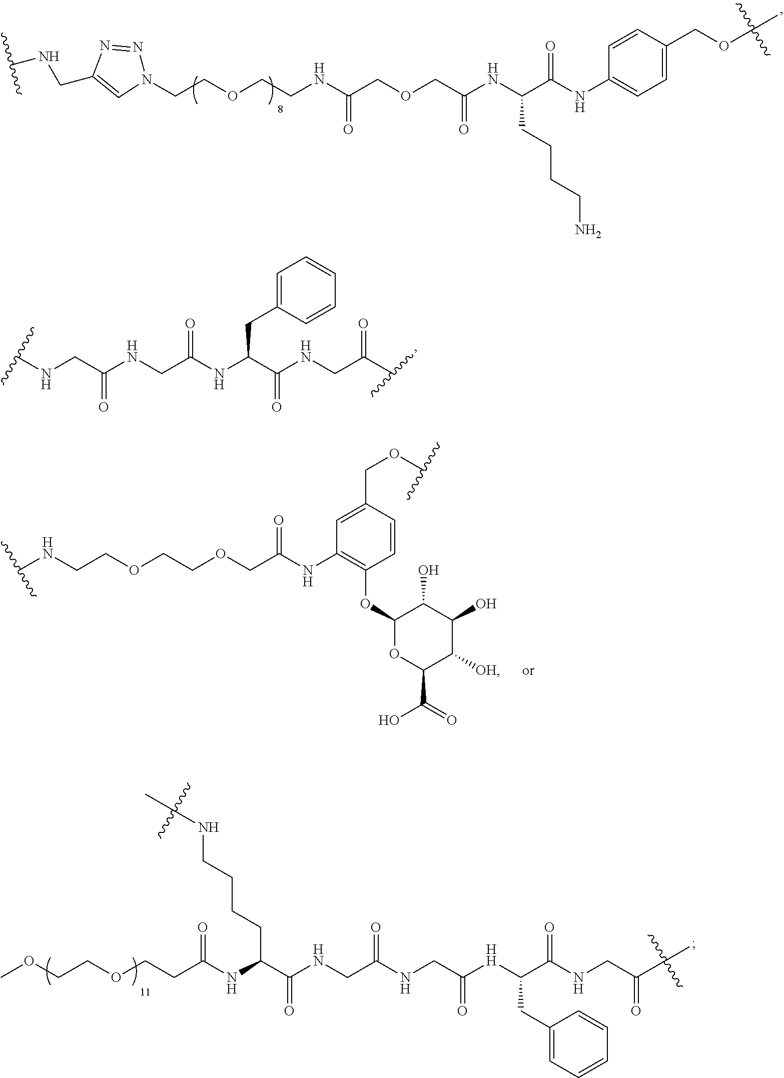
(5) L is selected from the following structures:

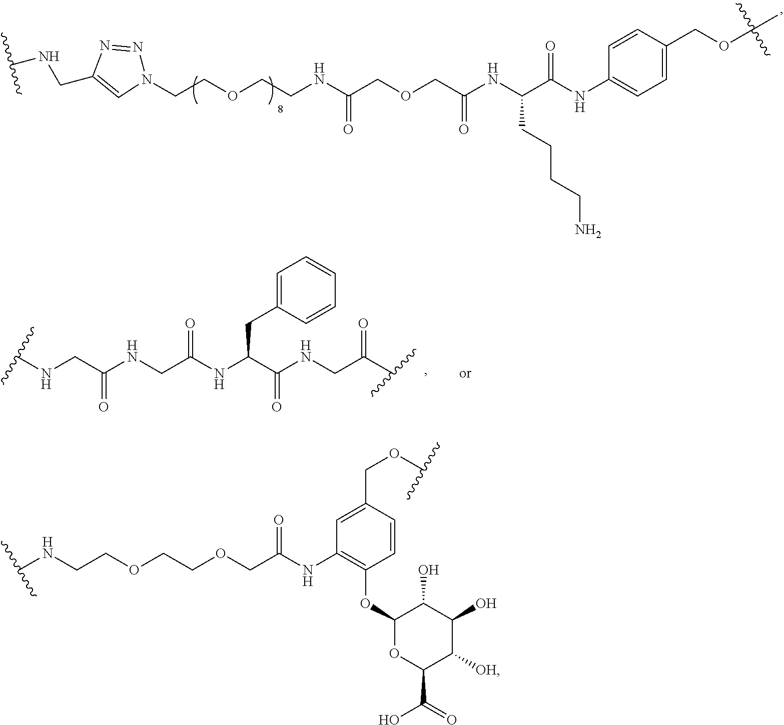

12-15. (canceled)

16. The antibody-drug conjugate according to claim 1, wherein E is characterized by any one of the following items (1) to (4);
(1) E is a single bond, —NH—CH₂—, —NH—CH₂—O—CH₂—CO—, or is selected from the following structures: 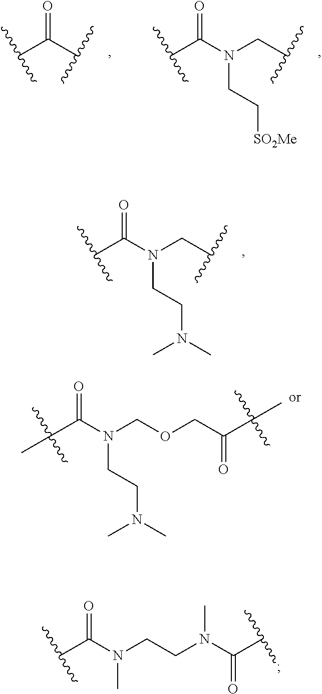
(2) E is a single bond, —NH—CH₂—, —NH—CH₂—O—CH₂—CO—, 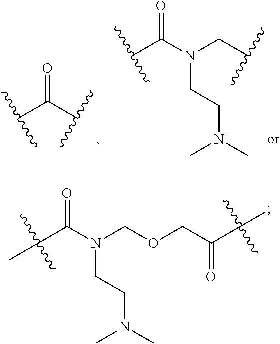
(3) E is-NH—CH₂—O—CH₂—CO—, 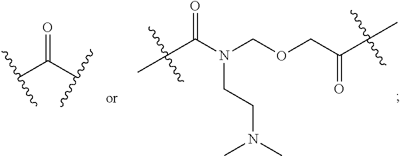
(4) E is-NH—CH₂—O—CH₂—CO— or

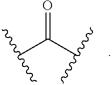 [SMILES: *C(*)=O ; "."]

17-19. (canceled)

20. The antibody-drug conjugate according to claim 1, wherein 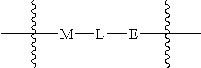
is selected from the following structures:

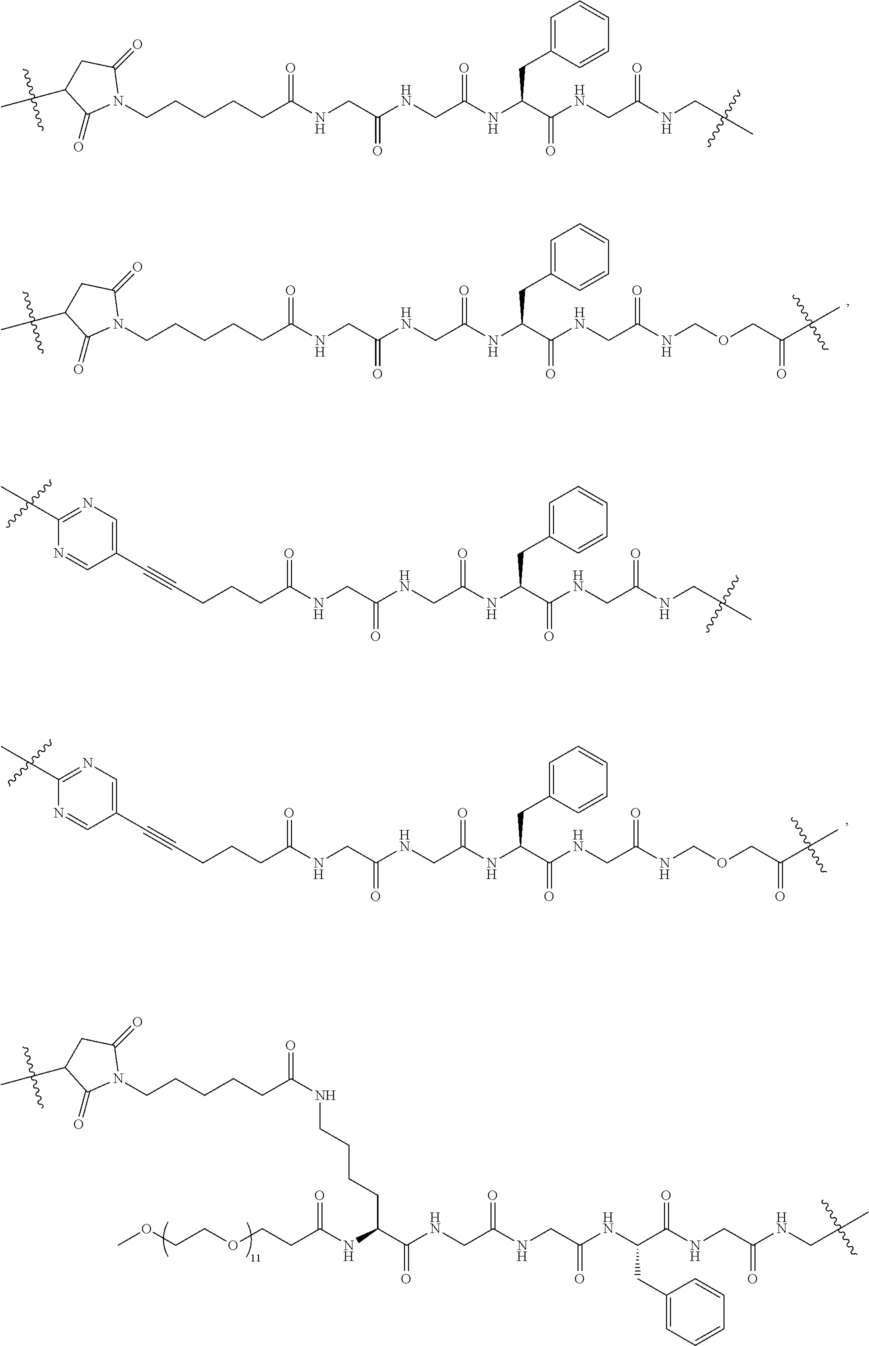

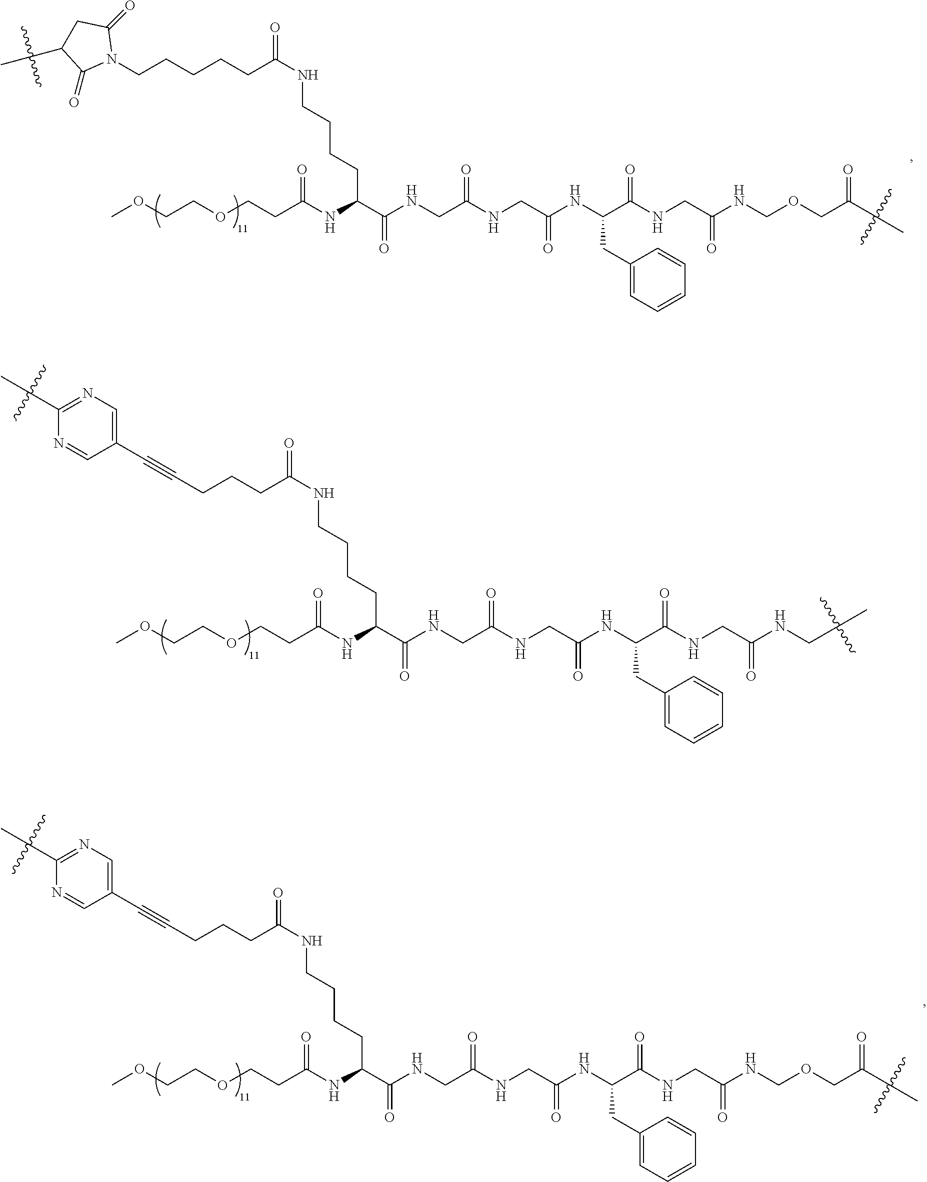

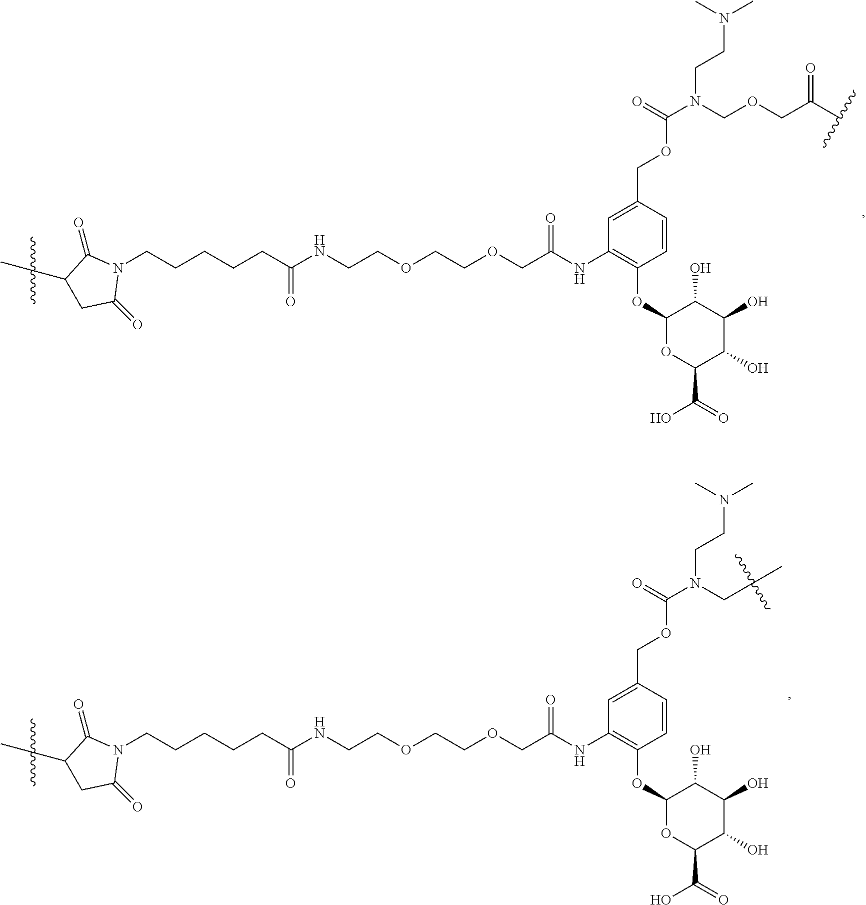 [SMILES: *C(=O)COCN(CCN(C)C)C(=O)OCc1ccc(O[C@@H]2O[C@H](C(=O)O)[C@@H](O)[C@H](O)[C@H]2O)c(NC(=O)COCCOCCNC(=O)CCCCCN2C(=O)CC(*)C2=O)c1 ; *CN(CCN(C)C)C(=O)OCc1ccc(O[C@@H]2O[C@H](C(=O)O)[C@@H](O)[C@H](O)[C@H]2O)c(NC(=O)COCCOCCNC(=O)CCCCCN2C(=O)CC(*)C2=O)c1]

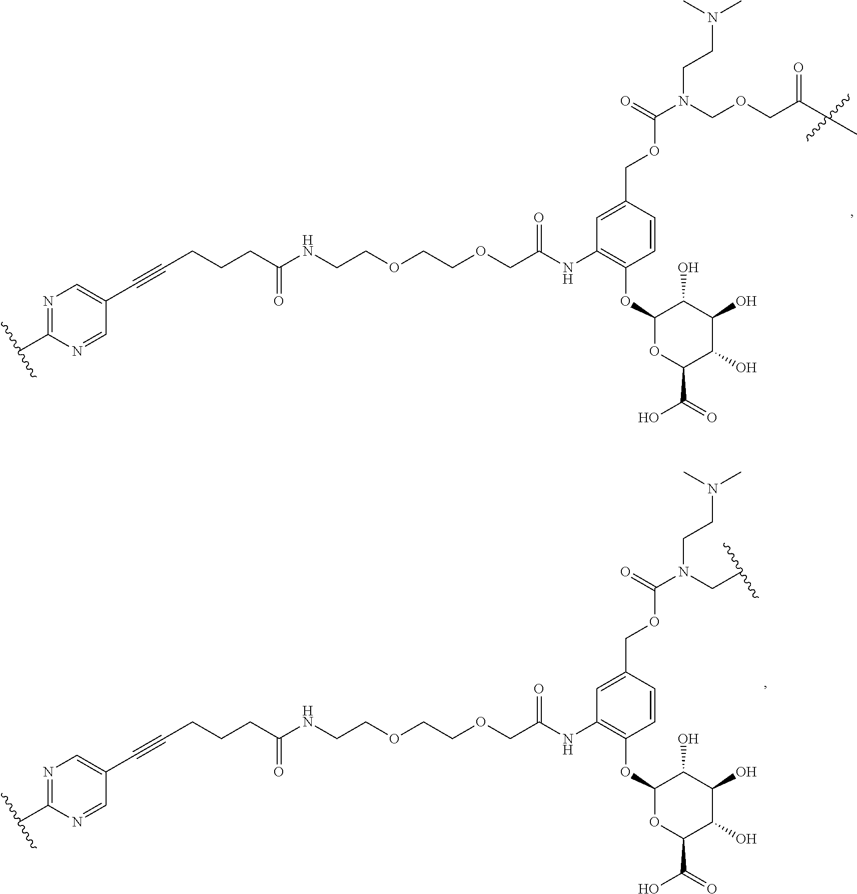 [SMILES: *C(=O)COCN(CCN(C)C)C(=O)OCc1ccc(O[C@@H]2O[C@H](C(=O)O)[C@@H](O)[C@H](O)[C@H]2O)c(NC(=O)COCCOCCNC(=O)CCCC#Cc2cnc(*)nc2)c1 ; *CN(CCN(C)C)C(=O)OCc1ccc(O[C@@H]2O[C@H](C(=O)O)[C@@H](O)[C@H](O)[C@H]2O)c(NC(=O)COCCOCCNC(=O)CCCC#Cc2cnc(*)nc2)c1]

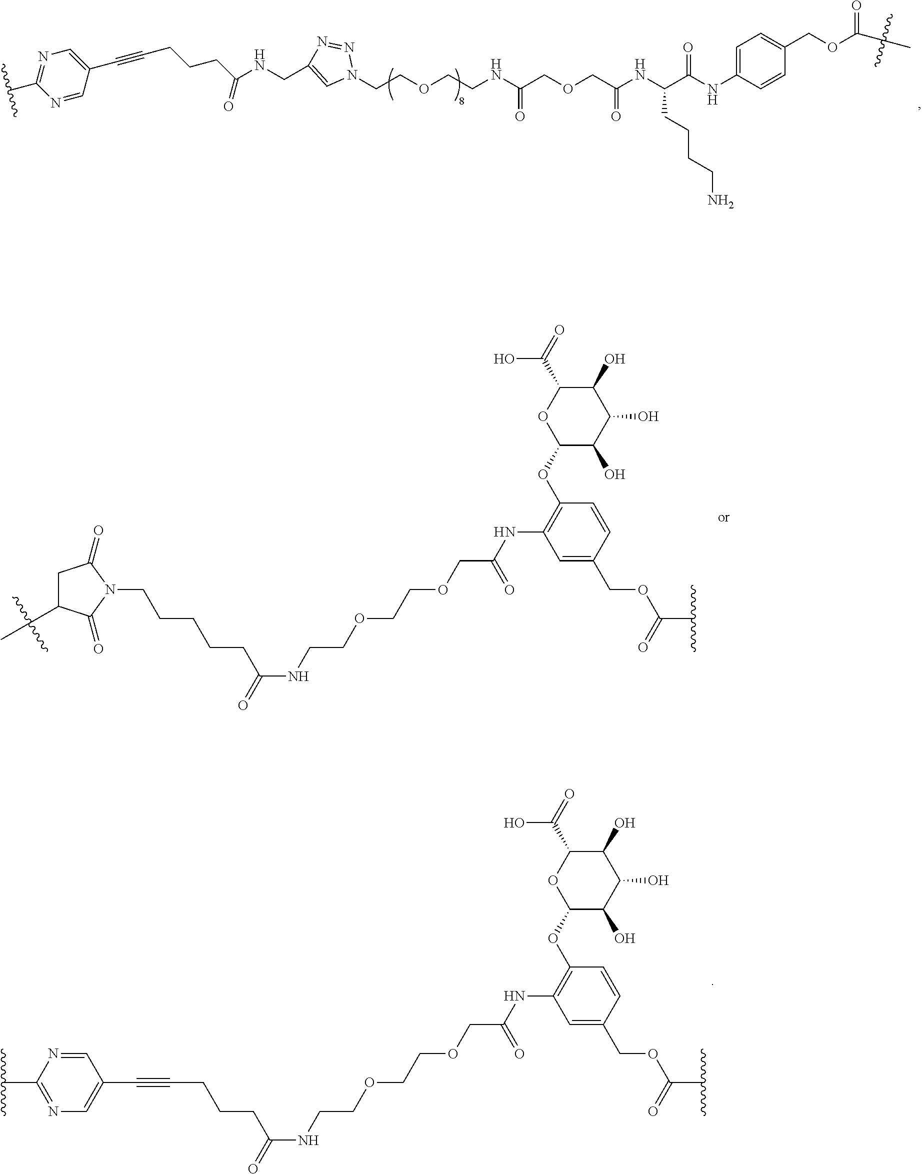

21. The antibody-drug conjugate according to claim 1, wherein the cytotoxic drug is selected from the group consisting of tubulin inhibitors, DNA intercalators, DNA topoisomerase inhibitors, and RNA polymerase inhibitors, or pharmaceutically acceptable salt, ester or analog thereof;
particularly, the tubulin inhibitor is an auristatin compound or a maytansine compound, or a pharmaceutically acceptable salt, ester or analog thereof;
the DNA intercalator is pyrrolobenzodiazepine (PBD), or a pharmaceutically acceptable salt, ester or analog thereof;
the DNA topoisomerase inhibitor is a topoisomerase I inhibitor or a topoisomerase II inhibitor, or a pharmaceutically acceptable salt, ester or analog thereof;
the topoisomerase I inhibitor is a camptothecin, hydroxycamptothecin, 9-aminocamptothecin, SN-38, irinotecan, topotecan, belotecan, or rubitecan, or a pharmaceutically acceptable salt, ester or analog thereof;
the topoisomerase II inhibitor is doxorubicin, PNU-159682, duocarmycin, daunorubicin, mitoxantrone, podophyllotoxin, or etoposide, or a pharmaceutically acceptable salt, ester or analog thereof; and/or
the RNA polymerase inhibitor is α-amanitin, or a pharmaceutically acceptable salt, ester or analog thereof;
more particularly, the cytotoxic drug is selected from the compounds as shown in Formulas I and II: 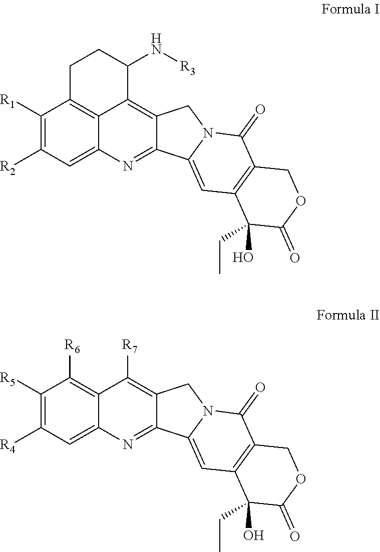
wherein, R₁ and R₂ are each independently selected from the group consisting of C₁₋₆ alkyl and halogen;
R₃ is selected from the group consisting of H and —CO—CH₂OH;
R₄ and R₅ are each independently selected from the group consisting of H, halogen and hydroxyl; or R₄ and R₅ together with the carbon atoms to which they are connected form a 5- to 6-membered oxygen-containing heterocycle;
R₆ is selected from the group consisting of hydrogen and —C₁₋₄ alkylene-NRₐR_{b};
R₇ is selected from the group consisting of C₁₋₆ alkyl and —C₁-4 alkylene-NRₐR_{b};
wherein, at each occurrence, Rₐ and R_{b} are each independently selected from the group consisting of H, C₁₋₆ alkyl, —SO₂—C₁₋₆ alkyl and —CO—C₁₋₆ alkyl;
further particularly, the cytotoxic drug is selected from the group consisting of the following compounds: 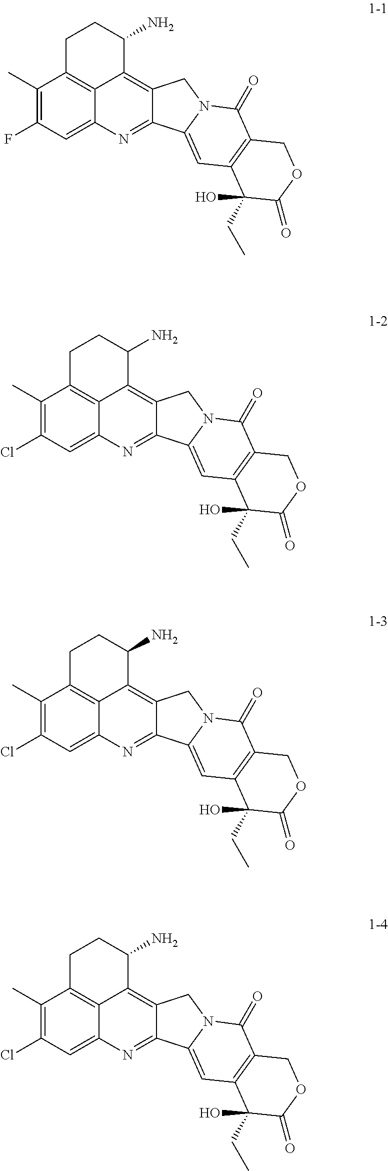 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(Cl)c(C)c3c1c2[C@H](N)CC3 ; CC[C@@]1(O)C(=O)OCc2c1cc1n(c2=O)Cc2c-1nc1cc(Cl)c(C)c3c1c2[C@@H](N)CC3] 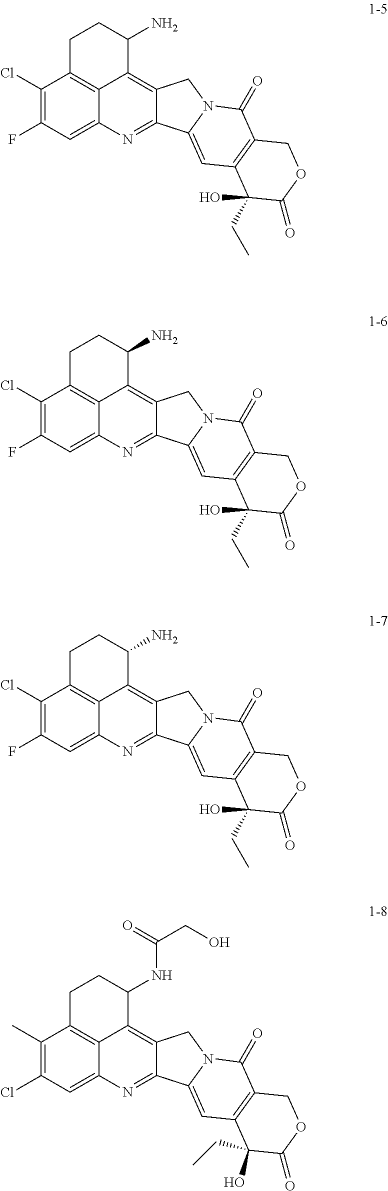 [SMILES: CC[C@@]1(O)C(=O)OCc2c1cc1n(c2=O)Cc2c-1nc1cc(F)c(Cl)c3c1c2C(N)CC3 ; CC[C@@]1(O)C(=O)OCc2c1cc1n(c2=O)Cc2c-1nc1cc(F)c(Cl)c3c1c2[C@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2C(NC(=O)CO)CC3] 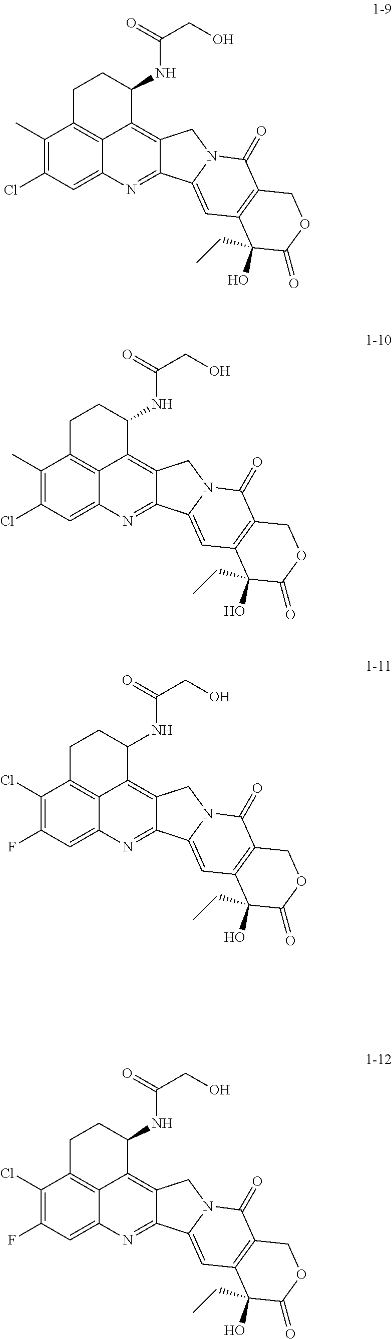 [SMILES: CC[C@@]1(O)C(=O)OCc2c1cc1n(c2=O)Cc2c-1nc1cc(Cl)c(C)c3c1c2[C@H](NC(=O)CO)CC3 ; CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2C(NC(=O)CO)CC3 ; CC[C@@]1(O)C(=O)OCc2c1cc1n(c2=O)Cc2c-1nc1cc(F)c(Cl)c3c1c2[C@H](NC(=O)CO)CC3] 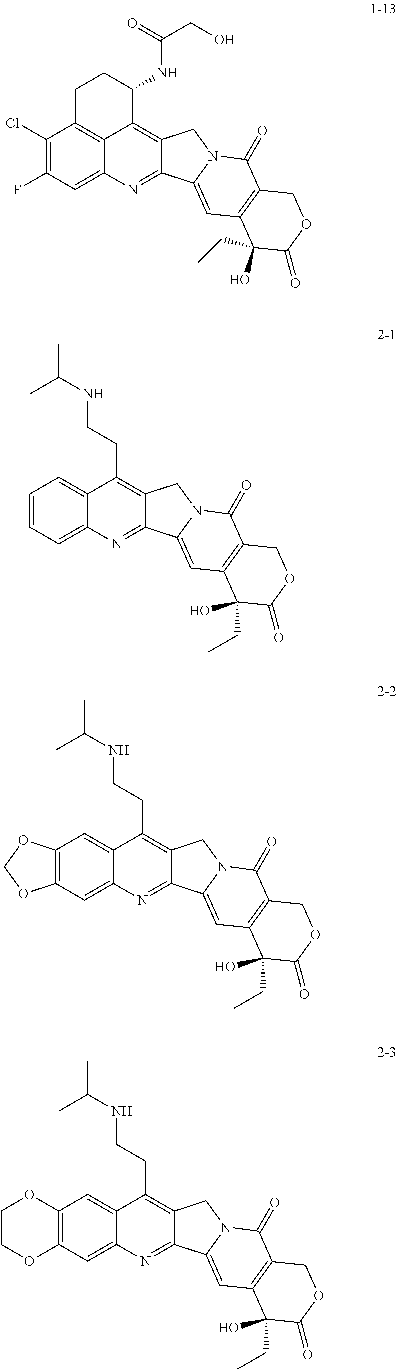 [SMILES: CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1ccccc1c2CCNC(C)C ; CC[C@@]1(O)C(=O)OCc2c1cc1n(c2=O)Cc2c-1nc1cc3c(cc1c2CCNC(C)C)OCO3 ; CC[C@@]1(O)C(=O)OCc2c1cc1n(c2=O)Cc2c-1nc1cc3c(cc1c2CCNC(C)C)OCCO3] 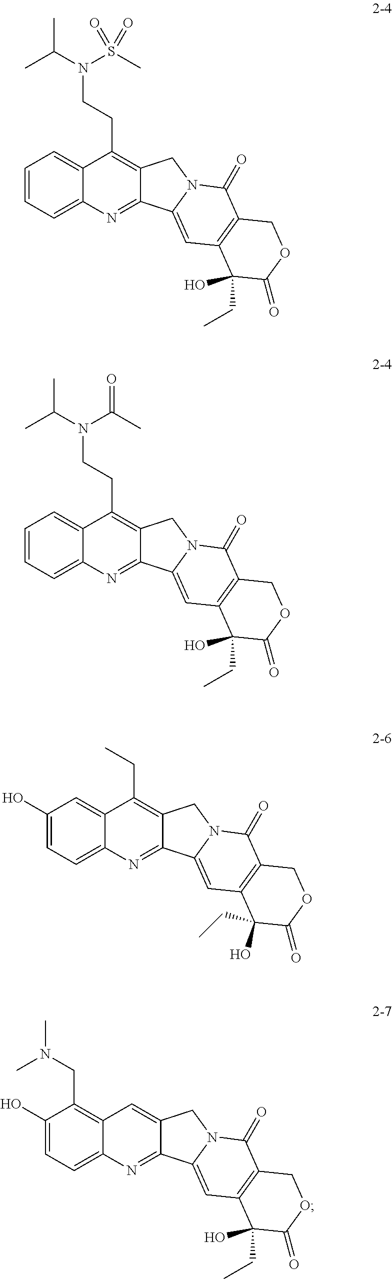 [SMILES: CC[C@@]1(O)C(=O)OCc2c1cc1n(c2=O)Cc2c-1nc1ccccc1c2CCN(C(C)C)S(C)(=O)=O ; CC[C@@]1(O)C(=O)OCc2c1cc1n(c2=O)Cc2c-1nc1ccccc1c2CCN(C(C)=O)C(C)C ; CCc1c2c(nc3ccc(O)cc13)-c1cc3c(c(=O)n1C2)COC(=O)[C@]3(O)CC ; CC[C@@]1(O)C(=O)OCc2c1cc1n(c2=O)Cc2cc3c(CN(C)C)c(O)ccc3nc2-1]
wherein the corresponding fragment of the cytotoxic drug after being connected with the linker D of the formula.

22-29. (canceled)

30. The antibody-drug conjugate according to claim 1, wherein D is a monovalent structure obtained by losing one H from —OH, —NH₂ or the secondary amine group on the cytotoxic drug;
particularly, D is selected from:

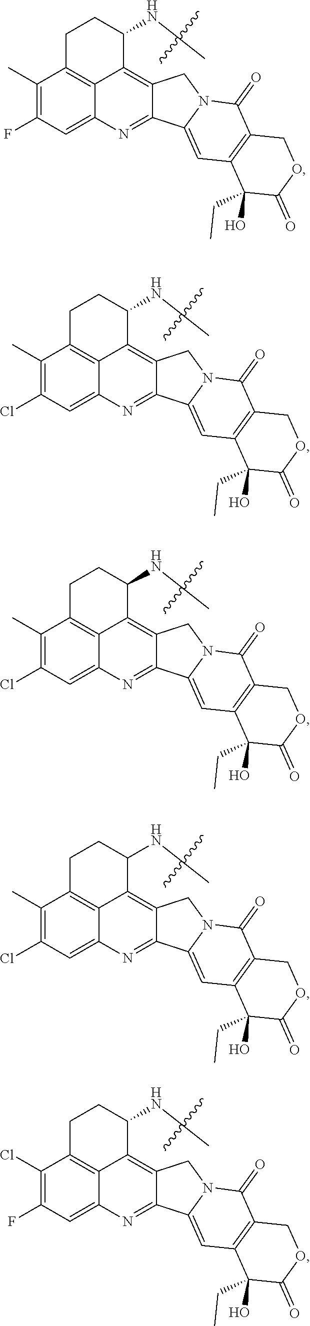 [SMILES: *N[C@H]1CCc2c(C)c(F)cc3nc4c(c1c23)Cn1c-4cc2c(c1=O)COC(=O)[C@]2(O)CC ; *N[C@H]1CCc2c(C)c(Cl)cc3nc4c(c1c23)Cn1c-4cc2c(c1=O)COC(=O)[C@]2(O)CC ; *N[C@@H]1CCc2c(C)c(Cl)cc3nc4c(c1c23)Cn1c-4cc2c(c1=O)COC(=O)[C@]2(O)CC ; *NC1CCc2c(C)c(Cl)cc3nc4c(c1c23)Cn1c-4cc2c(c1=O)COC(=O)[C@]2(O)CC ; *N[C@H]1CCc2c(Cl)c(F)cc3nc4c(c1c23)Cn1c-4cc2c(c1=O)COC(=O)[C@]2(O)CC]

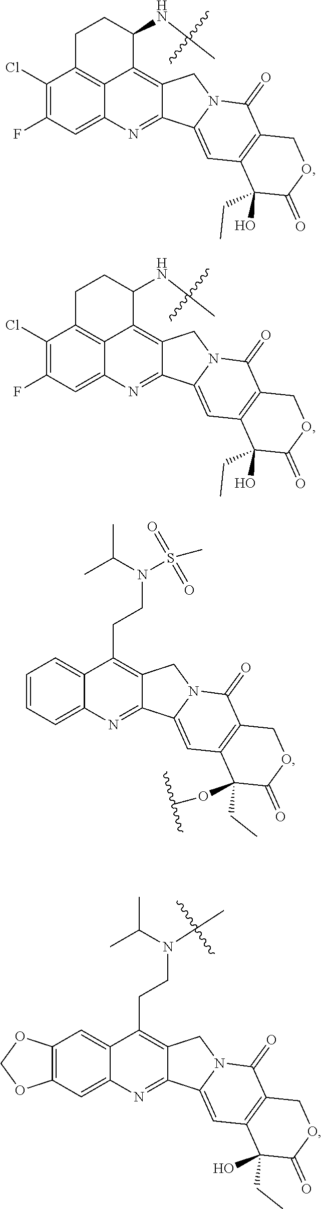 [SMILES: *N[C@@H]1CCc2c(Cl)c(F)cc3nc4c(c1c23)Cn1c-4cc2c(c1=O)COC(=O)[C@]2(O)CC ; *NC1CCc2c(Cl)c(F)cc3nc4c(c1c23)Cn1c-4cc2c(c1=O)COC(=O)[C@]2(O)CC ; *O[C@]1(CC)C(=O)OCc2c1cc1n(c2=O)Cc2c-1nc1ccccc1c2CCN(C(C)C)S(C)(=O)=O ; *N(CCc1c2c(nc3cc4c(cc13)OCO4)-c1cc3c(c(=O)n1C2)COC(=O)[C@]3(O)CC)C(C)C]

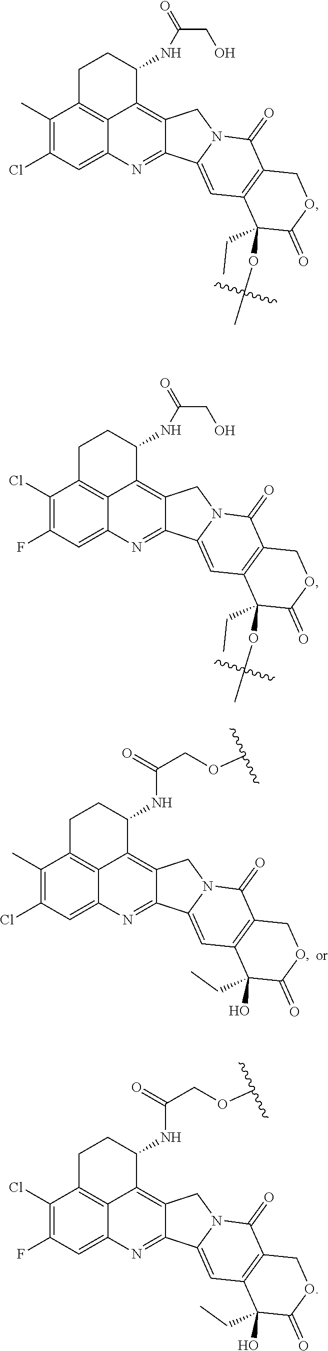 [SMILES: *O[C@]1(CC)C(=O)OCc2c1cc1n(c2=O)Cc2c-1nc1cc(Cl)c(C)c3c1c2[C@@H](NC(=O)CO)CC3 ; *O[C@]1(CC)C(=O)OCc2c1cc1n(c2=O)Cc2c-1nc1cc(F)c(Cl)c3c1c2[C@@H](NC(=O)CO)CC3 ; *OCC(=O)N[C@H]1CCc2c(C)c(Cl)cc3nc4c(c1c23)Cn1c-4cc2c(c1=O)COC(=O)[C@]2(O)CC ; *OCC(=O)N[C@H]1CCc2c(Cl)c(F)cc3nc4c(c1c23)Cn1c-4cc2c(c1=O)COC(=O)[C@]2(O)CC]

31. (canceled)

32. The antibody-drug conjugate according to claim 1, wherein the antibody-drug conjugate:
(1) is selected from the group consisting of ADC A-01 to ADC A-25, ADC B-01 to ADC B-05 as shown below: 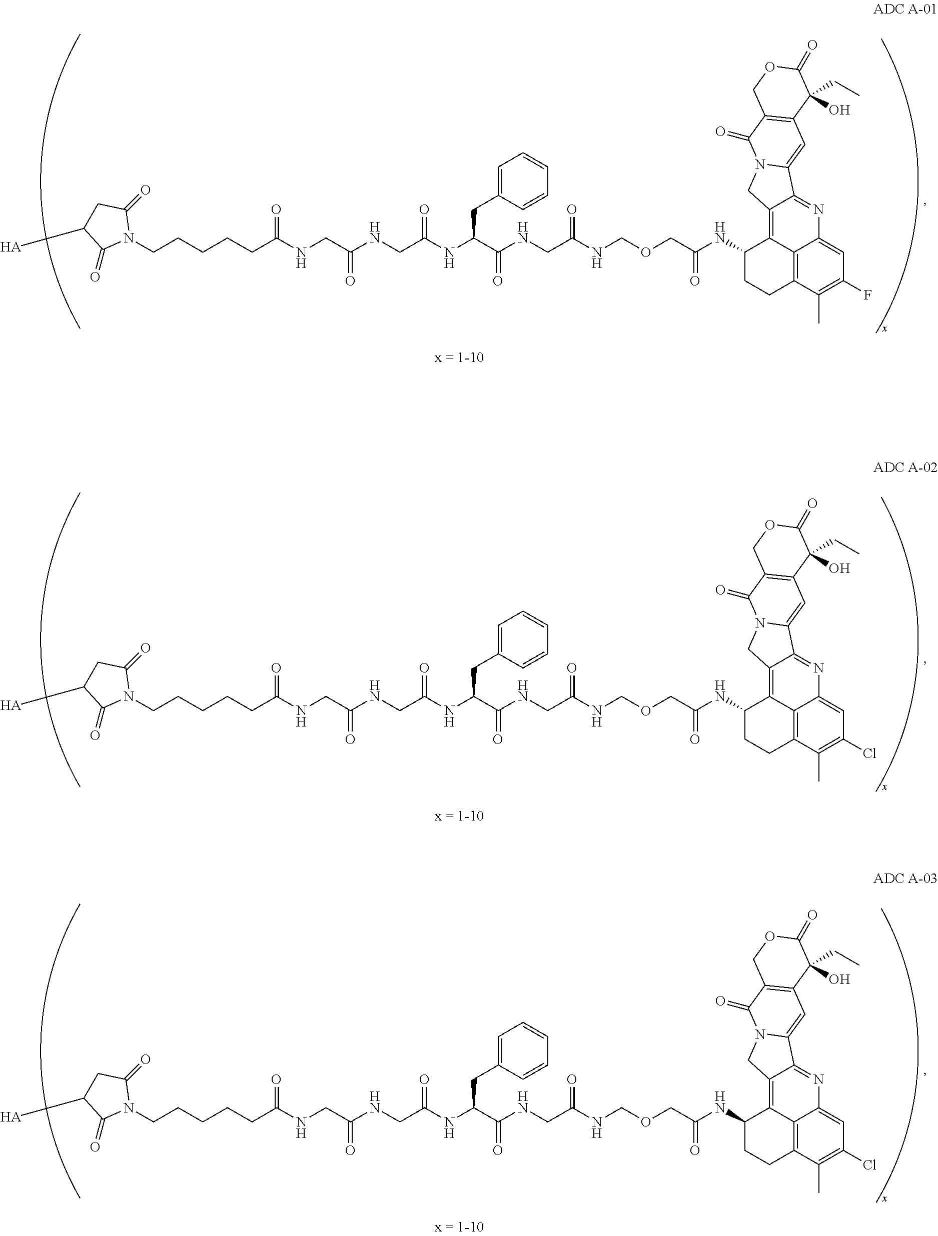 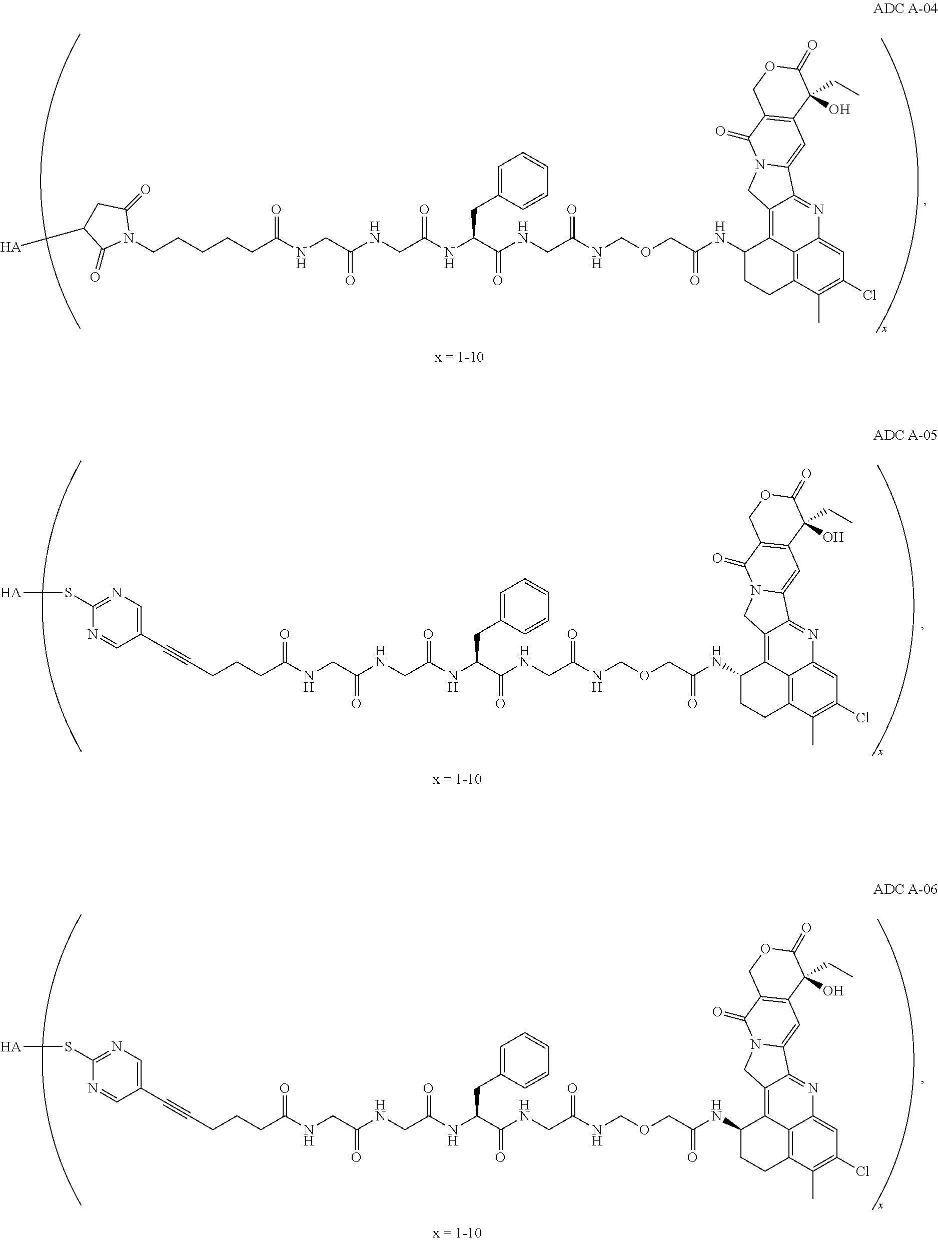 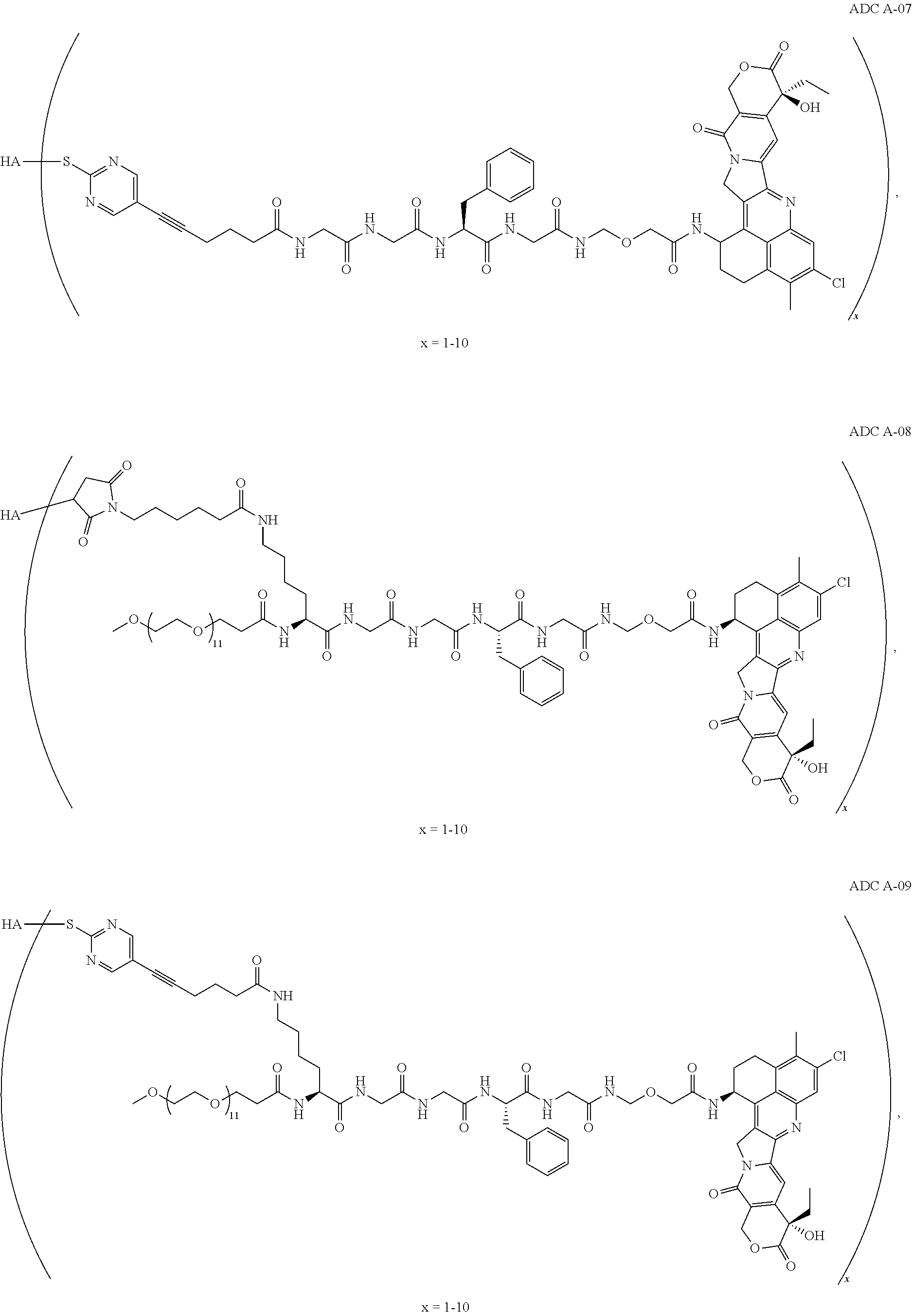 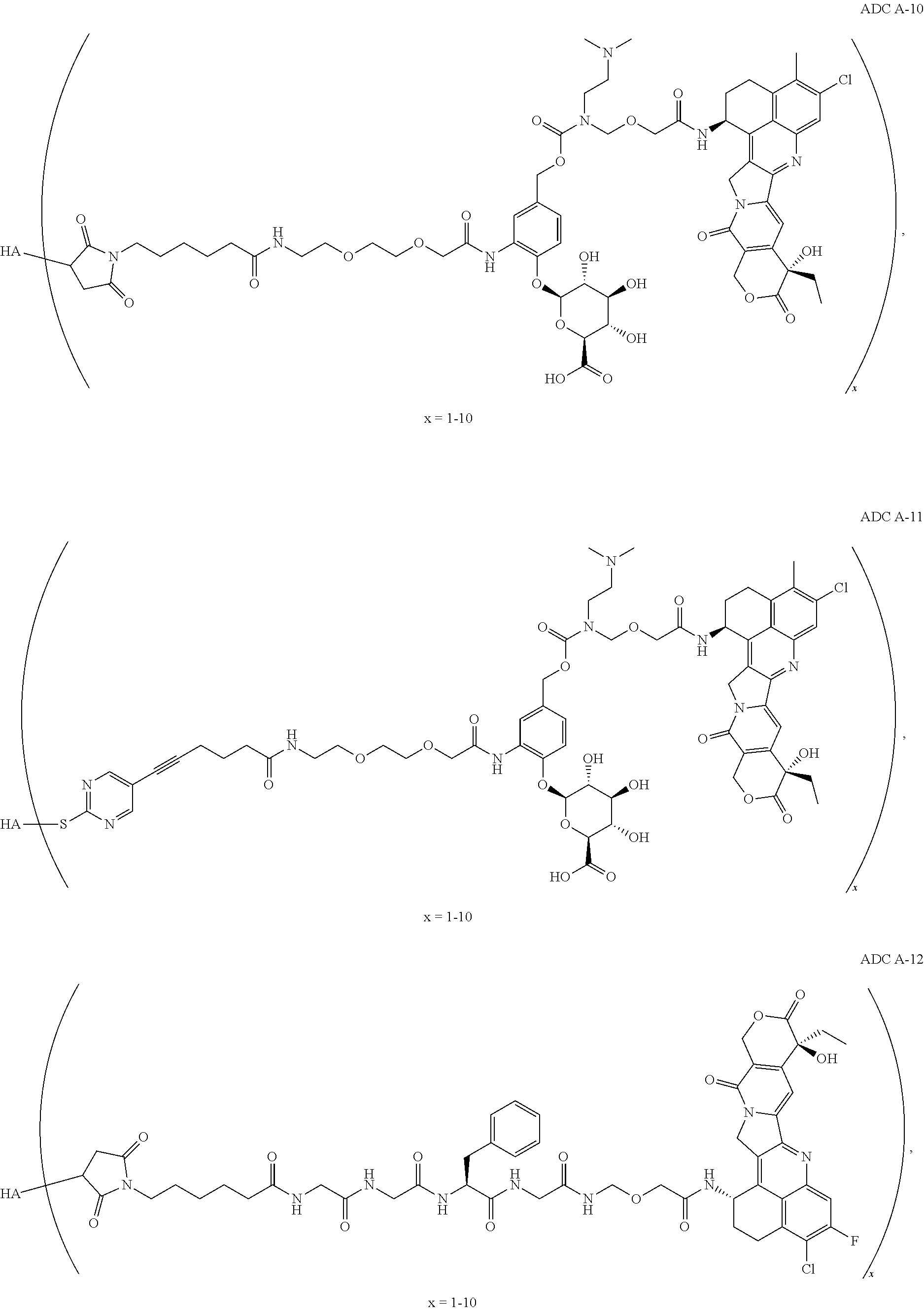 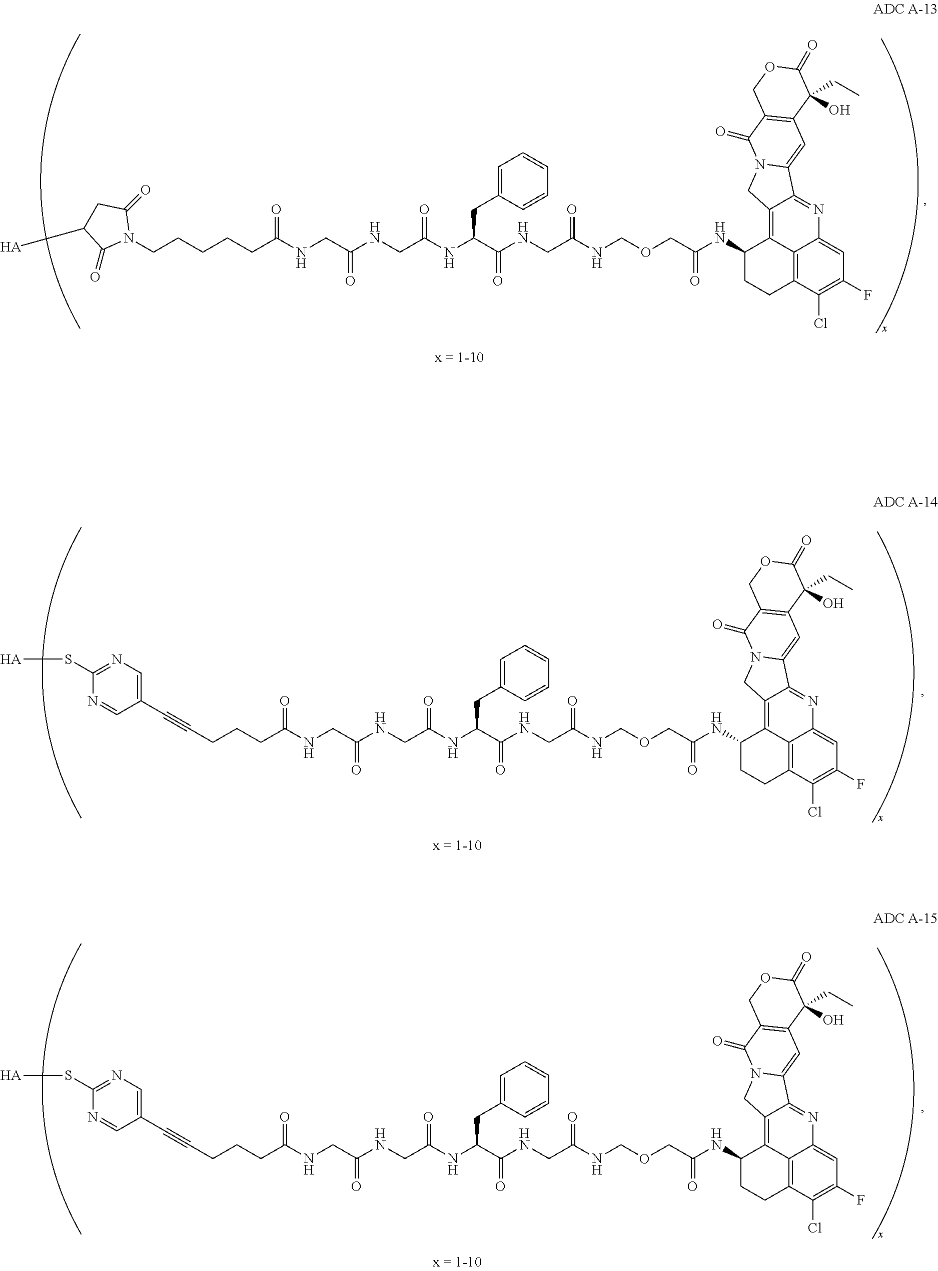 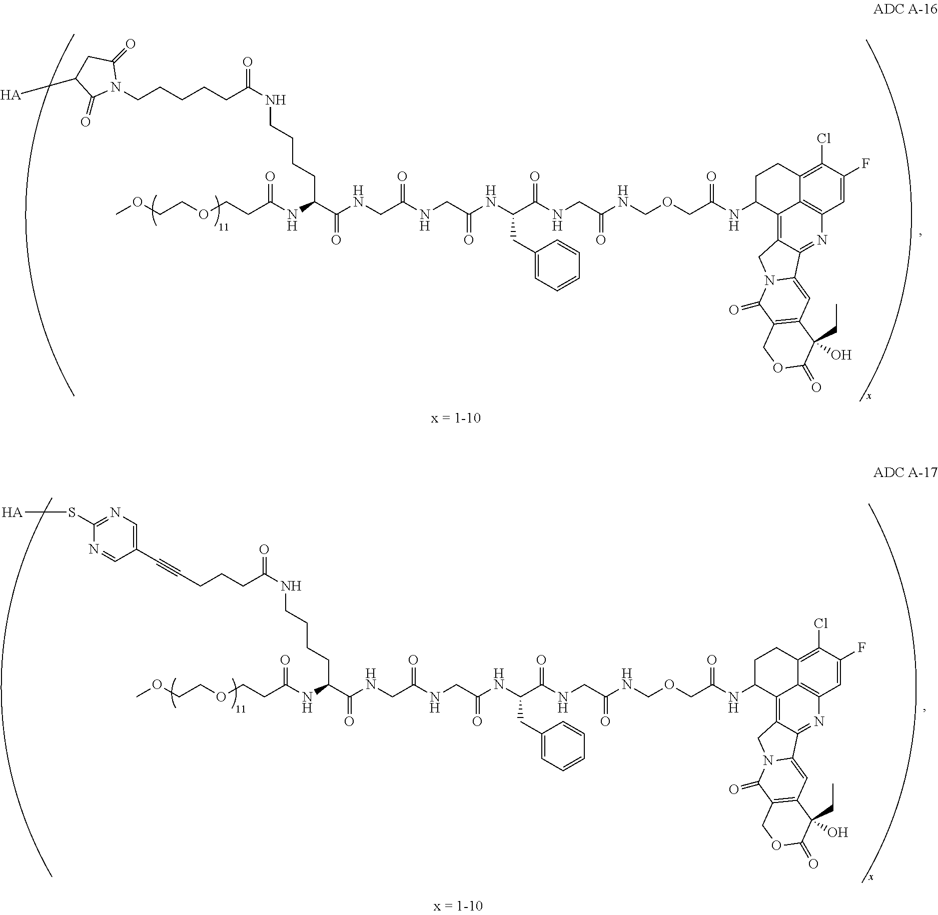 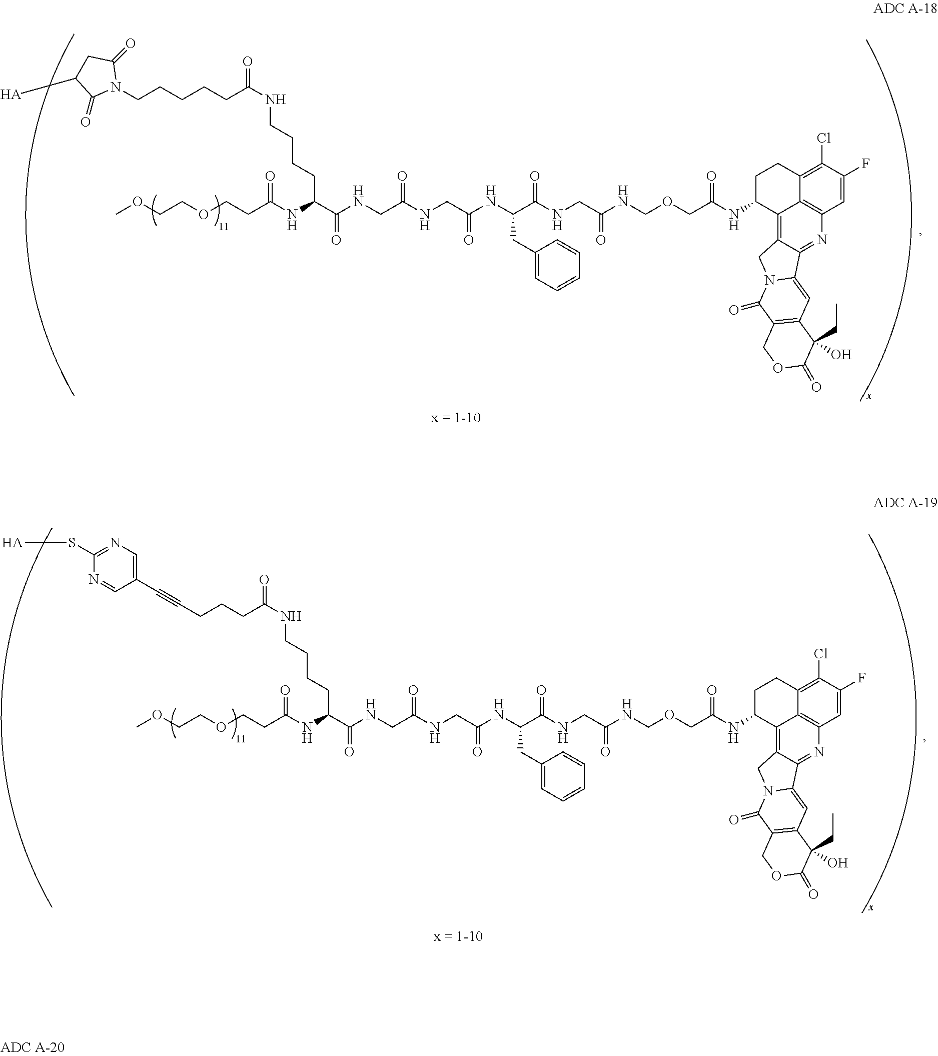 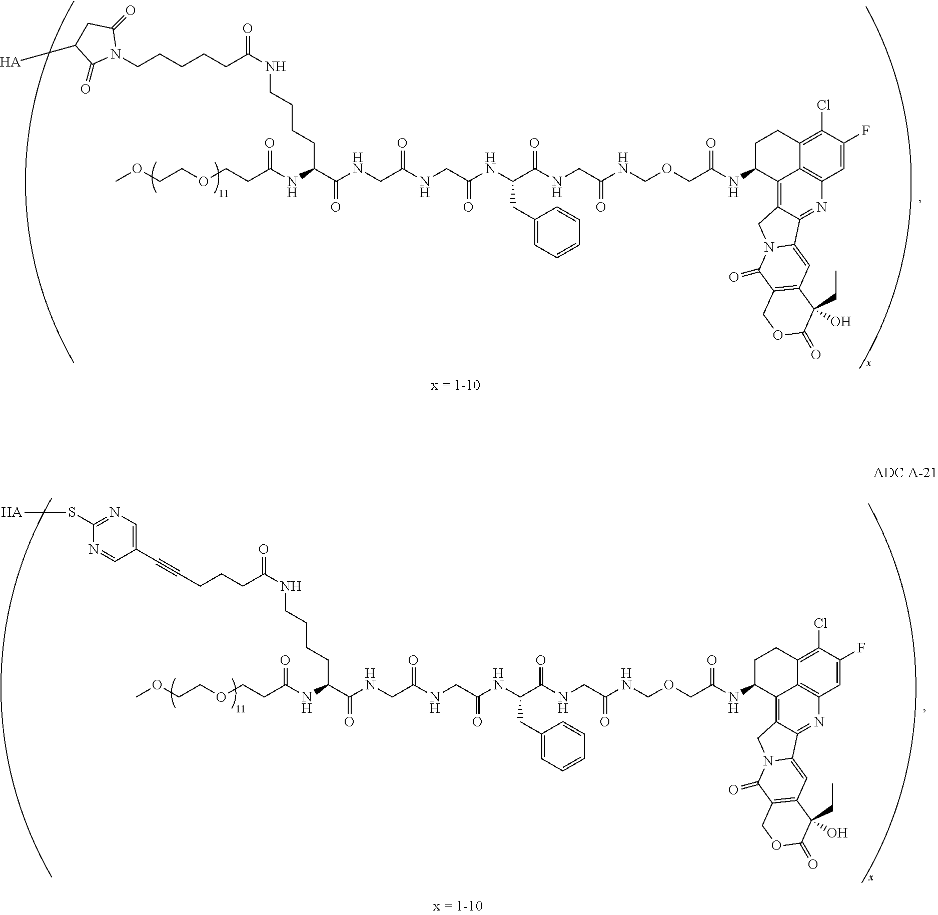 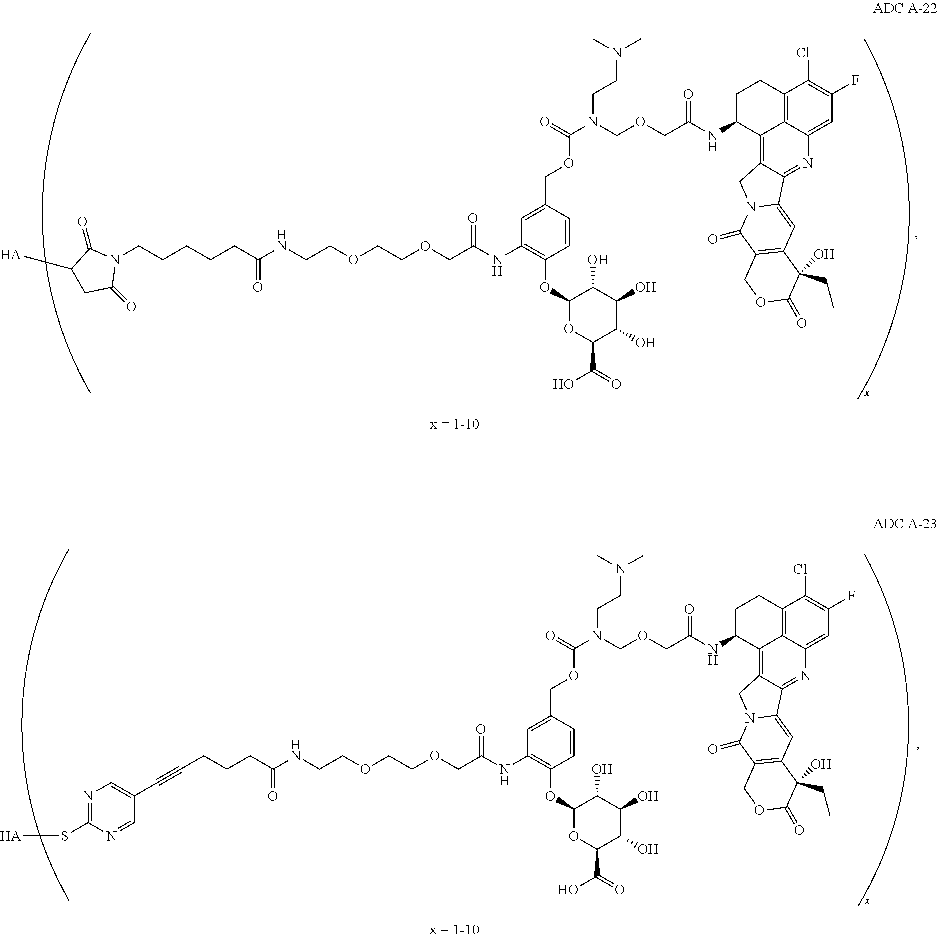 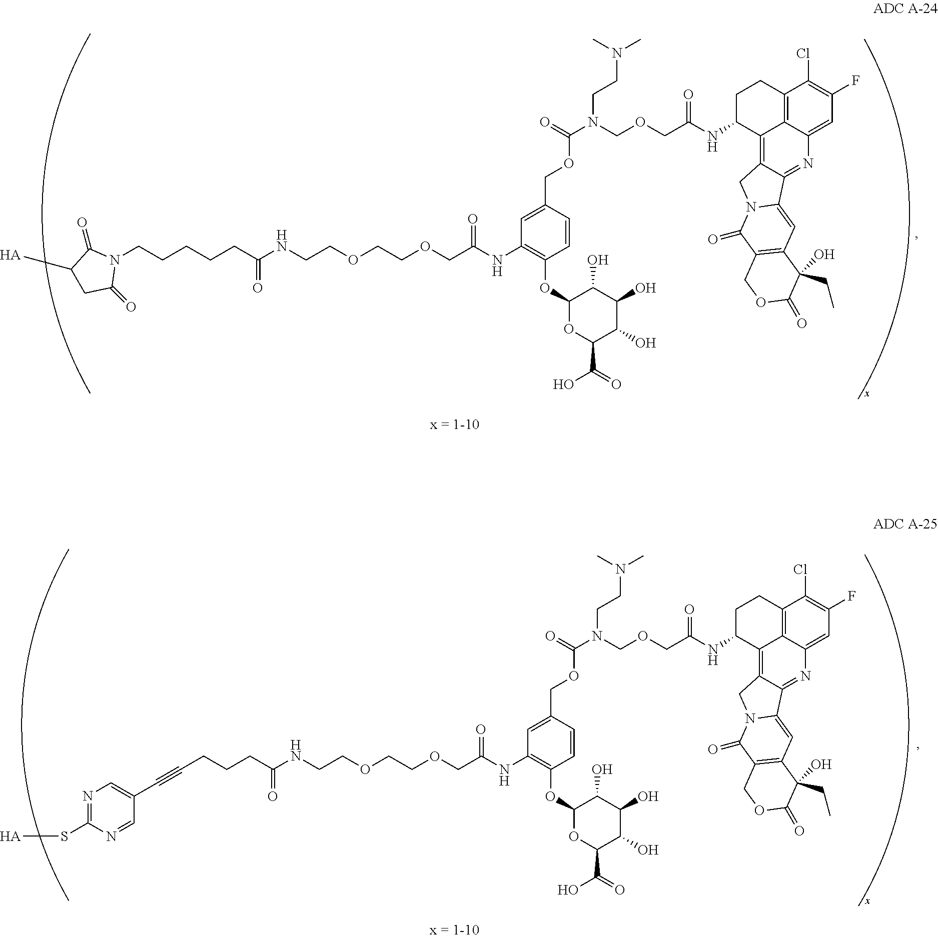 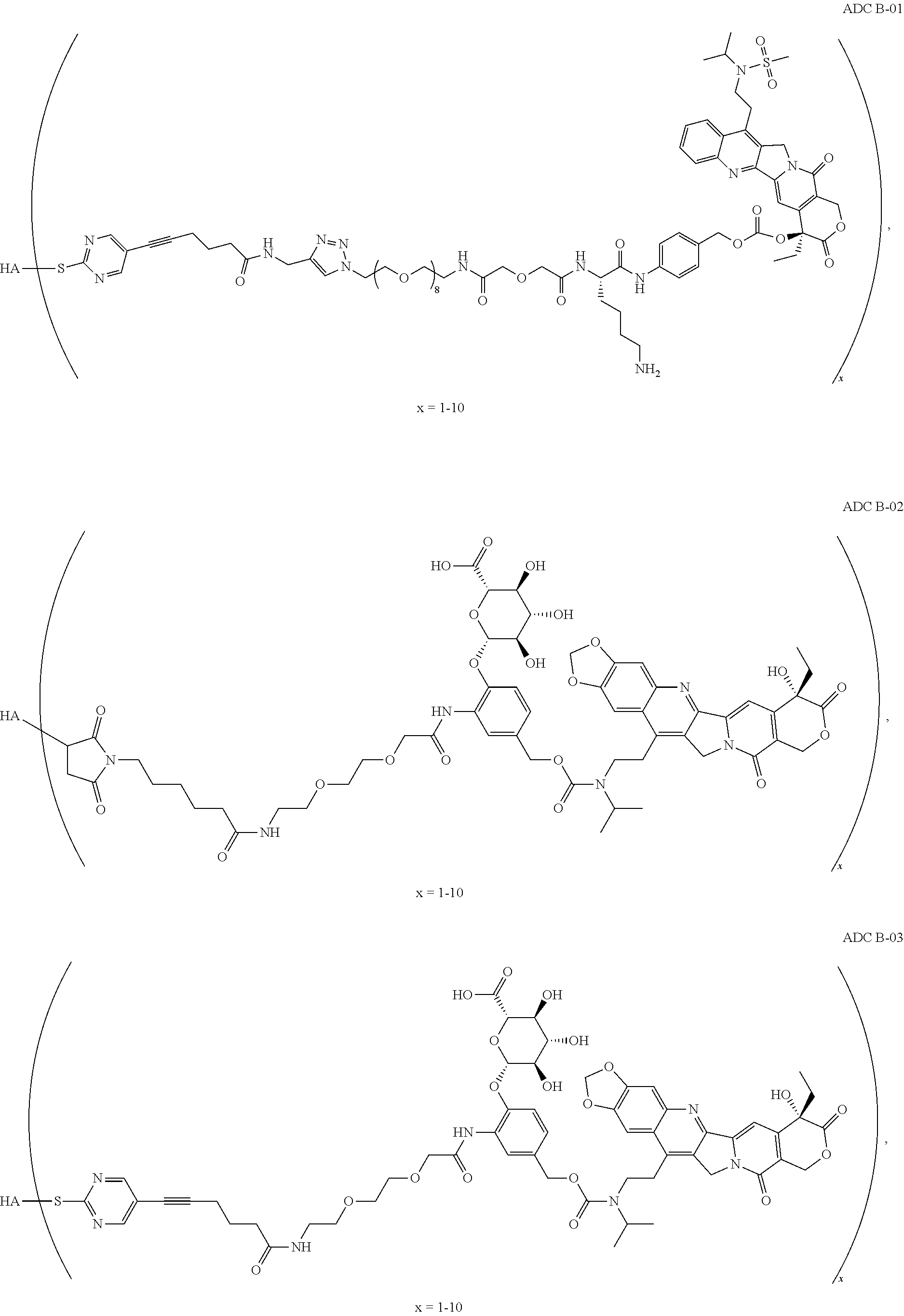 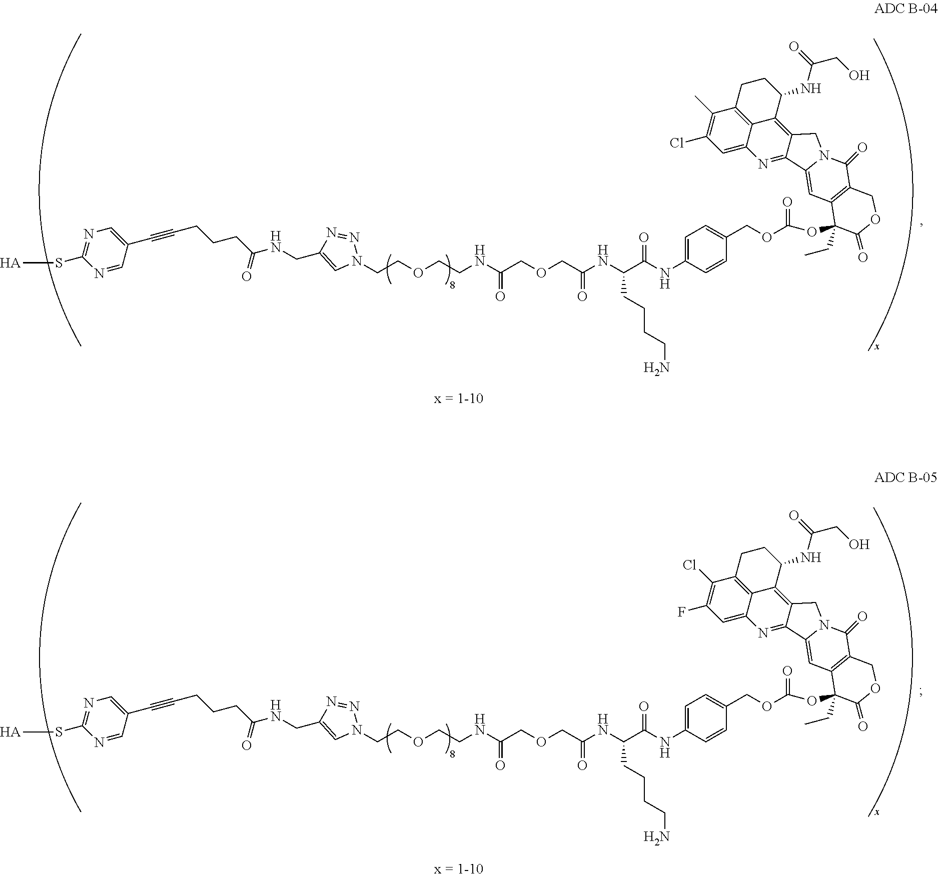
wherein, HA in each antibody-drug conjugate represents an antibody or antigen-binding fragment thereof comprising a VH comprising the amino acid sequence as set forth in SEQ ID NO: 15 and a VL comprising the amino acid sequence as set forth in SEQ ID NO: 16;
wherein, 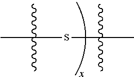
represents a specific way that a sulfhydryl group in the antibody or antigen-binding fragment thereof connects with the linker;
preferably, the antibody or antigen-binding fragment thereof comprises a VH comprising the amino acid sequence as set forth in SEQ ID NO: 15 and a CH comprising the amino acid sequence as set forth in SEQ ID NO: 50; and a VL comprising the amino acid sequence as set forth in SEQ ID NO: 16 and a CL comprising the amino acid sequence as set forth in SEQ ID NO: 51;
(2) comprises the formula: 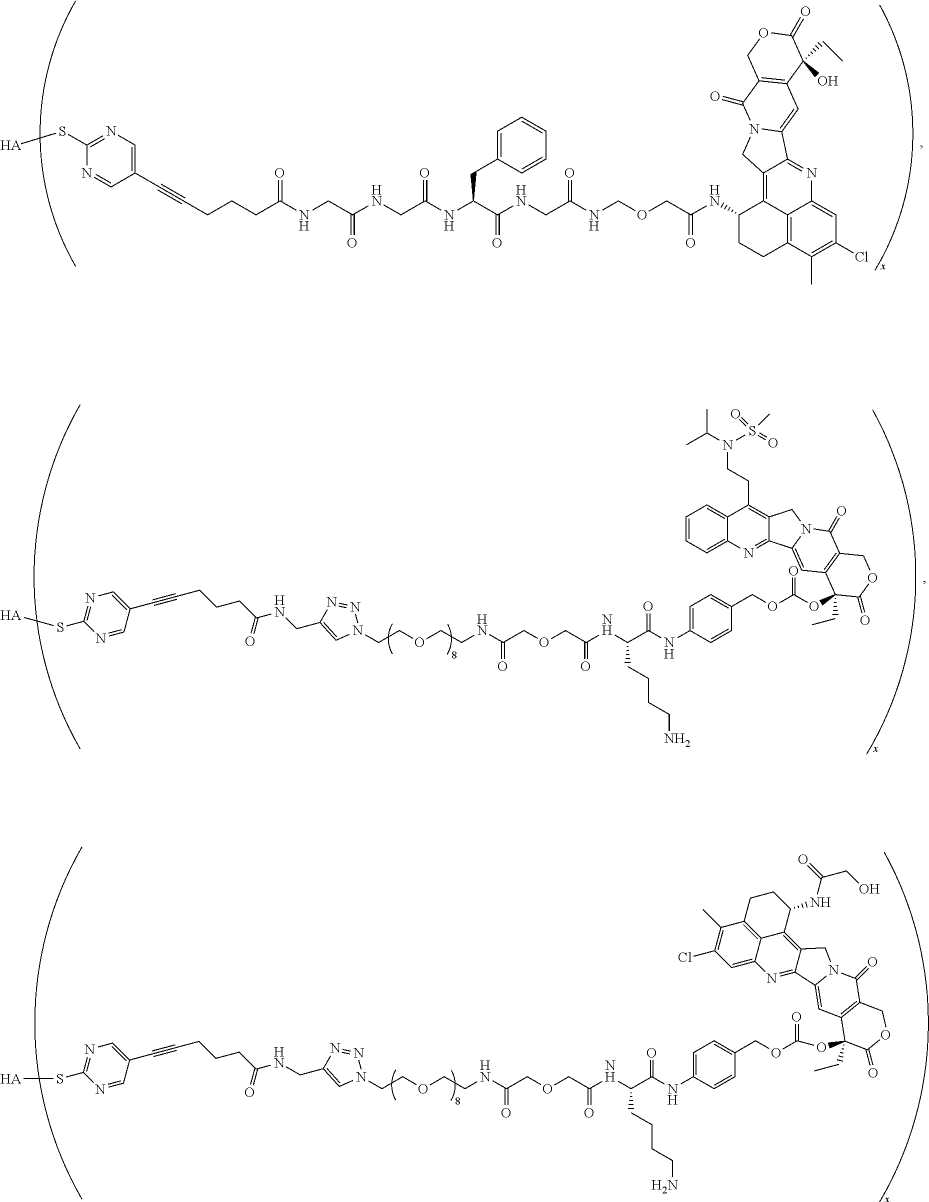 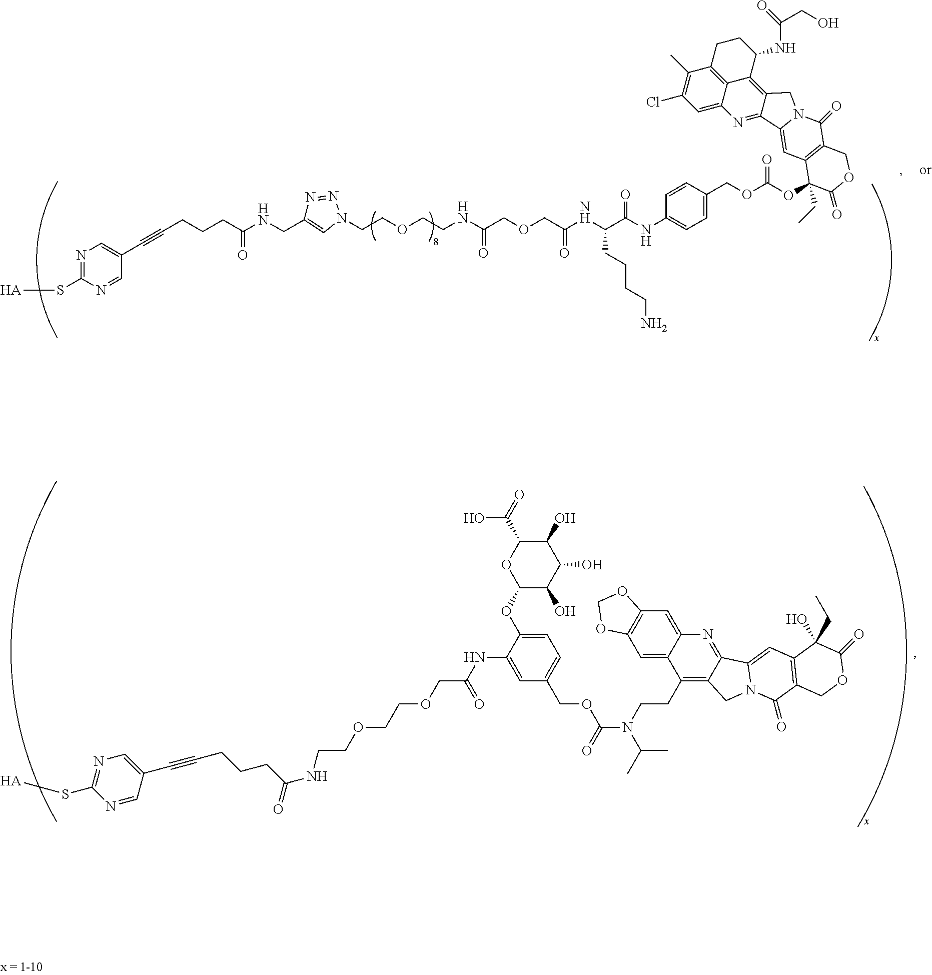
wherein, HA in each antibody-drug conjugate is selected from:
(1) an antibody or antigen-binding fragment thereof comprising VH as set forth in SEQ ID NO: 15 and VL as set forth in SEQ ID NO: 16, for example, an antibody or antigen-binding fragment thereof comprising VH as set forth in SEQ ID NO: 15 and CH as set forth in SEQ ID NO: 50, as well as VL as set forth in SEQ ID NO: 16 and CL as set forth in SEQ ID NO: 51;
(2) an antibody or antigen-binding fragment thereof comprising VH as set forth in SEQ ID NO: 32 and VL as set forth in SEQ ID NO: 33, for example, an antibody or antigen-binding fragment thereof comprising VH as set forth in SEQ ID NO: 32 and CH as set forth in SEQ ID NO: 50, as well as VL as set forth in SEQ ID NO: 33 and CL as set forth in SEQ ID NO: 51; and
(3) an antibody or antigen-binding fragment thereof comprising VH as set forth in SEQ ID NO: 46 and VL as set forth in SEQ ID NO: 47, for example, an antibody or antigen-binding fragment thereof comprising VH as set forth in SEQ ID NO: 46 and CH as set forth in SEQ ID NO: 50, as well as VL as set forth in SEQ ID NO: 47 and CL as set forth in SEQ ID NO: 51; and
x is 3 to 8; or
(3) comprises the formula: 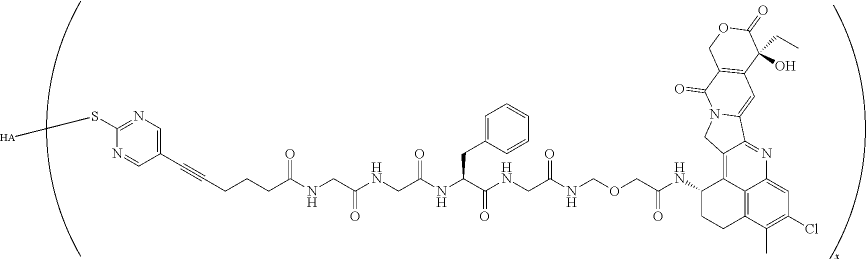
wherein, HA is an antibody or antigen-binding fragment thereof comprising HC as set forth in SEQ ID NO: 17 and LC as set forth in SEQ ID NO: 18; and
x is 7 to 8.

33-35. (canceled)

36. The antibody-drug conjugate of claim 1, wherein (i) the heavy chain C-terminus lacks a lysine residue; (ii) the heavy chain N-terminus is glutamine, glutamic acid, or pyroglutamate; or, (iii) the heavy chain C-terminus lacks a lysine residue and the heavy chain N-terminus is glutamine, glutamic acid, or pyroglutamate.

37. A drug-linker compound, which has a structure shown in formula M′-L-E-D, wherein:
M′ is 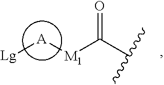
Lg is a leaving group for a nucleophilic substitution reaction,
or hydroxyl (—OH), sulfhydryl group (—SH) or amino (—NH₂); Ring A is a 5- to 6-membered heteroaliphatic ring, or a 5- to 20-membered aromatic ring system, and the heteroaliphatic ring and aromatic ring system are optionally substituted with one or more groups selected from the group consisting of oxy (═O), halogen, cyano, amino, carboxyl, sulfhydryl group and C₁₋₆ alkyl; M₁ is selected from the group consisting of single bond, C₁₋₂₀ alkylene, C₂₋₂₀ alkenylene and C₂₋₂₀ alkynylene;
L is a connector that connects the joint site M and E;
E is a fragment connecting L and D; and
D is a fragment of a cytotoxic drug;
preferably, Lg is halogen, methylsulfonyl, fluorophenol or 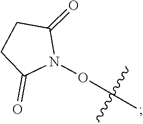
Lg together with an adjacent atom on Ring A forms an unsaturated double bond.

38-39. (canceled)

40. The drug-linker compound of claim 37, wherein M′ is characterized by any one of the following items (1) to (4):
(1) M′ is 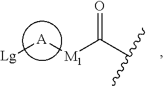
Lg is methylsulfonyl, or, Lg together with an adjacent atom on Ring A forms a carbon-carbon double bond; Ring A is a 5-membered heteroaliphatic ring, a 6-membered heteroaromatic ring, or a polycyclic ring formed by one or more 6-membered heteroaromatic rings and a benzene ring connected through a single bond, wherein the heteroaliphatic ring is optionally substituted with one or more groups selected from the group consisting of oxy (═O), halogen and C₁₋₄ alkyl; and M₁ is selected from the group consisting of single bond, C₃₋₁₀ alkylene, C₃₋₁₀ alkenylene and C₃₋₁₀ alkynylene;
(2) M′ is 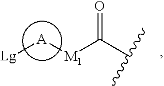
wherein 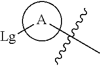
is selected from the group 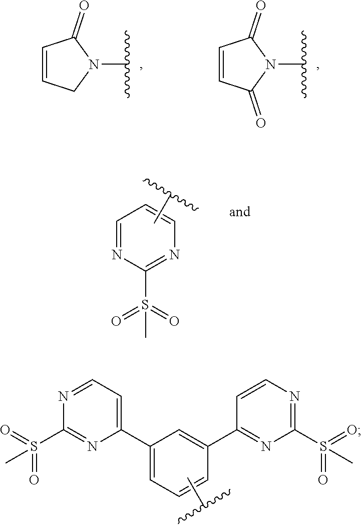
consisting of and M₁ is selected from the group consisting of single bond, C₅₋₈ alkylene, C₅₋₈ alkenylene and C₅-8 alkynylene;
(3) M′ is selected from the group consisting of 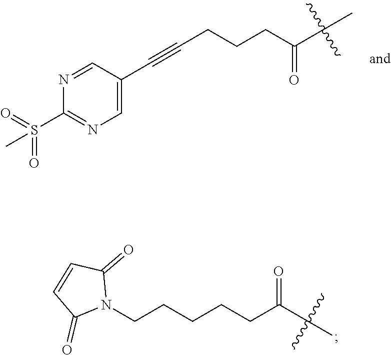
(4) M′ is

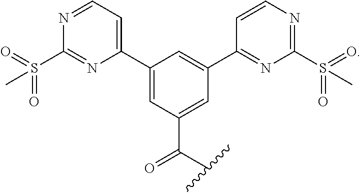 [SMILES: *C(=O)c1cc(-c2ccnc(S(C)(=O)=O)n2)cc(-c2ccnc(S(C)(=O)=O)n2)c1]

41-43. (canceled)

44. The drug-linker compound of claim 37, which is selected from the group consisting of A-01 to A-25, B-01 to B-05 as shown below:

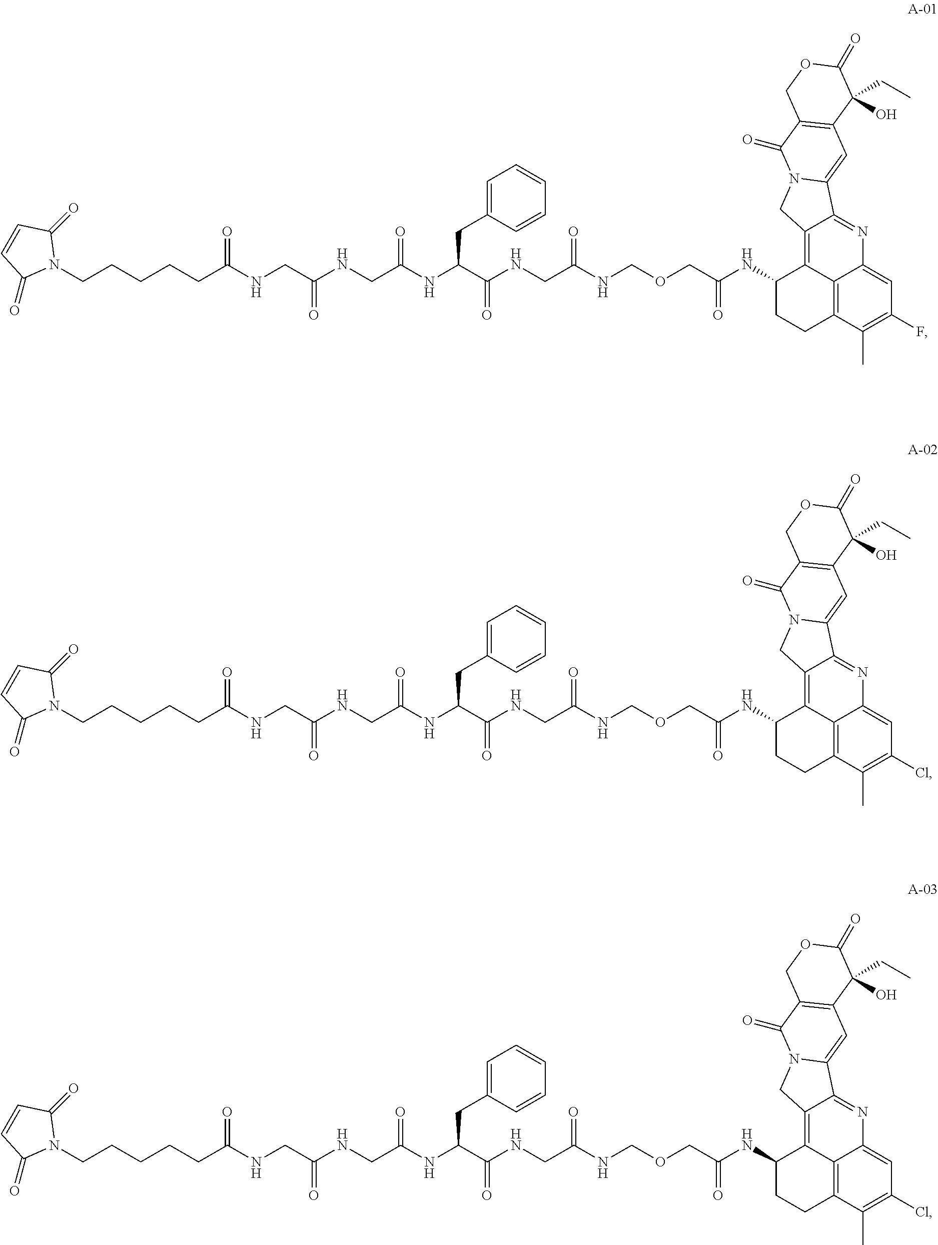 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3 ; CC[C@@]1(O)C(=O)OCc2c1cc1n(c2=O)Cc2c-1nc1cc(Cl)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3 ; CC[C@@]1(O)C(=O)OCc2c1cc1n(c2=O)Cc2c-1nc1cc(Cl)c(C)c3c1c2[C@H](NC(=O)COCNC(=O)CNC(=O)[C@H](Cc1ccccc1)NC(=O)CNC(=O)CNC(=O)CCCCCN1C(=O)C=CC1=O)CC3]

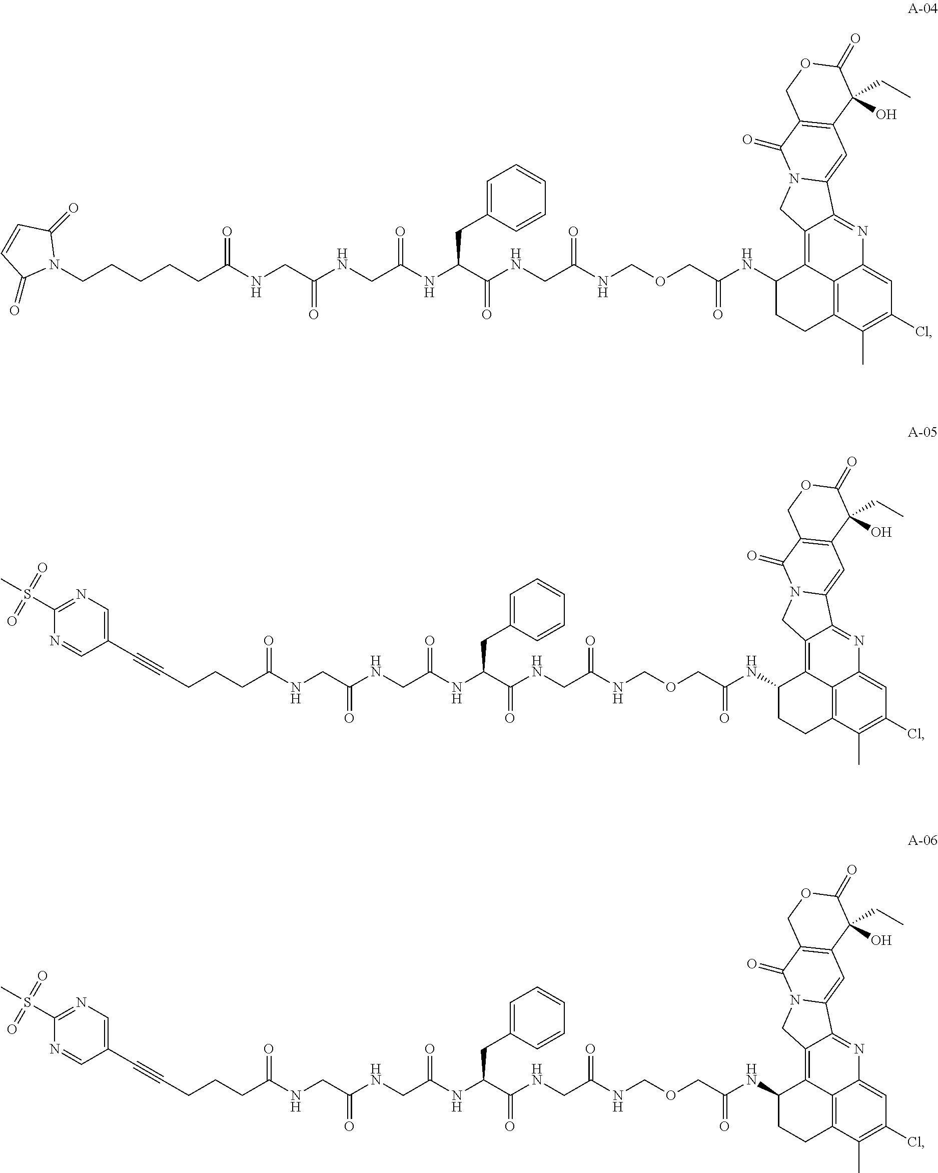 [SMILES: CC[C@@]1(O)C(=O)OCc2c1cc1n(c2=O)Cc2c-1nc1cc(Cl)c(C)c3c1c2C(NC(=O)COCNC(=O)CNC(=O)[C@H](Cc1ccccc1)NC(=O)CNC(=O)CNC(=O)CCCCCN1C(=O)C=CC1=O)CC3 ; CC[C@@]1(O)C(=O)OCc2c1cc1n(c2=O)Cc2c-1nc1cc(Cl)c(C)c3c1c2[C@@H](NC(=O)COCNC(=O)CNC(=O)[C@H](Cc1ccccc1)NC(=O)CNC(=O)CNC(=O)CCCC#Cc1cnc(S(C)(=O)=O)nc1)CC3 ; CC[C@@]1(O)C(=O)OCc2c1cc1n(c2=O)Cc2c-1nc1cc(Cl)c(C)c3c1c2[C@H](NC(=O)COCNC(=O)CNC(=O)[C@H](Cc1ccccc1)NC(=O)CNC(=O)CNC(=O)CCCC#Cc1cnc(S(C)(=O)=O)nc1)CC3]

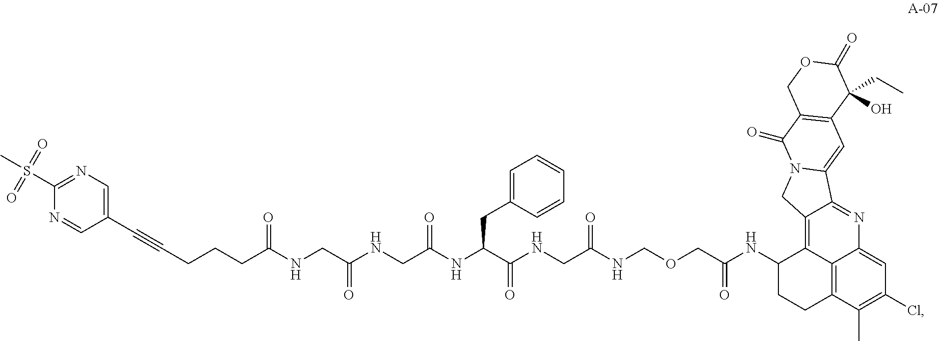 [SMILES: CC[C@@]1(O)C(=O)OCc2c1cc1n(c2=O)Cc2c-1nc1cc(Cl)c(C)c3c1c2C(NC(=O)COCNC(=O)CNC(=O)[C@H](Cc1ccccc1)NC(=O)CNC(=O)CNC(=O)CCCC#Cc1cnc(S(C)(=O)=O)nc1)CC3]

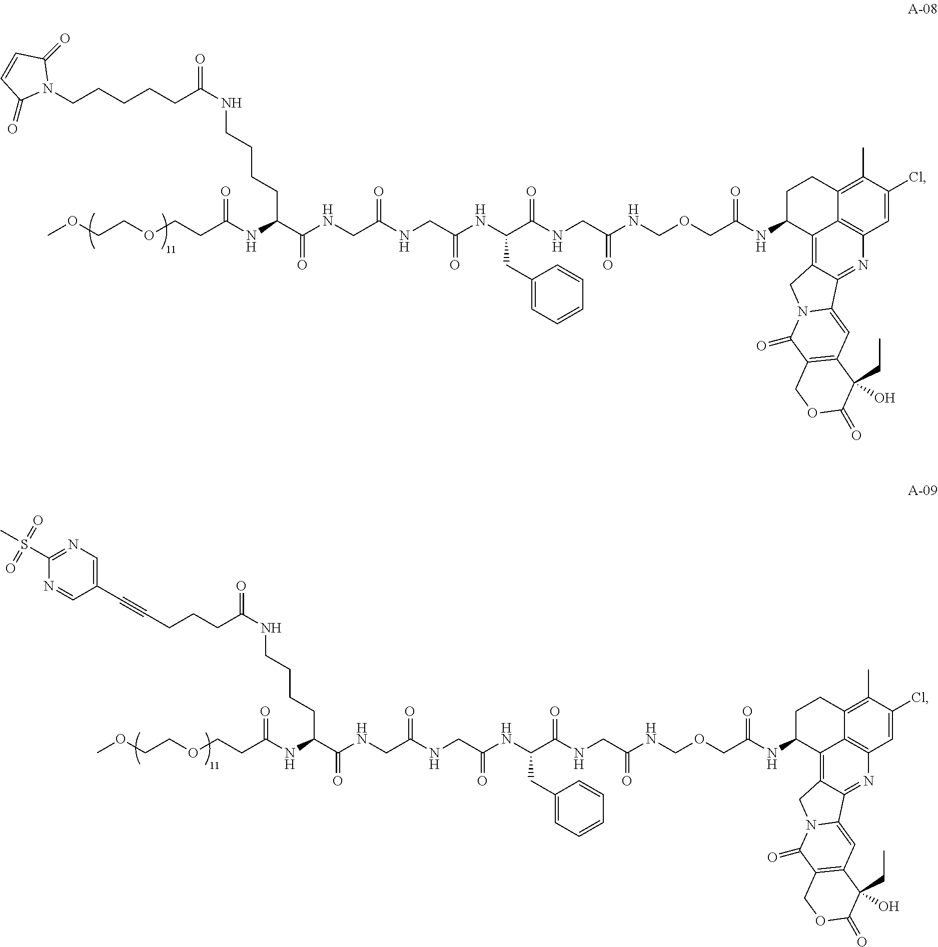

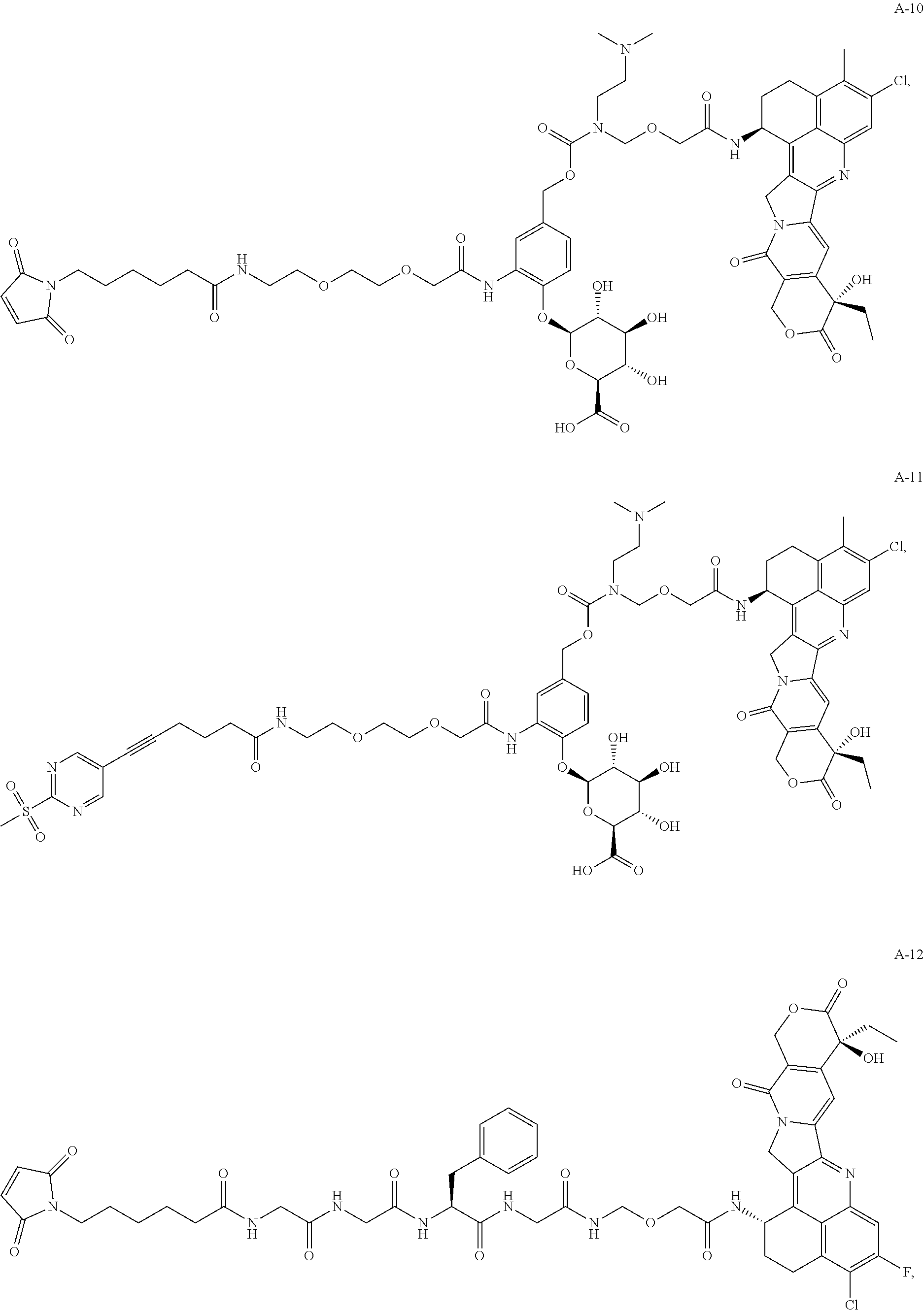

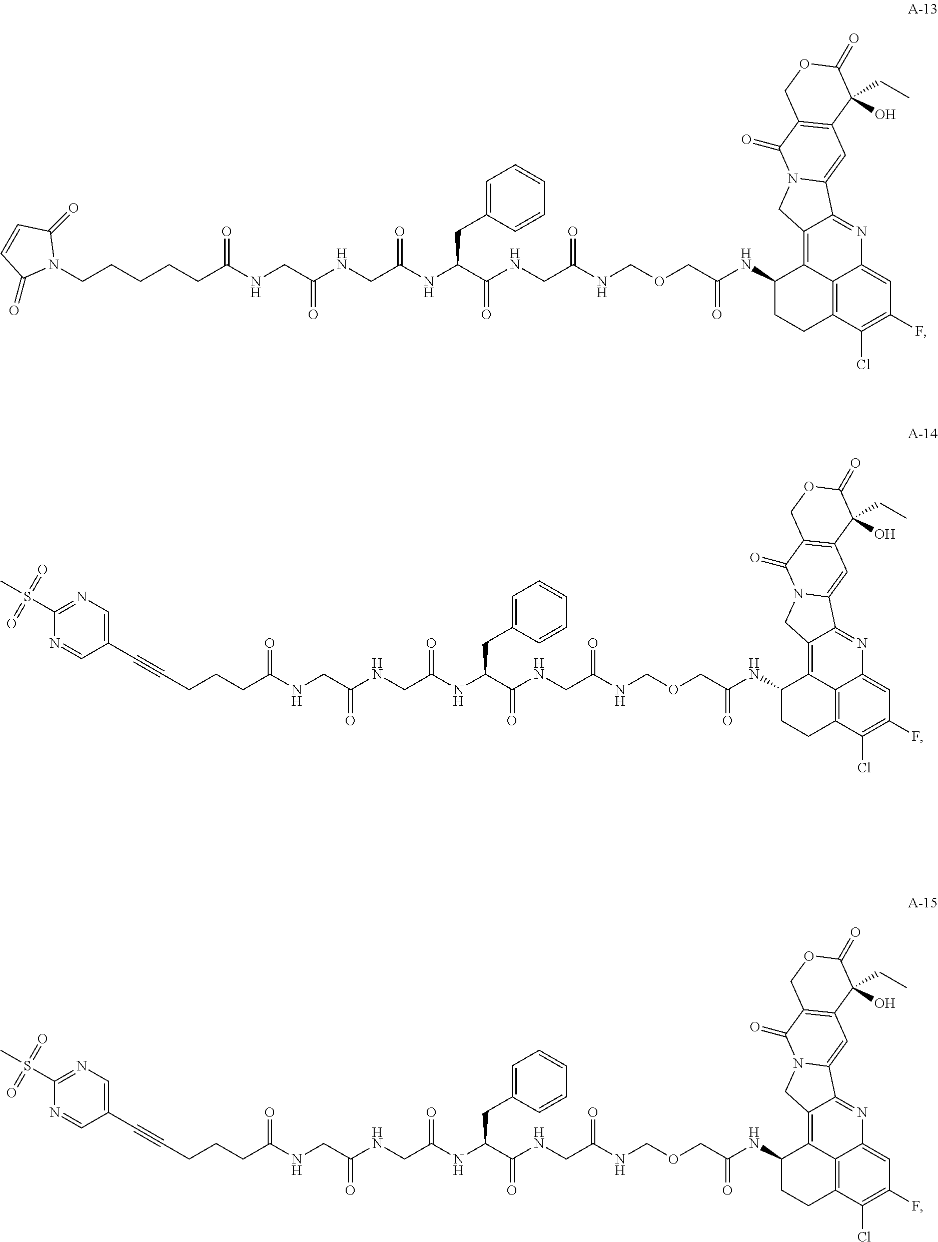 [SMILES: CC[C@@]1(O)C(=O)OCc2c1cc1n(c2=O)Cc2c-1nc1cc(F)c(Cl)c3c1c2[C@H](NC(=O)COCNC(=O)CNC(=O)[C@H](Cc1ccccc1)NC(=O)CNC(=O)CNC(=O)CCCCCN1C(=O)C=CC1=O)CC3 ; CC[C@@]1(O)C(=O)OCc2c1cc1n(c2=O)Cc2c-1nc1cc(F)c(Cl)c3c1c2[C@@H](NC(=O)COCNC(=O)CNC(=O)[C@H](Cc1ccccc1)NC(=O)CNC(=O)CNC(=O)CCCC#Cc1cnc(S(C)(=O)=O)nc1)CC3 ; CC[C@@]1(O)C(=O)OCc2c1cc1n(c2=O)Cc2c-1nc1cc(F)c(Cl)c3c1c2[C@H](NC(=O)COCNC(=O)CNC(=O)[C@H](Cc1ccccc1)NC(=O)CNC(=O)CNC(=O)CCCC#Cc1cnc(S(C)(=O)=O)nc1)CC3]

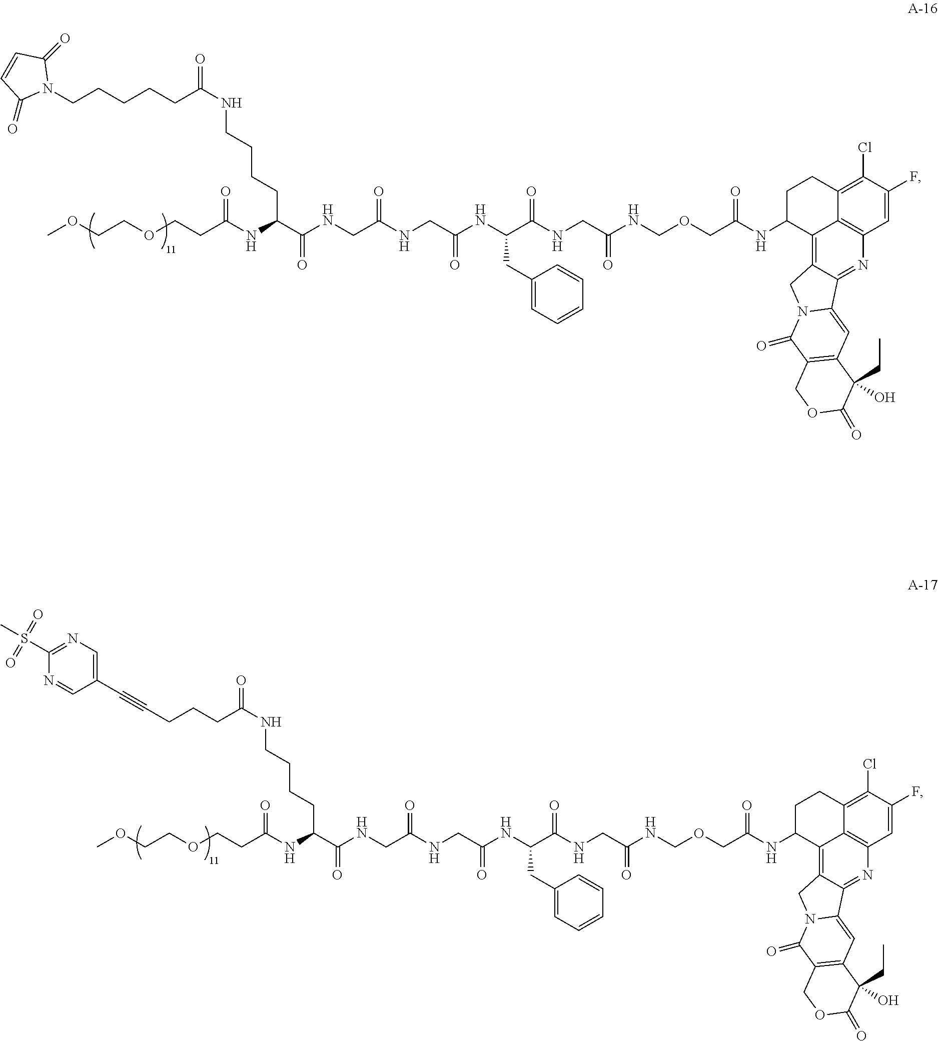

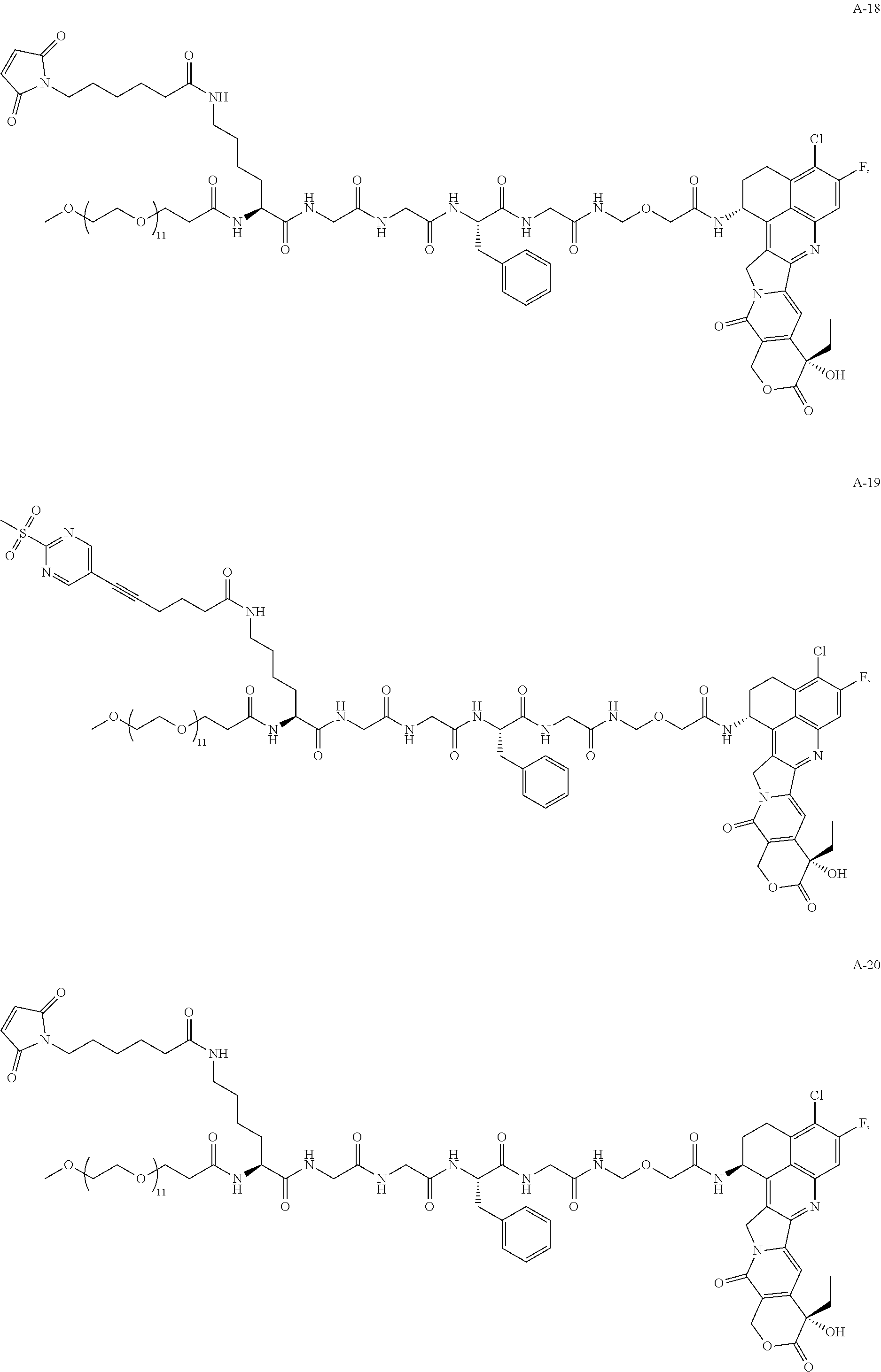

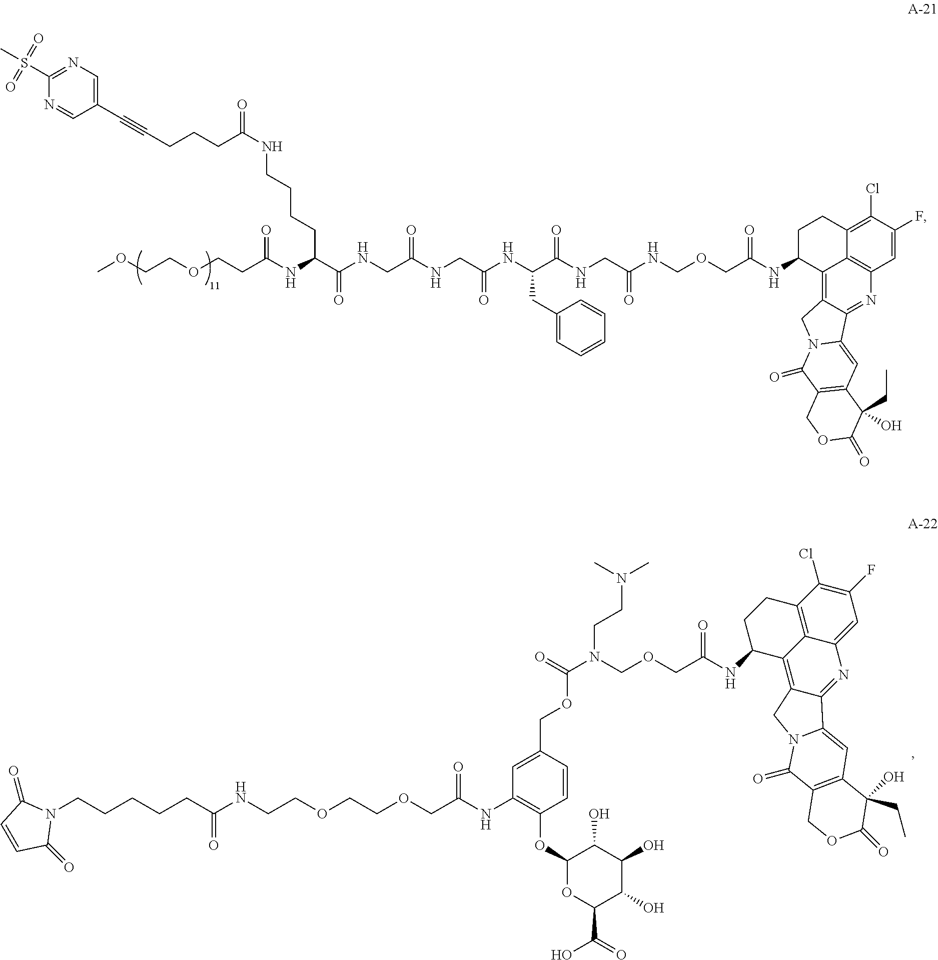

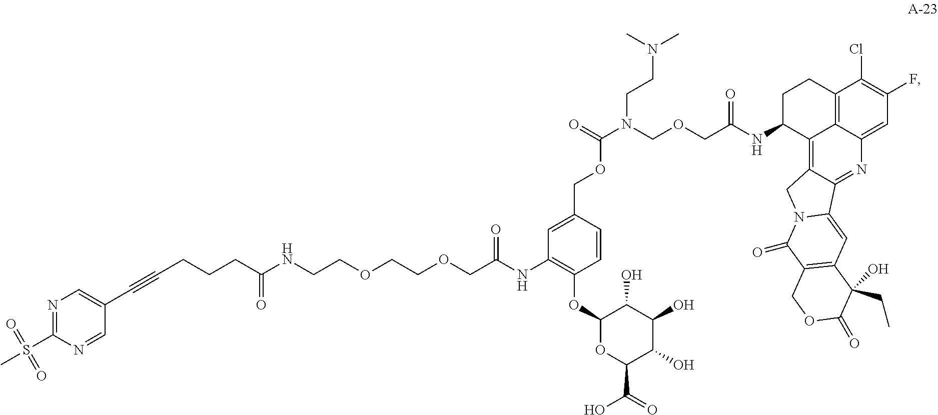 [SMILES: CC[C@@]1(O)C(=O)OCc2c1cc1n(c2=O)Cc2c-1nc1cc(F)c(Cl)c3c1c2[C@@H](NC(=O)COCN(CCN(C)C)C(=O)OCc1ccc(O[C@@H]2O[C@H](C(=O)O)[C@@H](O)[C@H](O)[C@H]2O)c(NC(=O)COCCOCCNC(=O)CCCC#Cc2cnc(S(C)(=O)=O)nc2)c1)CC3]

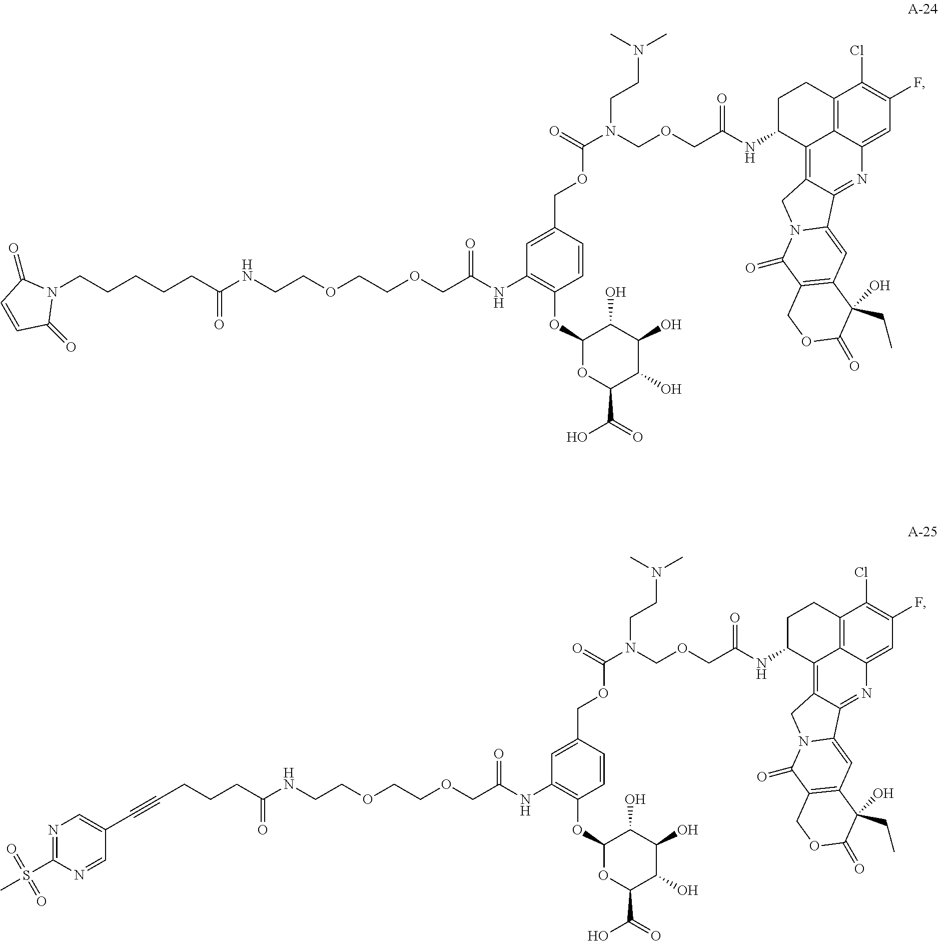 [SMILES: CC[C@@]1(O)C(=O)OCc2c1cc1n(c2=O)Cc2c-1nc1cc(F)c(Cl)c3c1c2[C@H](NC(=O)COCN(CCN(C)C)C(=O)OCc1ccc(O[C@@H]2O[C@H](C(=O)O)[C@@H](O)[C@H](O)[C@H]2O)c(NC(=O)COCCOCCNC(=O)CCCCCN2C(=O)C=CC2=O)c1)CC3 ; CC[C@@]1(O)C(=O)OCc2c1cc1n(c2=O)Cc2c-1nc1cc(F)c(Cl)c3c1c2[C@H](NC(=O)COCN(CCN(C)C)C(=O)OCc1ccc(O[C@@H]2O[C@H](C(=O)O)[C@@H](O)[C@H](O)[C@H]2O)c(NC(=O)COCCOCCNC(=O)CCCC#Cc2cnc(S(C)(=O)=O)nc2)c1)CC3]

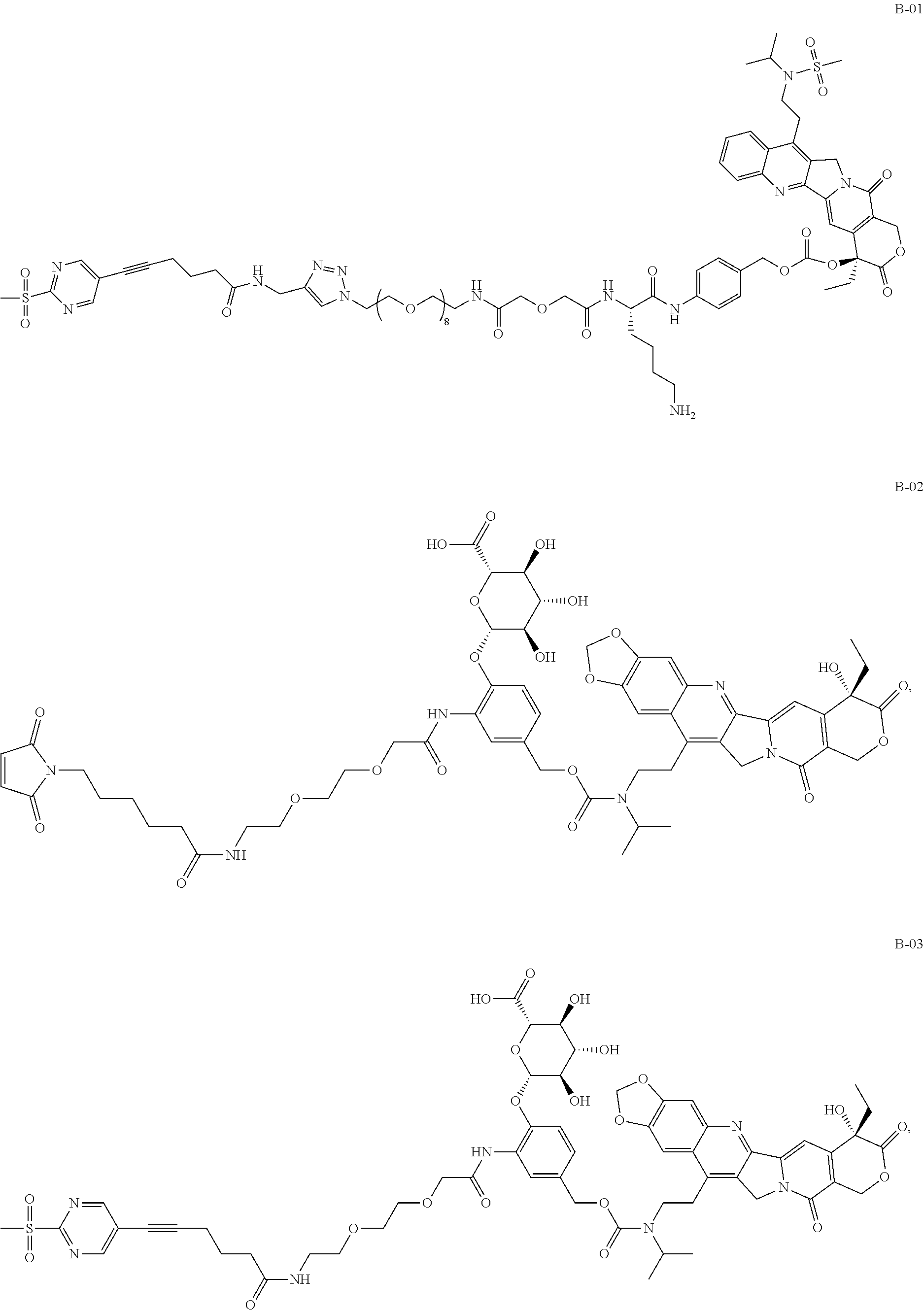

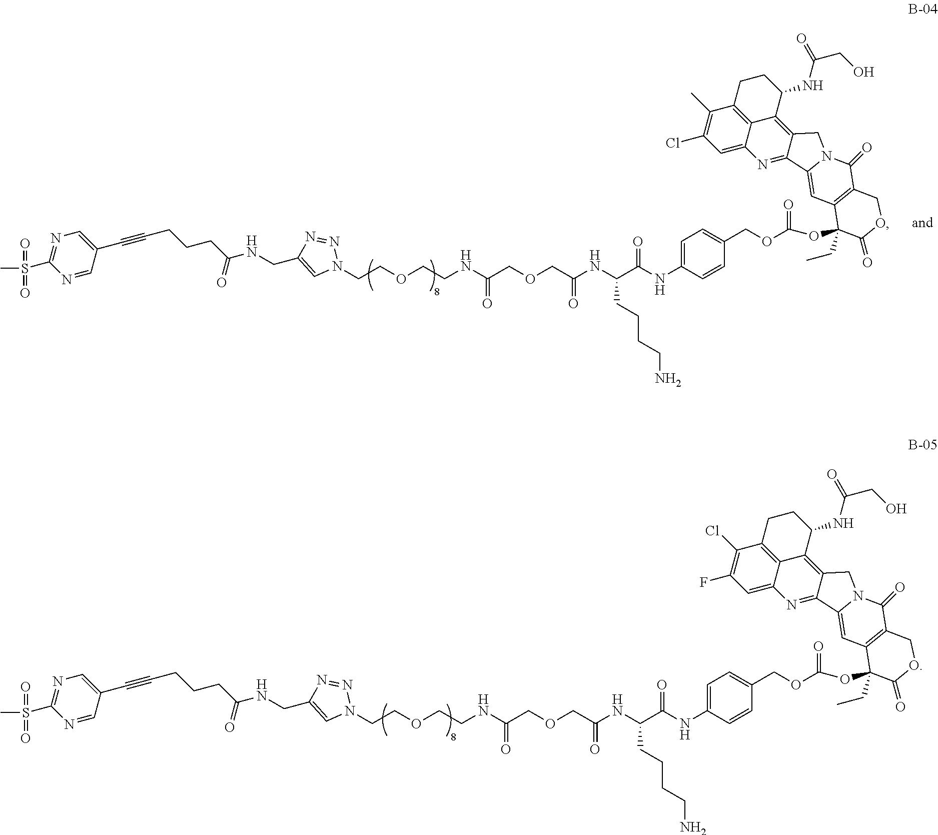

45. An antibody-drug conjugate comprising an antibody that binds HER3 conjugated via one or more cysteine residue of the antibody to a drug linker of claim 37.

46. An antibody-drug conjugate comprising an antibody that binds HER3 conjugated via one or more cysteine residue of the antibody to a drug-linker selected from the group consisting of A-01, A-02, A-03, A-04, A-05, A-06, A-07, A-08, A-09, A-10, A-12, A13, A-14, A15, A-16, A-17, A-18, A-19, A-20, A-21, A-22, A-23, A-24, A-25, B-01, B-02, B-03, B-04, and B-05.

47. The antibody-drug conjugate of claim 45, wherein the antibody that binds HER3 is selected from the group consisting of:
(1) a heavy chain variable region (VH) and/or a light chain variable region (VL) as follows, wherein the CDRs are defined by the Chothia numbering system:
(1a) a heavy chain variable region (VH) comprising the following 3 CDRs: CDR-H1 having the amino acid sequence as set forth in SEQ ID NO: 1 or a variant thereof, CDR-H2 having the amino acid sequence as set forth in SEQ ID NO: 2 or a variant thereof, CDR-H3 having the amino acid sequence as set forth in SEQ ID NO: 3 or a variant thereof; and/or, a light chain variable region (VL) comprising the following 3 CDRs: CDR-L1 having the amino acid sequence as set forth in SEQ ID NO: 4 or a variant thereof, CDR-L2 having the amino acid sequence as set forth in SEQ ID NO: 5 or a variant thereof, CDR-L3 having the amino acid sequence as set forth in SEQ ID NO: 6 or a variant thereof; or,
(1b) a heavy chain variable region (VH) comprising the following 3 CDRs: CDR-H1 having the amino acid sequence as set forth in SEQ ID NO: 19 or a variant thereof, CDR-H2 having the amino acid sequence as set forth in SEQ ID NO: 20 or a variant thereof, CDR-H3 having the amino acid sequence as set forth in SEQ ID NO: 21 or a variant thereof; and/or, a light chain variable region (VL) comprising the following 3 CDRs: CDR-L1 having the amino acid sequence as set forth in SEQ ID NO: 22 or a variant thereof, CDR-L2 having the amino acid sequence as set forth in SEQ ID NO: 23 or a variant thereof, CDR-L3 having the amino acid sequence as set forth in SEQ ID NO: 24 or a variant thereof; or,
(1c) a heavy chain variable region (VH) comprising the following 3 CDRs: CDR-H1 having the amino acid sequence as set forth in SEQ ID NO: 36 or a variant thereof, CDR-H2 having the amino acid sequence as set forth in SEQ ID NO: 37 or a variant thereof, CDR-H3 having the amino acid sequence as set forth in SEQ ID NO: 38 or a variant thereof; and/or, a light chain variable region (VL) comprising the following 3 CDRs: CDR-L1 having the amino acid sequence as set forth in SEQ ID NO: 45 or a variant thereof, CDR-L2 having the amino acid sequence as set forth in SEQ ID NO: 23 or a variant thereof, and CDR-L3 having the amino acid sequence as set forth in SEQ ID NO: 52 or a variant thereof;
wherein, the variant described in any item of (1a), (1b), and (1c) has the amino acid sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2 or 3 amino acids) as compared to the sequence from which it is derived;
preferably, the substitution is a conservative substitution with the proviso that the amino acid sequences of the CDRs of the variant have 100% sequence identity to the amino acid sequences of the respective CDRs of the VH and VL of (1a), (1b), and (1c) and the variant binds HER3;
or,
(2) a heavy chain variable region (VH) and/or a light chain variable region (VL) as follows, wherein the CDRs are defined according to the Kabat numbering system:
(2a) a heavy chain variable region (VH) comprising the following 3 CDRs: CDR-H1 having the amino acid sequence as set forth in SEQ ID NO: 7 or a variant thereof, CDR-H2 having the amino acid sequence as set forth in SEQ ID NO: 8 or a variant thereof, CDR-H3 having the amino acid sequence as set forth in SEQ ID NO: 9 or a variant thereof; and/or, a light chain variable region (VL) comprising the following 3 CDRs: CDR-L1 having the amino acid sequence as set forth in SEQ ID NO: 4 or a variant thereof, CDR-L2 having the amino acid sequence as set forth in SEQ ID NO: 5 or a variant thereof, CDR-L3 having the amino acid sequence as set forth in SEQ ID NO: 6 or a variant thereof, or,
(2b) a heavy chain variable region (VH) comprising the following 3 CDRs: CDR-H1 having the amino acid sequence as set forth in SEQ ID NO: 25 or a variant thereof, CDR-H2 having the amino acid sequence as set forth in SEQ ID NO: 26 or a variant thereof, CDR-H3 having the amino acid sequence as set forth in SEQ ID NO: 21 or a variant thereof; and/or, a light chain variable region (VL) comprising the following 3 CDRs: CDR-L1 having the amino acid sequence as set forth in SEQ ID NO: 22 or a variant thereof, CDR-L2 having the amino acid sequence as set forth in SEQ ID NO: 23 or a variant thereof, CDR-L3 having the amino acid sequence as set forth in SEQ ID NO: 24 or a variant thereof, or,
(2c) a heavy chain variable region (VH) comprising the following 3 CDRs: CDR-H1 having the amino acid sequence as set forth in SEQ ID NO: 39 or a variant thereof, CDR-H2 having the amino acid sequence as set forth in SEQ ID NO: 40 or a variant thereof, CDR-H3 having the amino acid sequence as set forth in SEQ ID NO: 38 or a variant thereof; and/or, a light chain variable region (VL) comprising the following 3 CDRs: CDR-L1 having the amino acid sequence as set forth in SEQ ID NO: 45 or a variant thereof, CDR-L2 having the amino acid sequence as set forth in SEQ ID NO: 23 or a variant thereof, and CDR-L3 having the amino acid sequence as set forth in SEQ ID NO: 52 or a variant thereof;
wherein, the variant described in any item of (1a), (1b), and (1c) has the amino acid sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2 or 3 amino acids) as compared to the sequence from which it is derived; preferably, the substitution is a conservative substitution with the proviso that the amino acid sequences of the CDRs of the variant have 100% sequence identity to the amino acid sequences of the respective CDRs of the VH and VL of (2a), (2b), and (2c) and the variant binds HER;
or,
(3) a heavy chain variable region (VH) and/or a light chain variable region (VL) as follows, wherein the CDRs are defined by the IMGT numbering system:
(3a) a heavy chain variable region (VH) comprising the following 3 CDRs: CDR-H1 having the amino acid sequence as set forth in SEQ ID NO: 10 or a variant thereof, CDR-H2 having the amino acid sequence as set forth in SEQ ID NO: 11 or a variant thereof, CDR-H3 having the amino acid sequence as set forth in SEQ ID NO: 12 or a variant thereof; and/or, a light chain variable region (VL) comprising the following 3 CDRs: CDR-L1 having the amino acid sequence as set forth in SEQ ID NO: 13 or a variant thereof, CDR-L2 having the amino acid sequence as set forth in SEQ ID NO: 14 or a variant thereof, CDR-L3 having the amino acid sequence as set forth in SEQ ID NO: 6 or a variant thereof, or,
(3b) a heavy chain variable region (VH) comprising the following 3 CDRs: CDR-H1 having the amino acid sequence as set forth in SEQ ID NO: 27 or a variant thereof, CDR-H2 having the amino acid sequence as set forth in SEQ ID NO: 28 or a variant thereof, CDR-H3 having the amino acid sequence as set forth in SEQ ID NO: 29 or a variant thereof; and/or, a light chain variable region (VL) comprising the following 3 CDRs: CDR-L1 having the amino acid sequence as set forth in SEQ ID NO: 30 or a variant thereof, CDR-L2 having the amino acid sequence as set forth in SEQ ID NO: 31 or a variant thereof, CDR-L3 having the amino acid sequence as set forth in SEQ ID NO: 24 or a variant thereof; or,
(3c) a heavy chain variable region (VH) comprising the following 3 CDRs: CDR-H1 having the amino acid sequence as set forth in SEQ ID NO: 41 or a variant thereof, CDR-H2 having the amino acid sequence as set forth in SEQ ID NO: 42 or a variant thereof, CDR-H3 having the amino acid sequence as set forth in SEQ ID NO: 43 or a variant thereof; and/or, a light chain variable region (VL) comprising the following 3 CDRs: CDR-L1 having the amino acid sequence as set forth in SEQ ID NO: 44 or a variant thereof, CDR-L2 having the amino acid sequence as set forth in SEQ ID NO: 31 or a variant thereof, and CDR-L3 having the amino acid sequence as set forth in SEQ ID NO: 52 or a variant thereof; wherein, the variant described in any item of (1a), (1b), and (1c) has the amino acid sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2 or 3 amino acids) as compared to the sequence from which it is derived; preferably, the substitution is a conservative substitution with the proviso that the amino acid sequences of the CDRs of the variant have 100% sequence identity to the amino acid sequences of the respective CDRs of the VH and VL of (3a), (3b), and (3c) and the variant binds HER3;
or the antibody that binds HER3 is selected from the group consisting of
(a) a heavy chain variable domain (VH) comprising the amino acid sequence as set forth in SEQ ID NO: 15 or a variant thereof, and/or, a light chain variable domain (VL) comprising the amino acid sequence as set forth in SEQ ID NO: 16 or a variant thereof;
(b) a VH comprising the amino acid sequence as set forth in SEQ ID NO: 32 or a variant thereof, and/or, a VL comprising the amino acid sequence as set forth in SEQ ID NO: 33 or a variant thereof; or
(c) a VH comprising the amino acid sequence as set forth in SEQ ID NO: 46 or a variant thereof, and/or, a VL comprising the amino acid sequence as set forth in SEQ ID NO: 47 or a variant thereof;
wherein, the variant described in any item of (1a), (1b), and (1c) has an amino acid sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2 or 3 amino acids) as compared to the sequence from which it is derived; preferably, the substitution is a conservative substitution with the proviso that the amino acid sequences of the CDRs of the variant have 100% sequence identity to the amino acid sequences of the respective CDRs of the VH and VL of (a), (b), and (c) and the variant binds HER3;
or the antibody that binds HER3 is selected from the group consisting of
(1) an antibody or antigen-binding fragment thereof comprising a heavy chain (HC) comprising (i) a heavy chain variable domain (VH) comprising the amino acid sequence as set forth in SEQ ID NO: 15 and a heavy chain constant domain (CH) comprising the amino acid sequence as set forth in SEQ ID NO: 50 and (ii) a light chain (LC) comprising a light chain variable domain (VL) comprising the amino acid sequence as set forth in SEQ ID NO: 16 and a light chain constant domain (CL) comprising the amino acid sequence as set forth in SEQ ID NO: 51;
(2) an antibody or antigen-binding fragment thereof comprising an HC comprising (i) a VH comprising the amino acid sequence as set forth in SEQ ID NO: 32 and a CH comprising the amino acid sequence as set forth in SEQ ID NO: 50, and (ii) a LC comprising a VL comprising the amino acid sequence as set forth in SEQ ID NO: 33 and a CL comprising the amino acid sequence as set forth in SEQ ID NO: 51; and
(3) an antibody or antigen-binding fragment thereof comprising an HC comprising (i) a VH comprising the amino acid sequence as set forth in SEQ ID NO: 46 and CH comprising the amino acid sequence as set forth in SEQ ID NO: 50, and (ii) a LC comprising a VL comprising the amino acid sequence as set forth in SEQ ID NO: 47 and CL comprising the amino acid sequence as set forth in SEQ ID NO: 51.

48-49. (canceled)

50. A pharmaceutical composition, which comprises the antibody-drug conjugate of claim 1, and one or more pharmaceutical acceptable excipients.

51. A pharmaceutical composition, which comprises the drug-linker compound of claim 37, and one or more pharmaceutically acceptable excipients.

52. A method of treating a cancer in a subject with high expression of HER3 comprising administering a therapeutic effective amount of the antibody-drug conjugate according of claim 1, or an pharmaceutical composition comprising the antibody-drug conjugate to the subject;
preferably, the cancer comprises solid tumors or hematological malignancies;
more preferably, the cancer is colon cancer, gastric cancer, breast cancer, lung cancer, or lymphoma;
further preferably, the lung cancer is a non-small cell lung cancer or lung adenocarcinoma.

53-71. (canceled)

72. The antibody-drug conjugate of claim 46, wherein the antibody that binds HER3 is selected from the group consisting of:
(1) a heavy chain variable region (VH) and/or a light chain variable region (VL) as follows, wherein the CDRs are defined by the Chothia numbering system:
(1a) a heavy chain variable region (VH) comprising the following 3 CDRs: CDR-H1 having the amino acid sequence as set forth in SEQ ID NO: 1 or a variant thereof, CDR-H2 having the amino acid sequence as set forth in SEQ ID NO: 2 or a variant thereof, CDR-H3 having the amino acid sequence as set forth in SEQ ID NO: 3 or a variant thereof; and/or, a light chain variable region (VL) comprising the following 3 CDRs: CDR-L1 having the amino acid sequence as set forth in SEQ ID NO: 4 or a variant thereof, CDR-L2 having the amino acid sequence as set forth in SEQ ID NO: 5 or a variant thereof, CDR-L3 having the amino acid sequence as set forth in SEQ ID NO: 6 or a variant thereof, or,
(1b) a heavy chain variable region (VH) comprising the following 3 CDRs: CDR-H1 having the amino acid sequence as set forth in SEQ ID NO: 19 or a variant thereof, CDR-H2 having the amino acid sequence as set forth in SEQ ID NO: 20 or a variant thereof, CDR-H3 having the amino acid sequence as set forth in SEQ ID NO: 21 or a variant thereof; and/or, a light chain variable region (VL) comprising the following 3 CDRs: CDR-L1 having the amino acid sequence as set forth in SEQ ID NO: 22 or a variant thereof, CDR-L2 having the amino acid sequence as set forth in SEQ ID NO: 23 or a variant thereof, CDR-L3 having the amino acid sequence as set forth in SEQ ID NO: 24 or a variant thereof; or,
(1c) a heavy chain variable region (VH) comprising the following 3 CDRs: CDR-H1 having the amino acid sequence as set forth in SEQ ID NO: 36 or a variant thereof, CDR-H2 having the amino acid sequence as set forth in SEQ ID NO: 37 or a variant thereof, CDR-H3 having the amino acid sequence as set forth in SEQ ID NO: 38 or a variant thereof; and/or, a light chain variable region (VL) comprising the following 3 CDRs: CDR-L1 having the amino acid sequence as set forth in SEQ ID NO: 45 or a variant thereof, CDR-L2 having the amino acid sequence as set forth in SEQ ID NO: 23 or a variant thereof, and CDR-L3 having the amino acid sequence as set forth in SEQ ID NO: 52 or a variant thereof;
wherein, the variant described in any item of (1a), (1b), and (1c) has the amino acid sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2 or 3 amino acids) as compared to the sequence from which it is derived; preferably, the substitution is a conservative substitution with the proviso that the amino acid sequences of the CDRs of the variant have 100% sequence identity to the amino acid sequences of the respective CDRs of the VH and VL of (1a), (1b), and (1c) and the variant binds HER3;
or,
(2) a heavy chain variable region (VH) and/or a light chain variable region (VL) as follows, wherein the CDRs are defined according to the Kabat numbering system:
(2a) a heavy chain variable region (VH) comprising the following 3 CDRs: CDR-H1 having the amino acid sequence as set forth in SEQ ID NO: 7 or a variant thereof, CDR-H2 having the amino acid sequence as set forth in SEQ ID NO: 8 or a variant thereof, CDR-H3 having the amino acid sequence as set forth in SEQ ID NO: 9 or a variant thereof; and/or, a light chain variable region (VL) comprising the following 3 CDRs: CDR-L1 having the amino acid sequence as set forth in SEQ ID NO: 4 or a variant thereof, CDR-L2 having the amino acid sequence as set forth in SEQ ID NO: 5 or a variant thereof, CDR-L3 having the amino acid sequence as set forth in SEQ ID NO: 6 or a variant thereof, or,
(2b) a heavy chain variable region (VH) comprising the following 3 CDRs: CDR-H1 having the amino acid sequence as set forth in SEQ ID NO: 25 or a variant thereof, CDR-H2 having the amino acid sequence as set forth in SEQ ID NO: 26 or a variant thereof, CDR-H3 having the amino acid sequence as set forth in SEQ ID NO: 21 or a variant thereof; and/or, a light chain variable region (VL) comprising the following 3 CDRs: CDR-L1 having the amino acid sequence as set forth in SEQ ID NO: 22 or a variant thereof, CDR-L2 having the amino acid sequence as set forth in SEQ ID NO: 23 or a variant thereof, CDR-L3 having the amino acid sequence as set forth in SEQ ID NO: 24 or a variant thereof; or,
(2c) a heavy chain variable region (VH) comprising the following 3 CDRs: CDR-H1 having the amino acid sequence as set forth in SEQ ID NO: 39 or a variant thereof, CDR-H2 having the amino acid sequence as set forth in SEQ ID NO: 40 or a variant thereof, CDR-H3 having the amino acid sequence as set forth in SEQ ID NO: 38 or a variant thereof; and/or, a light chain variable region (VL) comprising the following 3 CDRs: CDR-L1 having the amino acid sequence as set forth in SEQ ID NO: 45 or a variant thereof, CDR-L2 having the amino acid sequence as set forth in SEQ ID NO: 23 or a variant thereof, and CDR-L3 having the amino acid sequence as set forth in SEQ ID NO: 52 or a variant thereof;
wherein, the variant described in any item of (1a), (1b), and (1c) has the amino acid sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2 or 3 amino acids) as compared to the sequence from which it is derived; preferably, the substitution is a conservative substitution with the proviso that the amino acid sequences of the CDRs of the variant have 100% sequence identity to the amino acid sequences of the respective CDRs of the VH and VL of (2a), (2b), and (2c) and the variant binds HER;
or,
(3) a heavy chain variable region (VH) and/or a light chain variable region (VL) as follows, wherein the CDRs are defined by the IMGT numbering system:
(3a) a heavy chain variable region (VH) comprising the following 3 CDRs: CDR-H1 having the amino acid sequence as set forth in SEQ ID NO: 10 or a variant thereof, CDR-H2 having the amino acid sequence as set forth in SEQ ID NO: 11 or a variant thereof, CDR-H3 having the amino acid sequence as set forth in SEQ ID NO: 12 or a variant thereof; and/or, a light chain variable region (VL) comprising the following 3 CDRs: CDR-L1 having the amino acid sequence as set forth in SEQ ID NO: 13 or a variant thereof, CDR-L2 having the amino acid sequence as set forth in SEQ ID NO: 14 or a variant thereof, CDR-L3 having the amino acid sequence as set forth in SEQ ID NO: 6 or a variant thereof, or,
(3b) a heavy chain variable region (VH) comprising the following 3 CDRs: CDR-H1 having the amino acid sequence as set forth in SEQ ID NO: 27 or a variant thereof, CDR-H2 having the amino acid sequence as set forth in SEQ ID NO: 28 or a variant thereof, CDR-H3 having the amino acid sequence as set forth in SEQ ID NO: 29 or a variant thereof; and/or, a light chain variable region (VL) comprising the following 3 CDRs: CDR-L1 having the amino acid sequence as set forth in SEQ ID NO: 30 or a variant thereof, CDR-L2 having the amino acid sequence as set forth in SEQ ID NO: 31 or a variant thereof, CDR-L3 having the amino acid sequence as set forth in SEQ ID NO: 24 or a variant thereof; or,
(3c) a heavy chain variable region (VH) comprising the following 3 CDRs: CDR-H1 having the amino acid sequence as set forth in SEQ ID NO: 41 or a variant thereof, CDR-H2 having the amino acid sequence as set forth in SEQ ID NO: 42 or a variant thereof, CDR-H3 having the amino acid sequence as set forth in SEQ ID NO: 43 or a variant thereof; and/or, a light chain variable region (VL) comprising the following 3 CDRs: CDR-L1 having the amino acid sequence as set forth in SEQ ID NO: 44 or a variant thereof, CDR-L2 having the amino acid sequence as set forth in SEQ ID NO: 31 or a variant thereof, and CDR-L3 having the amino acid sequence as set forth in SEQ ID NO: 52 or a variant thereof;
wherein, the variant described in any item of (1a), (1b), and (1c) has the amino acid sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2 or 3 amino acids) as compared to the sequence from which it is derived; preferably, the substitution is a conservative substitution with the proviso that the amino acid sequences of the CDRs of the variant have 100% sequence identity to the amino acid sequences of the respective CDRs of the VH and VL of (3a), (3b), and (3c) and the variant binds HER3;
or the antibody that binds HER3 is selected from the group consisting of
(a) a heavy chain variable domain (VH) comprising the amino acid sequence as set forth in SEQ ID NO: 15 or a variant thereof, and/or, a light chain variable domain (VL) comprising the amino acid sequence as set forth in SEQ ID NO: 16 or a variant thereof;
(b) a VH comprising the amino acid sequence as set forth in SEQ ID NO: 32 or a variant thereof, and/or, a VL comprising the amino acid sequence as set forth in SEQ ID NO: 33 or a variant thereof; or
(c) a VH comprising the amino acid sequence as set forth in SEQ ID NO: 46 or a variant thereof, and/or, a VL comprising the amino acid sequence as set forth in SEQ ID NO: 47 or a variant thereof;
wherein, the variant described in any item of (1a), (1b), and (1c) has an amino acid sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or several amino acids (e.g., a substitution, deletion or addition of 1, 2 or 3 amino acids) as compared to the sequence from which it is derived; preferably, the substitution is a conservative substitution with the proviso that the amino acid sequences of the CDRs of the variant have 100% sequence identity to the amino acid sequences of the respective CDRs of the VH and VL of (a), (b), and (c) and the variant binds HER3;
or the antibody that binds HER3 is selected from the group consisting of
(1) an antibody or antigen-binding fragment thereof comprising a heavy chain (HC) comprising (i) a heavy chain variable domain (VH) comprising the amino acid sequence as set forth in SEQ ID NO: 15 and a heavy chain constant domain (CH) comprising the amino acid sequence as set forth in SEQ ID NO: 50 and (ii) a light chain (LC) comprising a light chain variable domain (VL) comprising the amino acid sequence as set forth in SEQ ID NO: 16 and a light chain constant domain (CL) comprising the amino acid sequence as set forth in SEQ ID NO: 51;
(2) an antibody or antigen-binding fragment thereof comprising an HC comprising (i) a VH comprising the amino acid sequence as set forth in SEQ ID NO: 32 and a CH comprising the amino acid sequence as set forth in SEQ ID NO: 50, and (ii) a LC comprising a VL comprising the amino acid sequence as set forth in SEQ ID NO: 33 and a CL comprising the amino acid sequence as set forth in SEQ ID NO: 51; and
(3) an antibody or antigen-binding fragment thereof comprising an HC comprising (i) a VH comprising the amino acid sequence as set forth in SEQ ID NO: 46 and CH comprising the amino acid sequence as set forth in SEQ ID NO: 50, and (ii) a LC comprising a VL comprising the amino acid sequence as set forth in SEQ ID NO: 47 and CL comprising the amino acid sequence as set forth in SEQ ID NO: 51.

73. A pharmaceutical composition, which comprises the antibody-drug conjugate of claim 45, and one or more pharmaceutical acceptable excipients.

74. A pharmaceutical composition, which comprises the antibody-drug conjugate of claim 46, and one or more pharmaceutical acceptable excipients.

75. A method of treating a cancer in a subject with high expression of HER3 comprising administering a therapeutic effective amount of the antibody-drug conjugate according of claim 45, or an pharmaceutical composition comprising the antibody-drug conjugate to the subject;
preferably, the cancer comprises solid tumors or hematological malignancies;
more preferably, the cancer is colon cancer, gastric cancer, breast cancer, lung cancer, or lymphoma;
further preferably, the lung cancer is a non-small cell lung cancer or lung adenocarcinoma.

76. A method of treating a cancer in a subject with high expression of HER3 comprising administering a therapeutic effective amount of the antibody-drug conjugate according of claim 46, or an pharmaceutical composition comprising the antibody-drug conjugate to the subject;
preferably, the cancer comprises solid tumors or hematological malignancies;
more preferably, the cancer is colon cancer, gastric cancer, breast cancer, lung cancer, or lymphoma;
further preferably, the lung cancer is a non-small cell lung cancer or lung adenocarcinoma.


SEQUENCE LISTING (56 sequences)

SEQ ID NO: 1 - protein, 7 aa, synthetic construct
  source 1..7: note = Chothia CDR H1 of 22B6D2-hIgG1
GFTFRSY

SEQ ID NO: 2 - protein, 6 aa, synthetic construct
  source 1..6: note = Chothia CDR H2 of 22B6D2-hIgG1
WYDGSN

SEQ ID NO: 3 - protein, 14 aa, synthetic construct
  source 1..14: note = Chothia CDR H3 of 22B6D2-hIgG1
YYYDSNGYYDAFDI

SEQ ID NO: 4 - protein, 11 aa, synthetic construct
  source 1..11: note = Chothia/Kabat CDR L1 of 22B6D2-hIgG1
RASQGISNYLA

SEQ ID NO: 5 - protein, 7 aa, synthetic construct
  source 1..7: note = Chothia/Kabat CDR L2 of 22B6D2-hIgG1
AASTLQS

SEQ ID NO: 6 - protein, 9 aa, synthetic construct
  source 1..9: note = Chothia/Kabat/IMGT CDR L3 of 22B6D2-hIgG1
QQLNSYPIT

SEQ ID NO: 7 - protein, 5 aa, synthetic construct
  source 1..5: note = Kabat CDR H1 of 22B6D2-hIgG1
SYGMH

SEQ ID NO: 8 - protein, 17 aa, synthetic construct
  source 1..17: note = Kabat CDR H2 of 22B6D2-hIgG1
VIWYDGSNKYYADSVKG

SEQ ID NO: 9 - protein, 14 aa, synthetic construct
  source 1..14: note = Kabat CDR H3 of 22B6D2-hIgG1
YYYDSNGYYDAFDI

SEQ ID NO: 10 - protein, 8 aa, synthetic construct
  source 1..8: note = IMGT CDR H1 of 22B6D2-hIgG1
GFTFRSYG

SEQ ID NO: 11 - protein, 8 aa, synthetic construct
  source 1..8: note = IMGT CDR H2 of 22B6D2-hIgG1
IWYDGSNK

SEQ ID NO: 12 - protein, 16 aa, synthetic construct
  source 1..16: note = IMGT CDR H3 of 22B6D2-hIgG1
ARYYYDSNGYYDAFDI

SEQ ID NO: 13 - protein, 6 aa, synthetic construct
  source 1..6: note = IMGT CDR L1 of 22B6D2-hIgG1
QGISNY

SEQ ID NO: 14 - skipped

SEQ ID NO: 15 - protein, 123 aa, synthetic construct
  source 1..123: note = VH of 22B6D2-hIgG1
EVHLVESGGGVVQPGRSLRLSCATSGFTFRSYGMHWVRQAPGKGLEWVAVIWYDGSNKYYADSVKGRFTISRDISKNTLYLQMNSLTAEDTAVYYCARYYYDSNGYYDAFDIWGQGTMVTVSS

SEQ ID NO: 16 - protein, 107 aa, synthetic construct
  source 1..107: note = VL of 22B6D2-hIgG1
DIQLTQSPSFLSASVGDRVTITCRASQGISNYLAWYQQKPGKAPKLLIFAASTLQSGVPSRFSGSGSGAEFTLTISSLEPEDFATYYCQQLNSYPITFGQGTRLEIK

SEQ ID NO: 17 - protein, 453 aa, synthetic construct
  source 1..453: note = heavy chain of 22B6D2-hIgG1
EVHLVESGGGVVQPGRSLRLSCATSGFTFRSYGMHWVRQAPGKGLEWVAVIWYDGSNKYYADSVKGRFTISRDISKNTLYLQMNSLTAEDTAVYYCARYYYDSNGYYDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 214 aa, synthetic construct
  source 1..214: note = light chain of 22B6D2-hIgG1
DIQLTQSPSFLSASVGDRVTITCRASQGISNYLAWYQQKPGKAPKLLIFAASTLQSGVPSRFSGSGSGAEFTLTISSLEPEDFATYYCQQLNSYPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 9 aa, synthetic construct
  source 1..9: note = Chothia CDR H1 of 47A3E3-hIgG1
GDSVSSSQS

SEQ ID NO: 20 - protein, 7 aa, synthetic construct
  source 1..7: note = Chothia CDR H2 of 47A3E3-hIgG1
FYRSRWY

SEQ ID NO: 21 - protein, 12 aa, synthetic construct
  source 1..12: note = Chothia/Kabat CDR H3 of 47A3E3-hIgG1
DTYYYGSGGFDY

SEQ ID NO: 22 - protein, 11 aa, synthetic construct
  source 1..11: note = Chothia/Kabat CDR L1 of 47A3E3-hIgG1
RASQSLSRWLA

SEQ ID NO: 23 - protein, 7 aa, synthetic construct
  source 1..7: note = Chothia/Kabat CDR L2 of 47A3E3-hIgG1/100H7D3-hIgG1
KASSLES

SEQ ID NO: 24 - protein, 8 aa, synthetic construct
  source 1..8: note = Chothia/Kabat/IMGT CDR L3 of 47A3E3-hIgG1
QQYKNYYS

SEQ ID NO: 25 - protein, 7 aa, synthetic construct
  source 1..7: note = Kabat CDR H1 of 47A3E3-hIgG1
SSQSTWN

SEQ ID NO: 26 - protein, 18 aa, synthetic construct
  source 1..18: note = Kabat CDR H2 of 47A3E3-hIgG1
RTFYRSRWYNDYALSMKS

SEQ ID NO: 27 - protein, 10 aa, synthetic construct
  source 1..10: note = IMGT CDR H1 of 47A3E3-hIgG1
GDSVSSSQST

SEQ ID NO: 28 - protein, 9 aa, synthetic construct
  source 1..9: note = IMGT CDR H2 of 47A3E3-hIgG1
TFYRSRWYN

SEQ ID NO: 29 - protein, 14 aa, synthetic construct
  source 1..14: note = IMGT CDR H3 of 47A3E3-hIgG1
ARDTYYYGSGGFDY

SEQ ID NO: 30 - protein, 6 aa, synthetic construct
  source 1..6: note = IMGT CDR L1 of 47A3E3-hIgG1
QSLSRW

SEQ ID NO: 31 - skipped

SEQ ID NO: 32 - protein, 124 aa, synthetic construct
  source 1..124: note = VH of 47A3E3-hIgG1
EVQLQESGPGLVKPSQTLSLTCAISGDSVSSSQSTWNWIRQSPSRGLEWLGRTFYRSRWYNDYALSMKSRITINPDTSKNQFSLQLNPVTPEDTAVYYCARDTYYYGSGGFDYWGQGSRVTVSS

SEQ ID NO: 33 - protein, 106 aa, synthetic construct
  source 1..106: note = VL of 47A3E3-hIgG1
DIQMTQSPSTLSASVGDRVTITCRASQSLSRWLAWYQQKPEKAPKLLIYKASSLESGVPSRFSGSGSGTEFTLTISTLQPDDFATYYCQQYKNYYSFGQGTKLEIK

SEQ ID NO: 34 - protein, 454 aa, synthetic construct
  source 1..454: note = heavy chain of 47A3E3-hIgG1
EVQLQESGPGLVKPSQTLSLTCAISGDSVSSSQSTWNWIRQSPSRGLEWLGRTFYRSRWYNDYALSMKSRITINPDTSKNQFSLQLNPVTPEDTAVYYCARDTYYYGSGGFDYWGQGSRVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 213 aa, synthetic construct
  source 1..213: note = light chain of 47A3E3-hIgG1
DIQMTQSPSTLSASVGDRVTITCRASQSLSRWLAWYQQKPEKAPKLLIYKASSLESGVPSRFSGSGSGTEFTLTISTLQPDDFATYYCQQYKNYYSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 7 aa, synthetic construct
  source 1..7: note = Chothia CDR H1 of 100H7D3-hIgG1
GFTFSRN

SEQ ID NO: 37 - protein, 6 aa, synthetic construct
  source 1..6: note = Chothia CDR H2 of 100H7D3-hIgG1
WHDGSN

SEQ ID NO: 38 - protein, 7 aa, synthetic construct
  source 1..7: note = Chothia/Kabat CDR H3 of 100H7D3-hIgG1
DRGASDF

SEQ ID NO: 39 - protein, 5 aa, synthetic construct
  source 1..5: note = Kabat CDR H1 of 100H7D3-hIgG1
RNAMH

SEQ ID NO: 40 - protein, 17 aa, synthetic construct
  source 1..17: note = Kabat CDR H2 of 100H7D3-hIgG1
VIWHDGSNKYYGDSVKG

SEQ ID NO: 41 - protein, 8 aa, synthetic construct
  source 1..8: note = IMGT CDR H1 of 100H7D3-hIgG1
GFTFSRNA

SEQ ID NO: 42 - protein, 8 aa, synthetic construct
  source 1..8: note = IMGT CDR H2 of 100H7D3-hIgG1
IWHDGSNK

SEQ ID NO: 43 - protein, 9 aa, synthetic construct
  source 1..9: note = IMGT CDR H3 of 100H7D3-hIgG1
ARDRGASDF

SEQ ID NO: 44 - protein, 6 aa, synthetic construct
  source 1..6: note = IMGT CDR L1 of 100H7D3-hIgG1
QSINNW

SEQ ID NO: 45 - protein, 11 aa, synthetic construct
  source 1..11: note = Chothia/Kabat CDR L1 of 100H7D3-hIgG1
RASQSINNWLA

SEQ ID NO: 46 - protein, 116 aa, synthetic construct
  source 1..116: note = VH of 100H7D3-hIgG1
EVQLVESGGGVVLPGRSLRLSCAASGFTFSRNAMHWVRQAPGKGLEWVAVIWHDGSNKYYGDSVKGRFTISRDNSKNTLYLQMDSLTAEDTAVYYCARDRGASDFRGQGTLVTVSS

SEQ ID NO: 47 - protein, 107 aa, synthetic construct
  source 1..107: note = VL of 100H7D3-hIgG1
DIQMTQSPSTLSASVGDRVTITCRASQSINNWLAWYQQKPEKAPKLLIYKASSLESGVPSRFSGSGSGTEFTLTISSLQPDDFATYYCQQYNSYSLTFGGGTKVEIK

SEQ ID NO: 48 - protein, 446 aa, synthetic construct
  source 1..446: note = heavy chain of 100H7D3-hIgG1
EVQLVESGGGVVLPGRSLRLSCAASGFTFSRNAMHWVRQAPGKGLEWVAVIWHDGSNKYYGDSVKGRFTISRDNSKNTLYLQMDSLTAEDTAVYYCARDRGASDFRGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 214 aa, synthetic construct
  source 1..214: note = light chain of 100H7D3-hIgG1
DIQMTQSPSTLSASVGDRVTITCRASQSINNWLAWYQQKPEKAPKLLIYKASSLESGVPSRFSGSGSGTEFTLTISSLQPDDFATYYCQQYNSYSLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 330 aa, synthetic construct
  source 1..330: note = IgG1 heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 107 aa, synthetic construct [sequence omitted]

SEQ ID NO: 52 - protein, 9 aa, synthetic construct
  source 1..9: note = Chothia/Kabat/IMGT CDR L3 of 100H7D3-hIgG1
QQYNSYSLT

SEQ ID NO: 53 - protein, 5 aa, synthetic construct
  source 1..5: note = linker
GGGGS

SEQ ID NO: 54 - protein, 20 aa, synthetic construct
  source 1..20: note = linker
GGGGSGGGGSGGGGSGGGGS

SEQ ID NO: 55 - protein, 4 aa, synthetic construct
  source 1..4: note = peptide 1
GGFG

SEQ ID NO: 56 - protein, 5 aa, synthetic construct
  source 1..5: note = peptide 2
GGGGG